(19) United States
(12) Patent Application Publication
(10) Pub. No.: US 2025/0346639 A1
(43) Pub. Date: Nov. 13, 2025

(54) BACTERIOCIN POLYPEPTIDES, NUCLEIC ACIDS ENCODING SAME, AND METHODS OF USE THEREOF

(71) Applicants: Syngulon SA, Seraing (BE); Complutense University of Madrid, MADRID (ES)

(72) Inventors: Juan Borrero del Pino, Madrid (ES); Philippe Gabant, Ottignies Louvain-La-Neuve (BE)

(21) Appl. No.: 18/868,688

(22) PCT Filed: May 26, 2023
(86) PCT No.: PCT/US2023/067567

§ 371 Date: Nov. 22, 2024

Related U.S. Application Data
(60) Provisional application No. 63/365,584, filed on May 31, 2022.

(51) Int. Cl. C07K 14/195; A01N 63/50; C12N 15/10; C12N 15/70; C40B 40/02

(52) U.S. Cl. CPC C07K 14/195; A01N 63/50; C12N 15/1082; C12N 15/70; C40B 40/02; C07K 2319/02; C07K 2319/20; C07K 2319/92; C07K 2319/95

(57) ABSTRACT

Provided herein are fusion polypeptides that include an amino acid sequence of a bacteriocin flanked at both the N- and C-termini by a split intein that circularizes the bacteriocin. Also provided are nucleic acids and genetic vectors encoding the fusion polypeptide, and microbial cells genetically engineered with the nucleic acids or genetic vectors. Further provided are methods of making a circular bacteriocin, methods of screening using a library of nucleic acids or genetic vectors encoding the fusion polypeptide, and methods of controlling the growth of an organism using circular bacteriocins made by the methods provided herein.

### REFERENCE TO RELATED APPLICATIONS

The present application claims benefit of U.S. Provisional Application No. 63/365,584, filed May 31, 2022. The entirety of this related application is incorporated herein by reference.

### REFERENCE TO SEQUENCE LISTING

The present application is being filed along with a Sequence Listing in electronic format. The Sequence Listing is provided as a file entitled SeqListSyng.012wo.xml, created on May 25, 2023, which is 1,024,021 bytes in size. The information in the electronic format of the Sequence Listing is incorporated herein by reference in its entirety.

### BACKGROUND

### Field

The present disclosure generally relates to antimicrobial peptides, such as bacteriocins.

Bacteriocins are ribosomally synthesized antimicrobial peptides produced by bacteria. Applications of bacteriocins have been traditionally focused on food preservation, mainly due to the widespread presence of these peptides within the lactic acid bacteria group, and the approval of nisin as food preservative by the regulatory agencies. The use of bacteriocins as antimicrobial agents in human and animal health and non-food industrial applications, among others, are also contemplated.

### SUMMARY

Circular bacteriocins are a class of antimicrobial peptides produced by Gram-positive bacteria that after production undergo a head to tail ligation. Compared to their linear counterparts, circular bacteriocins are, in general, quite stable to temperature and pH changes and more resistant to proteolytic enzymes, being considered as a promising group of antimicrobial peptides for industrial applications. A limited number of circular bacteriocins have been produced and fully characterized, although many operons potentially coding for new circular bacteriocins arc found in genomes in the databases. The activity of several proteins mediate the production and circularization of these bacteriocins and genes encoding these proteins are expressed by the native bacteriocin producing bacteria or can be expressed in a heterologous host. Provided herein are methods of carrying out bacteriocin circularization by using the split-intein circular ligation of peptides and proteins (SICCLOPPS) system. In some embodiments, methods of the present disclosure provide fast and efficient options for in vitro (by a cell-free protein system) and in vivo (by E. coli) production and correct circularization of characterized and/or novel circular bacteriocins. In some embodiments, the present disclosure provides intein-based synthetic biology tools for the production and characterization of new circular bacteriocins, the biosynthesis of variants and/or the production of these peptides in other hosts.

Provided herein is a fusion polypeptide comprising an amino acid sequence of a bacteriocin flanked at both the N- and C-termini by a split intein that circularizes the bacteriocin. Optionally, the bacteriocin is a natively circular bacteriocin. In some embodiments, the amino acid sequence of the bacteriocin is circularly permuted compared to a native amino acid sequence of the bacteriocin. In some embodiments, the first residue of the amino acid sequence of the bacteriocin is a serine or a cysteine that is present in the native amino acid sequence of the bacteriocin.

In some embodiments, the first residue of the amino acid sequence of the bacteriocin is a non-native serine or a non-native cysteine. Optionally, the non-native serine or the non-native cysteine substitutes a native amino acid residue in the amino acid sequence of the bacteriocin. Optionally, the length of the amino acid sequence of the bacteriocin is increased by one residue due to the non-native serine or the non-native cysteine compared to the length of the native amino acid sequence of the bacteriocin. In some embodiments, the native amino acid sequence of the bacteriocin does not comprise a serine or cysteine.

In some embodiments, the split intein is based on an intein from one of the following: Npu DnaE, Sce VMA, Ssp DnaE. In some embodiments, the split intein is a conditional split intein. In some embodiments, the conditional split intein is pH- or temperature-sensitive. In some embodiments, the split intein comprises a second amino acid sequence of a C-terminal intein fragment (I_{C}) at least 80% identical to the I_{C} shown in Table B, and a third amino acid sequence of a N-terminal intein fragment (I_{N}) at least 80% identical to the split intein I_{N} shown in Table B.

In some embodiments, the bacteriocin is selected from any one of the bacteriocins listed in Table A. In some embodiments, the amino acid sequence of the bacteriocin is at least 80% identical to any one of the sequences listed in Table A. In some embodiments, the amino acid sequence of the bacteriocin is selected from any one of the sequences listed in Table A.

In some embodiments, the bacteriocin is an engineered bacteriocin. In some embodiments, one or more amino acids of the polypeptide in the amino acid sequence is a non-natural amino acid.

In some embodiments, the fusion polypeptide further comprises a degradation tag. Optionally, the degradation tag is at the C-terminus of the fusion polypeptide. In some embodiments, the split intein comprises a C-terminal intein fragment (“I_{C}”) fused N-terminal to the amino acid sequence of the bacteriocin and a N-terminal intein fragment (“I_{N}”) fused C-terminal to the amino acid sequence of the bacteriocin, wherein the polypeptide further comprises a degradation tag C-terminal to the I_{N}. In some embodiments, the degradation tag comprises a sequence at least 80% identical to AANDENYALAA (SEQ ID NO: 873).

In some embodiments, the fusion polypeptide further comprises a signal peptide and/or a leader sequence.

Also provided herein is a nucleic acid comprising a nucleotide sequence encoding the fusion polypeptide of any one of the preceding claims. Optionally, the nucleotide sequence is operably linked to a promoter sequence. In some embodiments, the nucleic acid comprises DNA. Optionally, the nucleic acid comprises RNA. Also provided is a genetic vector comprising the nucleic acid of the present disclosure.

Also provided is a genetically engineered microbial cell comprising the nucleic acid of the present disclosure, or the genetic vector of the present disclosure. Optionally, the microbial cell is resistant to the bacteriocin. In some embodiments, the microbial cell comprises a second nucleic acid encoding an immunity modulator that confers resistant to the bacteriocin. Optionally, expression of the immunity modulator from the second nucleic acid is regulatable. In some embodiments, the microbial cell is a bacteria, fungi, or algae.

Also provided herein is a composition comprising the fusion polypeptide of the present disclosure. Provided herein is a composition comprising a circular bacteriocin and a split intein.

Further provided herein is a method of making a circular bacteriocin, comprising contacting the nucleic acid of the present disclosure, or the genetic vector of the present disclosure with an in vitro expression system under conditions sufficient to produce a circular bacteriocin. Also provided is a method of making a circular bacteriocin, comprising culturing the microbial cell of the present disclosure under conditions sufficient to produce a circular bacteriocin.

In some embodiments, the method further comprises purifying the circular bacteriocin. In some embodiments, the method further comprises purifying the fusion polypeptide. In some embodiments, the split intein is a conditional split intein that circularizes the bacteriocin under a permissive condition but not under a non-permissive condition, and wherein the method further comprises exposing the fusion polypeptide to the permissive condition, following exposure to the non-permissive condition, to induce circularization of the bacteriocin. In some embodiments, the method further comprises modifying the pH or temperature to induce circularization of the bacteriocin, wherein the split intein is pH- or temperature-sensitive, respectively. In some embodiments, the method further comprises allowing the split intein to be degraded after the circular bacteriocin is produced.

Also provided is a library comprising a plurality of genetic vectors, each genetic vector comprising the nucleic acid of the present disclosure, wherein at least two of the plurality of genetic vectors comprise nucleotide sequences encoding different bacteriocins. Optionally, the nucleotide sequences encode bacteriocins from different microbial species. Optionally, the nucleotide sequences comprise different sequence variants of a parent bacteriocin. Optionally, the parent bacteriocin is a natively circular bacteriocin, and the sequence variants comprise a first variant that abrogates natural circularization of the parent bacteriocin.

Also provided herein is a method of screening, comprising: providing the library of the present disclosure; expressing a plurality of polypeptides encoded by one of more genetic vectors of the library; generating a plurality of circular bacteriocins from the plurality of expressed polypeptides; and assaying the plurality of circular bacteriocins for a desired activity. Optionally, the desired activity comprises antimicrobial activity.

Further provided is a method of controlling the growth of a microorganism, comprising contacting a composition comprising and/or conducive to supporting the growth of a microorganism with the microbial cell of the present disclosure under conditions sufficient to produce a circular bacteriocin, to thereby control the growth of the microorganism. Also provided is a method of controlling the growth of a microorganism, comprising contacting a composition comprising and/or conducive to supporting the growth of a microorganism with a circular bacteriocin made by the method of the present disclosure, to thereby control the growth of the microorganism. Provided herein is a method of controlling the growth of a microorganism, comprising contacting a composition comprising and/or conducive to supporting the growth a microorganism with the fusion polypeptide of the present disclosure, to thereby control the growth of the microorganism.

In some embodiments, the microorganism is a bacteria. In some embodiments, the composition is a culture medium, feedstock, or a microbiome. In some embodiments, the split intein is a conditional split intein that circularizes the bacteriocin under a permissive condition but not under a non-permissive condition, and wherein the method further comprises providing the permissive condition to the composition to thereby induce circularization of the bacteriocin. In some embodiments, the method comprises modifying the pH or temperature of the composition to induce circularization of the bacteriocin, wherein the split intein is pH- or temperature-sensitive, respectively.

Further provided is a method of designing a nucleic acid encoding a polypeptide precursor of a bacteriocin, comprising: identifying a native amino acid sequence of a candidate bacteriocin, wherein the native amino acid sequence does not comprise a serine or cysteine at the N-terminus; providing a second amino acid sequence having a serine or cysteine at the N-terminus thereof by at least one of: circularly permuting the native amino acid sequence; or introducing a serine or cysteine to the native amino acid sequence; providing a nucleotide sequence encoding a polypeptide comprising the second amino acid sequence flanked at both the N- and C-termini by a split intein configured to circularize the bacteriocin; and expressing the polypeptide encoded by the nucleotide sequence. Optionally, the candidate bacteriocin is predicted to be a circular bacteriocin based on a genomic sequence of a microorganism that encodes the candidate bacteriocin in its genome. Optionally, the method includes: identifying a plurality of native amino acid sequences of a plurality of different candidate bacteriocins; for each of the plurality of native amino acid sequences: providing the second amino acid sequence; and providing the nucleotide sequence encoding a polypeptide comprising the second amino acid sequence flanked at both the N- and C-termini by a split intein configured to circularize the bacteriocin, thereby generating a library of nucleic acids representing each of the plurality of native amino acid sequences.

In some embodiments, the polypeptide further comprises a degradation tag. In some embodiments, the polypeptide further comprises a signal peptide and/or leader sequence. In some embodiments, the polypeptide is expressed in vitro. In some embodiments, the polypeptide is expressed from a genetically engineered microbial cell configured to express the polypeptide encoded by the nucleotide sequence.

### BRIEF DESCRIPTION OF THE DRAWINGS

FIG. 1 is a schematic diagram showing a polypeptide of a bacteriocin flanked by a split intein that is spliced to generate a circular bacteriocin, according to some non-limiting embodiments of the present disclosure.

FIG. 2A is a schematic diagram showing a circular bacteriocin, according to some non-limiting embodiments of the present disclosure.

FIG. 2B is a schematic diagram showing structure of nucleic acids encoding a bacteriocin with or without a functional split intein, according to some non-limiting embodiments of the present disclosure.

FIG. 2C depicts an amino acid sequence of a bacteriocin flanked by a split intein, according to some non-limiting embodiments of the present disclosure.

FIG. 2D is a schematic diagram showing a polypeptide of a bacteriocin flanked by a split intein that is spliced to generate a circular bacteriocin, according to some non-limiting embodiments of the present disclosure.

FIG. 3A is an image showing antimicrobial activity of a circular bacteriocin generated by bacteria genetically engineered with a nucleic acid encoding a bacteriocin flanked by a split intein, according to some non-limiting embodiments of the present disclosure.

FIG. 3B is an image showing antimicrobial activity of a circular bacteriocin generated by bacteria genetically engineered with a nucleic acid encoding a bacteriocin flanked by a split intein, according to some non-limiting embodiments of the present disclosure.

FIG. 4A is a schematic diagram showing in vitro and in vivo production, followed by evaluation of antimicrobial activity and mass spectrometry analysis of circular bacteriocin, according to some non-limiting embodiments of the present disclosure.

FIG. 4B is a collection of mass spectra from mass spectrometry analysis of purified circular bacteriocin produced by a genetically engineered bacteria, according to some non-limiting embodiments of the present disclosure.

FIG. 5 is a block diagram showing a method of screening, according to some non-limiting embodiments of the present disclosure.

### DETAILED DESCRIPTION

Bacteriocins can be divided in two main groups: class I bacteriocins that undergo post-translational modifications and class II or unmodified bacteriocins. Bacteriocins such as lantibiotics, thiopeptides, lassopeptides or sactibiotics belong to class I, and pediocin like bacteriocins, two peptide bacteriocins and linear non-pediocin like, single-peptide bacteriocins belong to class II. Some bacteriocins undergo enzymatic modification during biosynthesis, where an amide bond is formed between the N and C-terminal amino acid, thus acquiring a head-to-tail or circular structure. Without being bound by theory, the circular structure of these bacteriocins is thought to contribute to their higher stability against thermal stress, pH variation, and degradation by many proteolytic enzymes, compared to their linear counterparts. Thus, circular bacteriocins may have a variety of industrial applications.

Biosynthesis of circular bacteriocins involves the action of different proteins encoded by genes that are usually clustered together. Gene organization in head-to-tail cyclized bacteriocins clusters is well conserved and can include a minimum of 5 to 7 genes encoding the bacteriocin precursor peptide, immunity proteins, membrane DUF95 protein (presumably involved in circularization), and one or more other proteins [9][10].

A typical biosynthetic gene cluster for head-to-tail cyclized bacteriocins consists of genes encoding the bacteriocin precursor peptide, transporter protein(s), a SpoIIM (stage II sporulation protein M) membrane protein (previously known as DUF95), an immunity protein, and one or more unknown hydrophobic proteins. The inactive precursor peptide has an N-terminal leader sequence and C-terminal core peptide. During maturation, the leader peptide is cleaved, and a peptide bond is formed between the new N-terminal amino acid and the C-terminal residue, producing the active head-to-tail cyclized bacteriocin.

Advances in sequencing and bioinformatics have accelerated exponentially the discovery of novel circular bacteriocins. Numerous potential novel circular bacteriocins have been identified across a wide group of gram positive strains by identifying hypothetical circular bacteriocin clusters in microbial genomes.

Novel bacteriocins can be experimentally confirmed by production and purification of the antimicrobial peptide in the supernatant of either the native strain or an heterologous host carrying all the genes needed for biosynthesis of the mature bacteriocin. This process can be laborious, expensive and time consuming and in most cases requires the native bacteriocin producing bacteria. Alternatively, a cell-free protein synthesis approach can be used for the production of bacteriocins. In vitro production can allow testing of the properties of the bacteriocin including industrially relevant ones that may be more difficult by other approaches, such as by fermentation (see Gabant and Borrero 2019). In vitro production is also compatible with high throughput approaches to screen collection of genes of bacteriocins or collection of variants thereof. Suitable options of in vitro production include PARAGEN 1.0, as described by Gabant and Borrero (2019), which demonstrated the synthetic production of a collection 164 different class II bacteriocins (called PARAGEN 1.0) using a cell-free protein synthesis approach.

Split inteins (internal proteins) can be used to circularize peptides. Provided herein, in some embodiments, is a fast and reliable method for producing circular bacteriocins by combining the split intein circular ligation of peptides and proteins (SICLOPPS) method with cell-free protein synthesis. In some embodiments, fusion of the C and N-terminal intein fragments from Nostoc punctiforme (Npu) DnaE split intein to the mature peptide of bacteriocin garvicin ML allows for the production and circularization of this peptide, without any other protein involved in circularization of the peptide in the native context needed. In some embodiments, active garvicin ML is produced both in vitro (by cell-free synthesis) and in vivo (by E. coli). Purification and posterior analysis of garvicin ML has proved correct circularization of the peptide thus obtaining a peptide with the same molecular weight of the native one. In some embodiments, other circular bacteriocins both characterized or not yet characterized are produced. In some embodiments, new candidates can be tested, or libraries of circular bacteriocins can be generated.

Provided herein are fusion polypeptides and nucleic acids encoding same, for generating circular bacteriocins. In general terms, fusion polypeptides of the present disclosure include an amino acid sequence of a bacteriocin that is flanked on both ends of the amino acid sequence by a split intein that can circularize the bacteriocin. In some embodiments, the fusion polypeptides and nucleic acids of the present disclosure facilitate production of circular bacteriocins. The circular bacteriocins made from the fusion polypeptides of the present disclosure, or from the nucleic acid and genetic vectors encoding same, can have antimicrobial activity. In some embodiments, a circular bacteriocin made from the fusion polypeptide of the present disclosure, or from the nucleic acid and genetic vectors encoding same as disclosed herein, has substantial antimicrobial activity. In some embodiments, a circular bacteriocin made from the fusion polypeptide of the present disclosure, or from the nucleic acid and genetic vectors encoding same as disclosed herein, has at least about the same level of antimicrobial activity as that of the corresponding, natively produced circular bacteriocin. As the circularization of the bacteriocin by the split intein can be achieved without any additional components that may have been involved in a native context (e.g., other proteins encoded by genes of the bacteriocin cluster in the native microbial organism), the circular bactericion can be produced or expressed in a variety of heterologous contexts, e.g., in a heterologous organism that does not have the additional proteins, or in vitro in the absence of the additional components). In some embodiments, the fusion polypeptides and nucleic acids of the present disclosure provide for high-throughput expression of known or putative circular bacteriocins for screening. In some embodiments, the fusion polypeptides and nucleic acids of the present disclosure provide for expression of circular bacteriocins variants having mutations that would have affected circularization of the bacteriocin via the native mechanism, and thereby expand the mutational space for screening variant bacteriocins having a desired activity. In some embodiments, the fusion polypeptides and nucleic acids of the present disclosure provide for expression of circular bacteriocins variants that include non-natural amino acids, and thereby expand the mutational space for screening variant bacteriocins of interest. In some embodiments, use of a split intein to circularize bacteriocins allows for an additional level of control for regulating bacteriocin activity, by regulating the cyclizing activity of the split intein. In some embodiments, circularizing a bacteriocin (including circularizing a naturally linear bacteriocin) improves the stability of the bacteriocin, e.g., by making the bacteriocin more resistant to degradation by heat, pH, or protease.

### TERMS

Unless stated otherwise, terms used herein have their customary and ordinary meaning as understood by one of skill in the art in view of this disclosure.

As used herein, “bacteriocin,” and variations of this root term, has its customary and ordinary meaning as understood by one of skill in the art in view of this disclosure. It refers to a polypeptide that is secreted by a host cell and can neutralize at least one microbial organism other than the individual host cell in which the polypeptide is made, including cells clonally related to the host cell and other microbial cells. “Bacteriocin” refers to naturally circular bacteriocins and naturally linear bacteriocins, unless indicated otherwise. A “circular bacteriocin” denotes a bacteriocin that is circularized when expressed from the natural host from which the bacteriocin is derived, or that is predicted to be circularized based on sequence of the bacterial genome, or that has been designed or engineered to be active when circularized. A “linear bacteriocin” denotes a bacteriocin that is linear (and does not get circularized) when expressed from the natural host from which the bacteriocin is derived, or that is predicted to be linear based on the genomic context, or that has been designed or engineered to be active when in linear form. “Bacteriocin” also encompasses a cell-free or chemically synthesized version of such a polypeptide, for example an engineered bacteriocin in accordance with some embodiments herein. A host cell can exert cytotoxic or growth-inhibiting effects on one or a plurality of other microbial organisms by secreting bacteriocins.

“Circularized” and “cyclized” are used interchangeably and have their customary and ordinary meaning as understood by one of skill in the art in view of this disclosure, and are used to denote a polypeptide that has undergone head-to-tail circularization or cyclization of the peptide backbone, to form an amide bond between the N-terminal amino group and C-terminal carboxyl group of the polypeptide. “Linear” as used herein has its customary and ordinary meaning as understood by one of skill in the art in view of this disclosure, and denotes a polypeptide having a free (non-bonded) amino group at the N-terminus and/or a free (non-bonded) carboxyl group at the C-terminus.

As used herein “circularly permuted” denotes modification of a linear sequence of elements by shifting the position of the elements while preserving the position of each element relative to each other, where elements that are shifted past the first or last position in the linear sequence wrap around to the opposite end of the sequence. For example, circular permutation of the sequence “ABCDE” can result in any one of “BCDEA”, “CDEAB”, “DEABC”, and “EABCD”.

As used herein, the term “operably linked” has its customary and ordinary meaning as understood by one of skill in the art in view of this disclosure, and refers to a linkage of nucleic acid elements in a functional relationship. A nucleic acid is “operably linked” when it is placed into a functional relationship with another nucleic acid. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The terms “protein” or “polypeptide” have their customary and ordinary meaning as understood by one of skill in the art in view of this disclosure, and are used interchangeably and refer to molecules consisting of a chain of amino acids, without reference to a specific mode of action, size, 3-dimensional structure or origin.

The term “gene” has its customary and ordinary meaning as understood by one of skill in the art in view of this disclosure, and means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) e.g. comprising a polyadenylation- and/or transcription termination site.

In amino acid sequences as described herein, amino acids or “residues” are denoted by three-letter or one-letter symbols. These three-letter symbols as well as the corresponding one-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (lie) is isoleucine, K (Lys) is lysine, L (Leu) is leucine, M (Met) is methionine, N (Asn) is asparagine, P (Pro) is proline, Q (Gin) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid. As used herein “natural” and “non-natural” each has its ordinary and customary meaning as understood by one of ordinary skill in the art, in view of the present disclosure. A “natural” amino acid denotes an amino acid naturally occurring in nature. A “non-natural” amino acid denotes a non-genetically encoded amino acid, irrespective of whether it appears in nature or not. Non-natural amino acids that can be present in a peptidomimetic as described herein include: b-amino acids; p-acyl-L-phenylalanine; N-acetyl lysine; O-4-allyl-L-tyrosine; 2-aminoadipic acid; 3-aminoadipic acid; beta-alanine; 4-tert-butyl hydrogen 2-azidosuccinate; beta-aminopropionic acid; 2-aminobutyric acid; 4-aminobutyric acid; 2,4-diamino butyric acid; 6-aminocaproic acid; 2-aminoheptanoic acid; 2-aminoisobutyric acid; 3-aminoisobutyric acid; 2-aminopimelic acid; p-aminophenylalanine; 2,3-diaminobutyric acid; 2,3-diamino propionic acid; 2,2′-diaminopinnelic acid; p-amino-L-phenylalanine; p-azido-L-phenylalanine; D-allyl glycine; p-benzoyl-L-phenylalanine; 3-benzothienyl alanine p-bromophenylalanine; t-butylalanine; t-butylglycine; 4-chlorophenylalanine; cyclohexylalanine; cysteic acid; D-citrulline; thio-L-citrulline; desmosine; epsilon-amino hexanoic acid; N-ethylglycine; N-ethylasparagine; 2-fluorophenylalanine; 3-fluorophenylalanine; 4-fluorophenylalanine; homoarginine; homocysteine; homoserine; hydroxy lysine; alio-hydroxy lysine; 3-(3-methyl-4-nitrobenzyl)-L-histidine methyl ester; isodesmosine; allo-isoleucine; isopropyl-L-phenylalanine; 3-methyl-phenylalanine; N-methylglycine; N-methylisoleucine; 6-N-methyllysine; O-methyl-L-tyrosine; N-methylvaline; methionin sulfoxide; 2-napthylalanine; L-3-(2-naphthyl)alanine; isoserine; 3-phenylserine; norvaline; norleucine; 5,5,5-trifluoro-DL-leucine; ornithine; 3-chloro-tyrosine; N5-carbamoylornithine; penicillamine; phenylglycine; piperidinic acid; pyridylalanine; 1,2,3,4-tetrahydro-isoquinoline-3-carboxylic acid; beta-2-thienylalanine; y-carboxy-DL-glutamic acid; 4-fluoro-DL-glutamic acid; D-thyroxine; allo-threonine; 5-hydroxy-tryptophan; 5-methoxy-tryptophan; 5-fluoro-tryptophan; 3-fluoro-valine. In some embodiments, a natural amino acid of a fusion polypeptide of the present disclosure is substituted by a corresponding non-natural amino acid. As used herein, a “corresponding non-natural amino acid” refers to a non-natural amino acid that is a derivative of the reference natural amino acid. For instance, a natural amino acid can be substituted by the corresponding beta-amino acid, which have their amino group bonded to the beta-carbon rather than the alpha carbon.

The terms “homology”, “sequence identity” and the like have their customary and ordinary meaning as understood by one of skill in the art in view of this disclosure, and are used interchangeably herein. Sequence identity is described herein as a relationship between two or more amino acid (polypeptide or protein) sequences or two or more nucleic acid (nucleic acid) sequences, as determined by comparing the sequences. In an embodiment, sequence identity is calculated based on the full length of two given sequences, including those identified by SEQ ID NO's, or on a part thereof. Part thereof means at least 50%, 60%, 70%, 80%, 90%, or 100% of both SEQ ID NO's. “Identity” also refers to the degree of sequence relatedness between amino acid or nucleic acid sequences, as determined by the match between strings of such sequences. “Identity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004.

“Sequence identity” can be determined by alignment of two peptide or two nucleotide sequences using global or local alignment algorithms, depending on the length of the two sequences. In embodiments, sequences of similar lengths are aligned using a global alignment algorithms (e.g. Needleman-Wunsch) which aligns the sequences optimally over the entire length, while sequences of substantially different lengths are aligned using a local alignment algorithm (e.g. Smith-Waterman). Sequences may then be referred to as “substantially identical” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. In embodiments, when sequences have a substantially different overall length, local alignments, such as those using the Smith-Waterman algorithm, can be used. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919).

Percentage identity may be determined by searching against public databases, using algorithms such as FASTA, BLAST, etc. Thus, the nucleic acid and protein sequences of some embodiments of the present disclosure can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the present disclosure. BLAST protein searches can be performed with the BLASTx program, score=50, wordlength=3 to obtain amino acid sequences homologous to protein molecules of the present disclosure. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information accessible on the world wide web at ncbi.nlm.nih.gov/.

As used herein, “conservative” amino acid substitution has its customary and ordinary meaning as understood by one of skill in the art in view of this disclosure, and refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Suitable conservative amino acids substitution groups include: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. In some embodiments, the amino acid change is conservative. Suitable conservative substitutions for each of the naturally occurring amino acids include: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

As used herein, “microbial organism”, “microorganism”, “microbial cell” or “microbial host” and variations of these root terms (such as pluralizations and the like) have their customary and ordinary meanings as understood by one of skill in the art in view of this disclosure, including any naturally-occurring species or synthetic or fully synthetic prokaryotic or eukaryotic unicellular organism. Thus, this expression can refer to cells of any of the three domains Bacteria, Archaea and Eukarya.

“Comprise” and its conjugations is used herein in its non-limiting sense to mean that items following the word are included, but items not specifically mentioned are not excluded. In addition, “consist of” may be replaced by “consist essentially of” meaning that a feature as described herein may comprise additional feature(s) than the ones specifically identified, said additional feature(s) not altering the unique characteristic of the described features.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g., about 10) means that the value may be the given value (e.g., 10) more or less 10% of the value. As used herein, the term “and/or” indicates that one or more of the stated cases may occur, alone or in combination with at least one of the stated cases, up to with all of the stated cases.

Each embodiment as identified herein may be combined together unless otherwise indicated.

All patent applications, patents, and printed publications cited herein are incorporated herein by reference for at least the subject matter referenced and in their entireties, except for any definitions, subject matter disclaimers or disavowals, and except to the extent that the incorporated material is inconsistent with the express disclosure herein, in which case the language in this disclosure controls.

One skilled in the art will recognize many features similar or equivalent to those described herein, which could be used in the practice of the present subject matter. Indeed, the present disclosure is in no way limited to the features described.

### Polypeptides

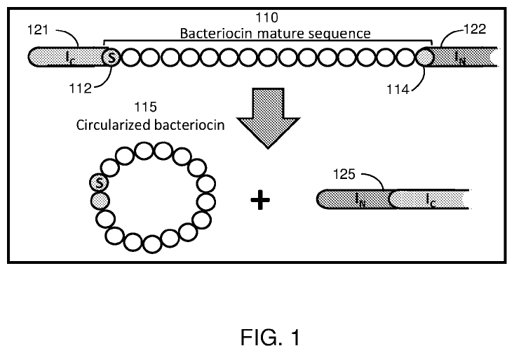

As discussed above, fusion polypeptides for generating circular bacteriocins are provided. With reference to FIG. 1, a schematic diagram of a fusion polypeptide of the present disclosure is provided. The fusion polypeptide can include an amino acid sequence 110 of a bacteriocin, which in some embodiments can be a mature sequence of the bacteriocin. A mature sequence typically includes a full sequence of the bacteriocin without the native signal peptide, leader sequence or other additional N- or C-terminal regulatory sequences (e.g., involved in processing and/or secretion). In some embodiments, the amino acid sequence is circularly permuted compared to the native mature sequence of the bacteriocin. The amino acid sequence 110 can be flanked by a split intein 121, 122 that is arranged such that the split intein circularizes the bacteriocin through cyclization of the peptide backbone. The amino acid sequence can be flanked at the N terminus by the C terminal intein fragment (“I_{C}”) 121 fused to the first amino acid residue 112 of the amino acid sequence 110 of the bacteriocin, and at the C-terminus by the N-terminal intein fragment (“I_{N}”) 122 fused to the last amino acid residue 114 of the amino acid sequence 110 of the bacteriocin. Thus, when the amino acid sequence 110 of the bacteriocin is flanked at both the N- and C-termini by the split intein, the split intein mediates formation of a peptide bond between the first amino acid residue 112 and the last amino acid residue 114 of the amino acid sequence 110 of the bacteriocin, to generate the circularized bacteriocin 115. The intein 125 after circularization can be cleaved from the circularized bacteriocin. In some embodiments, the N-terminal amino acid residue 112 of the bacteriocin is a serine (or cysteine) that is directly fused to the I_{C}. In some embodiments, the amino acid sequence 110 of the bacteriocin is modified (e.g., by circular permutation) from the native sequence (e.g., native mature sequence) such that the first amino acid residue in the sequence is a serine or cysteine, as further provided herein. In some embodiments, the intein 125 after circularization is removed via a C-terminal degradation tag.

In some embodiments, the fusion polypeptide includes an amino acid sequence of any suitable bacteriocin. In some embodiments, the amino acid sequence is that of a circular bacteriocin (e.g., a bacteriocin known to be circular as produced in a native context, a bacteriocin predicted to be circular based on the genomic context, a bacteriocin designed or engineered to be functional in circular form, etc.). In some embodiments, the bacteriocin has antimicrobial activity only when circularized. In some embodiments, the bacteriocin has substantial antimicrobial activity only when circularized. In some embodiments, the bacteriocin has antimicrobial activity when in linear form. In some embodiments, the bacteriocin has antimicrobial activity when circularized and when in linear form. In some embodiments, the bacteriocin has greater antimicrobial activity when circularized compared to when in linear form. In some embodiments, the bacteriocin has antimicrobial activity that is greater by at least about 10%, at least about 20%, at least about 30%, at least about 40%, at least about 50%, at least about 60%, at least about 70%, at least about 80%, at least about 90%, at least about 100%, at least about 120%, at least about 150%, or at least about 200% or more, or by a percentage in a range defined by any two of the preceding values, when circularized compared to when in linear form, for example, 10%-200%, 10%-100%, 50%-200%, 50%-100%, 70%-200%, or 50%-150%.

The fusion polypeptide can include any suitable amino acid sequence of a bacteriocin or a variant thereof (such as a circularly permuted variant thereof as described herein). In some embodiments, the amino acid sequence is or is derived from a naturally occurring bacteriocin. In some embodiments, the amino acid sequence is a mature sequence of a bacteriocin, or a variant thereof (such as a circularly permuted variant thereof as described herein). In some embodiments, the amino acid sequence is an amino acid sequence of a bacteriocin without a native signal peptide sequence. In some embodiments, the amino acid sequence is an amino acid sequence of a bacteriocin without any signal peptide sequence. In some embodiments, the amino acid sequence does not include any sequences that would have been required in a native context for processing of the bacteriocin (e.g., intracellular processing, circularization). In some embodiments, the amino acid sequence of a bacteriocin is modified from the native sequence (e.g., native mature sequence) to promote circularization by the split intein. In some embodiments, the amino acid sequence of the bacteriocin includes as the first amino acid residue an amino acid that is preferred by the split intein for circularization. In some embodiments, the amino acid that is preferred by the split intein for circularization depends on the type of split intein in the fusion polypeptide. In some embodiments, the amino acid that is preferred by the split intein for circularization is a cysteine or serine. In some embodiments, the amino acid sequence of the bacteriocin includes as the first amino acid residue a cysteine or serine. In some embodiments, the native amino acid sequence of the bacteriocin is circularly permuted, as disclosed herein, such that a cysteine or serine that is present in the native amino acid sequence is the first amino acid residue of the amino acid sequence of the bacteriocin of the fusion polypeptide.

In some embodiments, the amino acid sequence of the bacteriocin in the fusion polypeptide is circularly permuted compared to the native amino acid sequence (e.g., native mature sequence) of the bacteriocin. As used herein “circularly permuted” denotes modification of a linear sequence of elements by shifting the position of the elements while preserving the position of each element relative to each other, where elements that are shifted past the first or last position in the linear sequence wrap around to the opposite end of the sequence. For example, circular permutation of the sequence “ABCDE” can result in any one of “BCDEA”, “CDEAB”, “DEABC”, and “EABCD”. In some embodiments, an amino acid residue that is not the N-terminal residue in the native amino acid sequence (e.g., native mature sequence) of the bacteriocin is the first amino acid residue of the circularly permuted amino acid sequence of the bacteriocin in the fusion polypeptide. In some embodiments, the first amino acid residue of the circularly permuted amino acid sequence of the bacteriocin in the fusion polypeptide is an amino acid that is preferred by the split intein to be the first amino acid for circularization. In some embodiments, the preferred amino acid is a cysteine or serine. In some embodiments, where the native amino acid sequence of the bacteriocin includes a cysteine or serine, the amino acid sequence of a bacteriocin is circularly permuted compared to the native amino acid sequence such that the native cysteine or serine is the first amino acid residue of the amino acid sequence of the bacteriocin. In some embodiments, where the native amino acid sequence of the bacteriocin includes a cysteine or serine, the amino acid sequence of a bacteriocin is circularly permuted compared to the native amino acid sequence such that the native cysteine or serine is the first amino acid residue of the amino acid sequence of the bacteriocin, and is directly fused to the I_{C}. As used herein in the context of a fusion polypeptide, an “amino acid sequence of a bacteriocin” is intended to include circularly permuted sequences of the bacteriocin relative to its native sequence.

In some embodiments, the first amino acid residue of the amino acid sequence of the bacteriocin of the fusion polypeptide is a non-native amino acid residue. As used herein, “non-native” has its ordinary and customary meaning as understood by one of ordinary skill in the art in view of the present disclosure, and denotes an amino acid that is not present in a native amino acid sequence, or a circularly permuted sequence thereof. In some embodiments, the first amino acid residue of the amino acid sequence of the bacteriocin of the fusion polypeptide is a non-native amino acid residue that is a preferred amino acid for circularization by the split intein. In some embodiments, the native amino acid sequence of the bacteriocin is modified by adding the non-native amino acid residue to the native sequence, or by substituting a native amino acid residue with a non-native amino acid residue. In some embodiments, the native amino acid sequence of the bacteriocin is modified by adding the amino acid preferred by the split intein to a N-terminal of the native sequence, or by substituting the first amino acid residue of the native sequence with the amino acid preferred by the split intein to provide the amino acid sequence in the fusion polypeptide. In some embodiments, the native amino acid sequence of the bacteriocin does not include the amino acid preferred by the split intein. In some embodiments, the length of the amino acid sequence of the bacteriocin is increased by one residue due to addition of the non-native amino acid, compared to the length of the native amino acid sequence of the bacteriocin.

In some embodiments, the cysteine or serine that is the first amino acid residue of the amino acid sequence of the bacteriocin is a cysteine or serine that is not present in the native amino acid sequence of the bacteriocin (or in a circularly permuted sequence thereof). In some embodiments, the native amino acid sequence of the bacteriocin is modified by adding a cysteine or serine residue to the native sequence, or by substituting a native amino acid residue that is not a cysteine or serine with a cysteine or serine. In some embodiments, the native amino acid sequence of the bacteriocin is modified by adding a N-terminal cysteine or serine to the native sequence, or by substituting the first amino acid residue of the native sequence with a cysteine or serine to provide the amino acid sequence in the fusion polypeptide. In some embodiments, the native amino acid sequence of the bacteriocin can be modified by inserting a cysteine or serine into the native sequence, or substituting an amino acid of the native sequence (other than the first N-terminal residue) with a cysteine or serine to provide the amino acid sequence in the fusion polypeptide, and circularly permuting the modified sequence, as disclosed herein, such that the non-native cysteine or serine is the first amino acid residue of the amino acid sequence of the bacteriocin in the fusion polypeptide. In some embodiments, the native amino acid sequence of the bacteriocin does not include a serine or cysteine. In some embodiments, the length of the amino acid sequence of the bacteriocin is increased by one residue due to the non-native serine or the non-native cysteine compared to the length of the native amino acid sequence of the bacteriocin.

The amino acid sequence of the bacteriocin can have any suitable length. In some embodiments, the amino acid sequence of the bacteriocin in the fusion polypeptide is 20-30, 30-40, 40-50, 50-60, 60-70, 70-80, 80-90, 90-100, 100-110, 110-120, 120-130, 130-140, 140-150, 150-175, 175-200, 200-300, 300-400, 400-600, 600-800, 800-1000 amino acids long, or longer, or a length in a range defined by any two of the preceding values, for example 20-1000, 20-800, 20-600, 100-800, 20-150, 80-150, 40-130, 100-150, or 20-80 amino acids long.

Suitable amino acid sequences of a circular bacteriocin include, without limitation, any one of the sequences set forth in Table A. In some embodiments, the fusion polypeptide includes an amino acid sequence at least 70%, at least 75%, at least 80%, at least 85%, at least 90%, at least 95%, at least 97%, at least 98%, at least 99%, or about 100%, or by a percentage in a range defined by any two of the preceding values (e.g., 70-100%, 70-90%, 75-95%, 80-90%, 90-98%), identical to any one of the sequences in Table A. In some embodiments, the fusion polypeptide includes an amino acid sequence of any one of the sequences set forth in Table A, having up to 10, 9, 8, 7, 6, 5, 4, 3, 2, or 1 amino acid substitutions thereto. In some embodiments, the fusion polypeptide includes an amino acid sequence of any one of the sequences set forth in Table A, having up to 10, 9, 8, 7, 6, 5, 4, 3, 2, or 1 conservative amino acid substitutions thereto. In some embodiments, the fusion polypeptide includes an amino acid sequence of any one of the sequences set forth in Table A, having up to 10, 9, 8, 7, 6, 5, 4, 3, 2, or 1 amino acid additions or deletions thereto. In some embodiments, the fusion polypeptide includes an amino acid sequence of any one of the sequences set forth in Table A, having up to 10, 9, 8, 7, 6, 5, 4, 3, 2, or 1 amino acid substitutions, additions, and/or deletions thereto. In some embodiments, the fusion polypeptide includes an amino acid sequence of any one of the sequences set forth in Table A, having up to 10, 9, 8, 7, 6, 5, 4, 3, 2, or 1 amino acid conservative substitutions, additions, and/or deletions thereto. In some embodiments, the amino acid substitutions or additions include substitution with or addition of a non-natural amino acid. In some embodiments, the amino acid substitutions or additions include substitutions with or additions of natural amino acids only.

In Table A, each bacteriocin is represented by two amino acid sequences (except for Bacteriocin F9 from Staphylococcus felis, which is represented by three sequences), where for each bacteriocin entry, the native mature sequence is shown on the top and a modified form of the native mature sequence, each modified form having a serine as the first amino acid residue of the bacteriocin by circular permutation of the native sequence and/or insertion or substitution of a serine to the native sequence, is shown on the bottom. For example, for Amylocyclicin (Alc) from Bacillus amyloliquefaciens, the native mature sequence has the sequence:

(SEQ ID NO: 751)
LASTLGISTAAAKKAIDIIDAASTIASIISLIGIVTGAGAISYAIVATA
KTMIKKYGKKYAAAW,

and a circularly permuted form of the native mature sequence has the sequence:

(SEQ ID NO: 752)
STAAAKKAIDIIDAASTIASIISLIGIVTGAGAISYAIVATAKTMIKKY
GKKYAAAWLASTLGI.

In some embodiments, the fusion polypeptide includes an amino acid sequence of any one of the modified sequences (the bottom row of each bacteriocin entry) in Table A. In some embodiments, the fusion polypeptide includes an amino acid sequence at least 70%, at least 75%, at least 80%, at least 85%, at least 90%, at least 95%, at least 97%, at least 98%, at least 99%, or about 100%, or by a percentage in a range defined by any two of the preceding values (e.g., 70-100%, 80-100%, 90-95%, 85-95%, or 95-99%), identical to any one of the modified sequences (the bottom row of each bacteriocin entry) in Table A. In some embodiments, the fusion polypeptide includes an amino acid sequence ofany one of the modified sequences (the bottom row of each bacteriocin entry) in Table A, having up to 10, 9, 8, 7, 6, 5, 4, 3, 2, or 1 amino acid substitutions, additions, and/or deletions thereto. In some embodiments, the fusion polypeptide includes an amino acid sequence ofany one of the modified sequences (the bottom row of each bacteriocin entry) in Table A, having up to 10, 9, 8, 7, 6, 5, 4, 3, 2, or 1 conservative amino acid substitutions, additions, and/or deletions thereto. In some embodiments, the amino acid substitutions or additions include substitution with or addition of a non-natural amino acid. In some embodiments, the amino acid substitutions or additions include substitutions with or additions of natural amino acids only.

TABLE A

| Bacteriocin | Strain | Mature Sequence |
|---|---|---|
| Amylocyclicin | Bacillus | LASTLGISTAAAKKAIDIIDAASTIASIISLIGIVTGAGAISYAIVA |
| (Alc) | amyloliquefaciens | TAKTMIKKYGKKYAAAW |
| | FZB42 | (SEQ ID NO: 751) |
| | | STAAAKKAIDIIDAASTIASIISLIGIVTGAGAISYAIVATAKTMIK |
| | | KYGKKYAAAWLASTLGI |
| | | (SEQ ID NO: 752) |
| Bacteriocin | Staphylococcus | LTSTLGISSYAAKKAIDIIAAAGDVAAIVGLIGAVTGAGAIGAG |
| 3688STDY6124959 | aureus | ILFTAKKLIKSYGKKYAAAW |
| | 3688STDY6124959 | (SEQ ID NO: 753) |
| | | STLGISSYAAKKAIDIIAAAGDVAAIVGLIGAVTGAGAIGAGIL |
| | | FTAKKLIKSYGKKYAAAWLT |
| | | (SEQ ID NO: 754) |
| Enterocin AS-48 | Enterococcus | MAKEFGIPAAVAGTVLNVVEAGGWVTTIVSILTAVGSGGLS |
| (EntAS48) | faecalis S-48 | LLAAAGRESIKAYLKKEIKKKGKRAVIAW |
| | | (SEQ ID NO: 755) |
| | | SIKAYLKKEIKKKGKRAVIAWMAKEFGIPAAVAGTVLNVVEA |
| | | GGWVTTIVSILTAVGSGGLSLLAAAGRE |
| | | (SEQ ID NO: 756) |
| Bacteriocin BCW | Listeria | LAKEFGIPGGVAATVLNIVEAGGWATTILSILTAVGSGGLSLI |
| 2997 | monocytogenes | AAAGKESIKVYLKKQIKKKGRKAVIAW |
| | BCW 2997 | (SEQ ID NO: 757) |
| | | SIKVYLKKQIKKKGRKAVIAWLAKEFGIPGGVAATVLNIVEA |
| | | GGWATTILSILTAVGSGGLSLIAAAGKE |
| | | (SEQ ID NO: 758) |
| Bacteriocin CF11 | Clavibacter | LAQQFGIPSAVGGTILNLATAGGTAATIVGILVGLGSGGLGL |
| | michiganensis | LAAAGRETIKQFLLNEIKKKGRKAVIAW |
| | CF11 | (SEQ ID NO: 759) |
| | | SAVGGTILNLATAGGTAATIVGILVGLGSGGLGLLAAAGRET |
| | | IKQFLLNEIKKKGRKAVIAWLAQQFGIP |
| | | (SEQ ID NO: 760) |
| Bacteriocin NBRC | Paenibacillus | LAKEFGVPAVVGNALVQALDWGLTAATIASIIGAFITGGLSIV |
| 15376 | chondroitinus | AAAGTTALKTYLRNKLAEMGTKAFVAW |
| | NBRC 15376 | (SEQ ID NO: 761) |
| | | SIIGAFITGGLSIVAAAGTTALKTYLRNKLAEMGTKAFVAWLA |
| | | KEFGVPAVVGNALVQALDWGLTAATIA |
| | | (SEQ ID NO: 762) |
| Carnocyclin A | Carnobacterium | LVAYGIAQGTAEKVVSLINAGLTVGSIISILGGVTVGLSGVFT |
| | maltaromaticum | AVKAAIAKQGIKKAIQL |
| | UAL307 | (SEQ ID NO: 763) |
| | | SLINAGLTVGSIISILGGVTVGLSGVFTAVKAAIAKQGIKKAIQ |
| | | LLVAYGIAQGTAEKVV |
| | | (SEQ ID NO: 764) |
| Circularin A (CirA) | Clostridium | VAGALGVQTAAATTIVNVILNAGTLVTVLGIIASIASGGAGTL |
| | beijerinckii ATCC | MTIGWATFKATVQKLAKQSMARAIAY |
| | 25752 | (SEQ ID NO: 765) |
| | | SIASGGAGTLMTIGWATFKATVQKLAKQSMARAIAYVAGAL |
| | | GVQTAAATTIVNVILNAGTLVTVLGIIA |
| | | (SEQ ID NO: 766) |
| Bacteriocin YS111 | Streptococcus suis | VAAALGVPSAVASTVVNVILGAGTAVTILGIISSIASGGATTLL |
| | YS111 | TVGWAGFKATVQRLAKQSMARAIAY |
| | | (SEQ ID NO: 767) |
| | | STVVNVILGAGTAVTILGIISSIASGGATTLLTVGWAGFKATV |
| | | QRLAKQSMARAIAYVAAALGVPSAVA |
| | | (SEQ ID NO: 768) |
| Enterocin NKR-5-3B | Enterococcus | LTANLGISSYAAKKVIDIINTGSAVATIIALVTAVVGGGLITAGI |
| | faecium NKR-5-3 | VATAKSLIKKYGAKYAAAW |
| | | (SEQ ID NO: 769) |
| | | SSYAAKKVIDIINTGSAVATIIALVTAVVGGGLITAGIVATAKS |
| | | LIKKYGAKYAAAWLTANLGI |
| | | (SEQ ID NO: 770) |
| Bacteriocin DSM | Garciella | LASMLGISTVAAKKVIDIIDTASTVATIISLIGVIVGVGGITAGL |
| 15102 | nitratireducens | VTTAKAMIKKYGKKYATMW |
| | DSM 15102 | (SEQ ID NO: 771) |
| | | SMLGISTVAAKKVIDIIDTASTVATIISLIGVIVGVGGITAGLVT |
| | | TAKAMIKKYGKKYATMWLA |
| | | (SEQ ID NO: 772) |
| Garvicin ML | Lactococcus | LVATGMAAGVAKTIVNAVSAGMDIATALSLFSGAFTAAGGI |
| (GarML) | garvieae DCC43 | MALIKKYAQKKLWKQLIAA |
| | | (SEQ ID NO: 773) |
| | | SGAFTAAGGIMALIKKYAQKKLWKQLIAALVATGMAAGVAK |
| | | TIVNAVSAGMDIATALSLF |
| | | (SEQ ID NO: 774) |
| Leucocyclicin Q | Leuconostoc | LVNQLGISKSLANTILGAIAVGNLASWLLALVPGPGWATKAA |
| | mesenteroides | LATAETIVKHEGKAAAIAW |
| | TK41401 | (SEQ ID NO: 775) |
| | | SKSLANTILGAIAVGNLASWLLALVPGPGWATKAALATAETI |
| | | VKHEGKAAAIAWLVNQLGI |
| | | (SEQ ID NO: 776) |
| Bacteriocin | Bacillus | LINKLGISKSLAQDILTAIAVGNVASWLLVLIPGPGWATKAAISAAEI |
| AFS089278 | toyonensis | IVETSGEAAAVAY |
| | AFS089278 | (SEQ ID NO: 777) |
| | | SKSLAQDILTAIAVGNVASWLLVLIPGPGWATKAAISAAEIIVETSG |
| | | EAAAVAYLINKLGI |
| | | (SEQ ID NO: 778) |
| Uberolysin A | Streptococcus | LAGYTGIASGTAKKVVDAIDKGAAAFVIISIISTVISAGALGAVSASA |
| (UberA) | uberis 42 | DFIILTVKNYISRNLKAQAVIW |
| | | (SEQ ID NO: 779) |
| | | SGTAKKVVDAIDKGAAAFVIISIISTVISAGALGAVSASADFIILTVKN |
| | | YISRNLKAQAVIWLAGYTGIA |
| | | (SEQ ID NO: 780) |
| Bacteriocin TD3 | Bacillus | VAGVLSVDKGVATKVVNAVDAAGWAFVGGSTLLAIISGGTLSAAS |
| | vallismortis TD3 | ISIDALIYTIKNYLKRSLKAQAIAW |
| | | (SEQ ID NO: 781) |
| | | SVDKGVATKVVNAVDAAGWAFVGGSTLLAIISGGTLSAASISIDALI |
| | | YTIKNYLKRSLKAQAIAWVAGVL |
| | | (SEQ ID NO: 782) |
| Bacteriocin NRRL | Streptomyces | ATTPNVLTGYFGLTGATANRVLNAIEAGTDIAAALSVLGGVSAAG |
| B-24287 | pathocidini NRRL | GVAMWMLKQAIAKGGRKAVVA |
| | B-24287 | (SEQ ID NO: 783) |
| | | SVLGGVSAAGGVAMWMLKQAIAKGGRKAVVAATTPNVLTGYFG |
| | | LTGATANRVLNAIEAGTDIAAAL |
| | | (SEQ ID NO: 784) |
| Bacteriocin AK22 | Alkalibacterium | ILFVADLFGIPFTTDIVDQVWGYIEGGAGTVTAISWVLGVTIPAWA |
| | AK22 | VPIIGAVGVVSA |
| | | (SEQ ID NO: 785) |
| | | SWVLGVTIPAWAVPIIGAVGVVSAILFVADLFGIPFTTDIVDQVWG |
| | | YIEGGAGTVTAI |
| | | (SEQ ID NO: 786) |
| Bacteriocin 15828 | Gemella cuniculi | VYFIAGKLGITLAPGWYEKLVNYISAGGGAVDGVAMILGITLPGW |
| | DSM 15828 | AITVIGAFGLVSA |
| | | (SEQ ID NO: 787) |
| | | SAGGGAVDGVAMILGITLPGWAITVIGAFGLVSAVYFIAGKLGITL |
| | | APGWYEKLVNYI |
| | | (SEQ ID NO: 788) |
| Butyrivibriocin | Butyrivibrio | IYFIADKMGIQLAPAWYQDIVNWVSAGGTLTTGFAIIVGVTVPAW |
| AR10 | fibrisolvens AR10 | IAEAAAAFGIASA |
| | | (SEQ ID NO: 789) |
| | | SAGGTLTTGFAIIVGVTVPAWIAEAAAAFGIASAIYFIADKMGIQLA |
| | | PAWYQDIVNWV |
| | | (SEQ ID NO: 790) |
| Paracyclicin P | Lactobacillus. | IYFIANKLGIHLAPGWYQDMVNYVSAGGSLAGAFSVVAGVTLPA |
| (ParP) | paracesei subsp. | WIVPIATAFGAVSA |
| | paracasei JCM | (SEQ ID NO: 791) |
| | 8130/ | SAGGSLAGAFSVVAGVTLPAWIVPIATAFGAVSAIYFIANKLGIHLA |
| | DSM 5622 | PGWYQDMVNYV |
| | | (SEQ ID NO: 792) |
| Bacteriocin NCTC | Streptococcus | IYWICGKFGIRLAPGWYQDIVDFVSAGGSIVDAFAIIAGITLPAWVA |
| 12958 | thermophilus | PVLAGFGVVSA |
| | NCTC 12958 | (SEQ ID NO: 793) |
| | | SAGGSIVDAFAIIAGITLPAWVAPVLAGFGVVSAIYWICGKFGIRLA |
| | | PGWYQDIVDFV |
| | | (SEQ ID NO: 794) |
| Bacteriocin | Streptococcus | IYWVCGKFGIELAPGWYQDIVDFVSSGGTIVEAFAAVAGVTLPAW |
| UoS2029 | pneumoniae | VGPVLAAFGLASA |
| | UoS2029 | (SEQ ID NO: 795) |
| | | SSGGTIVEAFAAVAGVTLPAWVGPVLAAFGLASAIYWVCGKFGIEL |
| | | APGWYQDIVDFV |
| | | (SEQ ID NO: 796) |
| Bacteriocin | Staphylococcus | VTWVLDKFGIKIGTPTINEIINYVANGGAIVTAFAVIAGITLPAWVG |
| F9_V1* | felis F9 | PAVAAFATTAA |
| | | (SEQ ID NO: 797) |
| | | SVTWVLDKFGIKIGTPTINEIINYVANGGAIVTAFAVIAGITLPAWV |
| | | GPAVAAFATTAA |
| | | (SEQ ID NO: 798) |
| Bacteriocin | Staphylococcus | VTWVLDKFGIKIGTPTINEIINYVANGGAIVTAFAVIAGITLPAWVG |
| F9_V2* | felis F9 | PAVAAFATTAA |
| | | (SEQ ID NO: 799) |
| | | SAVTWVLDKFGIKIGTPTINEIINYVANGGAIVTAFAVIAGITLPAW |
| | | VGPAVAAFATT |
| | | (SEQ ID NO: 800) |
| Gassericin A | Lactobacillus | IYWIADQFGIHLATGTARKLLDAMASGASLGTAFAAILGVTLPAW |
| (GasA) | gasseri LA39 | ALAAAGALGATAA |
| | | (SEQ ID NO: 801) |
| | | SGASLGTAFAAILGVTLPAWALAAAGALGATAAIYWIADQFGIHLA |
| | | TGTARKLLDAMA |
| | | (SEQ ID NO: 802) |
| Plantaricyclin A | Lactobacillus | IVWIAKQFGVHLTTSLTQKALDLLSAGSSLGTVAAAVLGVTLPAWA |
| (PlcA) | plantarum NI326 | VAAAGALGGTAA |
| | | (SEQ ID NO: 803) |
| | | SLTQKALDLLSAGSSLGTVAAAVLGVTLPAWAVAAAGALGGTAAI |
| | | VWIAKQFGVHLTT |
| | | (SEQ ID NO: 804) |
| Cerecyclin | Bacillus sp. Xin1 | VVSKLGWTGINIGTANALIGALMTGSDIWTAISVAGLAFGGGIGT |
| | | AISTIGRKAIMEMVEKVGKKKAAQW |
| | | (SEQ ID NO: 805) |
| | | SKLGWTGINIGTANALIGALMTGSDIWTAISVAGLAFGGGIGTAIS |
| | | TIGRKAIMEMVEKVGKKKAAQWVV |
| | | (SEQ ID NO: 806) |
| Bacteriocin | Paeniclostridium | IYFIADKMGITLSTKMYRDIVDAVSAGGSIAGAFSLIVGVTLP |
| R26833 | sordellii R26833 | GWLVVAAAGFGLTSA |
| | | (SEQ ID NO: 807) |
| | | STKMYRDIVDAVSAGGSIAGAFSLIVGVTLPGWLVVAAAGF |
| | | GLTSAIYFIADKMGITL |
| | | (SEQ ID NO: 808) |

Other non-limiting examples of bacteriocins (e.g., linear bacteriocins) suitable for use in the present fusion polypeptides are set forth in the even numbered sequences of SEQ ID NOS: 4-450 and the odd numbered sequences of SEQ ID NOS: 699-737. Detailed descriptions of suitable bacteriocins can be found, for example, in U.S. Pat. No. 9,333,227 and International Publication No. WO2019/046577, each of which is hereby incorporated by reference in its entirety. Some examples of suitable bacteriocins and categories of bacteriocins are taught in Tables 1.1 and 1.2 of U.S. Pat. No. 9,333,227 and of International Publication No. WO2019/046577. The amino acid sequence of the bacteriocin in the fusion polypeptide can include a sequence that is at least 70%, at least 75%, at least 80%, at least 85%, at least 90%, at least 95%, at least 97%, at least 98%, at least 99%, or about 100%, or by a percentage in a range defined by any two of the preceding values (e.g., 70-100%, 70-90%, 75-95%, 80-90%, 90-98%), identical to any of the amino acid sequences of bacteriocins disclosed herein, including those above. Any of the amino acid sequences of bacteriocins disclosed herein, including those above, can be modified by any suitable option as disclosed herein, such that a serine or cysteine (e.g., a native serine or cysteine) is the first amino acid of the bacteriocin sequence. In some embodiments, any of the amino acid sequences of bacteriocins disclosed herein, including those above, can be circularly permuted to place a serine or cysteine as the first amino acid of the bacteriocin sequence in the fusion polypeptide. In some embodiments, a serine or cysteine can be added to or can substitute a native amino acid in any of the amino acid sequences of bacteriocins disclosed herein, including those above, and optionally can be further be circularly permuted, to place a non-native serine or cysteine as the first amino acid of the bacteriocin sequence in the fusion polypeptide.

In some embodiments the bacteriocin in the present fusion polypeptide is an engineered bacteriocin, e.g., a polypeptide engineered to have antimicrobial activity when circularized. In some embodiments, the fusion polypeptide includes a non-natural amino acid in the amino acid sequence of the bacteriocin and/or split intein. In some embodiments, the fusion polypeptide includes 1, 2, 3, 4, 5, or more non-natural amino acids in the amino acid sequence of the bacteriocin and/or split intein. In some embodiments, 1, 2, 3, 4, 5, or more amino acids in the amino acid sequence of the bacteriocin and/or split intein of the fusion polypeptide is substituted with a corresponding non-natural amino acid.

The split intein of the present fusion polypeptide can be any suitable intein that can mediate circularization of a bacteriocin. In some embodiments, the split intein includes a C- and N-terminal intein fragments (I_{C} and I_{N}, respectively) that flank the bacteriocin. In some embodiments, I_{C} is fused to the N-terminus of the amino acid sequence of the bacteriocin, and I_{N} is fused to C-terminus of the amino acid sequence of the bacteriocin. In some embodiments, the split intein is a constitutively active split intein (e.g., a split intein that can circularize the bacteriocin under conditions in which the fusion polypeptide is expressed from a nucleic acid encoding same). In some embodiments, the split intein is a conditional split intein, e.g., a split intein that circularizes the bacteriocin under permissive conditions, and not under non-permissive conditions. In some embodiments, the split intein circularizes the bacteriocin under permissive conditions, and does not substantially circularize the bacteriocin under non-permissive conditions. In some embodiments, the split intein circularizes the bacteriocin preferentially or specifically under a permissive condition. In some embodiments, the split intein circularizes the bacteriocin under a permissive condition at a faster rate than under a non-permissive condition. In some embodiments, the split intein circularizes the bacteriocin under a permissive condition to a greater extent than under a non-permissive condition. In some embodiments, the conditional split intein is sensitive to pH, temperature, light stimulation, and/or a small molecule ligand. As used herein, “sensitive” has its customary and ordinary meaning as understood by one of skill in the art in view of this disclosure, and with reference to an environmental condition of a conditional split intein, denotes that the split intein's circularization activity (e.g., rate and/or extent thereof) is affected by the environmental condition to which the split intein is exposed.

In some embodiments, the split intein is configured to circularize the bacteriocin preferentially or specifically under a permissive pH (or pH range). In some embodiments, the split intein is configured to circularize the bacteriocin preferentially or specifically under a permissive temperature (or temperature range). In some embodiments, the split intein is pH-sensitive. In some embodiments, the split intein circularizes the bacteriocin at pH below a threshold pH or above a threshold pH, or within a pH range. In some embodiments, the threshold pH is less than 3.0, or about 3.0, 4.0, 4.5, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.5, or about 10.0, or a pH value in a range defined by any two of the preceding values, for example pH 3-5, pH 4-7, pH 4-6, pH 5-6, pH 8-10, pH 8-9. In some embodiments, the pH range is bound by any two of the following pH values: 3.0, 4.0, 4.5, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.5, 10.0. In some embodiments, the split intein is temperature-sensitive. In some embodiments, the split intein circularizes the bacteriocin at a temperature below or above a threshold temperature, or within a temperature range. In some embodiments, the threshold temperature is less than 15° C., or about 15° C., 16.0° C., 17.0° C., 18.0° C., 19.0° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 45° C., 50° C., 55° C., or about 60° C., or a temperature in a range defined by any two of the preceding values, for example 15-20° C., 15-18° C., 30-40° C., 40-50° C., 25-35° C. In some embodiments, the temperature range is bound by any two of the following temperatures: 15° C., 16.0° C., 17.0° C., 18.0° C., 19.0° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 45° C., 50° C., 55° C., 60° C. In some embodiments, the split intein is configured to circularize the bacteriocin preferentially or specifically in the presence of a small molecule ligand. In some embodiments, the split intein is configured to circularize the bacteriocin preferentially or specifically by light stimulation. Non-limiting examples of suitable conditional inteins are disclosed in Di Ventura et al., (Biological Chemistry, vol. 400, no. 4, 2019, pp. 467-475), which is incorporated herein by reference in its entirety.

In some embodiments, the split intein is based on an intein from one of the following: Npu DnaE, Sce VMA, Ssp DnaE. In some embodiments, the split intein is a naturally split intein (e.g., is found as a split intein in the genome of the host microorganism). In some embodiments, the split intein is not a split intein in its native context, and is engineered to be a split intein. In some embodiments, the split intein is a constitutively active split intein (e.g., a split intein that can circularize the bacteriocin under conditions in which the fusion polypeptide is expressed from a nucleic acid encoding same) derived from any one of Npu DnaE, Sce VMA, Ssp DnaE. In some embodiments, the split intein is a conditional split intein derived from any one of Npu DnaE, Sce VMA, Ssp DnaE. In some embodiments, the split intein is derived from a Nostoc punctiforme (Npu) DnaE split intein. In some embodiments, the split intein includes C- and N-terminal intein fragments from the Npu DnaE split intein. In some embodiments, the I_{C} and I_{N} include the respective amino acid sequences set forth in Table B. In some embodiments, the I_{C} and I_{N} include an amino acid sequence at least 70%, at least 75%, at least 80%, at least 85%, at least 90%, at least 95%, at least 97%, at least 98%, at least 99%, or about 100%, or by a percentage in a range defined by any two of the preceding values (e.g., 70-100%, 70-90%, 75-95%, 80-90%, 90-98%), identical to the respective sequences set forth in Table B.

TABLE B

| Fragment | Description | Mature Sequence |
|---|---|---|
| I_{C} | C-terminal intein fragment from | MIKIATRKYLGKQNVYDIGVERYHNFALKNGFIASN |
| | Nostoc punctifrome (Npu) DnaE split | (SEQ ID NO: 809) |
| | intein | |
| I_{N} | N-terminal intein fragment from | CLSYDTEILTVEYGILPIGKIVEKRIECTVYSVDNNGNIYTQP |
| | Nostoc punctifrome (Npu) DnaE split | VAQWHDRGEQEVFEYCLEDGCLIRATKDHKFMTVDGQ |
| | intein | MMPIDEIFERELDLMRVDNLPNGT |
| | | (SEQ ID NO: 810) |

In some embodiments, the fusion polypeptide includes one or more additional functional sequences in addition to the bacteriocin flanked by the split intein. In some embodiments, the fusion polypeptide includes a degradation tag, e.g., configured to degrade the intein after the bacteriocin is circularized and the intein cleaved from the circularized bacteriocin. In some embodiments, the fusion polypeptide includes a C-terminal degradation tag that is fused C-terminal to the N-terminal intein fragment, I_{N}. In some embodiments, the split intein includes a C-terminal intein fragment (“I_{C}”) fused N-terminal to the amino acid sequence of the bacteriocin and a N-terminal intein fragment (“I_{N}”) fused C-terminal to the amino acid sequence of the bacteriocin, where the polypeptide further includes a degradation tag C-terminal to the I_{N}. The degradation tag can be any suitable peptide that can induce degradation of the split intein after the bacteriocin is circularized and the intein is cleaved from the circularized bacteriocin. In some embodiments, the degradation tag is an SsrA sequence. In some embodiments, the degradation tag includes the sequence: AANDENYALAA (SEQ ID NO: 873). In some embodiments, the degradation tag includes a sequence at least 70%, at least 75%, at least 80%, at least 85%, at least 90%, at least 95%, at least 97%, at least 98%, at least 99%, or about 100%, or by a percentage in a range defined by any two of the preceding values (e.g., 70-100%, 70-90%, 75-95%, 80-90%, 90-98%), identical to AANDENYALAA (SEQ ID NO: 873). In some embodiments, the degradation tag includes a sequence that differs from AANDENYALAA (SEQ ID NO: 873) by at most 4, 3, 2, or 1 amino acids (e.g., substitutions, additions, and/or deletions). In some embodiments, the degradation tag includes a sequence that differs from AANDENYALAA (SEQ ID NO:873) by at most 4, 3, 2, or 1 conservative amino acid substitutions, additions, and/or deletions. In some embodiments, the amino acid substitutions or additions include substitution with or addition of a non-natural amino acid. In some embodiments, the amino acid substitutions or additions include substitutions with or additions of natural amino acids only.

In some embodiments, the fusion polypeptide includes one or more affinity tags. In some embodiments, the affinity tag is associated with the split intein, which can facilitate purification of the fusion protein, but may be dissociated from the bacteriocin upon circularization. In some embodiments, the affinity tag is associated with the amino acid sequence of the bacteriocin, and may be incorporated into the circular bacteriocin. The affinity tag can be used to purify the circular bacteriocin after circularization. Optionally, one or more cleavage sites can be positioned between the affinity tag and the rest of the fusion polypeptide to facilitate removal of the affinity tag after affinity purification. Affinity tags can be used in purification, for example by contact with a molecule that binds the affinity tag immobilized on a solid phase, such as a bead. Example affinity tags suitable for fusion polypeptides of the present disclosure can comprise, consist essentially of, or consist of His-tags, glutathione-S-transferase (GST) tags, FLAG tags, strep tags, maltose binding protein (MBP), chitin binding protein (CBP), myc tags, HA tags, NE tags, and V5 tags, variants of any of these, or any combination of two or more of these. In some embodiments, the affinity tag is a chitin binding protein (CBP). In some embodiments, the fusion polypeptide or the circularized bacteriocin having a CBP affinity tag is purified using a chitin resin.

In some embodiments, the fusion polypeptide includes a signal peptide and/or leader sequence. In some embodiments, the signal peptide or leader sequence is configured to facilitate secretion of the fusion polypeptide from a microbial cell genetically engineered to express the fusion polypeptide, as disclosed herein. Any suitable signal peptide and/or leader sequence that may facilitate secretion of the fusion polypeptide or circular bacteriocin from the genetically engineered microbial cell may be used. In some embodiments, the fusion polypeptide further comprises a post-translational or co-translation modification, for example, glycosylation, acetylation, methylation, PEGylation, SUMOylation, ubiquitination, or two or more of any of these.

Also provided are compositions comprising the fusion polypeptide of the present disclosure. In some embodiments, the composition includes a physiologically compatible carrier, such as water or a buffer solution. In some embodiments, the fusion polypeptide is lyophilized in the composition. Also provided is a composition comprising a circular bacteriocin and a split intein. The circular bacteriocin can be any circular bacteriocin produced from the fusion polypeptide, including those described herein. In some embodiments, the split intein includes a C-terminal intein fragment (I_{C}) and an N-terminal intein fragment (I_{N}). In some embodiments, the I_{C} and I_{N} are associated with each other. The split intein can be any suitable split intein as provided herein. In some embodiments, the split intein further comprises a degradation tag.

### Nucleic Acids, Vectors, Genetically Engineered Microbial Cells

Also provided herein is a nucleic acid that includes a nucleotide sequence encoding a fusion polypeptide as described herein. In some embodiments, the nucleic acid (e.g., DNA or RNA) includes regulatory elements that drive expression of the fusion polypeptide under suitable conditions. In some embodiments, the nucleic acid includes DNA. In some embodiments, the nucleic acid (e.g., DNA) includes regulatory elements (e.g., promoter) that drive transcription from the nucleic acid under suitable conditions (e.g., in vivo expression or in vitro transcription). In some embodiments, the nucleotide sequence is operably linked to a promoter sequence, e.g., in a DNA vector, as disclosed herein. Any suitable promoter sequence can be used to drive transcription from the nucleic acid. In some embodiments, the promoter sequence is one suitable for driving transcription from the nucleic acid in vitro (e.g., in an in vitro transcription solution). In some embodiments, the promoter sequence is one suitable for expressing the fusion polypeptide from the nucleic acid in vivo (e.g., in a microbial cell). In some embodiments, the promoter is a constitutive promoter. In some embodiments, the promoter is a conditionally active promoter, e.g., depending on the presence or absence of an environmental condition, chemical compound, gene product, stage of the cell cycle, or the like.

Non-limiting, example nucleic acids encoding some of these bacteriocins (e.g., linear bacteriocins) are set forth in the odd numbered sequences of SEQ ID NOs: 5-451 and the even numbered sequences of 700-738. Detailed descriptions of suitable bacteriocins and some polynucleotide sequences that encode bacteriocins, including methods and compositions for using bacteriocins to control the growth of microbial cells can be found, for example, in U.S. Pat. No. 9,333,227 and International Publication No. WO2019/046577, each of which is hereby incorporated by reference in its entirety.

In some embodiments, the nucleic acid includes regulatory elements that drive translation of the fusion polypeptide from the nucleic acid under suitable conditions (e.g., in vivo expression or in vitro translation). In some embodiments, the nucleic acid is RNA. Generally, translation initiation for a particular transcript is regulated by particular sequences at or 5′ of the 5′ end of the coding sequence of a transcript. For example, a coding sequence can begin with a start codon configured to pair with an initiator tRNA. While naturally-occurring translation systems typically use Met (AUG) as a start codon, it will be readily appreciated that an initiator tRNA can be engineered to bind to any desired triplet or triplets, and accordingly, triplets other than AUG can also function as start codons in certain embodiments. Additionally, sequences near the start codon can facilitate ribosomal assembly, for example a Kozak sequence ((gcc)gccRccAUGG, SEQ ID NO: 542, in which R represents “A” or “G”) or Internal Ribosome Entry Site (IRES) in typical eukaryotic translational systems, or a Shine-Delgarno sequence (GGAGGU, SEQ ID NO: 543) in typical prokaryotic translation systems. As such in some embodiments, a transcript comprising a “coding” nucleotide sequence of the present disclosure includes an appropriate start codon and translational initiation sequence. In some embodiments, for example if two or more “coding” nucleotide sequences are positioned in cis on a transcript, each nucleotide sequence includes an appropriate start codon and translational initiation sequence(s). In some embodiments, for example, if two or more “coding” nucleotide sequences are positioned in cis on a transcript, the two sequences are under control of a single translation initiation sequence, and either provide a single polypeptide that can function with both encoded polypeptides in cis, or provide a means for separating two polypeptides encoded in cis, for example a 2A sequence or the like. In some embodiments, a translational initiator tRNA is regulatable, so as to regulate initiation of translation of a bacteriocin from the nucleic acid.

Also provided is a genetic vector that includes a nucleic acid of the present disclosure. Any suitable genetic vector can be used to include a nucleic acid having a nucleotide sequence encoding a fusion polypeptide as described herein. In some embodiments, the genetic vector is an expression vector. Suitable genetic vectors include, without limitation, plasmids, viruses (including bacteriophage), and transposable elements.

In some embodiments, a genetic vector can include one or more additional nucleotide sequences encoding a gene product of interest. In some embodiments, a genetic vector can include an additional nucleotide sequence encoding a gene product that confers resistance to the circularized bacteriocin in the microbial cell expressing the circularized bacteriocin from the genetic vector. In some embodiments, the gene product that confers resistance to the circularized bacteriocin is an immunity modulator. Any suitable immunity modulator can be encoded by the additional nucleotide sequence in the genetic vector. Suitable immunity modulators are provided, e.g., without limitation in U.S. Pat. No. 9,333,227. In some embodiments, the genetic vector is configured to express the gene product of interest under suitable conditions. In some embodiments, a promoter in the genetic vector drives transcription from both the nucleotide sequence encoding the bacteriocin, and from the additional nucleotide sequence encoding the gene product that confers resistance to the circularized bacteriocin in the microbial cell expressing the circularized bacteriocin from the genetic vector. In some embodiments, expression of the nucleotide sequence encoding the bacteriocin and the additional nucleotide sequence encoding the gene product that confers resistance to the circularized bacteriocin are under the control of different promoters. In some embodiments, either one or both of the promoters controlling expression of the nucleotide sequence encoding the bacteriocin and the additional nucleotide sequence encoding the gene product that confers resistance to the circularized bacteriocin is a conditional promoter. In some embodiments, expression from a conditional promoter operably linked to the nucleotide sequence encoding the bacteriocin is regulated by different conditions compared to expression from a conditional promoter operably linked to the additional nucleotide sequence encoding the gene product that confers resistance to the circularized bacteriocin.

Also provided is a genetically engineered microbial cell that includes a nucleic acid of the present disclosure, or a genetic vector as provided herein. The microbial cell can be genetically engineered by any suitable option. In some embodiments, the microbial cell is transformed with the genetic vector of the present disclosure. In some embodiments, nucleic acid is stably integrated into a chromosome, or can be a self-replicating unit that is independent of the chromosome (e.g., as a plasmid, extrachromosomal array, episome, minichromosome, or the like). In some embodiments, plasmid conjugation can be used to introduce a desired plasmid from a “donor” microbial cell to a recipient microbial cell.

Any suitable microbial cell can be genetically engineered to include the nucleic acid or genetic vector of the present disclosure. In some embodiments, the microbial cell is one that does not naturally produce the bacteriocin encoded by the nucleic acid or genetic vector. In some embodiments, the microbial cell is one that does not encode the bacteriocin encoded by the nucleic acid or genetic vector in its genome endogenously. In some embodiments, the microbial cell is resistant to the bacteriocin. In some embodiments, the microbial cell expresses a gene product (e.g., an immunity modulator) that confers resistance to the bacteriocin. In some embodiments, the microbial cell is genetically engineered to expresses the gene product (e.g., an immunity modulator) that confers resistance to the bacteriocin. In some embodiments, expression of the immunity modulator from the second nucleic acid is regulatable. In some embodiments, expression of the immunity modulator from the second nucleic acid is controlled by a conditional promoter.

Exemplary microbial cells that can be used in accordance with embodiments herein include, but are not limited to, bacteria, yeast, filamentous fungi, and algae, for example photosynthetic microalgae. Furthermore, fully synthetic microorganism genomes can be synthesized and transplanted into single microbial cells, to produce synthetic microorganisms capable of continuous self-replication (see Gibson et al. (2010), “Creation of a Bacterial Cell Controlled by a Chemically Synthesized Genome,” Science 329: 52-56, which is incorporated herein by reference). As such, in some embodiments, the microbial cell is fully synthetic. A desired combination of genetic elements, including elements that regulate gene expression, and elements encoding gene products (for example immunity modulators, poison, antidote, and industrially useful molecules also called product of interest) can be assembled on a desired chassis into a partially or fully synthetic microbial cell. Description of genetically engineered microbial organisms for industrial applications can also be found in Wright, et al. (2013) “Building-in biosafety for synthetic biology” Microbiology 159: 1221-1235, incorporated herein by reference.

A variety of bacterial species and strains can be used in accordance with embodiments herein, and genetically modified variants, or synthetic bacteria based on a “chassis” of a known species can be provided. Exemplary bacteria (including those with industrially applicable characteristics), which can be used in accordance with embodiments herein include, but are not limited to, Bacillus species (for example Bacillus coagulans, Bacillus subtilis, and Bacillus licheniformis), Paenibacillus species, Streptomyces species, Micrococcus species, Corynebacterium species, Acetobacter species, Cyanobacteria species, Salmonella species, Rhodococcus species, Pseudomonas species, Lactobacillus species, Enterococcus species, Alcaligenes species, Klebsiella species, Paenibacillus species, Arthrobacter species, Corynebacterium species, Brevibacierium species, Thermus aquaticus, Pseudomonas stutzeri, Clostridium thermocellus, and Escherichia coli.

A variety of yeast species and strains can be used in accordance with embodiments herein, and genetically modified variants, or synthetic yeast based on a “chassis” of a known species can be provided. Exemplary yeast with industrially applicable characteristics, which can be used in accordance with embodiments herein include, but are not limited to Saccharomyces species (for example, Saccharomyces cerevisiae, Saccharomyces bayanus, Saccharomyces boulardii), Candida species (for example, Candida utilis, Candida krusei), Schizosaccharomyces species (for example Schizosaccharomyces pombe, Schizosaccharomyces japonicus), Pichia or Hansenula species (for example, Pichia pastoris or Hansenula polymorpha) species, and Brettanomyces species (for example, Brettanomyces claussenii).

A variety of algae species and strains can be used in accordance with embodiments herein, and genetically modified variants, or synthetic algae based on a “chassis” of a known species can be created. In some embodiments, the algae comprises, consists essentially of, or consists of photosynthetic microalgae. Exemplary algae species that can be useful for biofuels, and can be used in accordance with some embodiments herein, include Botryococcus braunii, Chlorella species, Dunaliella tertiolecta, Gracilaria species, Pleurochrysis carterae, and Sargassum species. Additionally, many algae can be useful for food products, fertilizer products, waste neutralization, environmental remediation, and carbohydrate manufacturing (for example, biofuels).

A variety of filamentous fungal species and strains can be used in accordance with embodiments herein, and genetically modified variants, or synthetic filamentous fungi based on a “chassis” of a known species can be provided. Exemplary filamentous fungi (including those with industrially applicable characteristics), which can be used in accordance with embodiments herein include, but are not limited to an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocaffimastix, Neurospora, Paecilomyces, Peniciffium, Phanerochaele, Piromyces, Poitrasia, Pseudopleclania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria. In some embodiments, filamentous fungus species include, without limitation, Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride.

Also provided is a library that includes the nucleic acids or genetic vectors of the present disclosure. The library in some embodiments finds use in screening circular bacteriocins whose antimicrobial activity has not been characterized, or for screening circular bacteriocins from different strains for one having a desired antimicrobial activity. In some embodiments, the library finds use in screening different variants of a circular bacteriocin for desired or altered activity. In some embodiments, at least two of the genetic vectors in the library include nucleotide sequences encoding different bacteriocins. The bacteriocins encoded by the genetic vectors of the library can differ in any suitable manner. In some embodiments, the library is a mutational library that includes sequence variants of a bacteriocin that has one or more mutations compared to a parent sequence. The mutations in the sequence variants can include random mutations, in some embodiments. In some embodiments, the mutations in the sequence variants can include targeted mutations. The library can include, in some embodiments, sequence variants that would abolish or abrogate circularization of the bacteriocin in a native context. In some embodiments, the parent bacteriocin is a natively circular bacteriocin, and the sequence variants include a first variant that abrogates natural circularization of the parent bacteriocin.

In some embodiments, the library includes bacteriocins from different strains or species of microbial organisms (e.g., bacteria). In some embodiments, the library includes different previously uncharacterized bacteriocins, e.g., bacteriocins predicted based on sequence alone or bacteriocins for which antimicrobial activity has not been observed. In some embodiments, the library includes different bacteriocins known to have antimicrobial activity.

The library can include any suitable number of variants. In some embodiments, the library includes at least about 10, 20, 30, 40, 50, 60, 70, 80, 90, 100, 120, 140, 160, 180, 200, 300, 400, 500, 600, 700, 800, 900, 10³, 10⁴, 10⁵, 10⁶, 10⁷, 10⁸, 10⁹ or more variants, or a number of variants in a range defined by any two of the preceding values.

### Methods

Also provided are methods of making a circular bacteriocin (which for convenience may be referred to as production methods). In some embodiments, the method includes contacting a nucleic acid having a nucleotide sequence that encodes a fusion polypeptide as described herein with an in vitro expression system under conditions sufficient to produce a circular bacteriocin. In some embodiments, the in vitro expression system is a cell-free transcription/translation solution. In some embodiments, use of the in vitro expression system allows expression of a circular bacteriocin from a nucleic acid encoding the same, where the nucleic acid cannot be expressed in vivo, e.g., from a microbial cell genetically engineered with the nucleic acid. Without being bound by theory, in some cases, toxicity of the nucleic acid or the gene product encoded therein can prevent expression of the gene product from the nucleic acid.

Any suitable cell-free expression system can be used to transcribe and/or translate nucleic acids in vitro. In some embodiments, the in vitro expression system comprises, consists of, or consists essentially of cell extracts. In some embodiments, the in vitro expression system comprises an RNA polymerase, ribosomes, tRNAs (and the corresponding amino acids), an energy source, and enzymatic cofactors. In some embodiments, the in vitro expression system can further comprise enzymes for co- or post-translational modification, and/or cellular components that mediate protein folding such as heat shock proteins. By way of example, in embodiments in which the nucleic acid comprises RNA transcripts encoding the fusion polypeptide, it is contemplated that an in vitro expression system comprising, consisting essentially of, or consisting of a translation solution is sufficient (since it will be understood that the RNA is already a transcript). By way of example, in embodiments in which the nucleic acid comprises DNA encoding the fusion polypeptide, it is contemplated that an in vitro expression system comprises a transcription solution (for transcribing the DNAs into RNAs) and a translation solution (for translating the RNAs into polypeptides). In some embodiments, the transcription and translation solutions are together in a single solution (e.g., components of the transcription solution and translation solution are distributed evenly within the same volume). In some embodiments, the transcription and translation solutions are in separate solutions, for example in vesicles suspended in a single solution, and/or in separate solutions that are applied sequentially, and/or in separate compartments. In some embodiments, components of the in vitro transcription/translation solution are lyophilized, and configured to be reconstituted into the in vitro transcription/translation solution upon the addition of water. In some embodiments, the in vitro transcription/translation solution is reconstituted by adding water to lyophilized components.

Translation solutions can be useful for translating the nucleic acids as provided herein. Suitable translation solutions can comprise, consist essentially of, or consist of reagents for in vitro translation (which, for convenience, may be referred to herein as “translation reagents”), and as such can be configured for in vitro translation of a transcript such as an RNA. Some embodiments include a transcription solution comprising reagents for transcription (which, for convenience, may be referred to herein as “transcription reagents”), and thus is configured for in vitro transcription and translation, for example to transcribe and translate the nucleic acid encoding fusion polypeptides as provided herein. It is contemplated that in vitro transcription and translation in a single solution (such as a transcription solution further comprising a translation solution as described herein) can facilitate efficient in vitro production of fusion polypeptides of some embodiments. Thus, in accordance with some embodiments described herein, the in vitro expression system comprises an in vitro transcription reagent and/or an in vitro translation reagent.

In accordance with some embodiments described herein, the translation solution comprises, consists essentially of, or consists of one or more translation reagents or in vitro translation reagents. Examples of translation reagents include, but are not limited to, a ribosome, a buffer, an amino acid, a tRNA (which may be conjugated to an amino acid), a lysate or extract such as an E. coli lysate or E. coli extract, and a cofactor or metallic ion such as Mg²⁺, or a combination of two or more of any of the listed items. In accordance with some embodiments described herein the translation solution further comprises a transcription reagents, and thus is configured for in vitro transcription and translation. As described herein, a transcription solution further comprising translation reagents contemplates a single solution that is suitable for in vitro transcription and translation. As such, a transcription solution further comprising translation reagents encompasses a single transcription/translation solution. It will be appreciated that some components of a transcription and/or translation solution, for example ribosomes, may not be liquids, and could potentially be isolated from the transcription and/or translation solution, for example by filtration and/or centrifugation.

In some embodiments, the translation solution comprises a post-translational modification enzyme. Examples of post-translational modification enzymes include, but are not limited to a cleavage enzyme, a kinase, a phosphatase, a giycosyltransferase, or a mixture of any two of the listed items.

Transcription solutions of some embodiments described herein (and which can be comprised by translation solutions as described herein) can comprise, consist essentially or, or consist of one or more transcription reagents. Examples of transcription reagents include an RNA polymerase, a buffer, a nucleic acid mix (for example, NTPs including ATP, GTP, CTP, and UTP), a cofactor or metallic ion such as Mg²⁺, a transcription inducer (such as a transcription factor, IPTG, or lactose), a polyadenylation enzyme, a capping enzyme, a lysate or extract such as a bacterial lysate or extract such as an E. coli lysate or E. coli extract, an SP6 polymerase, a T3 polymerase, a T7 RNA polymerase, or a mixture of two or more of any of the listed items. The transcription solution can be useful for transcribing a template, such as a candidate nucleic acid as described herein. Translation solutions of some embodiments include one or more transcription reagents in combination with one or more translation reagents.

The in vitro expression system can be provided in any suitable volume. In accordance with some embodiments described herein, the in vitro expression system is provided in a volume of 1 μl-1000 μl, 1 μl-50 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, 500 μl-1000 μl, 1 ml-2 ml, 3 ml-5 ml, 5 ml-10 ml, 10 ml-20 ml, 20 ml-50 ml, 50 ml-100 ml, or more.

In accordance with some embodiments described herein, the in vitro transcription/translation solution is lyophilized. In some embodiments, the in vitro transcription/translation solution is configured be reconstituted in a solution such as water.

The contacting can be carried out for any suitable amount of time. In some embodiment, the contacting is done for at least about 10 minutes, at least about 20 minutes, at least about 30 minutes, at least about 45 minutes, at least about 60 minutes, at least about 1.5 hours, at least about 2 hours, at least about 3 hours, at least about 4 hours, at least about 6 hours, at least about 8 hours, at least about 10 hours, at least about 12 hours, at least about 16 hours, at least about 20 hours, at least about 24 hours, at least about 2 days, at least about 3 days, or more, or by a duration within a range defined by any two of the preceding time periods, for example 10-60 minutes, 1 hour-24 hours, 1-12 hours, 24-48 hours, 1-3 days.

In any production methods, in some embodiments, the method includes culturing a microbial cell genetically engineered with a nucleic acid or genetic vector encoding a fusion polypeptide as described herein under conditions sufficient to produce a circular bacteriocin. In some embodiments, the method includes culturing a second microbial cell in conjunction with the microbial cell genetically engineered with the nucleic acid or genetic vector encoding the fusion polypeptide. In some embodiments, the second microbial cell is an industrially useful microbial cell that is resistant to the circular bacteriocin.

In any production methods, in some embodiments, the method includes purifying the circular bacteriocin. In some embodiments, the circular bacteriocin is purified from the in vitro expression system. In some embodiments, the circular bacteriocin is purified after culturing the microbial cell genetically engineered with a nucleic acid or genetic vector encoding a fusion polypeptide as described herein. Any suitable option can be used to purify the circular bacteriocin. In some embodiments, the method includes purifying the fusion polypeptide, e.g., using an affinity tag associated therewith. In some embodiments, where the fusion polypeptide or the circular bacteriocin includes an affinity tag, the circular bacteriocin can be purified by contacting the fusion polypeptide or the circular bacteriocin with a support (e.g., a column, a bead, etc.) having a binding agent attached thereto, where the binding agent binds the affinity tag, and eluting the bound fusion polypeptide or circular bacteriocin. Any suitable affinity tag, such as those disclosed herein, can be used to purify the circular bacteriocin and/or the fusion polypeptide. In some embodiments, the affinity tag is CBP. In some embodiments, the affinity tag is CBP and purifying the circular bacteriocin and/or the fusion polypeptide includes using a chitin resin.

The contacting or culturing can be done under any suitable condition for producing the circular bacteriocin by the in vitro expression system or the genetically engineered microbial cell. In some embodiments, contacting the nucleic acid with the in vitro expression system involves incubating the nucleic acid in a transcription and/or translation solution at a suitable temperature. In some embodiments, the contacting is done at room temperature. In some embodiments, the contacting is done at less than 15° C., or about 15° C., about 18° C., about 20° C., about 22° C., about 25° C., about 27° C., about 30° C., about 34° C., about 36° C., about 38° C., or about 40° C., or higher, or at a temperature in a range defined by any two of the preceding values. In some embodiments, the culturing is done at a temperature suitable for growth of the genetically engineered microbial cell. In some embodiments, the culturing is done at less than 15° C., or about 15° C., about 18° C., about 20° C., about 22° C., about 25° C., about 27° C., about 30° C., about 34° C., about 36° C., about 38° C., or about 40° C., or higher, or at a temperature in a range defined by any two of the preceding values.

In any production methods, in some embodiments, contacting the nucleic acid with the in vitro expression system involves incubating the nucleic acid in a transcription and/or translation solution at a suitable pH. In some embodiments, the contacting is done at a pH of less than 3.0, or about 3.0, about 4.0, about 4.5, about 5.0, about 5.5, about 6.0, about 6.5, about 6.7, about 7.0, about 7.2, about 7.5, about 8.0, about 8.5, about 9.0, or about 10.0, or higher, or at a pH in a range defined by any two of the preceding values. In some embodiments, the culturing is done at a pH suitable for growth of the genetically engineered microbial cell. In some embodiments, the culturing is done at a pH of less than 3.0, or about 3.0, about 4.0, about 4.5, about 5.0, about 5.5, about 6.0, about 6.5, about 6.7, about 7.0, about 7.2, about 7.5, about 8.0, about 8.5, about 9.0, or about 10.0, or higher, or at a pH in a range defined by any two of the preceding values.

In any production methods, in some embodiments, where the split intein is a conditional intein, the method includes exposing the fusion polypeptide to the permissive condition, following exposure to the non-permissive condition, to induce circularization of the bacteriocin. In some embodiments, the method further includes modifying the temperature during (or after) the contacting or culturing. In some embodiments, where the split intein is temperature-sensitive, the method includes modifying the temperature, from a non-permissive temperature to a permissive temperature, or vice versa. In some embodiments, the method further includes shifting the pH during (or after) the contacting or culturing. In some embodiments, where the split intein is pH-sensitive, the method includes shifting the pH, from a pH which is non-permissive for circularization to a pH permissive for circularization, or vice versa.

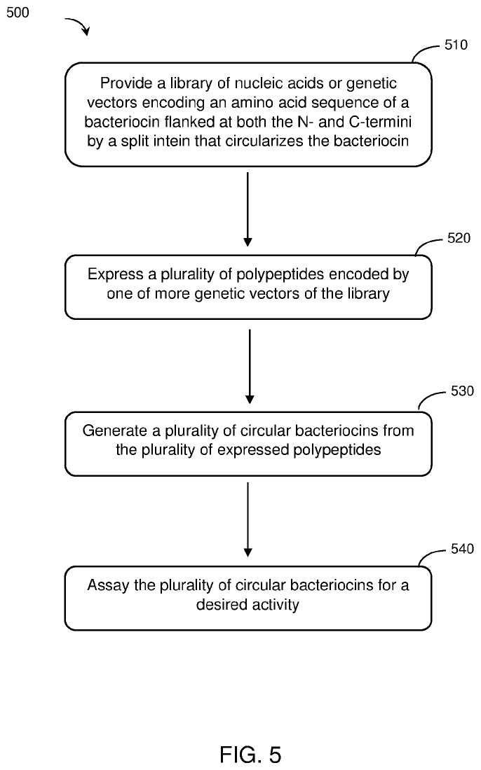

Also provided are methods of screening (which for convenience can be referred to as screening methods). With reference the FIG. 5, the method 500 can include providing a library of nucleic acids or genetic vectors of the present disclosure at block 510. The method can further include expressing a plurality of polypeptides encoded by one of more genetic vectors of the library, at block 520. The method can also include generating a plurality of circular bacteriocins from the plurality of expressed polypeptides, at block 530. Further, the method can include, at block 540, assaying the plurality of circular bacteriocins for a desired activity. The desired activity can be any suitable activity of the circular bacteriocins. In some embodiments, the desired activity is a change in activity relative to a reference, e.g., relative to the activity of a parent bacteriocin when screening a mutational library, or relative to a standard level of activity. In some embodiments, the desired activity is identification of an activity where none or substantially none was known previously, e.g., identifying a bacteriocin that is effective against a microbial species by screening a library of uncharacterized and/or predicted bacteriocins.

In any screening methods, in some embodiments, the desired activity includes antimicrobial activity. In some embodiments, the desired activity includes an increased antimicrobial activity, e.g., compared to the parent bacteriocin, against one or more microorganisms. In some embodiments, the desired activity includes antimicrobial activity against a specific species or strain of microorganism. In some embodiments, the desired activity includes resistance to degradation, such as, but not limited to, protease, heat, or pH degradation.

Also provided is a method of controlling the growth of a microorganism (which may be referred to herein as growth-controlling methods). In some embodiments, the method includes contacting a composition (e.g., culture medium, feedstock, a microbiome, etc.) that includes a microorganism (e.g., an undesirable microorganism) with the genetically engineered microbial cell of the present disclosure under conditions sufficient for the genetically engineered microbial cell to produce the circular bacteriocin, to inhibit or slow the growth of the microorganism. In some embodiments, the method includes contacting a composition (e.g., culture medium, feedstock, a microbiome, etc.) that is conducive to supporting the growth of a microorganism (e.g., an undesirable microorganism) with the genetically engineered microbial cell of the present disclosure under conditions sufficient for the genetically engineered microbial cell to produce the circular bacteriocin, to prevent the growth or delay the appearance of the microorganism in the composition.

In some embodiments, the method includes contacting a composition (e.g., culture medium, feedstock, a microbiome, etc.) that includes a microorganism (e.g., an undesirable microorganism) with a circular bacteriocin made by a production method, as disclosed herein, to inhibit or slow the growth of the microorganism. In some embodiments, the method includes contacting a composition (e.g., culture medium, feedstock, a microbiome, etc.) that is conducive to supporting the growth of a microorganism (e.g., an undesirable microorganism) with a circular bacteriocin made by a production method, as disclosed herein, to prevent the growth or delay the appearance of the microorganism in the composition. In some embodiments, the method includes contacting a composition (e.g., culture medium, feedstock, a microbiome, etc.) that includes a microorganism (e.g., an undesirable microorganism) with a fusion polypeptide, as disclosed herein, to inhibit or slow the growth of the microorganism. In some embodiments, the method includes contacting a composition (e.g., culture medium, feedstock, a microbiome, etc.) that is conducive to supporting the growth of a microorganism (e.g., an undesirable microorganism) with a fusion polypeptide, as disclosed herein, to prevent the growth or delay the appearance of the microorganism in the composition.

In any growth-controlling methods, in some embodiments, the microorganism targeted by the circular bacteriocin is any suitable microorganism for which restricting or preventing growth is desirable. In some embodiments, the microorganism is a bacteria. In some embodiments, the microorganism is a pathogenic microorganism.

In any growth-controlling methods, in some embodiments, the composition can be associated with any environment in which controlling the growth of microorganisms is desired. In some embodiments, the composition includes, without limitation, a culture medium, feedstock, or a microbiome. The microbiome can include any suitable collection of microorganisms associated with an environment. In some embodiments, the microbiome includes that of an animal, a human organ, a plant, a plant root, and/or soil. In some embodiments, the microbiome includes that of a subject, such as a skin, gut, gastrointestinal tract, mammary gland, placenta, tissue, biofluid, seminal fluid, uterus, vagina, ovarian follicle, lung, saliva, oral cavity, mucosa, conjunctiva, or biliary tract. In some embodiments, the composition is associated with a commercially relevant environment, such as, without limitation, an industrial feedstock, or in a fermenter, or in a food, pharmaceutical, or cosmetic manufacturing environment.

In any growth-controlling methods, in some embodiments, where the split intein is a conditional intein, the method includes exposing the fusion polypeptide to the permissive condition, following exposure to the non-permissive condition, to induce circularization of the bacteriocin. In any growth-controlling methods, in some embodiments, the method includes modifying the pH or temperature of the composition to induce circularization of the bacteriocin, where the split intein is pH- or temperature-sensitive, respectively, as disclosed herein. In some embodiments, the method includes modifying the temperature of the composition from a non-permissive temperature or pH to a permissive temperature or pH, respectively, to induce circularization of the bacteriocin.

Also provided is a method of designing a nucleic acid encoding a polypeptide precursor of a bacteriocin (which for convenience may be referred to as designing methods). The method can include identifying a native amino acid sequence of a candidate bacteriocin, wherein the native amino acid sequence does not comprise a serine or cysteine at the N-terminus; providing a second amino acid sequence having a serine or cysteine at the N-terminus thereof by at least one of: circularly permuting the native amino acid sequence; or introducing a non-native serine or cysteine to the native amino acid sequence; providing a nucleotide sequence encoding a polypeptide comprising the second amino acid sequence flanked at both the N- and C-termini by a split intein configured to circularize the bacteriocin; and expressing the polypeptide encoded by the nucleotide sequence. In some embodiments, the candidate bacteriocin is a bacteriocin that is predicted to be a circular bacteriocin, e.g., based on the sequence of the bacteriocin or the genomic context. The polypeptide comprising the second amino acid sequence flanked at both the N- and C-termini by a split intein configured to circularize the bacteriocin can be any suitable polypeptide, e.g., a fusion polypeptide as disclosed herein. In some embodiments, the candidate bacteriocin is one that is predicted to be a circular bacteriocin based on a genomic sequence of a microorganism that encodes the candidate bacteriocin in its genome. In some embodiments, introducing a non-native serine or cysteine to the native amino acid sequence includes substituting a native amino acid residue with a serine or cysteine, or adding or inserting a serine or cysteine to the native amino acid sequence.

In some embodiments, the nucleic acids encoding a polypeptide precursor of a bacteriocin finds use in generating a library of candidate bacteriocins for screening. In any designing methods, in some embodiments, the method includes: identifying a plurality of native amino acid sequences of a plurality of different candidate bacteriocins; for each of the plurality of native amino acid sequences: providing the second amino acid sequence; and providing the nucleotide sequence encoding a polypeptide comprising the second amino acid sequence flanked at both the N- and C-termini by a split intein configured to circularize the bacteriocin. In some embodiments, nucleic acids or genetic vectors that include the nucleotide sequences encoding the polypeptide can be provided in any suitable library, including a library as disclosed herein.

In some embodiments, the polypeptide further includes a degradation tag as disclosed herein. In some embodiments, the polypeptide further comprises a signal peptide and/or leader sequence as disclosed herein.

The split intein can be any suitable split intein as described herein. Expressing the polypeptide encoded by the nucleotide sequence can be done using any suitable option. In some embodiments, the polypeptide encoded by the nucleotide sequence is expressed in an in vitro expression system, as provide herein. In some embodiments, the polypeptide encoded by the nucleotide sequence is expressed by a microbial cell genetically engineered with a nucleic acid having the nucleotide sequence.

### Kits

Also provided is a kit for generating a circular bacteriocin. In some embodiments, the kit includes a fusion polypeptide of the present disclosure. In some embodiments, the kit includes: a lyophilized composition of a fusion polypeptide of the present disclosure; and a liquid (e.g., water or buffer) for reconstituting the lyophilized composition. In some embodiments, the kit includes a panel of fusion polypeptides as disclosed herein having different bacteriocin sequences. In some embodiments, the kit includes a nucleic acid or genetic vector that encodes the fusion polypeptide as disclosed herein. In some embodiments, the kit includes a library of nucleic acids or genetic vectors encoding a plurality of fusion polypeptides as disclosed herein having different bacteriocin sequences. In some embodiments, the kit includes: a nucleic acid or genetic vector that encodes the fusion polypeptide as disclosed herein; and an in vitro transcription solution (or one or more components thereof), or an in vitro transcription solution (or one or more components thereof) and an in vitro translation solution (or one or more components thereof). In some embodiments, the kit includes a microbial cell genetically engineered with the nucleic acid or genetic vector that encodes the fusion polypeptide, as disclosed herein. In some embodiments, the kit includes an indicator strain of microorganism that is known to be susceptible to the circular bacteriocin generated by the kit. In some embodiments, the kit further comprises instructions for generating the circular bacteriocin from the fusion polypeptide, nucleic acid, genetic vector, or genetically engineered microbial cell.

### ADDITIONAL EMBODIMENTS

Examples 1-3 below demonstrate circularization of bacteriocins from a fusion polypeptide that includes the bacteriocin flanked by a split intein. Circular bacteriocins are promising groups of antimicrobial peptides for industrial applications due to their higher stability compared to their linear counterparts. These peptides are, in general, more resistant to proteolytic enzymes and able to retain their full activity at different pH or temperatures. Until now, circular bacteriocins remained as a quite selective group with just 20 candidates discovered and fully characterized. Thanks to the recent advances in the omics technologies, especially in genomic sequencing, a high number of previously undescribed putatively circular bacteriocin clusters have been recently identified in different genomes of Gram positive bacteria from general databases, showing that these peptides are probably more prevalent in nature than previously appreciated. However, in some cases, in the absence of the producer strains it is difficult to predict the real functionality of these genes/clusters found.

Synthetic biology tools can be used for the fast and easy production of bacteriocins. Using cell-free protein synthesis (CFPS) techniques a collection of different mature bacteriocins, both fully characterized as hypothetical non-characterized candidates can be produced [13]. With just one single gene coding for the mature peptide (without the signal peptide or leader sequence), CFPS allows the production of different active bacteriocins outside the bacterial host in less than 4 hours. Due to the fact that protein production occurs outside the cell, there is no need for further leader sequence/signal peptide cleavage nor transport from dedicated proteins. Although this technique is suitable for the production of non-modified class II bacteriocins, production of bacteriocins requiring post-translational modifications (class I) may not be as efficient, without the activity of other dedicated proteins involved in the maturation of the peptides in the native bacterial host. Circular bacteriocins can be included in this last group, where several proteins are known to be involved in maturation (cleavage/circularization) and secretion outside the cell via different dedicated transporter systems in the native bacterial host.

Examples 1-3 below show that circularization of bacteriocins using split-inteins. Inteins can be used with tags for column purification or protein degradation. Split-intein mediated circular ligation of peptides and proteins (SICLOPPS) that incorporates different improvements, such as the use of an intein from Nostoc punctiforme (Npu), which is faster and also significantly more tolerant of amino acid diversity in the extein sequence and also a Ssra sequence in C-terminus to reduce the toxic effects of Npu by directing the Ssra-tagged protein to the ClpXP machinery for degradation [16] were used to circularize bacteriocins.

SICLOPPS was tested with Garvicin ML, a known circular bacteriocin from Lactococus garvieae DCC43, a strain isolated from Mallard Ducks [6]. It has been demonstrated that splicing with Npu intein is more efficient when a Cys or a Ser are put in position +1. Ser32 was selected over the other two serines present in GarML, but the other serines could have been chosen. As for other bacteriocins produced by CFPS, the recombinant gene was put under the control of a T7 promoter and terminator sequence [13]. After in vitro production antimicrobial activity was only observed when using the intact SICLOPPS construct, but not in the construct not carrying inteins (linear GarML) or in the construct with two point mutations in the inteins (preventing splicing). A higher activity was observed when the reaction was left overnight at room temperature. Splicing by Npu has been estimated to occur within the first 30 to 60 seconds, therefore the higher activity observed after 24 h is not likely to be explained by higher percentage of splicing over time. Instead this higher activity could be due to a higher production of recombinant protein by the in vitro reaction. It has been observed that in some cases, leaving the in vitro reaction longer than 2 hours, can benefit protein production. These results demonstrated that SICLOPPS was working and GarML had been produced and circularized spontaneously without any further accessory protein. In order to confirm the presence of spliced GarML after CFPS a MS analysis of the reaction was run, and a peak with a mass of 6004 Da, corresponding to the exact mass of original GarML, was observed, confirming correct splicing of the inteins from GarML.

Correct circularization of GarML was further confirmed by bringing the SICLOPPS construct to E. coli and producing recombinant GarML in vivo for higher scale protein production. After protein production and purification a single FPLC fraction with antimicrobial activity was obtained. MS analysis of this fractions revealed again a mass of 6004 Da, thus confirming the results previously obtained with the CFPS method and showing that splicing was also taking place in E. coli. Moreover, trypsin digestion of the purified peptide and LC-MS/MS analysis of the fragments obtained revealed the presence of 6 peptides covering the 100% of mature GarML. The presence in one of the peptide (TIVNAVSAGMDIATALSLFSGAFTAAGGIMALIK) of S1 and F60 linked together confirmed correct circularization of GarML in the selected location. These results show that GarML produced with SICLOPPS system not only circularizes and has the exact same sequence as GarML produced by its native producer, but that it most likely adopts the same three-dimensional conformation. This was also observed during the construction of the PARAGEN collection, where different pediocin-like bacteriocins, having 2 or 3 disulfide bonds in their native and active final conformation, were active after in vitro production and showed a similar spectrum of activity as the native ones. This is the first time that a circular bacteriocin is produced in E. coli. Some options to enhance production and facilitate purification might include the use of a different host for protein production, the addition of an immunity gene to the construct in order to prevent toxic effects of the bacteriocin, fusion of a signal peptide to the protein to promote secretion outside the cell, use of switchable inteins for conditional protein splicing or use of fusion tags to facilitate column purification.

Last, use of SICLOPPS for circularization of other circular bacteriocins was determined. The list included some fully characterized candidates such as enterocin AS-48 or gassericin A, two well studied bacteriocins potential as food biopreservatives or prebiotic compounds in animal health is well documented. Other bacteriocins selected in this study correspond to putative circular bacteriocins found in genome mining studies, but not yet confirmed in the lab. After CFPS most of SICLOPPS was observed to produce bacteriocins showing activity against at least one of the indicators used in the study, thus showing that, most likely, splicing and circularization of these bacteriocins was happening.

Examples 1-3 demonstrate that circularization of bacteriocins using split-inteins allow the fast production and circularization of bacteriocins, ready to be tested for antimicrobial activity. This is the first time that production and circularization of bacteriocins is carried out using split-inteins, the first time they are produced using CFPS and also the first time a functional circular bacteriocin is produced by E. coli.

Examples 1-3 below demonstrate an efficient synthetic biology method to carry out circularization of many bacteriocins, even in the absence of the original producer strain. This method also simplifies production of circular bacteriocins as just one single gene is necessary for production and circularization. This work provides for use of inteins with bacteriocins, including:
For production, circularization and functional test of other hypothetical circular bacteriocins.
For production and circularization of non-circular (linear) bacteriocins to enhance stability.
For the generation of circular bacteriocin libraries to test and obtain variants with enhanced features.
For heterologous production of circular bacteriocins in other host for scaling up bacteriocin production.

### REFERENCES

[1] M. Younes et al., “Safety of nisin (E 234) as a food additive in the light of new toxicological data and the proposed extension of use,” EFSA J., vol. 15, no. 12, December 2017.
[2] S. Soltani et al., “Bacteriocins as a new generation of antimicrobials: toxicity aspects and regulations,” FEMS Microbiol. Rev., vol. 45, no. 1, January 2021.
[3] M. L. Chikindas, R. Weeks, D. Drider, V. A. Chistyakov, and L. M. Dicks, “Functions and emerging applications of bacteriocins,” Curr. Opin. Biotechnol., vol. 49, p. 23, February 2018.
[4] M. Zimina et al., “Overview of Global Trends in Classification, Methods of Preparation and Application of Bacteriocins,” Antibiot. 2020, Vol. 9, Page 553, vol. 9, no. 9, p. 553, August 2020.
[5] P. D. Cotter, R. P. Ross, and C. Hill, “Bacteriocins—a viable alternative to antibiotics?,” Nat. Rev. Microbiol., vol. 11, no. 2, pp. 95-105, February 2013.
[6] J. Borrero et al., “Characterization of garvicin ML, a novel circular bacteriocin produced by Lactococcus garvieae DCC43, isolated from mallard ducks (Anas platyrhynchos).,” Appl. Environ. Microbiol., vol. 77, no. 1, pp. 369-373, January 2011.
[7] P. Alvarez-Sieiro, M. Montalbán-López, D. Mu, and O. P. Kuipers, “Bacteriocins of lactic acid bacteria: extending the family,” Appl. Microbiol. Biotechnol., vol. 100, no. 7, pp. 2939-2951, April 2016.
[8] J. Borrero et al., “Plantaricyclin A, a Novel Circular Bacteriocin Produced by Lactobacillus plantarum NI326: Purification, Characterization, and Heterologous Production.” Appl. Environ. Microbiol., vol. 84, no. 1, January 2018.
[9] R. H. Perez, T. Zendo, and K. Sonomoto, “Circular and Leaderless Bacteriocins: Biosynthesis, Mode of Action, Applications, and Prospects.,” Front. Microbiol., vol. 9, p. 2085, 2018.
[10] D. Major, L. Flanzbaum, L. Lussier, C. Davies, K. M. P. Caldo, and J. Z. Acedo, “Transporter Protein-Guided Genome Mining for Head-to-Tail Cyclized Bacteriocins,” Molecules, vol. 26, no. 23, December 2021.
[11] B. Xin et al., “In Silico Analysis Highlights the Diversity and Novelty of Circular Bacteriocins in Sequenced Microbial Genomes,” mSysterns, vol. 5, no. 3, June 2020.
[12] B. Vezina, B. H. A. Rehm, and A. T. Smith, “Bioinformatic prospecting and phylogenetic analysis reveals 94 undescribed circular bacteriocins and key motifs.,” BMC Microbiol., vol. 20, no. 1, p. 77, April 2020.
[13] P. Gabant and J. Borrero, “PARAGEN 1.0: A Standardized Synthetic Gene Library for Fast Cell-Free Bacteriocin Synthesis.,” Front. Bioeng. Biotechnol., vol. 7, p. 213, 2019.
[14] C. P. Scott, E. Abel-Santos, M. Wall, D. C. Wahnon, and S. J. Benkovic, “Production of cyclic peptides and proteins in vivo,” Proc. Natl. Acad. Sci. U.S.A., vol. 96, no. 24, pp. 13638-13643, November 1999.
[15] A. Tavassoli and S. J. Benkovic, “Split-intein mediated circular ligation used in the synthesis of cyclic peptide libraries in E. coli.,” Nat. Protoc., vol. 2, no. 5, pp. 1126-1133, 2007.
[16] J. E. Townend and A. Tavassoli, “Traceless Production of Cyclic Peptide Libraries in E. coli.,” ACS Chem. Biol., vol. 11, no. 6, pp. 1624-1630, June 2016.
[17] M. Cheriyan, C. S. Pedamallu, K. Tori, and F. Perler, “Faster protein splicing with the Nostoc punctiforme DnaE intein using non-native extein residues,” J. Biol. Chem., vol. 288, no. 9, pp. 6202-6211, March 2013.

### EXAMPLES

The following materials and methods were used in Examples 1-4.

### Bacterial Strains, Plasmids and Culture Conditions

For GarML production the microbial strains and plasmids used in this study are listed in Table 1.

TABLE 1

| | | | Source and/or |
|---|---|---|---|
| | Strain or plasmid | Description | reference^{a} |
| Strains | Escherichia coli DH5α | Stabilization of recombinant plasmids | ThermoFisher |
| | Escherichia coli BL21 | Production of Npu-GarvML | ThermoFisher |
| | Lactococcus garvieae | Garvicin ML producer | Borrero et |
| | DCC43 | | al., 2007 |
| | Lactococcus garvieae | Indicator sensitive to GarML | CECT |
| | 5806 | | |
| | Lactococcus lactis | Indicator sensitive to different | Chopin et al., |
| | IL1403 | bacteriocins | 1984^{b} |
| Plasmids | pUC-Npu-GarML | Ampr; T7 Plasmid carrying I_{C} - | Genewiz |
| | | garML - I_{N} - SsrA | |
| | pUC-GarML | Ampr; T7 Plasmid carrying garML | Genewiz |
| | pUC-Npu⁻-GarML | Ampr; T7 Plasmid carrying I_{C}⁻ - | Genewiz |
| | | garML - I_{N}⁻ - SsrA | |

^{a}CECT, Colección Española de Cultivos Tipo.
^{b}A. Chopin, M. C. Chopin, A. Moillo-Batt, and P. Langella, “Two plasmid-determined restriction and modification systems in Streptococcus lactis.,” Plasmid, vol. 11, no. 3, pp. 260-263, May 1984.

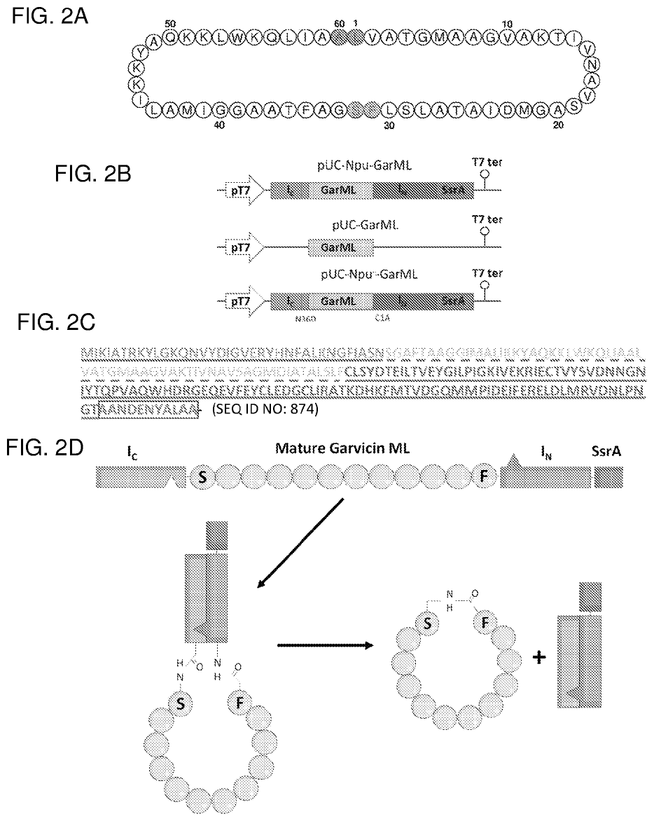

A schematic view of the design of the plasmids is shown in FIGS. 2A-2D. All amino acid sequences of the fusion polypeptides are shown in Table 2.1. All amino acid sequences, both native and as modified for use with SICLOPPS (split-intein circular ligation of peptides and proteins), of the characterized and uncharacterized circular bacteriocins with the SICLOPPS method and the controls are shown in Table 2.2. For plasmid construction, all amino acidic sequences were reverse-translated and codon optimized for Escherichia coli (world wide web at bioinformatics.org/sms2/rev_trans.html). The nucleotide sequences were included in a vector backbone containing the T7 promoter region, a start codon (ATG) a stop codon (TAA) and a T7 terminator region. Plasmid synthesis was carried out by Genewiz (New Jersey, USA).

TABLE 2.1

| Bacteriocin | Strain | Mature Sequence | Reference |
|---|---|---|---|
| Amylocyclicin | Bacillus | LASTLGISTAAAKKAIDIIDAASTIASIISLIGIVTGAGA | Scholz et al., |
| (Alc) | amyloliquefaciens | ISYAIVATAKTMIKKYGKKYAAAW | 2014 |
| | FZB42 | (SEQ ID NO: 811) | |
| | | STAAAKKAIDIIDAASTIASIISLIGIVTGAGAISYAIVA | |
| | | TAKTMIKKYGKKYAAAWLASTLGI | |
| | | (SEQ ID NO: 812) | |
| Bacteriocin | Staphylococcus | LTSTLGISSYAAKKAIDIIAAAGDVAAIVGLIGAVTGA | Vezina et al., |
| 3688STDY612 | aureus | GAIGAGILFTAKKLIKSYGKKYAAAW | 2020 |
| 4959 | 3688STDY6124959 | (SEQ ID NO: 813) | |
| | | STLGISSYAAKKAIDIIAAAGDVAAIVGLIGAVTGAG | |
| | | AIGAGILFTAKKLIKSYGKKYAAAWLT | |
| | | (SEQ ID NO: 814) | |
| Enterocin AS- | Enterococcus | MAKEFGIPAAVAGTVLNVVEAGGWVTTIVSILTAV | Galvez et al., |
| 48 (EntAS48) | faecalis S-48 | GSGGLSLLAAAGRESIKAYLKKEIKKKGKRAVIAW | 1989 |
| | | (SEQ ID NO: 815) | |
| | | SIKAYLKKEIKKKGKRAVIAWMAKEFGIPAAVAGTV | |
| | | LNVVEAGGWVTTIVSILTAVGSGGLSLLAAAGRE | |
| | | (SEQ ID NO: 816) | |
| Bacteriocin | Listeria | LAKEFGIPGGVAATVLNIVEAGGWATTILSILTAVG | Vezina et al., |
| BCW 2997 | monocytogenes | SGGLSLIAAAGKESIKVYLKKQIKKKGRKAVIAW | 2020 |
| | BCW 2997 | (SEQ ID NO: 817) | |
| | | SIKVYLKKQIKKKGRKAVIAWLAKEFGIPGGVAATV | |
| | | LNIVEAGGWATTILSILTAVGSGGLSLIAAAGKE | |
| | | (SEQ ID NO: 818) | |
| Bacteriocin | Clavibacter | LAQQFGIPSAVGGTILNLATAGGTAATIVGILVGLG | Vezina et al., |
| CF11 | michiganensis | SGGLGLLAAAGRETIKQFLLNEIKKKGRKAVIAW | 2020 |
| | CF11 | (SEQ ID NO: 819) | |
| | | SAVGGTILNLATAGGTAATIVGILVGLGSGGLGLLA | |
| | | AAGRETIKQFLLNEIKKKGRKAVIAWLAQQFGIP | |
| | | (SEQ ID NO: 820) | |
| Bacteriocin | Paenibacillus | LAKEFGVPAVVGNALVQALDWGLTAATIASIIGAFI | Vezina et al., |
| NBRC 15376 | chondroitinus | TGGLSIVAAAGTTALKTYLRNKLAEMGTKAFVAW | 2020 |
| | NBRC 15376 | (SEQ ID NO: 821) | |
| | | SIIGAFITGGLSIVAAAGTTALKTYLRNKLAEMGTKA | |
| | | FVAWLAKEFGVPAVVGNALVQALDWGLTAATIA | |
| | | (SEQ ID NO: 822) | |
| Carnocyclin A | Carnobacterium | LVAYGIAQGTAEKVVSLINAGLTVGSIISILGGVTVG | Leah et al., |
| | maltaromaticum | LSGVFTAVKAAIAKQGIKKAIQL | 2008 |
| | UAL307 | (SEQ ID NO: 823) | |
| | | SLINAGLTVGSIISILGGVTVGLSGVFTAVKAAIAKQ | |
| | | GIKKAIQLLVAYGIAQGTAEKVV | |
| | | (SEQ ID NO: 824) | |
| Circularin A | Clostridium | VAGALGVQTAAATTIVNVILNAGTLVTVLGIIASIAS | Kemperman et |
| (CirA) | beijerinckii ATCC | GGAGTLMTIGWATFKATVQKLAKQSMARAIAY | al., 2003 |
| | 25752 | (SEQ ID NO: 825) | |
| | | SIASGGAGTLMTIGWATFKATVQKLAKQSMARAIA | |
| | | YVAGALGVQTAAATTIVNVILNAGTLVTVLGIIA | |
| | | (SEQ ID NO: 826) | |
| Bacteriocin | Streptococcus suis | VAAALGVPSAVASTVVNVILGAGTAVTILGIISSIAS | Vezina et al., |
| YS111 | YS111 | GGATTLLTVGWAGFKATVQRLAKQSMARAIAY | 2020 |
| | | (SEQ ID NO: 827) | |
| | | STVVNVILGAGTAVTILGIISSIASGGATTLLTVGWA | |
| | | GFKATVQRLAKQSMARAIAYVAAALGVPSAVA | |
| | | (SEQ ID NO: 828) | |
| Enterocin | Enterococcus | LTANLGISSYAAKKVIDIINTGSAVATIIALVTAVVGG | Himeno et al., |
| NKR-5-3B | faecium NKR-5-3 | GLITAGIVATAKSLIKKYGAKYAAAW | 2015 |
| | | (SEQ ID NO: 829) | |
| | | SSYAAKKVIDIINTGSAVATIIALVTAVVGGGLITAGI | |
| | | VATAKSLIKKYGAKYAAAWLTANLGI | |
| | | (SEQ ID NO: 830) | |
| Bacteriocin | Garciella | LASMLGISTVAAKKVIDIIDTASTVATIISLIGVIVGVG | Vezina et al., |
| DSM 15102 | nitratireducens | GITAGLVTTAKAMIKKYGKKYATMW | 2020 |
| | DSM 15102 | (SEQ ID NO: 831) | |
| | | SMLGISTVAAKKVIDIIDTASTVATIISLIGVIVGVGGI | |
| | | TAGLVTTAKAMIKKYGKKYATMWLA | |
| | | (SEQ ID NO: 832) | |
| Garvicin ML | Lactococcus | LVATGMAAGVAKTIVNAVSAGMDIATALSLFSGAF | Borrero et al., |
| (GarML) | garvieae DCC43 | TAAGGIMALIKKYAQKKLWKQLIAA | 2011 |
| | | (SEQ ID NO: 833) | |
| | | SGAFTAAGGIMALIKKYAQKKLWKQLIAALVATGM | |
| | | AAGVAKTIVNAVSAGMDIATALSLF | |
| | | (SEQ ID NO: 834) | |
| Leucocyclicin | Leuconostoc | LVNQLGISKSLANTILGAIAVGNLASWLLALVPGPG | Masuda et al., |
| Q | mesenteroides | WATKAALATAETIVKHEGKAAAIAW | 2011 |
| | TK41401 | (SEQ ID NO: 835) | |
| | | SKSLANTILGAIAVGNLASWLLALVPGPGWATKAA | |
| | | LATAETIVKHEGKAAAIAWLVNQLGI | |
| | | (SEQ ID NO: 836) | |
| Bacteriocin | Bacillus | LINKLGISKSLAQDILTAIAVGNVASWLLVLIPGPGWATK | Vezina et al., |
| AFS089278 | toyonensis | AAISAAEIIVETSGEAAAVAY | 2020 |
| | AFS089278 | (SEQ ID NO: 837) | |
| | | SKSLAQDILTAIAVGNVASWLLVLIPGPGWATKAAISAA | |
| | | EIIVETSGEAAAVAYLINKLGI | |
| | | (SEQ ID NO: 838) | |
| Uberolysin A | Streptococcus | LAGYTGIASGTAKKVVDAIDKGAAAFVIISIISTVISAGALG | Wirawan et al., |
| (UberA) | uberis 42 | AVSASADFIILTVKNYISRNLKAQAVIW | 2007 |
| | | (SEQ ID NO: 839) | |
| | | SGTAKKVVDAIDKGAAAFVIISIISTVISAGALGAVSASAD | |
| | | FIILTVKNYISRNLKAQAVIWLAGYTGIA | |
| | | (SEQ ID NO: 840) | |
| Bacteriocin | Bacillus | VAGVLSVDKGVATKVVNAVDAAGWAFVGGSTLLAIISG | Vezina et al., |
| TD3 | vallismortis TD3 | GTLSAASISIDALIYTIKNYLKRSLKAQAIAW | 2020 |
| | | (SEQ ID NO: 841) | |
| | | SVDKGVATKVVNAVDAAGWAFVGGSTLLAIISGGTLSA | |
| | | ASISIDALIYTIKNYLKRSLKAQAIAWVAGVL | |
| | | (SEQ ID NO: 842) | |
| Bacteriocin | Streptomyces | ATTPNVLTGYFGLTGATANRVLNAIEAGTDIAAALSVLG | Vezina et al., |
| NRRL B-24287 | pathocidini NRRL | GVSAAGGVAMWMLKQAIAKGGRKAVVA | 2020 |
| | B-24287 | (SEQ ID NO: 843) | |
| | | SVLGGVSAAGGVAMWMLKQAIAKGGRKAVVAATTPN | |
| | | VLTGYFGLTGATANRVLNAIEAGTDIAAAL | |
| | | (SEQ ID NO: 844) | |
| Bacteriocin | Alkalibacterium | ILFVADLFGIPFTTDIVDQVWGYIEGGAGTVTAISWVLG | Vezina et al., |
| AK22 | AK22 | VTIPAWAVPIIGAVGVVSA | 2020 |
| | | (SEQ ID NO: 845) | |
| | | SWVLGVTIPAWAVPIIGAVGVVSAILFVADLFGIPFTTDI | |
| | | VDQVWGYIEGGAGTVTAI | |
| | | (SEQ ID NO: 846) | |
| Bacteriocin | Gemella cuniculi | VYFIAGKLGITLAPGWYEKLVNYISAGGGAVDGVAMILG | Vezina et al., |
| 15828 | DSM 15828 | ITLPGWAITVIGAFGLVSA | 2020 |
| | | (SEQ ID NO: 847) | |
| | | SAGGGAVDGVAMILGITLPGWAITVIGAFGLVSAVYFIA | |
| | | GKLGITLAPGWYEKLVNYI | |
| | | (SEQ ID NO: 848) | |
| Butyrivibriocin | Butyrivibrio | IYFIADKMGIQLAPAWYQDIVNWVSAGGTLTTGFAIIVG | Kalmokoff et |
| AR10 | fibrisolvens AR10 | VTVPAWIAEAAAAFGIASA | al., 2000 |
| | | (SEQ ID NO: 849) | |
| | | SAGGTLTTGFAIIVGVTVPAWIAEAAAAFGIASAIYFIADK | |
| | | MGIQLAPAWYQDIVNWV | |
| | | (SEQ ID NO: 850) | |
| Paracyclicin P | Lactobacillus. | IYFIANKLGIHLAPGWYQDMVNYVSAGGSLAGAFSVVA | Collins et al., |
| (ParP) | Paracesei subsp. | GVTLPAWIVPIATAFGAVSA | 2017 |
| | Paracasei JCM | (SEQ ID NO: 851) | |
| | 8130/ | SAGGSLAGAFSVVAGVTLPAWIVPIATAFGAVSAIYFIAN | |
| | DSM 5622 | KLGIHLAPGWYQDMVNYV | |
| | | (SEQ ID NO: 852) | |
| Bacteriocin | Streptococcus | IYWICGKFGIRLAPGWYQDIVDFVSAGGSIVDAFAIIAGI | Vezina et al., |
| NCTC 12958 | thermophilus | TLPAWVAPVLAGFGVVSA | 2020 |
| | NCTC 12958 | (SEQ ID NO: 853) | |
| | | SAGGSIVDAFAIIAGITLPAWVAPVLAGFGVVSAIYWICG | |
| | | KFGIRLAPGWYQDIVDFV | |
| | | (SEQ ID NO: 854) | |
| Bacteriocin | Streptococcus | IYWVCGKFGIELAPGWYQDIVDFVSSGGTIVEAFAAVAG | Vezina et al., |
| UoS2029 | pneumoniae | VTLPAWVGPVLAAFGLASA | 2020 |
| | UoS2029 | (SEQ ID NO: 855) | |
| | | SSGGTIVEAFAAVAGVTLPAWVGPVLAAFGLASAIYWV | |
| | | CGKFGIELAPGWYQDIVDFV | |
| | | (SEQ ID NO: 856) | |
| Bacteriocin | Staphylococcus | VTWVLDKFGIKIGTPTINEIINYVANGGAIVTAFAVIAGIT | Vezina et al., |
| F9_V1* | felis F9 | LPAWVGPAVAAFATTAA | 2020 |
| | | (SEQ ID NO: 857) | |
| | | SVTWVLDKFGIKIGTPTINEIINYVANGGAIVTAFAVIAGI | |
| | | TLPAWVGPAVAAFATTAA | |
| | | (SEQ ID NO: 858) | |
| Bacteriocin | Staphylococcus | VTWVLDKFGIKIGTPTINEIINYVANGGAIVTAFAVIAGIT | Vezina et al., |
| F9_V2* | felis F9 | LPAWVGPAVAAFATTAA | 2020 |
| | | (SEQ ID NO: 859) | |
| | | SAVTWVLDKFGIKIGTPTINEIINYVANGGAIVTAFAVIAG | |
| | | ITLPAWVGPAVAAFATT | |
| | | (SEQ ID NO: 860) | |
| Gassericin A | Lactobacillus | IYWIADQFGIHLATGTARKLLDAMASGASLGTAFAAILG | Toba et al., |
| (GasA) | gasseri LA39 | VTLPAWALAAAGALGATAA | 1991 |
| | | (SEQ ID NO: 861) | |
| | | SGASLGTAFAAILGVTLPAWALAAAGALGATAAIYWIAD | |
| | | QFGIHLATGTARKLLDAMA | |
| | | (SEQ ID NO: 862) | |
| Plantaricyclin | Lactobacillus | IVWIAKQFGVHLTTSLTQKALDLLSAGSSLGTVAAAVLG | Borrero et al., |
| A (PIcA) | plantarum NI326 | VTLPAWAVAAAGALGGTAA | 2017 |
| | | (SEQ ID NO: 863) | |
| | | SLTQKALDLLSAGSSLGTVAAAVLGVTLPAWAVAAAGA | |
| | | LGGTAAIVWIAKQFGVHLTT | |
| | | (SEQ ID NO: 864) | |
| Cerecyclin | Bacillus sp. Xin1 | VVSKLGWTGINIGTANALIGALMTGSDIWTAISVAGLAF | Xin et al., 2020 |
| | | GGGIGTAISTIGRKAIMEMVEKVGKKKAAQW | |
| | | (SEQ ID NO: 865) | |
| | | SKLGWTGINIGTANALIGALMTGSDIWTAISVAGLAFGG | |
| | | GIGTAISTIGRKAIMEMVEKVGKKKAAQWVV | |
| | | (SEQ ID NO: 866) | |
| Bacteriocin | Paeniclostridium | IYFIADKMGITLSTKMYRDIVDAVSAGGSIAGAFSLI | Vezina et al., |
| R26833 | sordellii R26833 | VGVTLPGWLVVAAAGFGLTSA | 2020 |
| | | (SEQ ID NO: 867) | |
| | | STKMYRDIVDAVSAGGSIAGAFSLIVGVTLPGWLV | |
| | | VAAAGFGLTSAIYFIADKMGITL | |
| | | (SEQ ID NO: 868) | |

List of bacteriocins used in this study. Bacteriocins have been grouped according to the classification made by Vezina et al., 2020. In bold are the name of those bacteriocins fully characterized. The first line of the mature amino acidic sequence corresponds to the described or hypothetical linear sequence originated after leader sequence cleavage and before head-to-tail circularization. The second line corresponds to the amino acidic sequence used in this study for circularization using the SICCLOPPS system. The serines used in position 1 are bolded and underlined in the original sequence. Bacteriocin F9 has no serine in its original amino acidic sequence. A serine was added in position one (bolded).

TABLE 2.2

| Fragment | Description | Mature Sequence | Reference |
|---|---|---|---|
| I_{C} | C-terminal intein fragment from | MIKIATRKYLGKQNVYDIGVERYHNFALKN | Iwai et |
| | Nostoc punctifrome (Npu) DnaE | GFIASN | al., 2006 |
| | split intein | (SEQ ID NO: 869) | |
| I_{N} | N-terminal intein fragment from | CLSYDTEILTVEYGILPIGKIVEKRIECTVYSVD | Iwai et |
| | Nostoc punctifrome (Npu) DnaE | NNGNIYTQPVAQWHDRGEQEVFEYCLEDG | al., 2006 |
| | split intein | CLIRATKDHKFMTVDGQMMPIDEIFERELD | |
| | | LMRVDNLPNGT | |
| | | (SEQ ID NO: 870) | |
| I_{C}⁻ | C-terminal intein fragment from | MIKIATRKYLGKQNVYDIGVERYHNFALKN | Cheriyan et |
| (N36D) | Nostoc punctifrome (Npu) DnaE | GFIASD | al., 2013 |
| | split intein with a point | (SEQ ID NO: 871) | |
| | substitution of essential residue | | |
| | D36 by an A | | |
| I_{N}⁻ | N-terminal intein fragment from | ALSYDTEILTVEYGILPIGKIVEKRIECTVYSVD | Cheriyan et |
| (C1A) | Nostoc punctifrome (Npu) DnaE | NNGNIYTQPVAQWHDRGEQEVFEYCLEDG | al., 2013 |
| | split intein with a point | CLIRATKDHKFMTVDGQMMPIDEIFERELD | |
| | substitution of essential residue | LMRVDNLPNGT | |
| | C1 by an A | (SEQ ID NO: 872) | |
| SsrA | Protein degradation tag | AANDENYALAA | Keiler et |
| | | (SEQ ID NO: 873) | al., 1996 |

Components of the SICLOPPS system used in this study. In bold are the point residue substitution used in order to generate non-functional inteins.

### Cell-Free Production of Bacteriocins

Recombinant vectors were stabilized and amplified in E. coli DH5a standard strain and used as templates for cell-free protein synthesis using PURExpress® in vitro Protein Synthesis Kit (New England Biolabs). All bacteriocins synthesized were tested on petri dishes for antimicrobial activity against an indicator strain. Bacteriocins showing a halo of inhibition against at least one of the indicators were considered as positive.

Production and Purification of Garvicin ML from E. coli BL21-GarvML

In order to purify the antimicrobial compound produced by E. coli BL21-pUC-Npu-GarvML, the culture was grown in 10 ml LB broth supplemented with Ampicillin at 100 μg/ml (LB-Amp) and grown in a shaking 37° C. incubator overnight. 500 ml of LB-Amp were inoculated with the overnight culture to an OD₆₀₀ of 0.1 and grown in a shaking 37° C. incubator. When the culture had reached an OD₆₀₀ of 0.4, IPTG was added to a final concentration of 0.5 mM. Culture was grown for another 3 hours and cells were pelleted by centrifugation (8,000 r.p.m.; 4° C.) for 15 minutes. Cells were resuspended in 20 ml ice-cold column buffer (20 mM Phosphate buffer pH 6 and 1 M NaCl) and lysed by sonication (6 cycles of 10 seconds at 45% with 1 minute incubation in ice in between the cycles). The insoluble debris was pelleted by centrifugation (8,000 r.p.m.; 4° C.) for 15 minutes and the soluble fraction (SF) obtained was filtered through a 0.45 nm filter.

SF was further subjected to hydrophobic-interaction (Octyl Sepharose CL-4B; Merck) chromatography. First Ammonium Sulfate was added to the SF (10% w/v). A column with 2 ml of Octyl Sepharose CL-4B was washed with H₂O and equilibrated with 15 ml equilibration buffer (EB; 20 mM Phosphate buffer pH6 with Ammonium Sulfate [1% w/v]). Then the SF was added to the column, which was washed with 10 ml EB. Bacteriocin was eluted with 10 ml 70% EtOH diluted in 20 mM Phosphate buffer pH 6.

Finally the fraction obtained was diluted 5 times in H2O+TFA (0.1% v/v) and subjected to reverse phase chromatography (Source 5 RPC ST4.6-150; GE Healthcare) in a fast-protein liquid chromatography system (AKTA, RP-FPLC). Samples were eluted with the mobile phase consisting of 0.1% (v/v) trifluoroacetic acid (TFA) in a mixture of water (eluent A) and isopropanol (eluent B). (Both reagents were HPLC grade). A gradient program was followed: samples were initially eluted with 100% A for 5 min, then with a linear gradient 0-70% B over 50 min, followed by a linear gradient to 100% B over 5 min and maintained at 100% B for 7 min. The flow rate was maintained at 1 ml/min, absorbance was monitored at 254 nm and the column was kept at room temperature. Column eluents and FPLC fractions were assayed for antimicrobial activity by spot on agar assays (SPA), using L. garvieae 5806 as the target organism. FPLC fractions displaying antimicrobial activity were subjected to a second run of FPLC under the same conditions.

Characterization of Garvivin ML by Directed Proteomics Combined with Mass Spectrometry

Active fractions from the second run of FPLC were concentrated with a Speed-vac and subjected to matrix-assisted laser desorption ionization-time of flight mass spectrometry (MALDI-TOF MS) on a 4800 Proteomics Analyzer with TOF/TOF (AB SCIEX) in positive reflectron mode (Unidad de Proteómica-Universidad Complutense de Madrid, Madrid, Spain).

After drying out and resuspending the sample in 25 μl TEAB 25 mM and buffer S-TRAP to equal parts, it was digested with trypsin in a S-Trap microcolumn (PROTIFITM) as recommended by the manufacturer. Shortly, protein was reduced with 10 DTT for 60 min at 56° C., and then alkylated with 25 mM iodocetamide for 60 min in darkness. Then 20% SDS, TEAB 1 M and Phosphoric Acid were added to final concentration of 10%, 100 mM and 1.2%, respectively. Next S-Trap binding buffer was added in a 6:1 ratio, applied to the column and digested, following the protocol, with 1.5 μg recombinant Trypsin sequencing grade (Roche Molecular Biochemicals) in TEAB 50 mM for 90 min at 47° C. in static conditions.

Finally, the peptides obtained were eluted and dried out on a SpeedVac (Savant), reconstituted in 15 μl 2% ACN, Formic Acid 0.1% and quantified on a Qubit (ThermoFisher Scientific). Then samples were analyzed through LC-MS/MS.

### Example 1

This non-limiting example shows designing a genetic vector encoding a bacteriocin flanked by a split intein, and cell-free production of an active bacteriocin therefrom.

### Design of the Expression Vector for Production of Garvicin ML

Based on the work describing the split intein circular ligation of peptides and proteins (SICLOPPS) system [16] a gene containing the C and N-terminal intein fragments from Npu DnaE split intein (I_{C} and I_{N}, respectively) fused to the mature peptide of bacteriocin garvicin ML (GarML) was synthesized. With reference to FIG. 2C, the split intein sequence is shown with solid underline, and the bacteriocin sequence is shown with dotted underline. Native Garvicin ML circularization occurs after a head-to-tail ligation between residues Leu1 and Ala60 after leader sequence cleavage [6](FIG. 2A), but the intein chemistry typically requires the first amino acid of the target peptide to be either a cysteine or a serine. GarML has no cysteine in its mature sequence, but it has 3 serines (Ser19, Ser29 and Ser32). Accordingly, the order of the residues in Garvicin ML was switched, choosing Ser32 as the first residue in the linear conformation (Ser1 in the new conformation) and Phe31 as the last residue (Phe60 in the new conformation). In addition, a protein degradation tag in C-terminus (SsrA) of the construct was included (rectangular box in FIG. 2C) to overcome potential toxicity of Npu intein after splicing (FIGS. 2B-2D) [16]. Based on the amino acid sequence designed, the synthetic gene with the codon usage of E. coli was produced, and was put under the control of promoter T7 in a pUC expression standard expression vector, called pUC-Npu-GarML. In parallel the same construct without the inteins (pUC-GarML) and with an inactive variant of the Npu DnaE inteins having a N36D substitution in I_{C} and a CIA substitution in I_{N} (pUC-Npu⁻-GarML) were produced [17](FIG. 2B).

### Cell-Free Production of GarML

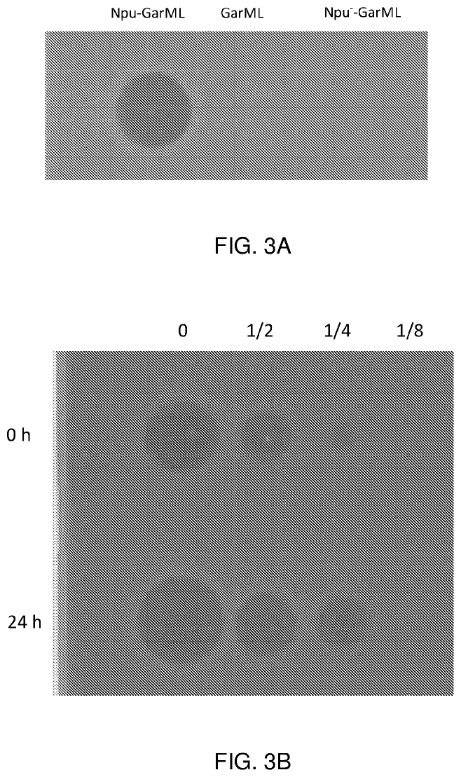

Plasmids pUC-Npu-GarML, pUC-GarML and pUC-Npu-GarML were used as templates for cell-free protein production of Npu-GarML, GarML and Npu-GarML, respectively. We then tested the activity of the products of the reactions against L. garvieae strain. Neither GarML nor Npu-C1A-GarML was active against the indicator, demonstrating that neither the linear GarML nor a linear version with the I_{C} and I_{N} at both sides of GarML was an active form. In contrast, Npu-GarML showed activity against the indicator (FIG. 3A), and this activity was higher when the product was left overnight at room temperature (FIG. 3B). These results demonstrated that in vitro production of GarML fused to Npu inteins allows for circularization of GarML (FIG. 2D) as the linear version of GarML with or without Npu inteins did not show any activity.

In some embodiments, designing a nucleic acid encoding a bacteriocin circularized by a split intein involves circularly permuting the amino acid sequence of a native, mature form of a circular bacteriocin such that a serine or cysteine in the native sequence is positioned as the first amino acid, fusing an N-terminal fragment of a split intein to the N-terminus of the circularly permuted bacteriocin sequence, and fusing a C-terminal fragment of the split intein to the C-terminus of the circularly permuted bacteriocin sequence. In some embodiments, a nucleic acid encoding a bacteriocin flanked at both the N- and C-termini by a split intein that circularizes the bacteriocin is expressed in vitro in a cell-free expression system to produce a gene product that exhibits antimicrobial activity of the encoded bacteriocin, where the encoded bacteriocin is a natively circular bacteriocin.

### Example 2

This non-limiting example shows expression of circular bacteriocin by a genetically engineered bacteria with a vector encoding a bacteriocin flanked by a split intein, and analysis of the expressed bactericin to confirm head-to-tail circularization by the split intein.

Purification and Mass Spectrometry (MS) and Multiple Reaction Monitoring (MRM) Analysis of Garvicin ML from the Soluble Fraction of Recombinant E. coli

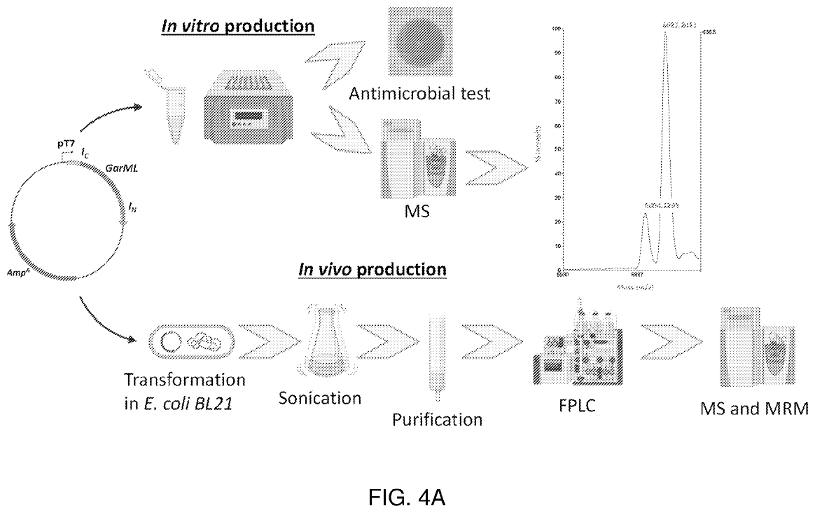

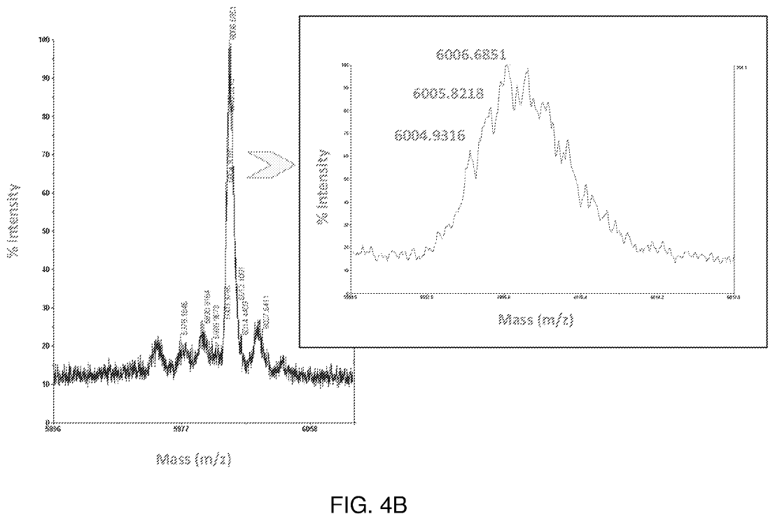

In order to demonstrate and prove correct splicing and circularization of GarML Npu-GarML production was scaled up using E. coli BL21 as producer (FIG. 4A). Soluble fraction of E. coli BL21 pUC-Npu-GarML showed antimicrobial activity against Lc. garvieae. The active peptide was further purified from the soluble fraction using hydrophobic interaction (HI) and reversed-phase fast-performance liquid chromatography (FPLC). Antimicrobial activity correlated with a peak eluting at 39% isopropanol in the FPLC chromatogram in two consecutive rounds of purification. Matrix-assisted laser desorption ionization-time of flight mass spectrometry (MALDI-TOF MS) analysis revealed that the corresponding fractions had a mass of 6,004.2 Da (FIG. 4B). This correlates with the mass from native circular garvicin ML [6]. This fraction was further subjected to trypsin digestion and fragments originated where analysed by LC-MRM-MS analysis. Knowing the mass and aminoacidic sequence of garvicin ML, it is possible to predict the precursor z and fragments m/z (MRM transition). Each targeted peptide has a set of accompanying transitions that are then selectively detected in a second stage of MS. All peptides were confirmed by MS/MS covering 100% of the complete GarML sequence. One of the peptides detected and confirmed by MS/MS (TIVNAVSAGMDIATALSFSGAFTAAGGIMALIKK) contained residues S1 and F60 from Npu-GarML linked together, thus confirming splicing and head-to-tail circularization of GarML.

### Example 3

This non-limiting example shows cell-free production of known and predicted circular bacteriocins using split inteins, and confirmation of antimicrobial activity thereof.

### Plasmid Design and Cell-Free Production of Characterized and Uncharacterized Circular Bacteriocins

Following the same scheme as for GarML, different plasmids containing genes coding for recombinant proteins containing Npu I_{C} and I_{N} fused to the mature amino acid sequence of different known and hypothetical circular bacteriocins were synthesized. The circularization point for all of them was changed with respect to the native peptides so they all would start with a Ser in position 1 (Table 2.1). The Ssra tag was also added to the C-terminus. As for GarML, an Npu inactive version was built for all peptides. Plasmids synthesized were used as templates for cell free protein production. Antimicrobial activity of the peptides obtained after cell free production and overnight incubation at room temperature were tested against indicator L. lactis IL1403. None of the proteins containing the inactive inteins showed antimicrobial activity. Most of the fractions containing native Npu inteins fused to the mature sequence of the characterized, and hypothetical circular bacteriocins showed activity against L. lactis IL1403, thus showing that SICLOPPS method is also applicable for circularization of other circular bacteriocins (Table 3).

TABLE 3
Antimicrobial activity of different bacteriocins circularized with inteins

| | | Indicator | | |
|---|---|---|---|---|
| | | L. lactis | P. pentosaceus | |
| Plasmid | Bacteriocin | IL1403 | HELA | L. inocua |
| pCirc-Alc | Amylocyclicin | + | − | − |
| pCirc-3688STDY | Bacteriocin 3688STDY6124959 | + | − | − |
| pCirc-EntAS48 | Enterocin AS-48 | + | − | − |
| pCirc-BCW_2997 | Bacteriocin BCW 2997 | + | | − |
| pCirc-CF11 | Bacteriocin CF11 | − | − | − |
| pCirc-NBRC_15376 | Bacteriocin NBRC 15376 | − | − | − |
| pCirc-CarA | Carnocyclin A | + | − | − |
| pCirc-CirA | Circularin A | + | − | − |
| pCirc-YS111 | Bacteriocin YS111 | + | | |
| pCirc-NKR_5_3B | Enterocin NKR-5-3B | + | − | + |
| pCirc-DSM_15102 | DSM 15102 | + | − | − |
| pCirc-GarML | Garvicin ML (GarML) | + | − | − |
| pCirc-LeuQ | Leucocyclicin Q | + | − | − |
| pCirc-AFS089278 | Bacteriocin AFS089278 | − | − | − |
| pCirc_UberA | Uberolysin A (UberA) | − | − | + |
| pCirc_TD3 | Bacteriocin TD3 | − | − | − |
| pCirc_NRLL_B_24287 | Bacteriocin NRRL B-24287 | − | − | − |
| pCirc_AK22 | Bacteriocin AK22 | − | − | − |
| pCirc_15828 | Bacteriocin 15828 | − | − | − |
| pCirc_ButAR10 | Butyrivibriocin AR10 | − | − | − |
| pCirc_ParP | Paracyclicin P (ParP) | + | − | − |
| pCirc_NCTC_12958 | Bacteriocin NCTC 12958 | + | − | − |
| pCirc_UoS2029 | Bacteriocin UoS2029 | − | − | − |
| pCirc_F9_V1 | Bacteriocin F9_V1* | − | − | − |
| pCirc_F9_V2 | Bacteriocin F9_V2* | − | − | − |
| pCirc_GasA | Gassericin A (GasA) | + | + | + |
| pCirc_PlcA | Plantaricyclin A (PlcA) | + | − | − |
| pCirc_Cer | Cerecyclin | − | − | − |
| pCirc_R26283 | Bacteriocin R26833 toxic | | | |

In some embodiments, bacteriocins known or predicted to be circular bacteriocins are expressed in a cell-free system by designing a nucleic acid encoding the bacteriocin flanked by a split intein, where the native amino acid sequence of the bacteriocin is circularly permuted, and/or is mutated to introduce a non-native serine, such that a serine is at position 1 of the bacteriocin encoded by the nucleic acid.

### Example 4

This non-limiting example shows screening a library for bacteriocins having a desired activity.

A nucleic acid encoding an amino acid sequence of a circular bacteriocin, Enterocin NKR-5-3B, is prepared. The amino acid sequence is modified relative to the native sequence of the circular bacteriocin such that a serine or cysteine is at position 1 of the amino acid sequence, e.g., by circularly permuting the native sequence to place a native serine or cysteine at position 1. The nucleic acid is amplified and mutations are introduced, e.g., by random mutagenesis or selective point mutation, to generate a collection of variants of the nucleic acid encoding the circular bacteriocin. The variants are cloned into an expression vector so that each variant of the nucleic acid encoding the circular bacteriocin is flanked by a split intein configured to circularize the bacteriocin, to generate a library of expression vectors having variant nucleic acids encoding the circular bacteriocin.

A cell-free expression system is used to express circular bacteriocins from the library of expression vectors, and the produced circular bacteriocins are tested for antimicrobial activity against one or more bacterial strains of interest, to identify those that exhibit a desired activity. For example, in one embodiment variant nucleic acids encoding Enterocin NKR-5-3B that retain antimicrobial activity against L. lactis, but do not retain antimicrobial activity against L. inocua are isolated and sequenced to identify the mutation(s) responsible for conferring the desired antimicrobial activity to the circular bacteriocin.

### Example 5

This non-limiting example shows controlling the growth of a microbial organism using a circular bacteriocin.

A polypeptide containing an amino acid sequence of a circular bacteriocin, for example Enterocin AS-48, flanked by a split intein is produced. The split intein is a conditionally active, pH-sensitive split intein, and is configured to circularize the bacteriocin when the pH is below 6.0. The polypeptide is introduced into a culture medium growing a microbial organism of interest, at pH 7.0. The bacteriocin is not circularized at pH 7.0, and does not exhibit antimicrobial activity. When a contaminating microbial species, L. lactis, is detected in the culture medium, the pH of the medium is reduced to below 6.0, which activates the split intein and causes the bacteriocin to circularize. Subsequently, the growth of the contaminating L. lactis in the culture medium is inhibited.

### Example 6

This non-limiting example shows controlling the growth of a microbial organism using a circular bacteriocin.

A microbial cell is genetically engineered with an expression vector encoding a circular bacteriocin, for example Leucocyclicin Q, flanked by a split intein. The genetically engineered microbial cell is introduced into a culture medium growing a microbial organism of interest. The microbial cell produces the bacteriocin in circularized form, and secretes it into the culture medium. Growth of a contaminating microbial species, L. lactis, is inhibited by the circular bacteriocin.

In at least some of the embodiments described herein, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A. B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

What is claimed is:

1. A fusion polypeptide comprising an amino acid sequence of a bacteriocin flanked at both the N- and C-termini by a split intein that circularizes the bacteriocin.

2. The fusion polypeptide of claim 1, wherein the bacteriocin is a natively circular bacteriocin.

3. The fusion polypeptide of claim 1 or 2, wherein the amino acid sequence of the bacteriocin is circularly permuted compared to a native amino acid sequence of the bacteriocin.

4. The fusion polypeptide of anyone of claims 1-3, wherein the first residue of the amino acid sequence of the bacteriocin is a serine or a cysteine that is present in the native amino acid sequence of the bacteriocin.

5. The fusion polypeptide of anyone of claims 1-3, wherein the first residue of the amino acid sequence of the bacteriocin is a non-native serine or a non-native cysteine.

6. The fusion polypeptide of claim 5, wherein the non-native serine or the non-native cysteine substitutes a native amino acid residue in the amino acid sequence of the bacteriocin.

7. The fusion polypeptide of claim 5, wherein the length of the amino acid sequence of the bacteriocin is increased by one residue due to the non-native serine or the non-native cysteine compared to the length of the native amino acid sequence of the bacteriocin.

8. The fusion polypeptide of any one of claims 5-7, wherein the native amino acid sequence of the bacteriocin does not comprise a serine or cysteine.

9. The fusion polypeptide of any one of the preceding claims, wherein the split intein is based on an intein from one of the following: Npu DnaE, Sce VMA, Ssp DnaE.

10. The fusion polypeptide of any one of the preceding claims, wherein the split intein is a conditional split intein.

11. The fusion polypeptide of claim 10, wherein the conditional split intein is pH- or temperature-sensitive.

12. The fusion polypeptide of any one of the preceding claims, wherein the split intein comprises a second amino acid sequence of a C-terminal intein fragment (I_{C}) at least 80% identical to the I_{C} shown in Table B, and a third amino acid sequence of a N-terminal intein fragment (I_{N}) at least 80% identical to the split intein I_{N} shown in Table B.

13. The fusion polypeptide of any one of the preceding claims, wherein the bacteriocin is selected from any one of the bacteriocins listed in Table A.

14. The fusion polypeptide of any one of the preceding claims, wherein the amino acid sequence of the bacteriocin is at least 80% identical to any one of the sequences listed in Table A.

15. The fusion polypeptide of any one of the preceding claims, wherein the amino acid sequence of the bacteriocin is selected from any one of the sequences listed in Table A.

16. The fusion polypeptide of any one of claims 1-14, wherein the bacteriocin is an engineered bacteriocin.

17. The fusion polypeptide of any one of the preceding claims, wherein one or more amino acids of the polypeptide in the amino acid sequence is a non-natural amino acid.

18. The fusion polypeptide of any one of the preceding claims, further comprising a degradation tag.

19. The fusion polypeptide of claim 18, wherein the degradation tag is at the C-terminus of the fusion polypeptide.

20. The fusion polypeptide of any one of the preceding claims, wherein the split intein comprises a C-terminal intein fragment (“I_{C}”) fused N-terminal to the amino acid sequence of the bacteriocin and a N-terminal intein fragment (“I_{N}”) fused C-terminal to the amino acid sequence of the bacteriocin, wherein the polypeptide further comprises a degradation tag C-terminal to the I_{N}.

21. The fusion polypeptide of any one of claims 18-20, wherein the degradation tag comprises a sequence at least 80% identical to AANDENYALAA (SEQ ID NO: 873).

22. The fusion polypeptide of any one of the preceding claims, further comprising a signal peptide and/or a leader sequence.

23. A nucleic acid comprising a nucleotide sequence encoding the fusion polypeptide of any one of the preceding claims.

24. The nucleic acid of claim 23, wherein the nucleotide sequence is operably linked to a promoter sequence.

25. The nucleic acid of claim 23 or 24, wherein the nucleic acid comprises DNA.

26. The nucleic acid of claim 23, wherein the nucleic acid comprises RNA.

27. A genetic vector comprising the nucleic acid of any one of claims 23-26.

28. A genetically engineered microbial cell comprising the nucleic acid of any one of claims 23-26, or the genetic vector of claim 27.

29. The microbial cell of claim 28, wherein the microbial cell is resistant to the bacteriocin.

30. The microbial cell of claim 28 or 29, wherein the microbial cell comprises a second nucleic acid encoding an immunity modulator that confers resistant to the bacteriocin.

31. The microbial cell of claim 30, wherein expression of the immunity modulator from the second nucleic acid is regulatable.

32. The microbial cell of any one of claims 28-31, wherein the microbial cell is a bacteria, fungi, or algae.

33. A composition comprising the fusion polypeptide of any one of claims 1-22.

34. A composition comprising a circular bacteriocin and a split intein.

35. A method of making a circular bacteriocin, comprising contacting the nucleic acid of any one of claims 22-26, or the genetic vector of claim 27 with an in vitro expression system under conditions sufficient to produce a circular bacteriocin.

36. A method of making a circular bacteriocin, comprising culturing the microbial cell of any one of claims 28-31 under conditions sufficient to produce a circular bacteriocin.

37. The method of claim 35 or 36, further comprising purifying the circular bacteriocin.

38. The method of any one of claims 35-37, further comprising purifying the fusion polypeptide.

39. The method of any one of claims 35-38, wherein the split intein is a conditional split intein that circularizes the bacteriocin under a permissive condition but not under a non-permissive condition, and wherein the method further comprises exposing the fusion polypeptide to the permissive condition, following exposure to the non-permissive condition, to induce circularization of the bacteriocin.

40. The method of any one of claims 35-39, further comprising modifying the pH or temperature to induce circularization of the bacteriocin, wherein the split intein is pH- or temperature-sensitive, respectively.

41. The method of any one of claims 35-40, further comprising allowing the split intein to be degraded after the circular bacteriocin is produced.

42. A library comprising a plurality of genetic vectors, each genetic vector comprising the nucleic acid of any one of claims 23-26, wherein at least two of the plurality of genetic vectors comprise nucleotide sequences encoding different bacteriocins.

43. The library of claim 41, wherein the nucleotide sequences encode bacteriocins from different microbial species.

44. The library of claim 41, wherein the nucleotide sequences comprise different sequence variants of a parent bacteriocin.

45. The library of claim 44, wherein the parent bacteriocin is a natively circular bacteriocin, and the sequence variants comprise a first variant that abrogates natural circularization of the parent bacteriocin.

46. A method of screening, comprising:
providing the library of any one of claims 42-45;
expressing a plurality of polypeptides encoded by one of more genetic vectors of the library;
generating a plurality of circular bacteriocins from the plurality of expressed polypeptides; and
assaying the plurality of circular bacteriocins for a desired activity.

47. The method of claim 46, wherein the desired activity comprises antimicrobial activity.

48. A method of controlling the growth of a microorganism, comprising contacting a composition comprising and/or conducive to supporting the growth of a microorganism with the microbial cell of any one of claims 28-32 under conditions sufficient to produce a circular bacteriocin, to thereby control the growth of the microorganism.

49. A method of controlling the growth of a microorganism, comprising contacting a composition comprising and/or conducive to supporting the growth of a microorganism with a circular bacteriocin made by the method of any one of claims 35-38, to thereby control the growth of the microorganism.

50. A method of controlling the growth of a microorganism, comprising contacting a composition comprising and/or conducive to supporting the growth a microorganism with the fusion polypeptide of any one of claims 1-22, to thereby control the growth of the microorganism.

51. The method of any one of claims 48-50, wherein the microorganism is a bacteria.

52. The method of any one of claims 48-51, wherein the composition is a culture medium, feedstock, or a microbiome.

53. The method of any one of claims 48-52, wherein the split intein is a conditional split intein that circularizes the bacteriocin under a permissive condition but not under a non-permissive condition, and wherein the method further comprises providing the permissive condition to the composition to thereby induce circularization of the bacteriocin.

54. The method of any one of claims 48-53, wherein the method comprises modifying the pH or temperature of the composition to induce circularization of the bacteriocin, wherein the split intein is pH- or temperature-sensitive, respectively.

55. A method of designing a nucleic acid encoding a polypeptide precursor of a bacteriocin, comprising:
identifying a native amino acid sequence of a candidate bacteriocin, wherein the native amino acid sequence does not comprise a serine or cysteine at the N-terminus;
providing a second amino acid sequence having a serine or cysteine at the N-terminus thereof by at least one of:
circularly permuting the native amino acid sequence; or
introducing a serine or cysteine to the native amino acid sequence;
providing a nucleotide sequence encoding a polypeptide comprising the second amino acid sequence flanked at both the N- and C-termini by a split intein configured to circularize the bacteriocin; and
expressing the polypeptide encoded by the nucleotide sequence.

56. The method of claim 55, wherein the candidate bacteriocin is predicted to be a circular bacteriocin based on a genomic sequence of a microorganism that encodes the candidate bacteriocin in its genome.

57. The method of claim 55 or 56, comprising:
identifying a plurality of native amino acid sequences of a plurality of different candidate bacteriocins;
for each of the plurality of native amino acid sequences:
providing the second amino acid sequence; and
providing the nucleotide sequence encoding a polypeptide comprising the second amino acid sequence flanked at both the N- and C-termini by a split intein configured to circularize the bacteriocin,
thereby generating a library of nucleic acids representing each of the plurality of native amino acid sequences.

58. The method of any one of claims 55-57, wherein the polypeptide further comprises a degradation tag.

59. The method of any one of claims 55-58, wherein the polypeptide further comprises a signal peptide and/or leader sequence.

60. The method of any one of claims 55-59, wherein the polypeptide is expressed in vitro.

61. The method of any one of claims 55-59, wherein the polypeptide is expressed from a genetically engineered microbial cell configured to express the polypeptide encoded by the nucleotide sequence.

62. The fusion protein, nucleic acid, vector, library, microbial cell, or method of any one of the preceding claims, wherein the polypeptide comprises an affinity tag, optionally wherein the affinity tag is chitin binding protein (CBP).


SEQUENCE LISTING (874 sequences)

SEQ ID NO: 1 - protein, 40 aa, Enterococcus faecalis
WLPPAGLLGRCGRWFRPWLLWLQSGAQYKWLGNLFGLGPK

SEQ ID NO: 2 - protein, 5 aa, synthetic construct
  REGION 1..5: note = N-terminal motif of class terminal IIa
  VARIANT 3: note = Xaa = any amino acid
YGXGV

SEQ ID NO: 3 - protein, 135 aa, synthetic construct
  REGION 1..135: note = Hybrid bacteriocin Ent35-MccV
GKYYGNGVSCNKKGCSVDWGRAIGIIGNNSAANLATGGAAGWKSGGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 4 - protein, 46 aa, Lactobacillus acidophilus
MISSHQKTLTDKELALISGGKTHYPTNAWKSLWKGFWESLRYTDGF

SEQ ID NO: 5 - DNA, 141 bp, Lactobacillus acidophilus
  source 1..141: mol_type = genomic DNA
atgatttcatctcatcaaaaaacgttaactgataaagaattagcattaatttctggggggaaaacgcactacccgactaatgcatggaaaagtctttggaaaggtttctgggaaagccttcgttatactgacggtttttag

SEQ ID NO: 6 - protein, 81 aa, Lactobacillus acidophilus
MISMISSHQKTLTDKELALISGGKTYYGTNGVHCTKKSLWGKVRLKNVIPGTLCRKQSLPIKQDLKILLGWATGAFGKTFH

SEQ ID NO: 7 - DNA, 246 bp, Lactobacillus acidophilus
  source 1..246: mol_type = genomic DNA
atgatttcaatgatttcatctcatcaaaaaacgttaactgataaagaattagcattaatttctggggggaaaacgtactatggtactaatggtgtgcattgtactaaaaagagtctttggggtaaagtacgcttaaaaaacgtgattcctggaactctttgtcgtaagcaatcgttgccgatcaaacaggatttaaaaattttactgggctgggctacaggtgcttttggcaagacatttcattaa

SEQ ID NO: 8 - protein, 60 aa, Lactobacillus acidophilus
MDKKTKILFEVLYIICIIGPQFILFVTAKNNMYQLVGSFVGIVWFSYIFWYIFFKQHKKM

SEQ ID NO: 9 - DNA, 183 bp, Lactobacillus acidophilus
  source 1..183: mol_type = genomic DNA
atggataagaaaacaaaaatattatttgaagtattatacatcatctgtataataggccctcaatttatattatttgtgactgcaaaaaacaatatgtatcagttggtgggttcgtttgttggaatagtatggttttcgtatattttttggtatatttttttcaaacaacataaaaaaatgtag

SEQ ID NO: 10 - protein, 65 aa, Lactobacillus gasseri
MALKTLEKHELRNVMGGNKWGNAVIGAATGATRGVSWCRGFGPWGMTACALGGAAIGGYLGYKSN

SEQ ID NO: 11 - DNA, 198 bp, Lactobacillus gasseri
  source 1..198: mol_type = genomic DNA
atggctttaaaaacattagaaaaacatgaattaagaaatgtaatgggtggaaacaagtgggggaatgctgtaataggagctgctacgggagctactcgcggagtaagttggtgcagaggattcggaccatggggaatgactgcctgtgcgttaggaggtgctgcaattggaggatatctgggatataagagtaattaa

SEQ ID NO: 12 - protein, 51 aa, Staphylococcus aureus [sequence omitted]

SEQ ID NO: 13 - DNA, 156 bp, Staphylococcus aureus
  source 1..156: mol_type = genomic DNA
atgagttggttaaattttttaaaatacatcgctaaatatggcaaaaaagcggtatctgctgcttggaagtacaaaggtaaagtattagaatggcttaatgttggtcctactcttgaatgggtatggcaaaaattaaagaaaattgctggattataa

SEQ ID NO: 14 - protein, 61 aa, Enterococcus avium
MTRSKKLNLREMKNVVGGTYYGNGVSCNKKGCSVDWGKAISIIGNNSAANLATGGAAGWKS

SEQ ID NO: 15 - DNA, 186 bp, Enterococcus avium
  source 1..186: mol_type = genomic DNA
atgacaagatcaaaaaaattaaatttacgcgaaatgaagaatgttgttggtggtacctactatggaaatggtgtatcttgtaacaagaaaggctgttcagttgactggggcaaagccatcagtattataggaaataattccgcagcaaacttagcaactggtggtgctgctggttggaagtcataa

SEQ ID NO: 16 - protein, 67 aa, Enterococcus faecalis
MKKKLVICGIIGIGFTALGTNVEAATYYGNGLYCNKQKCWVDWNKASREIGKIIVNGWVQHGPWAPR

SEQ ID NO: 17 - DNA, 204 bp, Enterococcus faecalis
  source 1..204: mol_type = genomic DNA
atgaaaaagaaattagttatttgtggcattattgggattggttttacagcattaggaacaaatgtagaagctgctacgtattacggaaatggtttatattgtaataagcaaaaatgttgggtagactggaataaagcttcaagggaaattggaaaaattattgttaatggttgggtacaacatggcccttgggctcctagatag

SEQ ID NO: 18 - protein, 51 aa, Lactococcus lactis
MKEQNSFNLLQEVTESELDLILGAKGGSGVIHTISHEVIYNSWNFVFTCCS

SEQ ID NO: 19 - DNA, 156 bp, Lactococcus lactis
  source 1..156: mol_type = genomic DNA
atgaaagaacaaaactcttttaatcttcttcaagaagtgacagaaagtgaattggaccttattttaggtgcaaaaggcggcagtggagttattcatacaatttctcatgaagtaatatataatagctggaactttgtatttacttgctgctcttaa

SEQ ID NO: 20 - protein, 74 aa, Enterococcus faecium
MKKKVLKHCVILGILGTCLAGIGTGIKVDAATYYGNGLYCNKEKCWVDWNQAKGEIGKIIVNGWVNHGPWAPRR

SEQ ID NO: 21 - DNA, 225 bp, Enterococcus faecium
  source 1..225: mol_type = genomic DNA
atgaaaaagaaagtattaaaacattgtgttattctaggaatattaggaacttgtctagctggcatcggtacaggaataaaagttgatgcagctacttactatggaaatggtctttattgtaacaaagaaaaatgttgggtagattggaatcaagctaaaggagaaattggaaaaattattgttaatggttgggttaatcatggtccatgggcacctagaaggtag

SEQ ID NO: 22 - protein, 50 aa, Clostridium botulinum
MQKPEIISADLGLCAVNEFVALAAIPGGAATFAVCQMPNLDEIVSNAAYV

SEQ ID NO: 23 - DNA, 153 bp, Clostridium botulinum
  source 1..153: mol_type = genomic DNA
atgcaaaaaccagaaattattagtgctgatttagggctttgtgcagttaatgaatttgtagctcttgctgccattcctggtggtgctgctacatttgcagtatgccaaatgccaaacttggatgagattgttagtaatgcagcatatgtttaa

SEQ ID NO: 24 - protein, 58 aa, Streptococcus equinus
MMNATENQIFVETVSDQELEMLIGGADRGWIKTLTKDCPNVISSICAGTIITACKNCA

SEQ ID NO: 25 - DNA, 177 bp, Streptococcus equinus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcttgtaaaaattgtgcttaa

SEQ ID NO: 26 - protein, 77 aa, Brochothrix campestris
MHKVKKLNNQELQQIVGGYSSKDCLKDIGKGIGAGTVAGAAGGGLAAGLGAIPGAFVGAHFGVIGGSAACIGGLLGN

SEQ ID NO: 27 - DNA, 234 bp, Brochothrix campestris
  source 1..234: mol_type = genomic DNA
atgcacaaggtaaaaaaattaaacaatcaagagttacaacagatcgtgggaggttacagttcaaaagattgtctaaaagatattggtaaaggaattggtgctggtacagtagctggggcagccggcggtggcctagctgcaggattaggtgctatcccaggagcattcgttggagcacattttggagtaatcggcggatctgccgcatgcattggtggattattaggtaactag

SEQ ID NO: 28 - protein, 80 aa, Butyrivibrio fibrisolvens
MSKKQIMSNCISIALLIALIPNIYFIADKMGIQLAPAWYQDIVNWVSAGGTLTTGFAIIVGVTVPAWIAEAAAAFGIASA

SEQ ID NO: 29 - DNA, 243 bp, Butyrivibrio fibrisolvens
  source 1..243: mol_type = genomic DNA
atgagtaaaaaacaaattatgagtaactgtatatcaattgcattattaatagcactaattcctaatatctattttattgcagataaaatgggaattcagttagcacctgcttggtatcaagatattgtgaattgggtatctgctggtggaacacttactactggttttgcgattattgtaggagttacagtaccggcatggatagcagaagcagctgcagcttttggtatagcttcagcatga

SEQ ID NO: 30 - protein, 48 aa, Butyrivibrio fibrisolvens
MNKELNALTNPIDEKELEQILGGGNGVIKTISHECHMNTWQFIFTCCS

SEQ ID NO: 31 - DNA, 147 bp, Butyrivibrio fibrisolvens
  source 1..147: mol_type = genomic DNA
atgaacaaagaacttaatgcacttacaaatcctattgacgagaaggagcttgagcagatcctcggtggtggcaatggtgtcatcaagacaatcagccacgagtgccacatgaacacatggcagttcattttcacatgttgctcttaa

SEQ ID NO: 32 - protein, 66 aa, Carnobacterium maltaromaticum
MNSVKELNVKEMKQLHGGVNYGNGVSCSKTKCSVNWGQAFQERYTAGINSFVSGVASGAGSIGRRP

SEQ ID NO: 33 - DNA, 201 bp, Carnobacterium maltaromaticum
  source 1..201: mol_type = genomic DNA
atgaatagcgtaaaagaattaaacgtgaaagaaatgaaacaattacacggtggagtaaattatggtaatggtgtttcttgcagtaaaacaaaatgttcagttaactggggacaagcctttcaagaaagatacacagctggaattaactcatttgtaagtggagtcgcttctggggcaggatccattggtaggagaccgtaa

SEQ ID NO: 34 - protein, 61 aa, Carnobacterium maltaromaticum
MKSVKELNKKEMQQINGGAISYGNGVYCNKEKCWVNKAENKQAITGIVIGGWASSLAGMGH

SEQ ID NO: 35 - DNA, 186 bp, Carnobacterium maltaromaticum
  source 1..186: mol_type = genomic DNA
atgaaaagcgttaaagaactaaataaaaaagaaatgcaacaaattaatggtggagctatctcttatggcaatggtgtttattgtaacaaagagaaatgttgggtaaacaaggcagaaaacaaacaagctattactggaatagttatcggtggatgggcttctagtttagcaggaatgggacattaa

SEQ ID NO: 36 - protein, 71 aa, Carnobacterium maltaromaticum
MNNVKELSIKEMQQVTGGDQMSDGVNYGKGSSLSKGGAKCGLGIVGGLATIPSGPLGWLAGAAGVINSCMK

SEQ ID NO: 37 - DNA, 216 bp, Carnobacterium maltaromaticum
  source 1..216: mol_type = genomic DNA
atgaataatgtaaaagagttaagtattaaagaaatgcaacaagttactggtggagaccaaatgtcagatggtgtaaattatggaaaaggctctagcttatcaaaaggtggtgccaaatgtggtttagggatcgtcggcggattagctactatcccttcaggtcctttaggctggttagccggagcagcaggtgtaattaatagctgtatgaaataa

SEQ ID NO: 38 - protein, 64 aa, Carnobacterium maltaromaticum
MLYELVAYGIAQGTAEKVVSLINAGLTVGSIISILGGVTVGLSGVFTAVKAAIAKQGIKKAIQL

SEQ ID NO: 39 - DNA, 195 bp, Carnobacterium maltaromaticum
  source 1..195: mol_type = genomic DNA
atgttatatgaattagttgcatatggtatcgcacaaggtacagctgaaaaggttgtaagtctaattaacgcaggtttaacagtagggtctattatttcaattttgggtggggtcacagtcggtttatcaggtgtcttcacagcagttaaagcagcaattgctaaacaaggaataaaaaaagcaattcaattataa

SEQ ID NO: 40 - protein, 836 aa, Pectobacterium carotovorum
  source 1..836: note = subsp. carotovorum
MIKYRLYAPNDGDTMTVSGGGGWVSNDDRKGGNDRDNGKGGSAVDFSKNPEKQAIVNPYLAIAIPMPVYPLYGKLGFTINTTAIETELANVRAAINTKLATLSAVIGRSLPVVGRVFGVTAAGMWPSSTAPSSLDSIYNQAHQQALAQLAAQQGVLNKGYNVTAMPAGFVSSLPVSEIKSLPTAPASLLAQSVINTELSQRQLALTQPTTNAPVANIPVVKAEKTAMPGVYSAKIIAGEPAFQIKVDNTKPALAQNPPKVKDDIQVSSFLSSPVADTHHAFIDFGSDHEPVYVSLSKIVTAEEEKKQVEEAKRREQEWLLRHPITAAERKLTEIRQVISFAQQLKESSVATISEKTKTVAVYQEQVNTAAKNRDNFYNQNRGLLSAGITGGPGYPIYLALWQTMNNFHQAYFRANNALEQESHVLNLARSDLAKAEQLLAENNRLQVETERTLAEEKEIKRNRVNVSTFGTVQTQLSKLLSDFYAVTSLSQSVPSGALASFSYNPQGMIGSGKIVGKDVDVLFSIPVKDIPGYKSPINLDDLAKKNGSLDLPIRLAFSDENGERVLRAFKADSLRIPSSVRGVAGSYDKNTGIFSAEIDGVSSRLVLENPAFPPTGNVGNTGNTAPDYKALLNTGVDVKPVDKITVTVTPVADPVDIDDYIIWLPTASGSGVEPIYVVFNSNPYGGTEKGKYSKRYYNPDKAGGPILELDWKNVKIDHAGVDNVKLHTGRFKASVENKVMIERLENILNGQITATDTDKRFYTHELRELNRYRNLGIKDGEVPSSIQEESAVWNDTHTATLEDYKINEKEQPLYTDAALQAAYEQELKDALGGKHG

SEQ ID NO: 41 - DNA, 2511 bp, Pectobacterium carotovorum
  source 1..2511: mol_type = genomic DNA; sub_species = carotovorum
atgattaaataccgtttatatgctccaaatgatggagacaccatgacagtgagtggtggtggtggttgggtttcaaacgatgatcgcaaaggtggtaatgacagggacaatggcaaaggtggttctgccgttgattttagtaaaaatccagaaaagcaggctatcgttaatccctatttggcaatcgcgataccgatgccggtctaccctctttatggaaagctagggttcacaataaatacgacggcaattgagactgaactcgcaaatgtcagagcagcaattaacactaaacttgcaacactcagtgcagtgattggcagatcacttccggtcgttgggcgggtatttggtgttactgccgccggaatgtggccttctagtaccgctcccagtagtctcgattctatatacaatcaagcacatcagcaggctttagcccagttagctgctcaacagggagtattaaataaagggtataacgttacagcaatgcctgcaggtttcgtcagcagtttgcctgttagtgaaatcaaatcattgccaacagctcccgccagtttactggcacaaagtgtgattaataccgaactttcccagcgtcaactggctcttactcagcccacgacgaatgcaccagtcgcgaatattcccgtagttaaagcagagaaaacagcaatgccaggtgtgtattcagcgaaaattattgctggtgagcctgcattccaaatcaaggtcgataataccaaacctgctttggcacagaatccgccgaaagtaaaagatgatattcaggtatcttctttcctttcctcgccagtagctgatacgcaccatgcatttattgattttggcagcgatcatgaaccggtatacgtgtctctttcaaagatcgtgacagccgaggaggagaaaaaacaggttgaagaggccaagcgccgtgagcaggagtggttgttgcgtcatccaattacagctgcggagcgaaaattaactgaaatccgccaagtgatctcttttgctcaacagctaaaagaaagctctgtcgcaaccatttcagaaaaaactaaaactgttgcggtttaccaagaacaggtgaataccgctgcaaaaaatcgcgacaatttttataatcaaaatagaggtctgttaagtgcgggtataactgggggaccgggatatcctatttatcttgctttatggcaaacgatgaataactttcatcaggcttatttcagagcaaataatgcattggaacaagagagtcatgttctgaacctggctcgttctgatctggctaaggctgagcaattgcttgctgagaataatcgacttcaggttgaaacggagcgaacgcttgccgaagaaaaagagataaaacgcaacagggttaatgtatcaacatttggcacagtgcaaactcaacttagtaaattgctgtcagatttttatgctgttacatcactttcccaaagtgttccttcgggggcattagcctctttttcatataatccacaagggatgattggcagcggtaagattgttgggaaggatgtcgatgttttattttccatcccagtaaaagatattccgggatataaatctcctattaacttggacgatttagccaagaaaaatggaagtctggatcttcccattcgtctggcattttctgatgagaatggagaaagggttcttcgggcattcaaagcggatagtctgcgaatcccttcgagtgtcagaggtgtagcgggcagttatgacaaaaatacgggtatttttagtgcagaaattgatggtgtttcatctcgccttgtactggaaaacccagcgtttcctccgaccggaaatgtcggtaatacgggtaatactgcacctgactataaagcattactgaatactggtgttgatgttaaacctgttgataaaatcacagttacggtaacaccagttgctgatccagtggatattgatgactatataatctggttgccaactgcgtctggttctggcgtggaacccatttatgtcgtgtttaacagtaatccgtatggtgggacggaaaaaggaaaatatagcaaacgttattataatccagataaggcaggcggtccgatcttggagctggattggaaaaacgttaagattgaccatgcaggtgtggacaatgttaaattacacacagggcgtttcaaagcgtcggttgaaaacaaagtgatgattgaacgtttggaaaacatactgaatggtcaaatcacggccacggatactgacaagcgattctatacgcatgaattaagagagttaaaccgctacagaaatttaggcatcaaagacggtgaagtgcctagtagcattcaagaagaaagcgctgtttggaacgacacacacacagcgacgcttgaagactacaaaattaatgagaaagagcaaccgttgtacactgatgctgctttgcaggcagcctacgaacaggaactcaaagacgcattaggagggaaacatggctaa

SEQ ID NO: 42 - protein, 74 aa, Bacillus cereus
MENLQMLTEEELMEIEGGGWWNSWGKCVAGTIGGAGTGGLGGAAAGSAVPVIGTGIGGAIGGVSGGLTGAATFC

SEQ ID NO: 43 - DNA, 225 bp, Bacillus cereus
  source 1..225: mol_type = genomic DNA
atggaaaacttacaaatgttaactgaagaagaattaatggaaattgaaggtggaggctggtggaatagctggggtaaatgtgttgctggaactatcggtggagctggaactggtggtttaggtggagctgctgcaggttcagctgttccggttattggtactggtattggtggcgctattggtggagttagcggtggccttacaggtgcagctactttttgctaa

SEQ ID NO: 44 - protein, 78 aa, Streptoverticillium griseoverticillatum
MTASILQQSVVDADFRAALLENPAAFGASAAALPTPVEAQDQASLDFWTKDIAATEAFACRQSCSFGPFTFVCDGNTK

SEQ ID NO: 45 - DNA, 237 bp, Streptoverticillium griseoverticillatum
  source 1..237: mol_type = genomic DNA
atgaccgcttccattcttcagcagtccgtcgtggacgccgacttccgcgcggcgctgcttgagaaccccgccgccttcggcgcttccgccgcggccctgcccacgcccgtcgaggcccaggaccaggcgtcccttgacttctggaccaaggacatcgccgccacggaagccttcgcctgccgccagagctgcagcttcggcccgttcaccttcgtgtgcgacggcaacaccaagtaa

SEQ ID NO: 46 - protein, 76 aa, Geobacillus kaustophilus
MSLLALVAGTLGVSQSIATTVVSIVLTGSTLISIILGITAILSGGVDAILEIGWSAFVATVKKIVAERGKAAAIAW

SEQ ID NO: 47 - DNA, 231 bp, Geobacillus kaustophilus
  source 1..231: mol_type = genomic DNA
atgagtttgctggcgcttgttgccgggacgctcggcgtgtcacagtcaatcgcgacgacggttgtttcgattgtgttgaccggctccactctcatttctattattcttgggatcaccgctattttgtcaggtggagtcgacgccattttggaaattgggtggtcagcttttgtcgcgacggtgaaaaaaatagtggcggaacgaggaaaagcggcagcgattgcatggtaa

SEQ ID NO: 48 - protein, 309 aa, Clostridium tyrobutyricum
MRKVFLRSIISTLVMCAFVSSSFSVNADESKPNDEKIINNIENVTTTKDIVKSNKNNIVYLDEGVMSIPLSGRKPIAIKDDNNKEDLTVTLPIKNTGDISKISSNGTILYKNNSSNSSNIALQPKNDGFKALININDKLANKEYEFTFNLPKNSKLISAATYLGKEYDTKEVFVVDKNNIITSIISPAWAKDANGHNVSTYYKIVSNNKLVQVVEFTENTAFPVVADPNWTKIGKCAGSIAWAIGSGLFGGAKLIKIKKYIAELGGLQKAAKLLVGATTWEEKLHAGGYALINLAAELTGVAGIQANCF

SEQ ID NO: 49 - DNA, 930 bp, Clostridium tyrobutyricum
  source 1..930: mol_type = genomic DNA
ttgagaaaagtatttttaagatcaataatttcaacattagttatgtgtgcatttgtttcaagcagcttttcagtaaatgcggatgaaagcaaaccaaatgatgaaaaaataattaataacatagaaaacgttactactactaaagatattgtaaaaagtaataaaaataatattgtatatttagatgaaggtgtaatgagtattccattgtctgggagaaaacccattgctattaaagatgataataataaagaagatttaactgttacattacctattaagaatactggagatatatctaaaattagtagtaatggtactattctgtataaaaataatagtagtaattcatctaatatagctttacaacctaaaaatgatggatttaaggctttaataaatattaatgataagttagctaataaagaatatgaatttacatttaatttacccaaaaacagtaaattaattagtgctgccacatatttgggtaaagaatatgatacaaaagaagtatttgtagtagacaaaaataatataattacgagtattattagtccagcttgggctaaagatgcaaatggacataatgtttctacttattataagatagtatcgaataataaattagtacaagttgttgaattcacagaaaatactgcattcccggtggtagctgatcctaattggactaaaattgggaaatgcgctgggtcaatagcatgggctataggttctggcctttttggtggagcaaagctaattaaaataaaaaaatatatagcagagcttggaggacttcaaaaagcagctaaattattagttggtgcaaccacttgggaagaaaaattacacgcaggcggttatgcattaattaacttagctgctgagctaacaggtgtagcaggtatacaagcaaattgtttttaa

SEQ ID NO: 50 - protein, 62 aa, Bacillus coagulans
MKKIEKLTEKEMANIIGGKYYGNGVTCGKHSCSVDWGKATTCIINNGAMAWATGGHQGTHKC

SEQ ID NO: 51 - DNA, 189 bp, Bacillus coagulans
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacctgcataatcaataatggagctatggcatgggctactggtggacatcaaggtactcataaatgctag

SEQ ID NO: 52 - protein, 490 aa, Escherichia coli
MDKVTDNSPDVESTESTEGSFPTVGVDTGDTITATLATGTENVGGGGGAFGGASESSAAIHATAKWSTAQLKKHQAEQAARAAAAEAALAKAKSQRDALTQRLKDIVNDALRANAARSPSVTDLAHANNMAMQAEAERLRLAKAEQKAREEAEAAEKALREAERQRDEIARQQAETAHLLAMAEAAEAEKNRQDSLDEEHRAVEVAEKKLAEAKAELAKAESDVQSKQAIVSRVAGELENAQKSVDVKVTGFPGWRDVQKKLERQLQDKKNEYSSVTNALNSAVSIRDAKKTEVQNAEIKLKEAKDALEKSQVKDSVDTMVGFYQYITEQYGEKYSRIAQDLAEKAKGSKFNSVDEALAAFEKYKNVLDKKFSKVDRDDIFNALESITYDEWAKHLEKISRALKVTGYLSFGYDVWDGTLKGLKTGDWKPLFVTLEKSAVDFGVAKIVALMFSFIVGAPLGFWGIAIITGIVSSYIGDDELNKLNELLGI

SEQ ID NO: 53 - DNA, 1473 bp, Escherichia coli
  source 1..1473: mol_type = genomic DNA
atggataaagtcactgataattctccagatgtggagagcacagaatctactgaggggtcattcccaactgttggggttgatactggcgatacgattacagcgacgcttgcaactggaactgaaaatgttggtggaggcggtggagcatttggtggggccagtgaaagttctgctgcgatacatgcaaccgctaaatggtctaccgcgcagttgaaaaaacatcaggctgaacaggctgcccgtgctgctgcggctgaggcagcattggcaaaagcgaaatctcagcgtgatgccctgactcaacgtctcaaggatattgttaatgacgctttacgtgctaatgccgctcgtagtccatcagtaactgaccttgctcatgccaataatatggcaatgcaggcagaggctgagcgtttgcgccttgcgaaggcagagcaaaaagcccgtgaagaagctgaagcagcagaaaaagcgctccgggaagcagaacgccaacgtgatgagattgcccgccaacaggctgaaaccgcgcatttgttagcaatggcggaggcagcagaggctgagaaaaatcgacaggattctcttgatgaagagcatcgggctgtggaagtggcagagaagaagctggctgaggctaaagctgaactggcgaaggccgaaagcgatgtacagagtaagcaagcgattgtttccagagttgcaggggagcttgaaaacgctcaaaaaagtgttgatgtgaaggttaccggatttcctggatggcgtgatgttcagaaaaaactggagagacaattgcaggataagaagaatgaatattcgtcagtgacgaatgctcttaattctgctgttagcattagagatgctaaaaaaacagaagttcagaatgctgagataaaattaaaagaagctaaggatgctcttgagaagagtcaggtaaaagactctgttgatactatggttgggttttatcaatatataaccgaacaatatggggaaaaatattccagaatagctcaggatttagctgaaaaggcgaagggtagtaaatttaatagtgttgatgaagcacttgctgcatttgaaaagtataaaaatgtactggataagaaattcagtaaggttgatagggatgatatttttaatgctttagagtctattacttatgatgagtgggccaagcatctagaaaagatctctagggctcttaaggttactggatatttgtctttcgggtatgatgtatgggatggtaccctaaagggattaaaaacaggagactggaagcctttatttgtcactctggagaagagcgcggtagatttcggcgtggcaaaaattgtggcattaatgtttagttttattgttggtgcgcctcttggcttctggggaattgcaattatcacaggtattgtttcttcttacataggggatgatgagttgaacaagcttaatgaattactaggtatttaa

SEQ ID NO: 54 - protein, 522 aa, Escherichia coli
METAVAYYKDGVPYDDKGQVIITLLNGTPDGSGSGGGGGKGGSKSESSAAIHATAKWSTAQLKKTQAEQAARAKAAAEAQAKAKANRDALTQRLKDIVNEALRHNASRTPSATELAHANNAAMQAEDERLRLAKAEEKARKEAEAAEKAFQEAEQRRKEIEREKAETERQLKLAEAEEKRLAALSEEAKAVEIAQKKLSAAQSEVVKMDGEIKTLNSRLSSSIHARDAEMKTLAGKRNELAQASAKYKELDELVKKLSPRANDPLQNRPFFEATRRRVGAGKIREEKQKQVTASETRINRINADITQIQKAISQVSNNRNAGIARVHEAEENLKKAQNNLLNSQIKDAVDATVSFYQTLTEKYGEKYSKMAQELADKSKGKKIGNVNEALAAFEKYKDVLNKKFSKADRDAIFNALASVKYDDWAKHLDQFAKYLKITGHVSFGYDVVSDILKIKDTGDWKPLFLTLEKKAADAGVSYVVALLFSLLAGTTLGIWGIAIVTGILCSYIDKNKLNTINEVLGI

SEQ ID NO: 55 - DNA, 1569 bp, Escherichia coli
  source 1..1569: mol_type = genomic DNA
atggaaaccgcggtagcgtactataaagatggtgttccttatgatgataagggacaggtaattattactcttttgaatggtactcctgacgggagtggctctggcggcggaggtggaaaaggaggcagtaaaagtgaaagttctgcagctattcatgcaactgctaaatggtctactgctcaattaaagaaaacacaggcagagcaggctgcccgggcaaaagctgcagcggaagcacaggcgaaagcaaaggcaaacagggatgcgctgactcagcgcctgaaggatatcgtgaatgaggctcttcgtcacaatgcctcacgtacgccttcagcaacagagcttgctcatgctaataatgcagctatgcaggcggaagacgagcgtttgcgccttgcgaaagcagaagaaaaagcccgtaaagaagcggaagcagcagaaaaggcttttcaggaagcagaacaacgacgtaaagagattgaacgggagaaggctgaaacagaacgccagttgaaactggctgaagctgaagagaaacgactggctgcattgagtgaagaagctaaagctgttgagatcgcccaaaaaaaactttctgctgcacaatctgaagtggtgaaaatggatggagagattaagactctcaattctcgtttaagctccagtatccatgcccgtgatgcagaaatgaaaacgctcgctggaaaacgaaatgaactggctcaggcatccgctaaatataaagaactggatgagctggtcaaaaaactatcaccaagagccaatgatccgcttcagaaccgtcctttttttgaagcaaccagacgacgggttggggccggtaagattagagaagaaaaacaaaaacaggtaacagcatcagaaacacgtattaaccggataaatgctgatataactcagatccagaaggctatttctcaggtcagtaataatcgtaatgccggtatcgctcgtgttcatgaagctgaagaaaatttgaaaaaagcacagaataatctccttaattcacagattaaggatgctgttgatgcaacagttagcttttatcaaacgctgactgaaaaatatggtgaaaaatattcgaaaatggcacaggaacttgctgataagtctaaaggtaagaaaatcggcaatgtgaatgaagctctcgctgcttttgaaaaatacaaggatgttttaaataagaaattcagcaaagccgatcgtgatgctatttttaatgcgttggcatcggtgaagtatgatgactgggctaaacatttagatcagtttgccaagtacttgaagattacggggcatgtttcttttggatatgatgtggtatctgatatcctaaaaattaaggatacaggtgactggaagccactatttcttacattagagaagaaagctgcagatgcaggggtgagttatgttgttgctttactttttagcttgcttgctggaactacattaggtatttggggtattgctattgttacaggaattctatgctcctatattgataagaataaacttaatactataaatgaggtgttagggatttaa

SEQ ID NO: 56 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGI

SEQ ID NO: 57 - DNA, 1881 bp, Escherichia coli
  source 1..1881: mol_type = genomic DNA
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatgattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatccgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttaa

SEQ ID NO: 58 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLKTARNALTRAEQQLTQQKNTPDGKTIVSPEKFPGRSSTNHSIVVSGDPRFAGTIKITTSAVIDNRANLNYLLTHSGLDYKRNILNDRNPVVTEDVEGDKKIYNAEVAEWDKLRQRLLDARNKITSAESAINSARNNVSARTNEQKHANDALNALLKEKENIRSQLADINQKIAEEKRKRDEINMVKDAIKLTSDFYRTIYDEFGKQASELAKELASVSQGKQIKSVDDALNAFDKFRNNLNKKYNIQDRMAISKALEAINQVHMAENFKLFSKAFGFTGKVIERYDVAVELQKAVKTDNWRPFFVKLESLAAGRAASAVTAWAFSVMLGTPVGILGFAIIMAAVSALVNDKFIEQVNKLIGI

SEQ ID NO: 59 - DNA, 1881 bp, Escherichia coli
  source 1..1881: mol_type = genomic DNA
atgtctgaccctgtacgtattacaaatcccggtgcagaatcgctgggatatgattcagatggccatgaaattatggccgttgatatttatgtaaaccctccacgtgtcgatgtctttcatggtaccccgcctgcatggagttccttcgggaacaaaaccatctggggtggaaacgagtgggtcgatgattccccaacccgaagtgatatcgaaaaaagggacaaggaaatcacagcgtacaaaaacacgctcagcgcgcagcagaaagagaatgagaataagcgtactgaagctggaaaacgcctttctgcggcaattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgatgccctccggctgcatacagagagccggatgctgtttgctgatgctgattctcttcgtatatctccccgcgaggccaggtcgttaatcgaacaggctgaaaaacggcagaaggatgcgcagaacgcagacaagaaggccgctgatatgcttgctgaatacgagcgcagaaaaggtattctggacacgcggttgtcagagctggaaaaaaatggcggggcagcccttgccgttcttgatgcacaacaggcccgtctgctcgggcagcagacacggaatgacagggccatttcagaggcccggaataaactcagttcggtgacggaatcgcttaagacggcccgtaatgcattaaccagagctgaacaacagctgacgcaacagaaaaacacgcctgacggcaaaacgatagtttcccctgaaaaattcccggggcgttcatcaacaaatcattctattgttgtgagtggtgatccgaggtttgccggtacgataaaaatcacaaccagcgcggtcatcgataaccgtgcaaacctgaattatcttctgacccattccggtctggactataaacgcaatattctgaatgaccggaatccggtggtgacagaggatgtggaaggtgacaagaaaatttataatgctgaagttgctgaatgggataagttacggcaacgattgcttgatgccagaaataaaatcacctctgctgaatctgcgataaattcggcgagaaataacgtcagtgccagaacaaatgaacaaaagcatgcaaatgacgctcttaatgccctgttgaaggaaaaagagaatatccgtagccagcttgctgacatcaatcagaaaatagctgaagagaaaagaaaaagggatgaaataaatatggtaaaggatgccataaaactcacctctgatttctacagaacgatatatgatgagttcggtaaacaagcatccgaacttgctaaggagctggcttctgtatctcaagggaaacagattaagagtgtggatgatgcactgaacgcttttgataaattccgtaataatctgaacaagaaatataacatacaagatcgcatggccatttctaaagccctggaagctattaatcaggtccatatggcggagaattttaagctgttcagtaaggcatttggttttaccggaaaagttattgaacgttatgatgttgctgtggagttacaaaaggctgtaaaaacggacaactggcgtccattttttgtaaaacttgaatcactggcagcaggaagagctgcttcagcagttacagcatgggcgttttccgtcatgctgggaacccctgtaggtattctgggttttgcaattattatggcggctgtgagtgcgcttgttaatgataagtttattgagcaggtcaataaacttattggtatctga

SEQ ID NO: 60 - protein, 271 aa, Escherichia coli
METLTVHAPSPSTNLPSYGNGAFSLSAPHVPGAGPLLVQVVYSFFQSPNMCLQALTQLEDYIKKHGASNPLTLQIISTNIGYFCNADRNLVLHPGISVYDAYHFAKPAPSQYDYRSMNMKQMSGNVTTPIVALAHYLWGNGAERSVNIANIGLKISPMKINQIKDIIKSGVVGTFPVSTKFTHATGDYNVITGAYLGNITLKTEGTLTISANGSWTYNGVVRSYDDKYDFNASTHRGIIGESLTRLGAMFSGKEYQILLPGEIHIKESGKR

SEQ ID NO: 61 - DNA, 816 bp, Escherichia coli
  source 1..816: mol_type = genomic DNA
atggaaaccttaactgttcatgcaccatcaccatcaactaacttaccaagttatggcaatggtgcattttctctttcagcaccacatgtgcctggtgctggccctcttttagtccaggttgtttatagttttttccagagtccaaacatgtgtcttcaggctttaactcaacttgaggattacatcaaaaaacatggggccagcaaccctctcacattgcagatcatatcgacaaatattggttacttctgtaacgccgaccgaaatctggttcttcaccctggaataagcgtttatgacgcttaccacttcgcaaaaccagcgccaagtcaatatgactatcgctcaatgaatatgaaacaaatgagcggtaatgtcactacaccaattgtggcgcttgctcactatttatggggtaatggcgctgaaaggagcgttaatatcgccaacattggtcttaaaatttcccctatgaaaattaatcagataaaagacattataaaatctggtgtagtaggcacattccctgtttctacaaagttcacacatgccactggtgattataatgttattaccggtgcatatcttggtaatatcacactgaaaacagaaggtactttaactatctctgccaatggctcctggacttacaatggcgttgttcgttcatatgatgataaatacgattttaacgccagcactcaccgtggcattatcggagagtcgctcacaaggctcggggcgatgttttctggtaaagagtaccagatactgcttcctggtgaaattcacattaaagaaagtggtaagcgataa

SEQ ID NO: 62 - protein, 387 aa, Escherichia coli
MGSNGADNAHNNAFGGGKNPGIGNTSGAGSNGSASSNRGNSNGWSWSNKPHKNDGFHSDGSYHITFHGDNNSKPKPGGNSGNRGNNGDGASAKVGEITITPDNSKPGRYISSNPEYSLLAKLIDAESIKGTEVYTFHTRKGQYVKVTVPDSNIDKMRVDYVNWKGPKYNNKLVKRFVSQFLLFRKEEKEKNEKEALLKASELVSGMGDKLGEYLGVKYKNVAKEVANDIKNFHGRNIRSYNEAMASLNKVLANPKMKVNKSDKDAIVNAWKQVNAKDMANKIGNLGKAFKVADLAIKVEKIREKSIEGYNTGNWGPLLLEVESWIIGGVVAGVAISLFGAVLSFLPISGLAVTALGVIGIMTISYLSSFIDANRVSNINNIISSVIR

SEQ ID NO: 63 - DNA, 51 bp, Escherichia coli
  source 1..51: mol_type = genomic DNA
gcaaatcgagtttcgaatataaataacattatatctagtgttattcgatga

SEQ ID NO: 64 - protein, 103 aa, Escherichia coli
MRTLTLNELDSVSGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 65 - DNA, 312 bp, Escherichia coli
  source 1..312: mol_type = genomic DNA
atgagaactctgactctaaatgaattagattctgtttctggtggtgcttcagggcgtgatattgcgatggctataggaacactatccggacaatttgttgcaggaggaattggagcagctgctgggggtgtggctggaggtgcaatatatgactatgcatccactcacaaacctaatcctgcaatgtctccatccggtttaggaggaacaattaagcaaaaacccgaagggataccttcagaagcatggaactatgctgcgggaagattgtgtaattggagtccaaataatcttagtgatgtttgtttataa

SEQ ID NO: 66 - protein, 58 aa, Enterococcus columbae
MMNATENQIFVETVSDQELEMLIGGAGRGWIKTLTKDCPNVISSICAGTIITACKNCA

SEQ ID NO: 67 - DNA, 177 bp, Enterococcus columbae
  source 1..177: mol_type = genomic DNA
atgatgaatgctactgaaaaccaaatttttgttgagactgtgagtgaccaagaattagaaatgttaattggtggtgcaggtcgtggatggattaagactttaacaaaagattgtccaaatgtgatttcttcaatttgtgcaggtacaattattacagcttgtaaaaattgtgcttaa

SEQ ID NO: 68 - protein, 59 aa, Lactobacillus curvatus
MNNVKELSMTELQTITGGARSYGNGVYCNNKKCWVNRGEATQSIIGGMISGWASGLAGM

SEQ ID NO: 69 - DNA, 180 bp, Lactobacillus curvatus
  source 1..180: mol_type = genomic DNA
atgaataatgtaaaagaattaagtatgacagaattacaaacaattaccggcggtgctagatcatatggcaacggtgtttactgtaataataaaaaatgttgggtaaatcggggtgaagcaacgcaaagtattattggtggtatgattagcggctgggctagtggtttagctggaatgtaa

SEQ ID NO: 70 - protein, 64 aa, Streptomyces sp.
MRSEMTLTSTNSAEALAAQDFANTVLSAAAPGFHADCETPAMATPATPTVAQFVIQGSTICLVC

SEQ ID NO: 71 - DNA, 195 bp, Streptomyces sp.
  source 1..195: mol_type = genomic DNA
gtgcgatctgagatgactcttacgagcacgaattccgctgaggctctggcggcgcaggactttgcgaacaccgttctcagcgcggcggccccgggcttccacgcggactgcgagacgccggccatggccaccccggccacgccgaccgtcgcccagttcgtgatccagggcagcacgatctgcctggtctgctga

SEQ ID NO: 72 - protein, 65 aa, Bacillus halodurans
MVNSKDLRNPEFRKAQGLQFVDEVNEKELSSLAGSGDVHAQTTWPCATVGVSVALCPTTKCTSQC

SEQ ID NO: 73 - DNA, 198 bp, Bacillus halodurans
  source 1..198: mol_type = genomic DNA
atggtaaattcaaaagatttgcgtaatcctgaattccgcaaagcccaaggtctacaattcgttgacgaggtgaacgagaaggaactttcgtctctagctggttcaggagatgtgcatgcacaaacaacttggccttgcgctacagttggtgtctccgtagccttgtgcccaactacaaagtgtacaagccagtgctaa

SEQ ID NO: 74 - protein, 66 aa, Carnobacterium divergens
MKNLKEGSYTAVNTDELKSINGGTKYYGNGVYCNSKKCWVDWGQASGCIGQTVVGGWLGGAIPGKC

SEQ ID NO: 75 - DNA, 201 bp, Carnobacterium divergens
  source 1..201: mol_type = genomic DNA
atgaaaaacttaaaagaaggttcatacactgctgttaatactgatgaattaaaaagtatcaatggtggaacaaaatattatgggaatggcgtttattgcaattctaaaaaatgttgggtagattggggacaagcttcaggttgtatcggtcaaactgttgttggcggatggctaggcggagctataccaggtaaatgctaa

SEQ ID NO: 76 - protein, 63 aa, Carnobacterium divergens
MIKREKNRTISSLGYEEISNHKLQEIQGGKGILGKLGVVQAGVDFVSGVWAGIKQSAKDHPNA

SEQ ID NO: 77 - DNA, 192 bp, Carnobacterium divergens
  source 1..192: mol_type = genomic DNA
atgattaaaagagaaaagaacagaacaatttcttcccttggttatgaagaaatttctaatcataaattgcaagaaatacaaggtggaaaaggaattcttggtaaactaggagtagtacaggcaggagtggattttgtatcaggagtgtgggctggaataaaacagtctgccaaagatcatcctaatgcgtaa

SEQ ID NO: 78 - protein, 75 aa, Carnobacterium divergens
MKKQILKGLVIVVCLSGATFFSTPQQASAAAPKITQKQKNCVNGQLGGMLAGALGGPGGVVLGGIGGAIAGGCFN

SEQ ID NO: 79 - DNA, 228 bp, Carnobacterium divergens
  source 1..228: mol_type = genomic DNA
atgaaaaaacaaattttaaaagggttggttatagttgtttgtttatctggggcaacatttttctcaacaccacaacaagcttctgctgctgcaccgaaaattactcaaaaacaaaaaaattgtgttaatggacaattaggtggaatgcttgctggagctttgggtggacctggcggagttgtgttaggtggtataggtggtgcaatagcaggaggttgttttaattaa

SEQ ID NO: 80 - protein, 72 aa, Enterococcus durans
MQTIKELNTMELQEIIGGENDHRMPYELNRPNNLSKGGAKCAAGILGAGLGAVGGGPGGFISAGISAVLGCM

SEQ ID NO: 81 - DNA, 219 bp, Enterococcus durans
  source 1..219: mol_type = genomic DNA
atgcaaacgatcaaagaattgaacacgatggaattacaagaaataattggaggtgaaaatgaccatcggatgccttacgaattgaaccgtccaaataatttatccaaaggtggggctaagtgtgctgctggaatacttggcgctggactaggcgcagtaggcggtggacctggcggatttattagtgccggaatcagtgctgttcttggttgtatgtaa

SEQ ID NO: 82 - protein, 72 aa, Enterococcus durans
MQTIKELNTMELQKIIGGENDHRMPYELNRPNNLSKGGAKCAAGILGAGLGAVGGGPGGFISAGISAVLGCM

SEQ ID NO: 83 - DNA, 219 bp, Enterococcus durans
  source 1..219: mol_type = genomic DNA
atgcaaacgatcaaagaattgaacacgatggaattacaaaaaataattggaggtgaaaatgaccatcggatgccttacgaattgaaccgtccaaataatttatccaaaggtggagctaagtgcgctgccggaatacttggtgctggattaggcgcagtaggcggtggacctggcggatttattagtgccggaatcagtgctgttcttggttgtatgtaa

SEQ ID NO: 84 - protein, 220 aa, Streptococcus dysgalactiae subsp. equisimilis
MKKLKRLVISLVTSLLVISSTVPALVYANETNNFAETQKEITTNSEATLTNEDYTKLTSEVKTIYTNLIQYDQTKNKFYVDEDKTEQYYNYDDESIKGVYLMKDSLNDELNNNNSSNYSEIINQKISEIDYVLQGNDINNLIPSNTRVKRSADFSWIQRCLEEAWGYAISLVTLKGIINLFKAGKFEAAAAKLASATAGRIAGMAALFAFVATCGATTVS

SEQ ID NO: 85 - DNA, 663 bp, Streptococcus dysgalactiae subsp. equisimilis
  source 1..663: mol_type = genomic DNA
atgaaaaaattaaaacgtcttgttatctctcttgttacttcattactagtaatttcaagtacagttccagcacttgtttacgctaatgaaacaaataactttgcagaaactcaaaaagaaattacaacaaattcagaagcaacattaaccaatgaagactacactaaattaacttccgaagtaaaaacaatttatacaaatctgattcaatacgaccaaacaaaaaacaaattttacgtcgatgaagacaaaactgaacaatattataactacgatgatgaaagtataaaaggggtttatctcatgaaagatagtttgaacgatgagttaaacaataataactcttcaaactattctgaaataattaatcaaaaaatctctgaaattgactatgtccttcaaggaaacgatataaataatttaattcctagcaataccagagtaaaaagatcagcagatttttcttggattcaaagatgtctagaagaagcatggggatatgctattagtctagttactctaaaaggaataatcaatctatttaaagcaggaaaatttgaagctgctgctgctaaattagcttctgctacagcaggtagaatcgctggaatggctgccttatttgctttcgtagcaacttgcggtgcgacaactgtatcataa

SEQ ID NO: 86 - protein, 57 aa, Enterococcus faecalis
MKQYKVLNEKEMKKPIGGESVFSKIGNAVGPAAYWILKGLGNMSDVNQADRINRKKH

SEQ ID NO: 87 - DNA, 174 bp, Enterococcus faecalis
  source 1..174: mol_type = genomic DNA
atgaagcaatataaagtattgaatgaaaaagaaatgaaaaaacctattgggggagagtcggtttttagtaaaataggtaatgctgtaggtccagctgcttattggattttaaaaggattaggtaatatgagtgatgtaaaccaagctgatagaattaatagaaagaaacattaa

SEQ ID NO: 88 - protein, 44 aa, Enterococcus faecalis
MGAIAKLVAKFGWPIVKKYYKQIMQFIGEGWAINKIIDWIKKHI

SEQ ID NO: 89 - DNA, 135 bp, Enterococcus faecalis
  source 1..135: mol_type = genomic DNA
atgggagcaatcgcaaaattagtagcaaagtttggatggccaattgttaaaaagtattacaaacaaattatgcaatttattggagaaggatgggcaattaacaaaattattgattggatcaaaaaacatatttaa

SEQ ID NO: 90 - protein, 43 aa, Enterococcus faecalis
MGAIAKLVAKFGWPFIKKFYKQIMQFIGQGWTIDQIEKWLKRH

SEQ ID NO: 91 - DNA, 132 bp, Enterococcus faecalis
  source 1..132: mol_type = genomic DNA
atgggagcaatcgcaaaattagtagcaaagtttggatggccatttattaaaaaattctacaaacaaattatgcagtttatcggacaaggatggacaatagatcaaattgaaaaatggttaaaaagacattga

SEQ ID NO: 92 - protein, 74 aa, Enterococcus faecalis
MLNKKLLENGVVNAVTIDELDAQFGGMSKRDCNLMKACCAGQAVTYAIHSLLNRLGGDSSDPAGCNDIVRKYCK

SEQ ID NO: 93 - DNA, 225 bp, Enterococcus faecalis
  source 1..225: mol_type = genomic DNA
atgttaaataaaaaattattagaaaatggtgtagtaaatgctgtaacaattgatgaacttgatgctcaatttggtggaatgagcaaacgtgattgtaacttgatgaaggcgtgttgtgctggacaagcagtaacatatgctattcatagtcttttaaatcgattaggtggagactctagtgatccagctggttgtaatgatattgtaagaaaatattgtaaataa

SEQ ID NO: 94 - protein, 65 aa, Enterococcus faecium
MKHLKILSIKETQLIYGGTTHSGKYYGNGVYCTKNKCTVDWAKATTCIAGMSIGGFLGGAIPGKC

SEQ ID NO: 95 - DNA, 195 bp, Enterococcus faecium
  source 1..195: mol_type = genomic DNA
atgaaacatttaaaaattttgtctattaaagagacacaacttatctatgggggtaccactcatagtggaaaatattatggaaatggagtgtattgcactaaaaataaatgtacggtcgattgggccaaggcaactacttgtattgcaggaatgtctataggtggttttttaggtggagcaattccagggaagtgc

SEQ ID NO: 96 - protein, 105 aa, Enterococcus faecalis
MVKENKFSKIFILMALSFLGLALFSASLQFLPIAHMAKEFGIPAAVAGTVLNVVEAGGWVTTIVSILTAVGSGGLSLLAAAGRESIKAYLKKEIKKKGKRAVIAW

SEQ ID NO: 97 - DNA, 318 bp, Enterococcus faecalis
  source 1..318: mol_type = genomic DNA
atggttaaagaaaataaattttctaagatttttattttaatggctttgagttttttggggttagccttgtttagtgcaagtcttcagtttttgcccattgcacatatggctaaagagttcggtataccagcagcagttgcaggaactgtgcttaatgtagttgaagctggtggatgggtcactactattgtatcaattcttactgctgtaggtagcggaggtctttctttactcgctgcagcaggaagagagtcaattaaagcataccttaagaaagaaattaagaaaaaaggaaaaagagcagttattgcttggtaa

SEQ ID NO: 98 - protein, 71 aa, Enterococcus faecium
MQNVKELSTKEMKQIIGGENDHRMPNELNRPNNLSKGGAKCGAAIAGGLFGIPKGPLAWAAGLANVYSKCN

SEQ ID NO: 99 - DNA, 216 bp, Enterococcus faecium
  source 1..216: mol_type = genomic DNA
atgcaaaatgtaaaagaattaagtacgaaagagatgaaacaaattatcggtggagaaaatgatcacagaatgcctaatgagttaaatagacctaacaacttatctaaaggtggagcaaaatgtggtgctgcaattgctgggggattatttggaatcccaaaaggaccactagcatgggctgctgggttagcaaatgtatactctaaatgcaactaa

SEQ ID NO: 100 - protein, 58 aa, Enterococcus mundtii
MKKLTSKEMAQVVGGKYYGNGVSCNKKGCSVDWGKAIGIIGNNSAANLATGGAAGWKS

SEQ ID NO: 101 - DNA, 177 bp, Enterococcus mundtii
  source 1..177: mol_type = genomic DNA
ttgaagaaattaacatcaaaagaaatggcacaagtagtaggtggaaaatactacggtaatggagtctcatgtaataaaaaagggtgcagtgttgattggggaaaagctattggcattattggaaataattctgctgcgaatttagctactggtggagcagctggttggaaaagttaa

SEQ ID NO: 102 - protein, 44 aa, Enterococcus faecalis
MLAKIKAMIKKFPNPYTLAAKLTTYEINWYKQQYGRYPWERPVA

SEQ ID NO: 103 - DNA, 135 bp, Enterococcus faecalis
  source 1..135: mol_type = genomic DNA
atgttagcaaaaattaaagcgatgattaagaagtttccgaacccttatactttagcagctaagctaacgacttacgaaattaattggtataaacaacaatacggtcgttatccttgggagcgccctgtagcataa

SEQ ID NO: 104 - protein, 71 aa, Enterococcus faecium
MRKKLFSLALIGIFGLVVTNFGTKVDAATRSYGNGVYCNNSKCWVNWGEAKENIAGIVISGWASGLAGMGH

SEQ ID NO: 105 - DNA, 216 bp, Enterococcus faecium
  source 1..216: mol_type = genomic DNA
atgagaaaaaaattatttagtttagctcttattggaatatttgggttagttgtgacaaattttggtacaaaagttgatgcagctacgcgttcatatggtaatggtgtttattgtaataatagtaaatgctgggttaactggggagaagctaaagagaatattgcaggaatcgttattagtggctgggcttctggtttggcaggtatgggacattaa

SEQ ID NO: 106 - protein, 34 aa, Enterococcus faecium
MNFLKNGIAKWMTGAELQAYKKKYGCLPWEKISC

SEQ ID NO: 107 - DNA, 105 bp, Enterococcus faecium
  source 1..105: mol_type = genomic DNA
atgaattttcttaaaaatggtatcgcaaaatggatgaccggtgctgaattgcaagcgtataaaaagaaatatggatgcttgccatgggaaaaaatttcttgttaa

SEQ ID NO: 108 - protein, 76 aa, Enterococcus faecalis
MKKKLVKGLVICGMIGIGFTALGTNVEAATYYGNGVYCNKQKCWVDWSRARSEIIDRGVKAYVNGFTKVLGGIGGR

SEQ ID NO: 109 - DNA, 231 bp, Enterococcus faecalis
  source 1..231: mol_type = genomic DNA
atgaaaaagaaattagttaaaggcttagttatttgtggcatgattgggattggttttacagcattaggaacaaatgtagaagccgccacgtattacggaaatggtgtctattgcaataagcaaaaatgttgggtagattggagtagagcacgttctgaaattatagacagaggcgtaaaagcatacgtcaatggatttacgaaagtgttaggtggtataggtggaagataa

SEQ ID NO: 110 - protein, 60 aa, Enterococcus faecalis
MKKEELVGMAKEDFLNVICENDNKLENSGAKCPWWNLSCHLGNDGKICTYSHECTAGCNA

SEQ ID NO: 111 - DNA, 183 bp, Enterococcus faecalis
  source 1..183: mol_type = genomic DNA
atgaaaaaagaagaattagtaggaatggctaaggaagactttttaaatgttatttgtgaaaatgacaacaaactagaaaatagtggagcaaaatgtccttggtggaatctttcttgtcatttaggcaatgatggtaaaatttgcacttattcacatgaatgtaccgcaggttgtaatgcataa

SEQ ID NO: 112 - protein, 61 aa, Enterococcus faecalis
MTELNKRLQLKRDVSTENSLKKISNTDETHGGVTTSIPCTVMVSAAVCPTLVCSNKCGGRG

SEQ ID NO: 113 - DNA, 186 bp, Enterococcus faecalis
  source 1..186: mol_type = genomic DNA
atgactgaacttaacaaaagattacaattaaaaagagatgtttcaacagaaaatagtttgaaaaaaatttctaatactgatgaaacacatgggggagttactacatcaattccatgtacagtaatggttagtgcggcagtatgtcctacccttgtttgctcgaataaatgtggcggtagaggctag

SEQ ID NO: 114 - protein, 58 aa, Enterococcus faecium
MQNVKEVSVKEMKQIIGGSNDSLWYGVGQFMGKQANCITNHPVKHMIIPGYCLSKILG

SEQ ID NO: 115 - DNA, 177 bp, Enterococcus faecium
  source 1..177: mol_type = genomic DNA
atgcaaaatgtaaaagaagtttctgtaaaagagatgaaacaaattatcggtggttctaatgatagtctttggtatggtgtaggacaatttatgggtaaacaagcaaactgtataacaaaccatcctgttaaacacatgataattcctggatattgtttatcgaaaattttagggtaa

SEQ ID NO: 116 - protein, 55 aa, Enterococcus faecium
MKKYNELSKKELLQIQGGIAPIIVAGLGYLVKDAWDHSDQIISGFKKGWNGGRRK

SEQ ID NO: 117 - DNA, 168 bp, Enterococcus faecium
  source 1..168: mol_type = genomic DNA
atgaaaaaatataatgagttatctaaaaaagaacttctacagattcaaggaggaatagcacctattatagttgctggccttggctatttagtaaaagatgcatgggatcactcagatcaaataatctcaggatttaaaaaaggttggaatggtggacgtagaaaataa

SEQ ID NO: 118 - protein, 343 aa, Enterococcus faecalis
MKNILLSILGVLSIVVSLAFSSYSVNAASNEWSWPLGKPYAGRYEEGQQFGNTAFNRGGTYFHDGFDFGSAIYGNGSVYAVHDGKILYAGWDPVGGGSLGAFIVLQAGNTNVIYQEFSRNVGDIKVSTGQTVKKGQLIGKFTSSHLHLGMTKKEWRSAHSSWNKDDGTWFNPIPILQGGSTPTPPNPGPKNFTTNVRYGLRVLGGSWLPEVTNFNNTNDGFAGYPNRQHDMLYIKVDKGQMKYRVHTAQSGWLPWVSKGDKSDTVNGAAGMPGQAIDGVQLNYITPKGEKLSQAYYRSQTTKRSGWLKVSADNGSIPGLDSYAGIFGEPLDRLQIGISQSNPF

SEQ ID NO: 119 - DNA, 1032 bp, Enterococcus faecalis
  source 1..1032: mol_type = genomic DNA
atgaaaaatattttactttctattctaggggtattatctatcgttgtttctttggcgttttcttcttattctgtcaacgcagcttctaatgagtggtcgtggccactgggcaaaccatatgcgggaagatatgaagaaggacaacaattcgggaacactgcatttaaccgaggaggtacttatttccatgatgggtttgactttggttctgctatttatggaaatggcagtgtgtatgctgtgcatgatggtaaaattttatatgctggttgggatcctgtaggtggaggctcattaggtgcatttattgtactacaagcgggaaacacaaatgtgatttatcaagaatttagccgaaatgttggagatattaaagttagcactggacaaactgttaaaaaaggacagctgataggaaagtttacttctagtcatttacatttaggaatgacaaaaaaagaatggcgttctgctcattcttcttggaataaagatgatggcacttggtttaacccaattcctatacttcaaggaggatctacgcctacgcctccaaatccaggaccaaaaaatttcacaacaaatgttcgttacggattgcgggtcctcggaggttcatggttaccagaagtaaccaactttaacaataccaatgatggtttcgcaggttaccctaatcgtcaacatgatatgctttatataaaggtagataaagggcaaatgaaatatcgtgttcacacggctcaaagtggatggttgccttgggtaagtaaaggggataagagcgatacagtaaatggagcggcaggtatgcctggacaagcaattgatggtgttcagctaaactatataactcctaagggagaaaaattatcacaggcttactatcgttcacaaactacgaaacgatcaggctggttaaaagtaagtgcagataatggttctattcctggactagacagttatgcaggaatctttggagaaccgttggatcgcttgcaaataggtatttcacagtcaaatccattttaa

SEQ ID NO: 120 - protein, 56 aa, Staphylococcus epidermidis
MENKKDLFDLEIKKDNMENNNELEAQSLGPAIKATRQVCPKATRFVTVSCKKSDCQ

SEQ ID NO: 121 - DNA, 171 bp, Staphylococcus epidermidis
  source 1..171: mol_type = genomic DNA
atggaaaacaaaaaagatttatttgatttagaaatcaaaaaagataatatggaaaataataatgaattagaagctcaatctcttggtcctgcaattaaggcaactagacaggtatgtcctaaagcaacacgttttgttacagtttcttgtaaaaaaagtgattgtcaatag

SEQ ID NO: 122 - protein, 51 aa, Staphylococcus epidermidis
MAAFMKLIQFLATKGQKYVSLAWKHKGTILKWINAGQSFEWIYKQIKKLWA

SEQ ID NO: 123 - DNA, 156 bp, Staphylococcus epidermidis
  source 1..156: mol_type = genomic DNA
atggcagcatttatgaagttaattcagttcttagcaactaaaggtcaaaagtatgtttcacttgcatggaaacataaaggtactattttaaaatggattaacgccggtcaaagttttgaatggatttataaacaaatcaaaaaattatgggcataa

SEQ ID NO: 124 - protein, 52 aa, Staphylococcus epidermidis
MEAVKEKNDLFNLDVKVNAKESNDSGAEPRIASKFICTPGCAKTGSFNSYCC

SEQ ID NO: 125 - DNA, 159 bp, Staphylococcus epidermidis
  source 1..159: mol_type = genomic DNA
atggaagcagtaaaagaaaaaaatgatctttttaatcttgatgttaaagttaatgcaaaagaatctaacgattcaggagctgaaccaagaattgctagtaaatttatatgtactcctggatgtgcaaaaacaggtagttttaacagttattgttgttaa

SEQ ID NO: 126 - protein, 55 aa, Staphylococcus epidermidis
MNNSLFDLNLNKGVETQKSDLSPQSASVLKTSIKVSKKYCKGVTLTCGCNITGGK

SEQ ID NO: 127 - DNA, 168 bp, Staphylococcus epidermidis
  source 1..168: mol_type = genomic DNA
atgaataactcattattcgatttaaacctaaacaaaggtgtagaaactcaaaagagtgatttaagtccgcaatctgctagtgtcttgaagacttctattaaagtatctaaaaaatattgtaaaggtgttactttaacatgcggttgcaatattactggtggtaaataa

SEQ ID NO: 128 - protein, 52 aa, Staphylococcus gallinarum
MEAVKEKNELFDLDVKVNAKESNDSGAEPRIASKFLCTPGCAKTGSFNSYCC

SEQ ID NO: 129 - DNA, 159 bp, Staphylococcus gallinarum
  source 1..159: mol_type = genomic DNA
atggaagcagtaaaagagaaaaatgaactttttgatcttgacgttaaagtaaatgcaaaagagtctaatgattcaggcgcagaaccacgaattgctagtaaatttttatgtactcctggatgtgccaaaacaggtagcttcaatagctactgttgttaa

SEQ ID NO: 130 - protein, 63 aa, Lactococcus garvieae
MENNNYTVLSDEELQKIDGGIGGALGNALNGLGTWANMMNGGGFVNQWQVYANKGKINQYRPY

SEQ ID NO: 131 - DNA, 192 bp, Lactococcus garvieae
  source 1..192: mol_type = genomic DNA
atggaaaacaacaattacacagtactttcagatgaagaactacaaaaaattgatggtggaatcggcggggctcttggtaatgctctcaacggattaggtacctgggcaaacatgatgaacggtggaggatttgttaatcagtggcaagtttatgctaataaaggaaaaataaatcaataccgtccgtattaa

SEQ ID NO: 132 - protein, 63 aa, Lactococcus garvieae
MFDLVATGMAAGVAKTIVNAVSAGMDIATALSLFSGAFTAAGGIMALIKKYAQKKLWKQLIAA

SEQ ID NO: 133 - DNA, 192 bp, Lactococcus garvieae
  source 1..192: mol_type = genomic DNA
atgtttgatttagtcgcgactggaatggctgcaggtgtagcaaaaactattgttaatgccgttagtgctggtatggatattgccactgctttatcattgttctcaggagcttttactgcagctgggggaattatggcactcattaaaaaatatgctcaaaagaaattatggaaacagcttattgctgcataa

SEQ ID NO: 134 - protein, 91 aa, Lactobacillus gasseri
MVTKYGRNLGLNKVELFAIWAVLVVALLLTTANIYWIADQFGIHLATGTARKLLDAMASGASLGTAFAAILGVTLPAWALAAAGALGATAA

SEQ ID NO: 135 - DNA, 276 bp, Lactobacillus gasseri
  source 1..276: mol_type = genomic DNA
atggttactaagtacggacgtaatttaggtttgaacaaggtagagttgtttgcaatttgggcggttttagtagttgctcttttattgaccacagcgaacatttattggattgctgatcaattcgggattcatttagcgactggaacagcccgtaagttattagatgcaatggcttctggtgcctcattgggaactgcctttgctgctattttgggcgtgacattacctgcatgggctttggcagctgcaggagcattgggagcgactgcagcctag

SEQ ID NO: 136 - protein, 75 aa, Lactobacillus gasseri
MKNFNTLSFETLANIVGGRNNWAANIGGVGGATVAGWALGNAVCGPACGFVGAHYVPIAWAGVTAATGGFGKIRK

SEQ ID NO: 137 - DNA, 228 bp, Lactobacillus gasseri
  source 1..228: mol_type = genomic DNA
atgaaaaattttaatacattatcatttgaaacattggctaacatagttggtgggagaaataattgggctgctaatataggtggagtaggtggagcgacagtcgctggatgggctcttggaaatgcagtttgcggtcctgcttgtggctttgttggagcacactatgttccaatagcatgggctggcgtaacggcagctactggtggattcggaaagataagaaagtag

SEQ ID NO: 138 - protein, 64 aa, Lactobacillus plantarum
MSKLVKTLTISEISKAQNNGGKPAWCWYTLAMCGAGYDSGTCDYMYSHCFGIKHHSSGSSSYHC

SEQ ID NO: 139 - DNA, 195 bp, Lactobacillus plantarum
  source 1..195: mol_type = genomic DNA
atgagtaaattggttaagacacttactataagtgaaatttctaaggctcaaaacaacggtggaaaacctgcatggtgttggtatactttagcaatgtgtggtgctggttatgattcgggaacctgtgattatatgtattcgcattgttttggtataaagcatcatagtagtggtagtagcagttatcattgttag

SEQ ID NO: 140 - protein, 359 aa, Haloferax mediterranei
MSKDRDGRRTSRRGTLKKIGGFSLGALSFGAVGRTQAATGSSVTTADIAPPGPNGDPKSVQIDDKYTGAEMYGEGDFRVGLGTDLTMYPPVYRESLGNGSGGWEFDFTVCGSTACRFVDSNGDVKEDDKAKEMWWQEINFNDINQDLYSRNDSDWVGSTPADTQPEFDYTEFALARDGVTLALTALNPAMGSLALGATYFLSDMVNWIASQHEDDSSLKRKWDYDGLSGPLYADSSTYLLARDEMTSNSYESFTIDNIAVAFPEFPVRTKYYVTFTAPDDPSTQSISTLEEEGIYRVPATEVAAARPPGSRRSKSAADEMVYVADPKKFIEVEPVKNPSIPDRIYEEIEQKKKQRSRKQ

SEQ ID NO: 141 - DNA, 1080 bp, Haloferax mediterranei
  source 1..1080: mol_type = genomic DNA
atgtcgaaagacagagatgggagaaggacaagtcggcgaggcacgttaaagaaaatcggcggtttcagtctcggagcgcttagtttcggggcagtcggacgaactcaagcggcgaccggctcatcggttacgaccgctgatatcgcacctcccggaccgaacggagacccgaagagtgttcagatagatgataaatacaccggagccgagatgtacggcgagggtgacttcagagtcggtctcggaactgacctgacgatgtatccgcccgtgtaccgtgagagtcttggaaatggaagcgggggttgggaattcgacttcaccgtttgtgggtccactgcctgtcgatttgtggacagtaacggtgacgtcaaagaggacgacaaggcgaaagaaatgtggtggcaggaaattaacttcaacgacataaatcaggatttatacagtcggaacgattccgactgggtcgggtcgacccctgccgatacccaaccggagttcgattacaccgactttgcgctcgctcgggacggagtgacgctcgctctcacggcactcaaccccgcaatggggagtcttgcactcggtgccacgtacttcctcagcgacatggtgaactggattgcgagccagcacgaagacgacagttcgctcaagagaaaatgggattacgacgggctaagtgggccgttgtacgccgattcgtcgacgtacctactggcacgcgacgagatgacttcgaactcgtacgaatcattcacgatcgataacatcgccgttgccttcccagagttccccgtccggaccaagtactacgtcacattcactgcgccggatgacccgtcaacgcagtcgatatctacgctcgaagaggagggaatctaccgagtgcccgctacggaagtggctgcggccagaccaccggggtcccgacgttccaaatcggcagccgacgagatggtgtacgttgccgatccgaagaagttcatagaggtcgagccggtgaagaacccaagtatcccggaccgaatctacgaggagatagagcaaaaaaagaaacaacggagtaggaaacagtag

SEQ ID NO: 142 - protein, 311 aa, Haloarchaeon sp.
  source 1..311: note = S8a
MSDKDSINRRNVLRKIGGIGVASAVGFSGLASGESLSDDEKQDVIDTIYKSQRVEQIKKKFGGVNIEPKKVQSVTTNQSGDLVTAKLSVSDGDLVYSSVKDTTVIVQFDRSASEIGESWPKNTEAFIKSTSSGVDLLRTATDEEIKDVTEGVNTSEIESADAVNIFIDPESQTYYMEKYDFNNKVLEMFELATGGTSSGKISPTREDQNHEYNVREHKVFNSEKQNIQLQSDCNINSNTAADVILCFNQVGSCALCSPTLVGGPVPTVACLLVVCFGTPNAVSAILEEVDNSCFNLIKDVISCWDEWTSFW

SEQ ID NO: 143 - DNA, 936 bp, Haloarchaeon sp.
  source 1..936: mol_type = genomic DNA; note = S8a
atgtcggataaagacagcattaacagaagaaatgtattaagaaaaattggcggtatcggtgtggcttcagctgtcggattttctggtttggcaagcggggaaagtcttagcgatgatgagaaacaagatgttattgacacaatttacaaatcacaaagagttgaacagataaagaaaaagttcggaggagtgaatattgagccgaaaaaggttcaatctgtaacgaccaatcagagcggagatcttgttacggcgaagctgtcggttagtgatggggatttggtatattcgagtgtcaaagatacaactgtaatagttcagttcgatagatcggcttctgaaattggtgaaagttggcccaagaatactgaggcattcatcaaatcgacgtcctctggggtcgatcttctacgtacagcaactgatgaagaaataaaggacgttactgagggagtcaacacatctgaaattgaatctgcggatgctgttaacatatttattgatcctgaatcacagacatactatatggagaaatatgactttaataataaggtacttgagatgtttgaattagcgacaggtgggacaagtagtggtaaaatctcccccacacgtgaagaccagaatcacgaatataatgttagggaacataaagtatttaactcagaaaaacagaatatacaacttcagagtgactgtaatataaacagtaacaccgctgctgatgttattctatgcttcaaccaggttggttcttgtgcactctgctccccgactttagtcggaggtccagtccctacagttgcatgtctcttagtcgtctgtttcggcactccaaatgctgtgtccgcgatacttgaagaagtcgataattcttgctttaacttgatcaaggatgtaatttcgtgttgggatgaatggactagcttctggtga

SEQ ID NO: 144 - protein, 333 aa, Lactobacillus helveticus
MKHLNETTNVRILSQFDMDTGYQAVVQKGNVGSKYVYGLQLRKGATTILRGYRGSKINNPILELSGQAGGHTQTWEFAGDRKDINGEERAGQWFIGVKPSKIEGSKIIWAKQIARVDLRNQMGPHYSNTDFPRLSYLNRAGSNPFAGNKMTHAEAAVSPDYTKFLIATVENNCIGHFTIYNLDTINEKLDEKGNSEDVNLETVKYEDSFIIDNLYGDDNNSIVNSIQGYDLDNDGNIYISSQKAPDFDGSYYAHHKQIVKIPYYARSKESEDQWRAVNLSEFGGLDIPGKHSEVESIQIIGENHCYLTVAYHSKNKAGENKTTLNEIYELSWN

SEQ ID NO: 145 - DNA, 1002 bp, Lactobacillus helveticus
  source 1..1002: mol_type = genomic DNA
atgaagcatttaaatgaaacaactaatgttagaattttaagtcaatttgatatggatactggctatcaagcagtagttcaaaaaggcaatgtaggttcaaaatatgtatatggattacaacttcgcaaaggtgctactactatcttgcgtggttaccgtggaagtaaaattaataaccctattcttgaattatctggtcaagcaggtggtcacacacagacatgggaatttgctggtgatcgtaaagacattaatggtgaagaaagagcaggtcaatggtttataggtgttaaaccatcgaaaattgaaggaagcaaaattatttgggcaaagcaaattgcaagagttgatcttagaaatcaaatgggacctcattattcaaatactgactttcctcgattatcctacttgaatcgcgccggttctaatccatttgctggtaataagatgacgcatgccgaagccgcagtatcacctgattatactaagtttttaattgctactgttgaaaataactgtattggtcattttactatatacaatttagatacaattaatgaaaaacttgatgaaaagggaaatagtgaagatgttaatctcgaaactgttaaatacgaagatagttttatcattgataatttatatggtgatgataataattctattgtaaattcaattcaagggtatgatttggataatgatggaaatatttatatttccagtcaaaaagcgccagattttgatggctcttattatgcacatcataagcagattgttaagattccatattatgctcggtctaaagaaagcgaagaccaatggagagctgtaaatttaagcgaattcggtggcttggatattccaggtaaacatagtgaagttgaaagcatccaaattattggtgagaatcattgttacttaactgttgcatatcattctaaaaataaagcgggtgaaaataaaactactttgaatgagatttatgaattatcttggaattag

SEQ ID NO: 146 - protein, 74 aa, Enterococcus hirae; identical to SEQ ID NO: 20

SEQ ID NO: 147 - DNA, 225 bp, Enterococcus hirae; identical to SEQ ID NO: 21
  source 1..225: mol_type = genomic DNA

SEQ ID NO: 148 - protein, 75 aa, Lactobacillus johnsonii
MKQFNYLSHKDLAVVVGGRNNWQTNVGGAVGSAMIGATVGGTICGPACAVAGAHYLPILWTAVTAATGGFGKIRK

SEQ ID NO: 149 - DNA, 228 bp, Lactobacillus johnsonii
  source 1..228: mol_type = genomic DNA
atgaaacaatttaattatttatcacataaagatttagcagtcgttgttggtggaagaaataattggcaaacaaatgtgggaggagcagtgggatcagctatgattggggctacagttggtggtacaatttgtggacctgcatgtgctgtagctggtgcccattatcttcctattttatggacagcggttacagctgcaacaggtggttttggcaagataagaaagtag

SEQ ID NO: 150 - protein, 62 aa, Lactobacillus johnsonii
MKLNDKELSKIVGGNRWGDTVLSAASGAGTGIKACKSFGPWGMAICGVGGAAIGGYFGYTHN

SEQ ID NO: 151 - DNA, 189 bp, Lactobacillus johnsonii
  source 1..189: mol_type = genomic DNA
atgaaattaaatgacaaagaattatcaaagattgttggtggaaatcgatggggagatactgttttatcagctgctagtggcgcaggaactggtattaaagcatgtaaaagttttggcccatggggaatggcaatttgtggtgtaggaggtgcagcaataggaggttattttggctatactcataattaa

SEQ ID NO: 152 - protein, 59 aa, Lactococcus lactis
  source 1..59: note = subsp. lactis
MNKNEIETQPVTWLEEVSDQNFDEDVFGACSTNTFSLSDYWGNNGAWCTLTHECMAWCK

SEQ ID NO: 153 - DNA, 180 bp, Lactococcus lactis
  source 1..180: mol_type = genomic DNA; note = subsp. lactis
atgaacaaaaatgaaattgaaacacaaccagttacatggttggaagaagtatctgatcaaaattttgatgaagatgtatttggtgcgtgtagtactaacacattctcgctcagtgattactggggaaataacggggcttggtgtacactcactcatgaatgtatggcttggtgtaaataa

SEQ ID NO: 154 - protein, 65 aa, Lactococcus lactis
  source 1..65: note = subsp. lactis
MKEKNMKKNDTIELQLGKYLEDDMIELAEGDESHGGTTPATPAISILSAYISTNTCPTTKCTRAC

SEQ ID NO: 155 - DNA, 198 bp, Lactococcus lactis
  source 1..198: mol_type = genomic DNA; note = subsp. lactis
atgaaagaaaaaaatatgaaaaagaatgacactattgaattacaattgggaaaataccttgaagatgatatgattgaattagctgaaggggatgagtctcatggaggaacaacaccagcaactcctgcaatctctattctcagtgcatatattagtaccaatacttgtccaacaacaaaatgtacacgtgcttgttaa

SEQ ID NO: 156 - protein, 51 aa, Lactococcus lactis
  source 1..51: note = subsp. lactis
MKEQNSFNLLQEVTESELDLILGAKGGSGVIHTISHECNMNSWQFVFTCCS

SEQ ID NO: 157 - DNA, 156 bp, Lactococcus lactis
  source 1..156: mol_type = genomic DNA; note = subsp. lactis
atgaaagaacaaaactcttttaatcttcttcaagaagtgacagaaagtgaattggaccttattttaggtgcaaaaggcggcagtggagttattcatacaatttctcatgaatgtaatatgaatagctggcaatttgtatttacttgctgctcttaa

SEQ ID NO: 158 - protein, 53 aa, Lactococcus lactis
MAGFLKVVQLLAKYGSKAVQWAWANKGKILDWLNAGQAIDWVVSKIKQILGIK

SEQ ID NO: 159 - DNA, 162 bp, Lactococcus lactis
  source 1..162: mol_type = genomic DNA
atggcagggtttttaaaagtagttcaattactagctaaatatggttctaaagctgtacaatgggcttgggcaaacaagggtaagattttagattggcttaatgcaggtcaggctattgattgggtagtttcgaaaattaagcaaattttaggtattaagtaa

SEQ ID NO: 160 - protein, 53 aa, Lactococcus lactis
MAGFLKVVQILAKYGSKAVQWAWANKGKILDWINAGQAIDWVVEKIKQILGIK

SEQ ID NO: 161 - DNA, 162 bp, Lactococcus lactis
  source 1..162: mol_type = genomic DNA
atggcagggtttttaaaagtagtccaaattttggctaagtatggttctaaagccgtacaatgggcatgggcaaataaaggaaaaatcttagattggattaatgcaggtcaagctattgactgggtagttgaaaagattaagcaaattttgggtattaaataa

SEQ ID NO: 162 - protein, 65 aa, Lactobacillus amylovorus
MKQLNSEQLQNIIGGNRWTNAYSAALGCAVPGVKYGKKLGGVWGAVIGGVGGAAVCGLAGYVRKG

SEQ ID NO: 163 - DNA, 198 bp, Lactobacillus amylovorus
  source 1..198: mol_type = genomic DNA
atgaaacaattgaattcagaacaattacaaaatattatcggtggaaatagatggactaatgcatacagcgcagctttgggatgcgctgtccctggagttaaatatggaaaaaaacttggtggcgtatggggtgctgtaattggtggcgtaggcggtgcagcagtctgtggcttggcgggttatgttcgtaaaggctaa

SEQ ID NO: 164 - protein, 68 aa, Lactobacillus sakei
  source 1..68: note = L45
MKTEKKVLDELSLHASAKMGARDVESSMNADSTPVLASVAVSMELLPTASVLYSDVAGCFKYSAKHHC

SEQ ID NO: 165 - DNA, 207 bp, Lactobacillus sakei
  source 1..207: mol_type = genomic DNA; note = L45
atgaaaacagaaaaaaaggttttagatgaactgagcttacacgcttctgcaaaaatgggagcacgtgatgttgaatccagcatgaatgcagactcaacaccagttttagcatcagtcgctgtatccatggaattattgccaactgcgtctgttctttattcggatgttgcaggttgcttcaaatattctgcaaaacatcattgttag

SEQ ID NO: 166 - protein, 91 aa, Lactococcus lactis
  source 1..91: note = subsp. lactis
MKTKSLVLALSAVTLFSAGGIVAQAEGTWQHGYGVSSAYSNYHHGSKTHSATVVNNNTGRQGKDTQRAGVWAKATVGRNLTEKASFYYNFW

SEQ ID NO: 167 - DNA, 276 bp, Lactococcus lactis
  source 1..276: mol_type = genomic DNA; note = subsp. lactis
atgaaaaccaagtctctcgtattggcattatctgcggttacgttattctctgccggaggaattgtagctcaagctgaaggaacatggcaacatggatatggtgttagttcggcatattcaaattatcatcatggtagcaaaactcattcagccacagttgtaaataataatactggccgacaaggtaaggatacacaacgtgccggtgtttgggcaaaagctactgttggacgtaacttaactgaaaaagcttcattttattataacttttggtaa

SEQ ID NO: 168 - protein, 75 aa, Lactococcus lactis
  source 1..75: note = subsp. cremoris
MKNQLNFNIVSDEELSEANGGKLTFIQSTAAGDLYYNTNTHKYVYQQTQNAFGAAANTIVNGWMGGAAGGFGLHH

SEQ ID NO: 169 - DNA, 228 bp, Lactococcus lactis
  source 1..228: mol_type = genomic DNA; note = subsp. cremoris
atgaaaaatcaattaaattttaatattgtttcagatgaagaactttcagaagctaacggaggaaaattaacatttattcaatcgacagcggctggagatttatattacaatactaatacacacaaatatgtttaccaacaaactcaaaacgcttttggggctgctgctaataccattgttaatggatggatgggtggcgctgctggaggtttcgggttgcaccattga

SEQ ID NO: 170 - protein, 68 aa, Lactococcus lactis
  source 1..68: note = subsp. cremoris
MKNQLNFNIVSDEELAEVNGGSLQYVMSAGPYTWYKDTRTGKTICKQTIDTASYTFGVMAEGWGKTFH

SEQ ID NO: 171 - DNA, 207 bp, Lactococcus lactis
  source 1..207: mol_type = genomic DNA; note = subsp. cremoris
atgaaaaatcaattaaattttaatattgtttctgatgaagaacttgcagaagttaatggaggaagcttgcagtatgttatgagtgctggaccatatacttggtataaagatactagaacaggaaaaacaatatgtaaacagacaattgacacagcaagttatacatttggtgtaatggcagaaggatggggaaaaacattccactaa

SEQ ID NO: 172 - protein, 63 aa, Lactococcus sp.
  source 1..63: note = QU 12
MKLIDHLGAPRWAVDTILGAIAVGNLASWVLALVPGPGWAVKAGLATAAAIVKHQGKAAAAAW

SEQ ID NO: 173 - DNA, 192 bp, Lactococcus sp.
  source 1..192: mol_type = genomic DNA; note = QU 12
atgaaattaattgatcatttaggtgctccaagatgggccgttgatactattttaggtgcaatcgcagttgggaacttagcaagttgggttctagcgcttgtccctggtccagggtgggcagtaaaagctggtttagcaactgctgctgccatcgttaaacatcaaggtaaagctgccgctgctgcttggtaa

SEQ ID NO: 174 - protein, 50 aa, Brevibacillus sp.
  source 1..50: note = GI-9
MACQCPDAISGWTHTDYQCHGLENKMYRHVYAICMNGTQVYCRTEWGSSC

SEQ ID NO: 175 - DNA, 153 bp, Brevibacillus sp.
  source 1..153: mol_type = genomic DNA; note = GI-9
atggcttgccaatgtccagatgcgatctcaggttggacgcatacagattaccagtgtcacggtttggagaataaaatgtatagacatgtttatgcaatttgcatgaacggtactcaagtatattgcagaacagagtggggtagcagctgctag

SEQ ID NO: 176 - protein, 57 aa, Leuconostoc pseudomesenteroides
MNKEYNSISNFKKITNKDLQNINGGFIGRAIGDFVYFGAKGLRESGKLLNYYYKHKH

SEQ ID NO: 177 - DNA, 174 bp, Leuconostoc pseudomesenteroides
  source 1..174: mol_type = genomic DNA
atgaataaagaatataatagcattagcaattttaaaaaaattactaataaagacttgcaaaacataaatggtggatttattggtagggcaataggtgactttgtgtactttggagcgaagggactaagagaatctggtaaactacttaattattactataagcataagcattga

SEQ ID NO: 178 - protein, 53 aa, Leuconostoc pseudomesenteroides
MKNQLMSFEVISEKELSTVQGGKGLGKLIGIDWLLGQAKDAVKQYKKDYKRWH

SEQ ID NO: 179 - DNA, 162 bp, Leuconostoc pseudomesenteroides
  source 1..162: mol_type = genomic DNA
atgaaaaatcagttaatgtctttcgaagtgatatcagaaaaagaattgtccacggtacaaggtggcaaaggcttaggtaaactcataggaattgattggcttttgggtcaagctaaggacgctgttaaacagtacaagaaggattacaaacgttggcactaa

SEQ ID NO: 180 - protein, 61 aa, Leuconostoc gelidum
MMNMKPTESYEQLDNSALEQVVGGKYYGNGVHCTKSGCSVNWGEAFSAGVHRLANGGNGFW

SEQ ID NO: 181 - DNA, 186 bp, Leuconostoc gelidum
  source 1..186: mol_type = genomic DNA
atgatgaacatgaaacctacggaaagctatgagcaattggataatagtgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 182 - protein, 61 aa, Leuconostoc carnosum
MNNMKSADNYQQLDNNALEQVVGGKYYGNGVHCTKSGCSVNWGEAFSAGVHRLANGGNGFW

SEQ ID NO: 183 - DNA, 186 bp, Leuconostoc carnosum
  source 1..186: mol_type = genomic DNA
atgaataacatgaaatctgcggataattatcagcaattggataataatgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 184 - protein, 63 aa, Leuconostoc mesenteroides
MFLVNQLGISKSLANTILGAIAVGNLASWLLALVPGPGWATKAALATAETIVKHEGKAAAIAW

SEQ ID NO: 185 - DNA, 192 bp, Leuconostoc mesenteroides
  source 1..192: mol_type = genomic DNA
atgttcttggtaaatcagttagggatttcaaaatcgttagctaatactattcttggtgcaattgctgttggtaatttggccagttggttattagctttggttcctggtccgggttgggcaacaaaagcagcacttgcgacagctgaaacaattgtgaagcatgaaggaaaagcagctgctattgcgtggtaa

SEQ ID NO: 186 - protein, 74 aa, Bacillus licheniformis
MSKKEMILSWKNPMYRTESSYHPAGNILKELQEEEQHSIAGGTITLSTCAILSKPLGNNGYLCTVTKECMPSCN

SEQ ID NO: 187 - DNA, 225 bp, Bacillus licheniformis
  source 1..225: mol_type = genomic DNA
atgtcaaaaaaggaaatgattctttcatggaaaaatcctatgtatcgcactgaatcttcttatcatccagcagggaacatccttaaagaactccaggaagaggaacagcacagcatcgccggaggcacaatcacgctcagcacttgtgccatcttgagcaagccgttaggaaataacggatacctgtgtacagtgacaaaagaatgcatgccaagctgtaactaa

SEQ ID NO: 188 - protein, 266 aa, Brevibacterium linens
MNNLYRELAPIPGPAWAEIEEEARRTFKRNIAGRRIVDVAGPTGFETSAVTTGHIRDVQSETSGLQVKQRIVQEYIELRTPFTVTRQAIDDVARGSGDSDWQPVKDAATTIAMAEDRAILHGLDAAGIGGIVPGSSNAAVAIPDAVEDFADAVAQALSVLRTVGVDGPYSLLLSSAEYTKVSESTDHGYPIREHLSRQLGAGEIIWAPALEGALLVSTRGGDYELHLGQDLSIGYYSHDSETVELYLQETFGFLALTDESSVPLSL

SEQ ID NO: 189 - DNA, 801 bp, Brevibacterium linens
  source 1..801: mol_type = genomic DNA
gtgaataacctctatcgcgagcttgcccccatccccggcccggcctgggcggagatcgaggaggaggctcgacggacattcaaacgcaatatcgccggccgccggatcgtcgatgtcgcagggcccacgggcttcgagacctccgcggtgaccactggccacatccgagacgtccagtcggagacgagcggactgcaggttaagcagcgcatcgtgcaggaatacatcgagctgcggaccccattcaccgtgactcggcaggccatcgatgacgtggcccgcgggtccggtgactcggactggcagcccgtcaaggatgcggccacgacgatcgcgatggctgaagatcgggccattctccacgggctcgatgcggccgggatcggcggaatcgttcccggcagctcgaatgccgcagtggccatccccgacgccgtcgaggacttcgcggacgccgtcgcccaggcgctgagtgtgctgcgcacggtgggagtcgacgggccctacagcctgttgctctcctccgcggagtacaccaaggtctccgagtccaccgaccacggctacccgatccgcgagcacctctcccggcagctcggcgccggagagatcatctgggcgcccgcgctcgaaggggcgctgctcgtctccacgcgcgggggtgactacgagctccacctcggccaggacctgtcgatcggttactacagccacgacagcgagaccgtcgaactctatctgcaggagaccttcggattcctcgcgctgaccgacgaatccagtgtgcctttgagcctctga

SEQ ID NO: 190 - protein, 71 aa, Listeria innocua
MKKAALKFIIVIAILGFSFSFFSIQSEAKSYGNGVQCNKKKCWVDWGSAISTIGNNSAANWATGGAAGWKS

SEQ ID NO: 191 - DNA, 216 bp, Listeria innocua
  source 1..216: mol_type = genomic DNA
ttgaagaaggcagcgttaaaatttattattgttattgctattctaggtttcagtttttctttctttagcatacaatctgaagctaaatcttatggaaatggagttcagtgtaataagaaaaaatgttgggtagattggggtagtgctataagtactattggaaataattctgcagcgaattgggctacaggtggagcagctggttggaaaagctga

SEQ ID NO: 192 - protein, 68 aa, Bacillus sp.
MSQEAIIRSWKDPFSRENSTQNPAGNPFSELKEAQMDKLVGAGDMEAACTFTLPGGGGVCTLTSECIC

SEQ ID NO: 193 - DNA, 207 bp, Bacillus sp.
  source 1..207: mol_type = genomic DNA
atgagtcaagaagctatcattcgttcatggaaagatcctttttcccgtgaaaattctacacaaaatccagctggtaacccattcagtgagctgaaagaagcacaaatggataagttagtaggtgcgggagacatggaagcagcatgtacttttacattgcctggtggcggcggtgtttgtactctaacttctgaatgtatttgttaa

SEQ ID NO: 194 - protein, 61 aa, Leuconostoc mesenteroides
MTNMKSVEAYQQLDNQNLKKVVGGKYYGNGVHCTKSGCSVNWGEAASAGIHRLANGGNGFW

SEQ ID NO: 195 - DNA, 186 bp, Leuconostoc mesenteroides
  source 1..186: mol_type = genomic DNA
atgacgaatatgaagtctgtggaagcatatcagcaattagataaccagaatctcaagaaagttgttggtggaaagtattatgggaatggtgttcactgtacaaaaagtggatgctctgttaactggggagaagctgcctcagctggcatacatcgtttggccaatggtggaaatggattttggtaa

SEQ ID NO: 196 - protein, 68 aa, Clavibacter michiganensis
  source 1..68: note = subsp. michiganensis
MNDILETETPVMVSPRWDMLLDAGEDTSPSVQTQIDAEFRRVVSPYMSSSGWLCTLTIECGTIICACR

SEQ ID NO: 197 - DNA, 207 bp, Clavibacter michiganensis
  source 1..207: mol_type = genomic DNA; note = subsp. michiganensis
atgaacgacatcctcgagacggagacccccgtcatggtcagcccccggtgggacatgctgctcgacgcgggcgaggacaccagcccgtccgtccagacccagatcgacgcggagttccgtcgcgtcgtgagcccgtacatgtccagcagcggctggctctgcacgctcaccatcgaatgtggcaccatcatctgcgcgtgtcgctga

SEQ ID NO: 198 - protein, 69 aa, Escherichia coli
MELKASEFGVVLSVDALKLSRQSPLGVGIGGGGGGGGGGSCGGQGGGCGGCSNGCSGGNGGSGGSGSHI

SEQ ID NO: 199 - DNA, 207 bp, Escherichia coli
  source 1..207: mol_type = genomic DNA
atggaattaaaagcgagtgaatttggtgtagttttgtccgttgatgctcttaaattatcacgccagtctccattaggtgttggcattggtggtggtggcggcggcggcggcggcggtagctgcggtggtcaaggtggcggttgtggtggttgcagcaacggttgtagtggtggaaacggtggcagcggcggaagtggttcacatatc

SEQ ID NO: 200 - protein, 7 aa, Escherichia coli
MRTGNAN

SEQ ID NO: 201 - DNA, 24 bp, Escherichia coli
  source 1..24: mol_type = genomic DNA
atgcgtactggtaatgcaaactaa

SEQ ID NO: 202 - protein, 99 aa, Klebsiella pneumoniae
MREISQKDLNLAFGAGETDPNTQLLNDLGNNMAWGAALGAPGGLGSAALGAAGGALQTVGQGLIDHGPVNVPIPVLIGPSWNGSGSGYNSATSSSGSGS

SEQ ID NO: 203 - DNA, 300 bp, Klebsiella pneumoniae
  source 1..300: mol_type = genomic DNA
atgagagaaattagtcaaaaggacttaaatcttgcttttggtgcaggagagaccgatccaaatactcaacttctaaacgaccttggaaataatatggcatggggtgctgctcttggcgctcctggcggattaggatcagcagctttgggggccgcgggaggtgcattacaaactgtagggcaaggattaattgaccatggtcctgtaaatgtccccatccctgtactcatcgggccaagctggaatggtagcggtagtggttataacagcgcaacatccagttccggtagtggtagttaa

SEQ ID NO: 204 - protein, 75 aa, Escherichia coli
MREITESQLRYISGAGGAPATSANAAGAAAIVGALAGIPGGPLGVVVGAVSAGLTTAIGSTVGSGSASSSAGGGS

SEQ ID NO: 205 - DNA, 228 bp, Escherichia coli
  source 1..228: mol_type = genomic DNA
atgcgagaaataacagaatcacagttaagatatatttccggggcgggaggtgcgccagcgacttcagctaatgccgcaggtgctgcagctattgttggagctctcgccggaatacctggtggtccacttggggttgtagttggagccgtatctgccggtttgacaacagcaattggctcgaccgtgggaagtggtagtgccagttcttctgctggtggcggtagctaa

SEQ ID NO: 206 - protein, 58 aa, Escherichia coli
MIKHFHFNKLSSGKKNNVPSPAKGVIQIKKSASQLTKGGAGHVPEYFVGIGTPISFYG

SEQ ID NO: 207 - DNA, 177 bp, Escherichia coli
  source 1..177: mol_type = genomic DNA
atgattaagcattttcattttaataaactgtcttctggtaaaaaaaataatgttccatctcctgcaaagggggttatacaaataaaaaaatcagcatcgcaactcacaaaaggtggtgcaggacatgtgcctgagtattttgtggggattggtacacctatatctttctatggctga

SEQ ID NO: 208 - protein, 90 aa, Escherichia coli
MYMRELDREELNCVGGAGDPLADPNSQIVRQIMSNAAWGPPLVPERFRGMAVGAAGGVTQTVLQGAAAHMPVNVPIPKVPMGPSWNGSKG

SEQ ID NO: 209 - DNA, 273 bp, Escherichia coli
  source 1..273: mol_type = genomic DNA
atgtatatgagagagttagatagagaggaattaaattgcgttggtggggctggagatccgcttgcagatcctaattcccaaattgtaagacagataatgtctaatgcggcatggggcccgcctttggtgccagagcggtttaggggaatggctgttggagccgcaggtggggttacgcagacagttcttcaaggagcagcagctcatatgccggttaatgtccctatacctaaagttccgatgggaccctcatggaacggaagtaaaggataa

SEQ ID NO: 210 - protein, 50 aa, Enterococcus mundtii
MSQVVGGKYYGNGVSCNKKGCSVDWGKAIGIIGNNSAANLATGGAAGWKS

SEQ ID NO: 211 - DNA, 153 bp, Enterococcus mundtii
  source 1..153: mol_type = genomic DNA
atgtcacaggtagtaggtggaaaatactacggtaatggagtctcatgtaataaaaaagggtgcagtgttgattggggaaaagcgattggcattattggaaataattctgctgcgaatttagctactggtggagcagctggttggaaaagttaa

SEQ ID NO: 212 - protein, 58 aa, Enterococcus mundtii
MKKLTSKEMAQVVGGKYYGNGLSCNKKGCSVDWGKAIGIIGNNSAANLATGGAAGWKS

SEQ ID NO: 213 - DNA, 177 bp, Enterococcus mundtii
  source 1..177: mol_type = genomic DNA
ttgaagaaattaacatcaaaagaaatggcacaagtagtaggtgggaaatactacggtaatggattatcatgtaataaaaaagggtgcagtgttgattggggaaaagctattggcattattggaaataattctgctgcgaatttagctactggtggagcagctggttggaaaagttaa

SEQ ID NO: 214 - protein, 63 aa, Streptococcus mutans
MSNTQLLEVLGTETFDVQEDLFAFDTTDTTIVASNDDPDTRFKSWSLCTPGCARTGSFNSYCC

SEQ ID NO: 215 - DNA, 192 bp, Streptococcus mutans
  source 1..192: mol_type = genomic DNA
atgtcaaacacacaattattagaagtccttggtactgaaacttttgatgttcaagaagatctctttgcttttgatacaacagatactactattgtggcaagcaacgacgatccagatactcgtttcaaaagttggagcctttgtacgcctggttgtgcaaggacaggtagtttcaatagttactgttgctga

SEQ ID NO: 216 - protein, 53 aa, Streptococcus mutans
MNKLNSNAVVSLNEVSDSELDTILGGNRWWQGVVPTVSYECRMNSWQHVFTCC

SEQ ID NO: 217 - DNA, 162 bp, Streptococcus mutans
  source 1..162: mol_type = genomic DNA
atgaacaagttaaacagtaacgcagtagtttctttgaatgaagtttcagattctgaattggatactattttgggtggtaatcgttggtggcaaggtgttgtgccaacggtctcatatgagtgtcgcatgaattcatggcaacatgttttcacttgctgttaa

SEQ ID NO: 218 - protein, 57 aa, Lactococcus lactis
  source 1..57: note = subsp. lactis
MSTKDFNLDLVSVSKKDSGASPRITSISLCTPGCKTGALMGCNMKTATCHCSIHVSK

SEQ ID NO: 219 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataa

SEQ ID NO: 220 - protein, 57 aa, Lactococcus lactis
MSTKDFNLDLVSVSKKDSGASPRITSISLCTPGCKTGALMGCNMKTATCNCSVHVSK

SEQ ID NO: 221 - DNA, 171 bp, Lactococcus lactis
  source 1..171: mol_type = genomic DNA
atgagtacaaaagatttcaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagcgttcacgtaagcaaa

SEQ ID NO: 222 - protein, 57 aa, Lactococcus lactis
MSTKDFNLDLVSVSKTDSGASTRITSISLCTPGCKTGVLMGCNLKTATCNCSVHVSK

SEQ ID NO: 223 - DNA, 174 bp, Lactococcus lactis
  source 1..174: mol_type = genomic DNA
atgagtacaaaagatttcaacttagatttggtatctgtttcaaaaacagattctggcgcttcaacacgtattaccagcatttcgctttgtacaccaggttgtaaaacaggtgttctgatgggatgtaacctgaaaacagcaacttgtaattgtagcgttcacgtaagcaaataa

SEQ ID NO: 224 - protein, 55 aa, Streptococcus uberis
MNNEDFNLDLIKISKENNSGASPRITSKSLCTPGCKTGILMTCPLKTATCGCHFG

SEQ ID NO: 225 - DNA, 168 bp, Streptococcus uberis
  source 1..168: mol_type = genomic DNA
atgaacaatgaagattttaatttggatctcatcaaaatctcaaaggaaaacaactcaggagcttcacctcgaataactagtaaatcattatgtactcctggatgtaagacgggtattttgatgacttgtccactaaaaactgcaacctgtggttgtcattttggataa

SEQ ID NO: 226 - protein, 57 aa, Lactococcus lactis
  source 1..57: note = subsp. lactis
MSTKDFNLDLVSVSKKDSGASPRITSISLCTPGCKTGALMGCNMKTATCNCSIHVSK

SEQ ID NO: 227 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagtattcacgtaagcaaataa

SEQ ID NO: 228 - protein, 57 aa, Staphylococcus warneri
MENSKVMKDIEVANLLEEVQEDELNEVLGAKKKSGVIPTVSHDCHMNSFQFVFTCCS

SEQ ID NO: 229 - DNA, 174 bp, Staphylococcus warneri
  source 1..174: mol_type = genomic DNA
atggaaaattctaaagttatgaaggacattgaagtagcaaatttattagaagaggttcaagaagatgaattgaatgaagtcttaggagctaagaaaaagtcaggagtaatcccaactgtgtcacacgattgccatatgaattctttccaatttgtatttacttgttgttcataa

SEQ ID NO: 230 - protein, 58 aa, Paenibacillus polymyxa
MAENLFDLDIQVNKSQGSVEPQVLSIVACSSGCGSGKTAASCVETCGNRCFTNVGSLC

SEQ ID NO: 231 - DNA, 177 bp, Paenibacillus polymyxa
  source 1..177: mol_type = genomic DNA
atggctgaaaacttatttgatctggacattcaagtaaacaaatctcaaggttctgtagagcctcaggttctgagcattgttgcatgttctagcggatgtggtagcggtaaaacagctgccagttgtgttgaaacttgtggcaaccggtgctttactaacgttggttcactctgctaa

SEQ ID NO: 232 - protein, 62 aa, Pediococcus acidilactici
MKKIEKLTEKEMANIIGGKYYGNGVTCGKHSCSVDWGKATTCIINNGAMAWATGGHQGNHKC

SEQ ID NO: 233 - DNA, 189 bp, Pediococcus acidilactici
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacttgcataatcaataatggagctatggcatgggctactggtggacatcaaggtaatcataaatgctag

SEQ ID NO: 234 - protein, 60 aa, Pediococcus pentosaceus
MTEIKVLNDKELKNVVGGKYYGNGVHCGKKTCYVDWGQATASIGKIIVNGWTQHGPWAHR

SEQ ID NO: 235 - DNA, 183 bp, Pediococcus pentosaceus
  source 1..183: mol_type = genomic DNA
atgactgaaattaaagtactaaacgataaggaactaaaaaatgtcgtaggaggaaagtattacggtaacggagtgcattgtggtaaaaagacttgctatgtggactggggacaagctacagctagcattggaaaaattatagtgaacggatggacacaacacgggccttgggcacatagataa

SEQ ID NO: 236 - protein, 60 aa, Staphylococcus epidermidis
MKNNKNLFDLEIKKETSQNTDELEPQTAGPAIRASVKQCQKTLKATRLFTVSCKGKNGCK

SEQ ID NO: 237 - DNA, 183 bp, Staphylococcus epidermidis
  source 1..183: mol_type = genomic DNA
atgaaaaataacaaaaatttatttgatttagaaattaaaaaagaaacaagtcaaaacactgatgaacttgaacctcaaactgctggaccagcgattagagcttctgtgaaacaatgtcagaaaactttgaaagctacgcgtttatttacagtgtcttgcaaaggaaaaaacggatgtaaatag

SEQ ID NO: 238 - protein, 62 aa, Carnobacterium maltaromaticum
MKTVKELSVKEMQLTTGGKYYGNGVSCNKNGCTVDWSKAIGIIGNNAAANLTTGGAAGWNKG

SEQ ID NO: 239 - DNA, 189 bp, Carnobacterium maltaromaticum
  source 1..189: mol_type = genomic DNA
atgaaaactgttaaagaacttagcgttaaagaaatgcaactaactacaggaggtaagtattacggaaatggcgtttcctgtaataaaaatggttgtactgtagattggagcaaagctattgggattataggaaacaatgcagcagcaaatttgactacaggtggagccgctggttggaacaaaggataa

SEQ ID NO: 240 - protein, 69 aa, Lactobacillus plantarum
  VARIANT 40: note = Xaa = any amino acid
MYKELTVDELALIDGGKKKKKKVACTWGNAATAAASGAVXGILGGPTGALAGAIWGVSQCASNNLHGMH

SEQ ID NO: 241 - DNA, 210 bp, Lactobacillus plantarum
  misc_feature 119: note = n = a, c, t or g
  source 1..210: mol_type = genomic DNA
atgtataaagaattaacagttgatgaattagcattgattgatggaggaaaaaagaagaagaaaaaagtagcttgtacttggggaaatgcagcaacagccgctgcttctggtgcagttangggtattcttggtgggcctactggtgcactggctggagctatctggggcgtttcacaatgcgcgtctaacaacttacacggcatgcactaa

SEQ ID NO: 242 - protein, 56 aa, Lactobacillus plantarum
MMKKIEKLTEKEMANIIGGKYYGNGVTCGKHSCSVNWGQAFSCSVSHLANFGHGKC

SEQ ID NO: 243 - DNA, 171 bp, Lactobacillus plantarum
  source 1..171: mol_type = genomic DNA
atgatgaaaaaaattgaaaaattaactgaaaaagaaatggccaatatcattggtggtaaatactatggtaatggggttacttgtggtaaacattcctgctctgttaactggggccaagcattttcttgtagtgtgtcacatttagctaacttcggtcatggaaagtgctaa

SEQ ID NO: 244 - protein, 64 aa, Lactobacillus plantarum
MSKLVKTLTVDEISKIQTNGGKPAWCWYTLAMCGAGYDSGTCDYMYSHCFGVKHSSGGGGSYHC

SEQ ID NO: 245 - DNA, 195 bp, Lactobacillus plantarum
  source 1..195: mol_type = genomic DNA
atgagtaaactagttaaaacattaactgtcgatgaaatctctaagattcaaaccaatggtggaaaacctgcatggtgttggtacacattggcaatgtgcggtgctggttatgattcaggcacttgtgattatatgtattcacactgctttggtgtaaaacactctagcggtggtggcggtagctaccattgttag

SEQ ID NO: 246 - protein, 56 aa, Lactobacillus plantarum
MLQFEKLQYSRLPQKKLAKISGGFNRGGYNFGKSVRHVVDAIGSVAGIRGILKSIR

SEQ ID NO: 247 - DNA, 171 bp, Lactobacillus plantarum
  source 1..171: mol_type = genomic DNA
atgctacagtttgagaaattacaatattccaggttgccgcaaaaaaagcttgccaaaatatctggtggttttaatcggggcggttataactttggtaaaagtgttcgacatgttgttgatgcaattggttcagttgcaggcattcgtggtattttgaaaagtattcgttaa

SEQ ID NO: 248 - protein, 52 aa, Lactobacillus plantarum
MKKFLVLRDRELNAISGGVFHAYSARGVRNNYKSAVGPADWVISAVRGFIHG

SEQ ID NO: 249 - DNA, 159 bp, Lactobacillus plantarum
  source 1..159: mol_type = genomic DNA
atgaaaaaatttctagttttgcgtgaccgtgaattaaatgctatttcaggtggcgttttccatgcctatagcgcgcgtggcgttcggaataattataaaagtgctgttgggcctgccgattgggtcattagcgctgtccgaggattcatccacggatag

SEQ ID NO: 250 - protein, 55 aa, Lactobacillus plantarum
MTVNKMIKDLDVVDAFAPISNNKLNGVVGGGAWKNFWSSLRKGFYDGEAGRAIRR

SEQ ID NO: 251 - DNA, 168 bp, Lactobacillus plantarum
  source 1..168: mol_type = genomic DNA
atgactgtgaacaaaatgattaaggatttggatgtagtagatgcatttgcacctatttctaataataagttgaacggggttgttgggggaggcgcttggaaaaatttctggtctagtttaagaaaaggattttatgatggcgaagctggcagagcaatccgtcgttaa

SEQ ID NO: 252 - protein, 57 aa, Lactobacillus plantarum
MKIKLTVLNEFEELTADAEKNISGGRRSRKNGIGYAIGYAFGAVERAVLGGSRDYNK

SEQ ID NO: 253 - DNA, 174 bp, Lactobacillus plantarum
  source 1..174: mol_type = genomic DNA
atgaaaattaaattaactgttttaaatgaatttgaagaattaactgctgacgctgaaaagaatatttctggtggccgtcggagtcgtaaaaatggaattggatacgctattggttatgcgtttggcgcggttgaacgggccgtgcttggtggttcaagggattataataagtga

SEQ ID NO: 254 - protein, 47 aa, Lactobacillus plantarum
MDKFEKISTSNLEKISGGDLTTKLWSSWGYYLGKKARWNLKHPYVQF

SEQ ID NO: 255 - DNA, 141 bp, Lactobacillus plantarum
  source 1..141: mol_type = genomic DNA
atggataaatttgaaaaaattagtacatctaacctagaaaagatctctggcggtgatttaacaaccaagttatggagctcttggggatattatcttggcaagaaagcacgttggaatttaaagcacccatatgttcaattt

SEQ ID NO: 256 - protein, 55 aa, Lactobacillus plantarum
MNNLNKFSTLGKSSLSQIEGGSVPTSVYTLGIKILWSAYKHRKTIEKSFNKGFYH

SEQ ID NO: 257 - DNA, 168 bp, Lactobacillus plantarum
  source 1..168: mol_type = genomic DNA
atgaataacttgaataaattttctactctaggcaagagtagcttgtctcaaattgagggcggatcagtcccaacttcagtatatacgcttggaattaaaattctatggtctgcgtataagcatcgcaaaacgattgaaaaaagttttaataaaggcttttatcattaa

SEQ ID NO: 258 - protein, 55 aa, Lactobacillus plantarum
MNNALSFEQQFTDFSTLSDSELESVEGGRNKLAYNMGHYAGKATIFGLAAWALLA

SEQ ID NO: 259 - DNA, 168 bp, Lactobacillus plantarum
  source 1..168: mol_type = genomic DNA
atgaataacgcattaagttttgaacaacaatttacagacttcagcaccttatcggactctgaattagaatccgttgagggtggccgaaataagcttgcatataatatggggcattacgctggtaaggcaaccatttttggacttgcagcatgggcactccttgcatga

SEQ ID NO: 260 - protein, 47 aa, Lactobacillus plantarum
MDKIIKFQGISDDQLNAVIGGKKKKQSWYAAAGDAIVSFGEGFLNAW

SEQ ID NO: 261 - DNA, 144 bp, Lactobacillus plantarum
  source 1..144: mol_type = genomic DNA
atggataagattattaagtttcaagggatttctgatgatcaattaaatgctgttatcggtgggaaaaagaaaaaacaatcttggtacgcagcagctggtgatgcaatcgttagttttggtgaaggatttttaaatgcttggtaa

SEQ ID NO: 262 - protein, 59 aa, Lactobacillus plantarum
MKISKIEAQARKDFFKKIDTNSNLLNVNGAKCKWWNISCDLGNNGHVCTLSHECQVSCN

SEQ ID NO: 263 - DNA, 180 bp, Lactobacillus plantarum
  source 1..180: mol_type = genomic DNA
atgaaaatttctaagattgaagctcaggctcgtaaagatttttttaaaaaaatcgatactaactcgaacttattaaatgtaaatggtgccaaatgcaagtggtggaatatttcgtgtgatttaggaaataatggccatgtttgtaccttgtcacatgaatgccaagtatcttgtaactaa

SEQ ID NO: 264 - protein, 67 aa, Lactobacillus plantarum
MTKTSRRKNAIANYLEPVDEKSINESFGAGDPEARSGIPCTIGAAVAASIAVCPTTKCSKRCGKRKK

SEQ ID NO: 265 - DNA, 204 bp, Lactobacillus plantarum
  source 1..204: mol_type = genomic DNA
atgactaaaactagtcgtcgtaagaatgctattgctaattatttagaaccagtcgacgaaaaaagtattaatgaatcttttggggctggggatccggaagcaagatccggaattccatgtacaatcggcgcagctgtcgcagcatcaattgcagtttgtccaactactaagtgtagtaaacgttgtggcaagcgtaagaaataa

SEQ ID NO: 266 - protein, 48 aa, Lactobacillus plantarum
MKIQIKGMKQLSNKEMQKIVGGKSSAYSLQMGATAIKQVKKLFKKWGW

SEQ ID NO: 267 - DNA, 147 bp, Lactobacillus plantarum
  source 1..147: mol_type = genomic DNA
atgaaaattcaaattaaaggtatgaagcaacttagtaataaggaaatgcaaaaaatagtaggtggaaagagtagtgcgtattctttgcagatgggggcaactgcaattaaacaggtaaagaaactgtttaaaaaatggggatggtaa

SEQ ID NO: 268 - protein, 207 aa, Propionibacterium jensenii
MNKTHKMATLVIAAILAAGMTAPTAYADSPGNTRITASEQSVLTQILGHKPTQTEYNRYVETYGSVPTEADINAYIEASESEGSSSQTAAHDDSTSPGTSTEIYTQAAPARFSMFFLSGTWITRSGVVSLSLKPRKGGIGNEGDERTWKTVYDKFHNAGQWTRYKNNGVDASMKKQYMCHFKYGMVKTPWNLEPHKKAADVSPVKCN

SEQ ID NO: 269 - DNA, 624 bp, Propionibacterium jensenii
  source 1..624: mol_type = genomic DNA
atgaacaaaacacacaaaatggcgacgctggtaattgccgcgatcttggccgccggaatgaccgcaccaactgcctatgcagattctcctggaaacaccagaattacagccagcgagcaaagcgtccttacccagatactcggccacaaacctacacaaactgaatataaccgatacgttgagacttacggaagcgtaccgaccgaagcagacatcaacgcatatatagaagcgtctgaatctgagggatcatcaagtcaaacggctgctcacgatgactcgacatcacccggcacgagtaccgaaatctacacgcaggcagcccctgccaggttctcaatgtttttcctgtccggaacttggatcactaggagtggtgtagtatcgctctccttgaagccaaggaagggtggtattggcaacgagggggacgagcgtacctggaagactgtatacgacaaattccataacgctgggcaatggacacgatacaagaacaacggcgtagacgccagcatgaaaaagcagtacatgtgccacttcaagtacgggatggtgaagacgccatggaatctggagccccacaagaaggctgcagacgtcagtccagtcaagtgcaactag

SEQ ID NO: 270 - protein, 96 aa, Propionibacterium thoenii
MKKTLLRSGTIALATAAAFGASLAAAPSAMAVPGGCTYTRSNRDVIGTCKTGSGQFRIRLDCNNAPDKTSVWAKPKVMVSVHCLVGQPRSISFETK

SEQ ID NO: 271 - DNA, 291 bp, Propionibacterium thoenii
  source 1..291: mol_type = genomic DNA
atgaagaagaccctcctgcgaagtggaacgatcgcactggcgaccgcggctgcatttggcgcatcattggcagccgccccatctgccatggccgttcctggtggttgcacgtacacaagaagcaatcgcgacgtcatcggtacctgcaagactggaagcggccagttccgaatccgacttgactgcaacaacgctccagacaaaacttcagtctgggccaagcccaaggtaatggtgtcggttcactgtcttgttggtcaaccgaggtccatctcgttcgagaccaagtga

SEQ ID NO: 272 - protein, 255 aa, Propionibacterium freudenreichii
  source 1..255: note = subsp. freudenreii
MNTKAVNLKSENTTKLVSYLTENQLDEFIRRIRIDGALVEEVSQNAKQALDNTGLNGWINTDCDEGLLSDFISKIASARWIPLAESIRPAVTDRDKYRVSCWFYQGMNIAIYANIGGVANIIGYTEAAVATLLGAVVAVAPVVPGTPTPPKDKSSQYKEVPLAVRLSETYHEEGVRGLFDELNYSESRMISTLRRASTDGVLINSWNDGQDTILLKKYNFQDLQLTVRSRIVGNQTIIEECKITDGRKTLSDETV

SEQ ID NO: 273 - DNA, 768 bp, Propionibacterium freudenreichii
  source 1..768: mol_type = genomic DNA; note = subsp. freudenreii
atgaataccaaagctgtaaatctgaagtcagaaaacacgactaagttggtgagctaccttacggaaaatcaattggatgagtttattagaaggattcgcattgatggcgctcttgtggaagaggtcagtcaaaatgctaagcaggccttagataatactgggctcaatggctggataaatactgattgcgatgaaggccttctctctgatttcatttcaaagatagcaagtgctagatggattccattagctgagtcaattcgacctgcggtgactgacagggataagtatcgagtaagttgctggttctaccaggggatgaatatagcaatttacgcaaatatcggtggcgtggccaatattatcggctatacggaggccgcagtcgcaacactccttggtgcagttgtggcggtagctcctgtggtccctggaactccaacccctccaaaggacaagagttcgcaatataaggaggttccccttgccgttcgtctttccgaaacataccacgaagagggagtacgaggtctattcgacgagctgaactactccgagagccgtatgatctctactctaaggcgagcatcaaccgatggagtcctaattaattcttggaacgatgggcaggatacaattctgcttaagaagtacaatttccaagacttgcaactgactgtcaggagccgcattgttgggaatcaaacaataattgaagaatgcaaaatcactgatggtagaaaaactctttcagacgagactgtgtag

SEQ ID NO: 274 - protein, 618 aa, Pseudomonas aeruginosa
MARPIADLIHFNSTTVTASGDVYYGPGGGTGIGPIARPIEHGLDSSTENGWQEFESYADVGVDPRRYVPLQVKEKRREIELQFRDAEKKLEASVQAELDKADAALGPAKNLAPLDVINRSLTIVGNALQQKNQKLLLNQKKITSLGAKNFLTRTAEEIGEQAVREGNINGPEAYMRFLDREMEGLTAAYNVKLFTEAISSLQIRMNTLTAAKASIEAAAANKAREQAAAEAKRKAEEQARQQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRVADGGGVYNMGNLVAVTPKRHIEIHKGGK

SEQ ID NO: 275 - DNA, 1857 bp, Pseudomonas aeruginosa
  source 1..1857: mol_type = genomic DNA
atggcacgacccattgctgaccttatccacttcaactctacaactgtcacggcaagcggagacgtttattacggccctgggggaggtaccggcattggccccattgccagacctatagagcacggcttggattcgtccactgaaaatggctggcaagagtttgaaagttatgctgatgtgggcgttgaccccagacgctatgttcctcttcaggttaaagaaaaacgcagggagatcgagcttcagttccgagatgccgagaaaaaacttgaggcgtcggtacaagccgagctggataaggctgatgccgctcttggtccggcaaagaatcttgcaccattggacgtcatcaaccgcagtctgaccatcgttggaaacgccctccagcaaaagaatcaaaaactactgctgaatcagaagaagattaccagcctgggtgcaaagaatttccttacccgtacggcggaagagatcggtgaacaagcggtgcgagaaggcaatattaacgggcctgaagcctatatgcgcttcctcgacagggaaatggaaggtctcacggcagcttataacgtaaaactcttcaccgaagcgatcagtagtctccagatccgcatgaatacgttgaccgccgccaaagcaagtattgaggcggccgcagcaaacaaggcgcgtgaacaagcagcggctgaggccaaacgcaaagccgaagagcaggcccgccagcaagcggcgataagagctgccaatacctatgccatgccggccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgagtagcagatggaggcggcgtttacaatatggggaaccttgttgcagtaacgccaaaacgtcatatagaaatccacaagggagggaagtga

SEQ ID NO: 276 - protein, 689 aa, Pseudomonas aeruginosa
MAVNDYEPGSMVITHVQGGGRDIIQYIPARSSYGTPPFVPPGPSPYVGTGMQEYRKLRSTLDKSHSELKKNLKNETLKEVDELKSEAGLPGKAVSANDIRDEKSIVDALMDAKAKSLKAIEDRPANLYTASDFPQKSESMYQSQLLASRKFYGEFLDRHMSELAKAYSADIYKAQIAILKQTSQELENKARSLEAEAQRAAAEVEADYKARKANVEKKVQSELDQAGNALPQLTNPTPEQWLERATQLVTQAIANKKKLQTANNALIAKAPNALEKQKATYNADLLVDEIASLQARLDKLNAETARRKEIARQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRIADGGGVYNMGNLVAVTPKRHIEIHKGGK

SEQ ID NO: 277 - DNA, 2070 bp, Pseudomonas aeruginosa
  source 1..2070: mol_type = genomic DNA
atggctgtcaatgattacgaacctggttcgatggttattacacatgtgcagggtggtgggcgtgacataatccagtatattcctgctcgatcaagctacggtactccaccatttgtcccaccaggaccaagtccgtatgtcggtactggaatgcaggagtacaggaagctaagaagtacgcttgataagtcccattcagaactcaagaaaaacctgaaaaatgaaaccctgaaggaggttgatgaactcaagagtgaagcggggttgccaggtaaagcggtcagtgccaatgacatccgcgatgaaaagagtatcgttgatgcactcatggatgccaaagcaaaatcgctaaaggccattgaggatcgcccggccaatctttatacggcttcagactttcctcagaagtcagagtcgatgtaccagagtcagttgctggccagccgaaaattctatggagagttcctggatcgccatatgagtgagctggccaaagcgtacagcgccgatatctataaggcgcaaatcgctatcttgaaacaaacgtctcaagagctggagaataaagcccggtcattggaagcagaagcccagcgagccgctgctgaggtggaggcggactacaaggccaggaaggcaaatgtcgagaaaaaagtgcagtccgagcttgaccaggctgggaatgctttgcctcaactgaccaatccaacgccagagcagtggcttgaacgcgctactcaactggttacgcaggcgatcgccaataagaagaaattgcagactgcaaacaatgccttgattgccaaggcacccaatgcactggagaaacaaaaggcaacctacaacgccgatctcctagtggatgaaatcgccagcctgcaagcacggctggacaagctgaacgccgaaacggcaaggcgcaaggaaatcgctcgtcaagcggcgatcagggctgccaatacttatgccatgccagccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgaatagcagatggaggcggcgtttacaatatggggaaccttgttgcagtaacgccaaaacgtcatatagaaatccacaagggagggaagtga

SEQ ID NO: 278 - protein, 47 aa, Ruminococcus gnavus
MRNDVLTLTNPMEEKELEQILGGGNGVLKTISHECNMNTWQFLFTCC

SEQ ID NO: 279 - DNA, 144 bp, Ruminococcus gnavus
  source 1..144: mol_type = genomic DNA
atgagaaatgacgtattaacattaacaaacccaatggaagagaacgaactggagcagatcttaggtggtggcaatggtgtgttaaaaacgattagccacgaatgcaatatgaacacatggcagttcctgtttacttgttgctaa

SEQ ID NO: 280 - protein, 55 aa, Lactobacillus sakei
MKNAKSLTIQEMKSITGGKYYGNGVSCNSHGCSVNWGQAWTCGVNHLANGGHGVC

SEQ ID NO: 281 - DNA, 168 bp, Lactobacillus sakei
  source 1..168: mol_type = genomic DNA
atgaaaaacgcaaaaagcctaacaattcaagaaatgaaatctattacaggtggtaaatactatggtaatggcgttagctgtaactctcacggctgttcagtaaattgggggcaagcatggacttgtggagtaaaccatctagctaatggcggtcatggagtttgttaa

SEQ ID NO: 282 - protein, 59 aa, Lactobacillus sakei; identical to SEQ ID NO: 68

SEQ ID NO: 283 - DNA, 180 bp, Lactobacillus sakei; identical to SEQ ID NO: 69
  source 1..180: mol_type = genomic DNA

SEQ ID NO: 284 - protein, 61 aa, Lactobacillus sakei
MEKFIELSLKEVTAITGGKYYGNGVHCGKHSCTVDWGTAIGNIGNNAAANWATGGNAGWNK

SEQ ID NO: 285 - DNA, 186 bp, Lactobacillus sakei
  source 1..186: mol_type = genomic DNA
atggaaaagtttattgaattatctttaaaagaagtaacagcaattacaggtggaaaatattatggtaacggtgtacactgtggaaaacattcatgtaccgtagactggggaacagctattggaaatatcggaaataatgcagctgcaaactgggccacaggcggaaacgctggctggaataaataa

SEQ ID NO: 286 - protein, 56 aa, Streptococcus salivarius
MKSTNNQSIAEIAAVNSLQEVSMEELDQIIGAGNGVVLTLTHECNLATWTKKLKCC

SEQ ID NO: 287 - DNA, 171 bp, Streptococcus salivarius
  source 1..171: mol_type = genomic DNA
atgaaatcaacaaataatcaaagtatcgcagaaattgcagcagtaaactcactacaagaagtaagtatggaggaactagaccaaattattggtgccggaaacggagtggttcttactcttactcatgaatgtaacctagcaacttggacaaaaaaactaaaatgttgctaa

SEQ ID NO: 288 - protein, 51 aa, Streptococcus pyogenes
  source 1..51: note = serotype M28
MSFMKNSKDILTNAIEEVSEKELMEVAGGKKGSGWFATITDDCPNSVFVCC

SEQ ID NO: 289 - DNA, 156 bp, Streptococcus pyogenes
  source 1..156: mol_type = genomic DNA; note = serotype M28
atgagttttatgaaaaattcaaaggatattttgactaatgctatcgaagaagtttctgaaaaagaacttatggaagtagctggtggtaaaaaaggttccggttggtttgcaactattactgatgactgtccgaactcagtattcgtttgttgttaa

SEQ ID NO: 290 - protein, 48 aa, Streptococcus salivarius
MKNSKDVLNNAIEEVSEKELMEVAGGKKGPGWIATITDDCPNSIFVCC

SEQ ID NO: 291 - DNA, 147 bp, Streptococcus salivarius
  source 1..147: mol_type = genomic DNA
atgaaaaactcaaaagatgttttgaacaatgctatcgaagaggtttctgaaaaagaacttatggaagtagctggtggtaaaaaaggtccaggttggattgcaactattactgatgactgtccaaactcaatattcgtttgttgttaa

SEQ ID NO: 292 - protein, 48 aa, Streptococcus salivarius
MKNSKDILNNAIEEVSEKELMEVAGGKRGSGWIATITDDCPNSVFVCC

SEQ ID NO: 293 - DNA, 147 bp, Streptococcus salivarius
  source 1..147: mol_type = genomic DNA
atgaaaaactcaaaagatattttgaacaatgctatcgaagaagtttctgaaaaagaacttatggaagtagctggtggtaaaagaggttcaggttggattgcaactattactgatgactgtccaaactcagtattcgtttgttgttaa

SEQ ID NO: 294 - protein, 62 aa, Staphylococcus aureus
MKSSFLEKDIEEQVTWFEEVSEQEFDDDIFGACSTNTFSLSDYWGNKGNWCTATHECMSWCK

SEQ ID NO: 295 - DNA, 189 bp, Staphylococcus aureus
  source 1..189: mol_type = genomic DNA
atgaaaagttcttttttagaaaaagatatagaagaacaagtgacatggttcgaggaagtttcagaacaagaatttgacgatgatatttttggagcttgtagtacaaacactttttctttgagtgactattggggtaataaaggaaattggtgtactgctactcacgaatgtatgtcttggtgtaaataa

SEQ ID NO: 296 - protein, 67 aa, Staphylococcus aureus
MKNELGKFLEENELELGKFSESDMLEITDDEVYAAGTPLALLGGAATGVIGYISNQTCPTTACTRAC

SEQ ID NO: 297 - DNA, 204 bp, Staphylococcus aureus
  source 1..204: mol_type = genomic DNA
atgaaaaatgaattaggtaagtttttagaagaaaacgaattagagttaggtaaattttcagaatcagacatgctagaaattactgatgatgaagtatatgcagctggaacacctttagccttattgggtggagctgccaccggggtgataggttatatttctaaccaaacatgtccaacaactgcttgtacacgcgcttgctag

SEQ ID NO: 298 - protein, 46 aa, Streptococcus pyogenes
MNNTIKDFDLDLKTNKKDTATPYVGSRYLCTPGSCWKLVCFTTTVK

SEQ ID NO: 299 - DNA, 141 bp, Streptococcus pyogenes
  source 1..141: mol_type = genomic DNA
atgaataacacaattaaagactttgatctcgatttgaaaacaaataaaaaagacactgctacaccttatgttggtagccgttacctatgtacccctggttcttgttggaaattagtttgctttacaacaactgttaaataa

SEQ ID NO: 300 - protein, 51 aa, Streptococcus pyogenes
MEKNNEVINSIQEVSLEELDQIIGAGKNGVFKTISHECHLNTWAFLATCCS

SEQ ID NO: 301 - DNA, 156 bp, Streptococcus pyogenes
  source 1..156: mol_type = genomic DNA
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgttcataa

SEQ ID NO: 302 - protein, 51 aa, Streptococcus pyogenes
  source 1..51: note = serotype M49
MTKEHEIINSIQEVSLEELDQIIGAGKNGVFKTISHECHLNTWAFLATCCS

SEQ ID NO: 303 - DNA, 156 bp, Streptococcus pyogenes
  source 1..156: mol_type = genomic DNA; note = serotype M49
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgctcataa

SEQ ID NO: 304 - protein, 56 aa, Bacillus subtilis
MEKLFKEVKLEELENQKGSGLGKAQCAALWLQCASGGTIGCGGGAVACQNYRQFCR

SEQ ID NO: 305 - DNA, 171 bp, Bacillus subtilis
  source 1..171: mol_type = genomic DNA
atggaaaagctatttaaagaagttaaactagaggaactcgaaaaccaaaaaggtagtggattaggaaaagctcagtgtgctgcgttgtggctacaatgtgctagtggcggtacaattggttgtggtggcggagctgttgcttgtcaaaactatcgtcaattctgcagataa

SEQ ID NO: 306 - protein, 56 aa, Bacillus subtilis
MSKFDDFDLDVVKVSKQDSKITPQWKSESLCTPGCVTGALQTCFLQTLTCNCKISK

SEQ ID NO: 307 - DNA, 171 bp, Bacillus subtilis
  source 1..171: mol_type = genomic DNA
atgtcaaagttcgatgatttcgatttggatgttgtgaaagtctctaaacaagactcaaaaatcactccgcaatggaaaagtgaatcactttgtacaccaggatgtgtaactggtgcattgcaaacttgcttccttcaaacactaacttgtaactgcaaaatctctaaataa

SEQ ID NO: 308 - protein, 50 aa, Bacillus subtilis
MKLPVQQVYSVYGGKDLPKGHSHSTMPFLSKLQFLTKIYLLDIHTQPFFI

SEQ ID NO: 309 - DNA, 153 bp, Bacillus subtilis
  source 1..153: mol_type = genomic DNA
ttgaaattgccggtgcaacaggtctattcggtctatgggggtaaggatctcccaaaagggcatagtcattctactatgccctttttaagtaaattacaatttttaactaaaatctacctcttggatatacatacacaaccgtttttcatttga

SEQ ID NO: 310 - protein, 43 aa, Bacillus subtilis
MKKAVIVENKGCATCSIGAACLVDGPIPDFEIAGATGLFGLWG

SEQ ID NO: 311 - DNA, 132 bp, Bacillus subtilis
  source 1..132: mol_type = genomic DNA
atgaaaaaagctgtcattgtagaaaacaaaggttgtgcaacatgctcgatcggagccgcttgtctagtggacggtcctatccctgattttgaaattgccggtgcaacaggtctattcggtctatgggggtaa

SEQ ID NO: 312 - protein, 58 aa, Streptococcus thermophilus; identical to SEQ ID NO: 24

SEQ ID NO: 313 - DNA, 177 bp, Streptococcus thermophilus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcctgtaaaaattgtgcttaa

SEQ ID NO: 314 - protein, 64 aa, Streptococcus thermophilus
MKQYNGFEVLHELDLANVTGGQINWGSVVGHCIGGAIIGGAFSGGAAAGVGCLVGSGKAIINGL

SEQ ID NO: 315 - DNA, 195 bp, Streptococcus thermophilus
  source 1..195: mol_type = genomic DNA
atgaagcagtataatggttttgaggttctacatgaacttgacttagcaaatgtaactggcggtcaaattaattggggatcagttgtaggacactgtataggtggagctattatcggaggtgcattttcaggaggtgcagcggctggagtaggatgccttgttgggagcggaaaggcaatcataaatggattataa

SEQ ID NO: 316 - protein, 85 aa, Streptococcus thermophilus
MNTITICKFDVLDAELLSTVEGGYSGKDCLKDMGGYALAGAGSGALWGAPAGGVGALPGAFVGAHVGAIAGGFACMGGMIGNKFN

SEQ ID NO: 317 - DNA, 258 bp, Streptococcus thermophilus
  source 1..258: mol_type = genomic DNA
atgaatacaataactatttgtaaatttgatgttttagatgctgaacttctttcgacagttgagggtggatactctggtaaggattgtttaaaagacatgggaggatatgcattggcaggagctggaagtggagctctgtggggagctccagcaggaggtgttggagcacttccaggtgcatttgtcggagctcatgttggggcaattgcaggaggctttgcatgtatgggtggaatgattggtaataagtttaactaa

SEQ ID NO: 318 - protein, 52 aa, Bacillus cereus
MSEIKKALNTLEIEDFDAIEMVDVDAMPENEALEIMGASCTTCVCTCSCCTT

SEQ ID NO: 319 - DNA, 159 bp, Bacillus cereus
  source 1..159: mol_type = genomic DNA
atgagtgaaattaaaaaagcattaaatacgcttgaaattgaagattttgatgcaattgaaatggttgatgttgatgctatgccagaaaacgaagcgcttgaaattatgggagcgtcatgtacgacatgcgtatgtacatgcagttgttgtacaacttga

SEQ ID NO: 320 - protein, 47 aa, Bacillus cereus
MEVMNNALITKVDEEIGGNAACVIGCIGSCVISEGIGSLVGTAFTLG

SEQ ID NO: 321 - DNA, 144 bp, Bacillus cereus
  source 1..144: mol_type = genomic DNA
atggaagttatgaacaatgctttaattacaaaagtagatgaggagattggaggaaacgctgcttgtgtaattggttgtattggcagttgcgtaattagtgaaggaattggttcacttgtaggaacagcatttactttaggttaa

SEQ ID NO: 322 - protein, 49 aa, Bacillus cereus
MEVLNKQNVNIIPESEEVGGWVACVGACGTVCLASGGVGTEFAAASYFL

SEQ ID NO: 323 - DNA, 150 bp, Bacillus cereus
  source 1..150: mol_type = genomic DNA
atggaagttttaaacaaacaaaatgtaaatattattccagaatctgaagaagtaggtggatgggtagcatgtgttggagcatgtggtacagtatgtcttgctagtggtggtgttggaacagagtttgcagctgcatcttatttcctataa

SEQ ID NO: 324 - protein, 40 aa, Bacillus thuringiensis
METPVVQPRDWTCWSCLVCAACSVELLNLVTAATGASTAS

SEQ ID NO: 325 - DNA, 123 bp, Bacillus thuringiensis
  source 1..123: mol_type = genomic DNA
atggaaacaccagtagtacaaccaagggattggacttgttggagttgcttagtatgtgcagcatgttctgtggaattattaaatttagttactgcggcaacaggggctagtactgcaagctaa

SEQ ID NO: 326 - protein, 42 aa, Rhizobium leguminosarum
  source 1..42: note = bv. trifolii
MDNKVAKNVEVKKGSIKATFKAAVLKSKTKVDIGGSRQGCVA

SEQ ID NO: 327 - DNA, 129 bp, Rhizobium leguminosarum
  source 1..129: mol_type = genomic DNA; note = bv. trifolii
atggataacaaggttgcgaagaatgtcgaagtgaagaagggctccatcaaggcgaccttcaaggctgctgttctgaagtcgaagacgaaggtcgacatcggaggtagccgtcagggctgcgtcgcttaa

SEQ ID NO: 328 - protein, 70 aa, Streptococcus uberis
MNTIEKFENIKLFSLKKIIGGKTVNYGNGLYCNQKKCWVNWSETATTIVNNSIMNGLTGGNAGWHSGGRA

SEQ ID NO: 329 - DNA, 213 bp, Streptococcus uberis
  source 1..213: mol_type = genomic DNA
atgaatacaattgaaaaatttgaaaatattaaacttttttcactaaagaaaattatcggtggcaaaactgtaaattatggtaatggcctttattgtaaccaaaaaaaatgctgggtaaactggtcagaaactgctacaacaatagtaaataattccatcatgaacgggctcacaggtggtaatgcgggttggcactcaggcgggagagcataa

SEQ ID NO: 330 - protein, 76 aa, Streptococcus uberis
MDILLELAGYTGIASGTAKKVVDAIDKGAAAFVIISIISTVISAGALGAVSASADFIILTVKNYISRNLKAQAVIW

SEQ ID NO: 331 - DNA, 231 bp, Streptococcus uberis
  source 1..231: mol_type = genomic DNA
atggacattttattagaactcgcaggatatactgggatagcctcaggtactgcaaaaaaagttgttgatgccattgataaaggagctgcagcctttgttattatttcaattatctcaacagtaattagtgcgggagcattgggagcagtttcagcctcagctgattttattattttaactgtaaaaaattacattagtagaaatttaaaagcacaagctgtcatttggtaa

SEQ ID NO: 332 - protein, 64 aa, Clostridium perfringens
MDSELFKLMATQGAFAILFSYLLFYVLKENSKREDKYQNIIEELTELLPKIKEDVEDIKEKLNK

SEQ ID NO: 333 - DNA, 195 bp, Clostridium perfringens
  source 1..195: mol_type = genomic DNA
atggatagtgaattatttaagttaatggcaacacaaggagcctttgcaatattattttcgtatttattgttttatgttttaaaagagaatagtaaaagagaagataagtatcaaaatataatagaggagcttacagaattattgccaaaaataaaagaagatgtagaagatataaaagaaaaacttaataaatag

SEQ ID NO: 334 - protein, 47 aa, Micrococcus varians
MTNAFQALDEVTDAELDAILGGGSGVIPTISHECHMNSFQFVFTCCS

SEQ ID NO: 335 - DNA, 144 bp, Micrococcus varians
  source 1..144: mol_type = genomic DNA
atgacgaacgcatttcaggcactggacgaagtcacggacgccgagctcgacgccatccttggcgggggcagtggtgttattcccacgatcagccacgagtgccacatgaactccttccagttcgtgttcacctgctgctcctga

SEQ ID NO: 336 - protein, 285 aa, Streptococcus equi
  source 1..285: note = subsp. zooepidemicus
MKRIFFAFLSLCLFIFGTQTVSAATYTRPLDTGNITTGFNGYPGHVGVDYAVPVGTPVRAVANGTVKFAGNGANHPWMLWMAGNCVLIQHADGMHTGYAHLSKISVSTDSTVKQGQIIGYTGATGQVTGPHLHFEMLPANPNWQNGFSGRIDPTGYIANAPVFNGTTPTEPTTPTTNLKIYKVDDLQKINGIWQVRNNILVPTDFTWVDNGIAADDVIEVTSNGTRTSDQVLQKGGYFVINPNNVKSVGTPMKGSGGLSWAQVNFTTGGNVWLNTTSKDNLLYGK

SEQ ID NO: 337 - DNA, 858 bp, Streptococcus equi
  source 1..858: mol_type = genomic DNA; note = subsp. zooepidemicus
atgaaacgtatattttttgctttcttaagtttatgcttatttatattcggaacacaaacggtatctgcagctacttatactcggccattagatacgggaaatatcactacagggtttaacggataccctggtcatgttggagtcgattatgcagtacccgttggaactccggttagagcagttgcaaatggtacagtcaaatttgcaggtaatggggctaatcacccatggatgctttggatggctggaaactgtgttctaattcaacatgctgacgggatgcatactggatatgcacacttatcaaaaatttcagttagcacagatagtacagttaaacaaggacaaatcataggttatactggtgccaccggccaagttaccggtccacatttgcattttgaaatgttgccagcaaatcctaactggcaaaatggtttttctggaagaatagatccaaccggatacatcgctaatgcccctgtatttaatggaacaacacctacagaacctactactcctacaacaaatttaaaaatctataaagttgatgatttacaaaaaattaatggtatttggcaagtaagaaataacatacttgtaccaactgatttcacatgggttgataatggaattgcagcagatgatgtaattgaagtaactagcaatggaacaagaacctctgaccaagttcttcaaaaaggtggttattttgtcatcaatcctaataatgttaaaagtgttggaactccgatgaaaggtagtggtggtctatcttgggctcaagtaaactttacaacaggtggaaatgtctggttaaatactactagcaaagacaacttactttacggaaaataa

SEQ ID NO: 338 - protein, 45 aa, Myxococcus fulvus
ANCSCSTASDYCPILTFCTTGTACSYTPTGCGTGWVYCACNGNFY

SEQ ID NO: 339 - DNA, 135 bp, synthetic construct
  misc_feature 1..135: note = Polynucleotide encoding the polypeptide of SEQ ID NO: 338
  source 1..135: mol_type = other DNA
gcgaactgcagctgcagcaccgcgagcgattattgcccgattctgaccttttgcaccaccggcaccgcgtgcagctataccccgaccggctgcggcaccggctgggtgtattgcgcgtgcaacggcaacttttat

SEQ ID NO: 340 - protein, 19 aa, Streptomyces griseoluteus
CANSCSYGPLTWSCDGNTK

SEQ ID NO: 341 - DNA, 57 bp, synthetic construct
  misc_feature 1..57: note = Polynucleotide encoding the polypeptide of SEQ ID NO: 340
  source 1..57: mol_type = other DNA
tgcgcgaacagctgcagctatggcccgctgacctggagctgcgatggcaacaccaaa

SEQ ID NO: 342 - protein, 19 aa, Streptoverticillium griseoverticillatum
CKQSCSFGPFTFVCDGNTK

SEQ ID NO: 343 - DNA, 57 bp, synthetic construct
  misc_feature 1..57: note = Polynucleotide encoding the polypeptide of SEQ ID NO: 342
  source 1..57: mol_type = other DNA
tgcaaacagagctgcagctttggcccgtttacctttgtgtgcgatggcaacaccaaa

SEQ ID NO: 344 - protein, 7 aa, Carnobacterium sp.
GSEIQPR

SEQ ID NO: 345 - DNA, 21 bp, synthetic construct
  misc_feature 1..21: note = Polynucleotide encoding the polypeptide of SEQ ID NO: 344
  source 1..21: mol_type = other DNA
ggcagcgaaattcagccgcgc

SEQ ID NO: 346 - protein, 39 aa, Lactococcus lactis
  source 1..39: note = subsp. lactis
GTWDDIGQGIGRVAYWVGKAMGNMSDVNQASRINRKKKH

SEQ ID NO: 347 - DNA, 117 bp, synthetic construct
  misc_feature 1..117: note = Polynucleotide encoding the polypeptide of SEQ ID NO: 346
  source 1..117: mol_type = other DNA
ggcacctgggatgatattggccagggcattggccgcgtggcgtattgggtgggcaaagcgatgggcaacatgagcgatgtgaaccaggcgagccgcattaaccgcaaaaaaaaacat

SEQ ID NO: 348 - protein, 35 aa, Lactococcus lactis
  source 1..35: note = subsp. lactis
KKWGWLAWVDPAYEFIKGFGKGAIKEGNKDKWKNI

SEQ ID NO: 349 - DNA, 105 bp, synthetic construct
  misc_feature 1..105: note = Polynucleotide encoding the polypeptide of SEQ ID NO: 348
  source 1..105: mol_type = other DNA
aaaaaatggggctggctggcgtgggtggatccggcgtatgaatttattaaaggctttggcaaaggcgcgattaaagaaggcaacaaagataaatggaaaaacatt

SEQ ID NO: 350 - protein, 19 aa, Streptomyces sp.
CVQSCSFGPLTWSCDGNTK

SEQ ID NO: 351 - DNA, 57 bp, synthetic construct
  misc_feature 1..57: note = Polynucleotide encoding the polypeptide of SEQ ID NO: 350
  source 1..57: mol_type = other DNA
tgcgtgcagagctgcagctttggcccgctgacctggagctgcgatggcaacaccaaa

SEQ ID NO: 352 - protein, 19 aa, Actinoplanes liguriae
SSGWVCTLTIECGTVICAC

SEQ ID NO: 353 - DNA, 57 bp, synthetic construct
  misc_feature 1..57: note = Polynucleotide encoding the polypeptide of SEQ ID NO: 352
  source 1..57: mol_type = other DNA
agcagcggctgggtgtgcaccctgaccattgaatgcggcaccgtgatttgcgcgtgc

SEQ ID NO: 354 - protein, 38 aa, Lactobacillus curvatus
  VARIANT 34: note = Xaa = any amino acid
  VARIANT 37: note = Xaa = any amino acid
YTAKQCLQAIGSCGIAGTGAGAAGGPAGAFVGAXVVXI

SEQ ID NO: 355 - DNA, 114 bp, synthetic construct
  misc_feature 1..114: note = Polynucleotide encoding the polypeptide of SEQ ID NO: 354
  misc_feature 100..111: note = n = A,T,C or G
  misc_feature 100..102: note = nnn = a codon other than a stop codon
  misc_feature 109..111: note = nnn = a codon other than a stop codon
  source 1..114: mol_type = other DNA
tataccgcgaaacagtgcctgcaggcgattggcagctgcggcattgcgggcaccggcgcgggcgcggcgggcggcccggcgggcgcgtttgtgggcgcgnnngtggtgnnnatt

SEQ ID NO: 356 - protein, 42 aa, Lactobacillus sakei
  VARIANT 32: note = Xaa = any amino acid
TKYYGNGVYCNSKKCWVDWGQAAGGIGQTVVXGWLGGAIPGK

SEQ ID NO: 357 - DNA, 126 bp, synthetic construct
  misc_feature 1..126: note = Polynucleotide encoding the polypeptide of SEQ ID NO: 356
  misc_feature 94..96: note = nnn = any amino acid-coding triplet
  source 1..126: mol_type = other DNA
accaaatattatggcaacggcgtgtattgcaacagcaaaaaatgctgggtggattggggccaggcggcgggcggcattggccagaccgtggtgnnnggctggctgggcggcgcgattccgggcaaa

SEQ ID NO: 358 - protein, 22 aa, Streptococcus mutans
FKSWSFCTPGCAKTGSFNSYCC

SEQ ID NO: 359 - DNA, 132 bp, synthetic construct
  misc_feature 1..132: note = Polynucleotide encoding the polypeptide of SEQ ID NO: 358
  source 1..132: mol_type = other DNA
tttaaaagctggagcttttgcaccccgggctgcgcgaaaaccggcagctttaacagctattgctgctttaaaagctggagcttttgcaccccgggctgcgcgaaaaccggcagctttaacagctattgctgc

SEQ ID NO: 360 - protein, 43 aa, Enterococcus mundtii
KYYGNGVSCNKKGCSVDWGKAIGIIGNNSAANLATGGAAGWSK

SEQ ID NO: 361 - DNA, 129 bp, synthetic construct
  misc_feature 1..129: note = Polynucleotide encoding the polypeptide of SEQ ID NO: 360
  source 1..129: mol_type = other DNA
aaatattatggcaacggcgtgagctgcaacaaaaaaggctgcagcgtggattggggcaaagcgattggcattattggcaacaacagcgcggcgaacctggcgaccggcggcgcggcgggctggagcaaa

SEQ ID NO: 362 - protein, 41 aa, Lactobacillus sakei
  VARIANT 9..37: note = Xaa = any amino acid
KYYGNGVHXGKHSXTVDWGTAIGNIGNNAAANXATGXNAGG

SEQ ID NO: 363 - DNA, 123 bp, synthetic construct
  misc_feature 1..123: note = Polynucleotide encoding the polypeptide of SEQ ID NO: 362
  misc_feature 25..27: note = nnn = any amino acid-coding triplet
  misc_feature 40..42: note = nnn = any amino acid-coding triplet
  misc_feature 97..99: note = nnn = any amino acid-coding triplet
  misc_feature 109..111: note = nnn = any amino acid-coding triplet
  source 1..123: mol_type = other DNA
aaatattatggcaacggcgtgcatnnnggcaaacatagcnnnaccgtggattggggcaccgcgattggcaacattggcaacaacgcggcggcgaacnnngcgaccggcnnnaacgcgggcggc

SEQ ID NO: 364 - protein, 31 aa, Lactobacillus paracasei
GMSGYIQGIPDFLKGYLHGISAANKHKKGRL

SEQ ID NO: 365 - DNA, 93 bp, synthetic construct
  misc_feature 1..93: note = Polynucleotide encoding the polypeptide of SEQ ID NO: 364
  source 1..93: mol_type = other DNA
ggcatgagcggctatattcagggcattccggattttctgaaaggctatctgcatggcattagcgcggcgaacaaacataaaaaaggccgcctg

SEQ ID NO: 366 - protein, 31 aa, Leuconostoc mesenteroides
KGKGFWSWASKATSWLTGPQQPGSPLLKKHR

SEQ ID NO: 367 - DNA, 93 bp, synthetic construct
  misc_feature 1..93: note = Polynucleotide encoding the polypeptide of SEQ ID NO: 366
  source 1..93: mol_type = other DNA
aaaggcaaaggcttttggagctgggcgagcaaagcgaccagctggctgaccggcccgcagcagccgggcagcccgctgctgaaaaaacatcgc

SEQ ID NO: 368 - protein, 43 aa, Leuconostoc mesenteroides
KNYGNGVHCTKKGCSVDWGYAWTNIANNSVMNGLTGGNAGWHN

SEQ ID NO: 369 - DNA, 129 bp, synthetic construct
  misc_feature 1..129: note = Polynucleotide encoding the polypeptide of SEQ ID NO: 368
  source 1..129: mol_type = other DNA
aaaaactatggcaacggcgtgcattgcaccaaaaaaggctgcagcgtggattggggctatgcgtggaccaacattgcgaacaacagcgtgatgaacggcctgaccggcggcaacgcgggctggcataac

SEQ ID NO: 370 - protein, 47 aa, Bacillus subtilis
AIKLVQSPNGNFAASFVLDGTKWIFKSKYYDSSKGYWVGIYEVWDRK

SEQ ID NO: 371 - DNA, 141 bp, synthetic construct
  misc_feature 1..141: note = Polynucleotide encoding the polypeptide of SEQ ID NO: 370
  source 1..141: mol_type = other DNA
gcgattaaactggtgcagagcccgaacggcaactttgcggcgagctttgtgctggatggcaccaaatggatttttaaaagcaaatattatgatagcagcaaaggctattgggtgggcatttatgaagtgtgggatcgcaaa

SEQ ID NO: 372 - protein, 12 aa, Bacillus licheniformis
  VARIANT 7: note = Xaa = any amino acid
ISLEICXIFHDN

SEQ ID NO: 373 - DNA, 36 bp, synthetic construct
  misc_feature 1..36: note = Polynucleotide encoding the polypeptide of SEQ ID NO: 372
  misc_feature 19..21: note = nnn = any amino acid-coding triplet
  source 1..36: mol_type = other DNA
attagcctggaaatttgcnnnatttttcatgataac

SEQ ID NO: 374 - protein, 37 aa, Lactococcus lactis
  source 1..37: note = subsp. lactis
TSYGNGVHCNKSKCWIDVSELETYKAGTVSNPKDILW

SEQ ID NO: 375 - DNA, 111 bp, synthetic construct
  misc_feature 1..111: note = Polynucleotide encoding the polypeptide of SEQ ID NO: 374
  source 1..111: mol_type = other DNA
accagctatggcaacggcgtgcattgcaacaaaagcaaatgctggattgatgtgagcgaactggaaacctataaagcgggcaccgtgagcaacccgaaagatattctgtgg

SEQ ID NO: 376 - protein, 9 aa, Serratia plymuthica
DYHHGVRVL

SEQ ID NO: 377 - DNA, 27 bp, synthetic construct
  misc_feature 1..27: note = Polynucleotide encoding the polypeptide of SEQ ID NO: 376
  source 1..27: mol_type = other DNA
gattatcatcatggcgtgcgcgtgctg

SEQ ID NO: 378 - protein, 15 aa, Halobacterium sp.
DIDITGCSACKYAAG

SEQ ID NO: 379 - DNA, 45 bp, synthetic construct
  misc_feature 1..45: note = Polynucleotide encoding the polypeptide of SEQ ID NO: 378
  source 1..45: mol_type = other DNA
gatattgatattaccggctgcagcgcgtgcaaatatgcggcgggc

SEQ ID NO: 380 - protein, 12 aa, Bacillus subtilis
  VARIANT 1..6: note = Xaa = any amino acid
XXKEIXHIFHDN

SEQ ID NO: 381 - DNA, 36 bp, synthetic construct
  misc_feature 1..36: note = Polynucleotide encoding the polypeptide of SEQ ID NO: 380
  misc_feature 1..3: note = nnn = any amino acid-coding triplet
  misc_feature 4..6: note = nnn = any amino acid-coding triplet
  misc_feature 16..18: note = nnn = any amino acid-coding triplet
  source 1..36: mol_type = other DNA
nnnnnnaaagaaattnnncatatttttcatgataac

SEQ ID NO: 382 - protein, 12 aa, Lactobacillus curvatus
TPVVNPPFLQQT

SEQ ID NO: 383 - DNA, 36 bp, synthetic construct
  misc_feature 1..36: note = Polynucleotide encoding the polypeptide of SEQ ID NO: 382
  source 1..36: mol_type = other DNA
accccggtggtgaacccgccgtttctgcagcagacc

SEQ ID NO: 384 - protein, 10 aa, Lactobacillus curvatus
  VARIANT 10: note = Xaa = any amino acid
VAPFPEQFLX

SEQ ID NO: 385 - DNA, 30 bp, synthetic construct
  misc_feature 1..30: note = Polynucleotide encoding the polypeptide of SEQ ID NO: 384
  misc_feature 28..30: note = nnn = any amino acid-coding triplet
  source 1..30: mol_type = other DNA
gtggcgccgtttccggaacagtttctgnnn

SEQ ID NO: 386 - protein, 9 aa, Lactobacillus curvatus
NIPQLTPTP

SEQ ID NO: 387 - DNA, 27 bp, synthetic construct
  misc_feature 1..27: note = Polynucleotide encoding the polypeptide of SEQ ID NO: 386
  source 1..27: mol_type = other DNA
aacattccgcagctgaccccgaccccg

SEQ ID NO: 388 - protein, 18 aa, Bacillus thuringiensis
  VARIANT 4..10: note = Xaa = any amino acid
  source 1..18: note = subsp. entomocidus
DWTXWSXLVXAACSVELL

SEQ ID NO: 389 - DNA, 54 bp, synthetic construct
  misc_feature 1..54: note = Polynucleotide encoding the polypeptide of SEQ ID NO: 388
  misc_feature 10..12: note = nnn = any amino acid-coding triplet
  misc_feature 19..21: note = nnn = any amino acid-coding triplet
  misc_feature 28..30: note = nnn = any amino acid-coding triplet
  source 1..54: mol_type = other DNA
gattggaccnnntggagcnnnctggtgnnngcggcgtgcagcgtggaactgctg

SEQ ID NO: 390 - protein, 30 aa, Lactobacillus curvatus
AYPGNGVHCGKYSCTVDKQTAIGNIGNNAA

SEQ ID NO: 391 - DNA, 90 bp, synthetic construct
  misc_feature 1..90: note = Polynucleotide encoding the polypeptide of SEQ ID NO: 390
  source 1..90: mol_type = other DNA
gcgtatccgggcaacggcgtgcattgcggcaaatatagctgcaccgtggataaacagaccgcgattggcaacattggcaacaacgcggcg

SEQ ID NO: 392 - protein, 43 aa, Carnobacterium divergens
TKYYGNGVYCNSKKCWVDWGTAQGCIDVVIGQLGGGIPGKGKC

SEQ ID NO: 393 - DNA, 129 bp, synthetic construct
  misc_feature 1..129: note = Polynucleotide encoding the polypeptide of SEQ ID NO: 392
  source 1..129: mol_type = other DNA
accaaatattatggcaacggcgtgtattgcaacagcaaaaaatgctgggtggattggggcaccgcgcagggctgcattgatgtggtgattggccagctgggcggcggcattccgggcaaaggcaaatgc

SEQ ID NO: 394 - protein, 62 aa, Enterococcus sp.
NRWYCNSAAGGVGGAAVCGLAGYVGEAKENIAGEVRKGWGMAGGFTHNKACKSFPGSGWASG

SEQ ID NO: 395 - DNA, 186 bp, synthetic construct
  misc_feature 1..186: note = Polynucleotide encoding the polypeptide of SEQ ID NO: 394
  source 1..186: mol_type = other DNA
aaccgctggtattgcaacagcgcggcgggcggcgtgggcggcgcggcggtgtgcggcctggcgggctatgtgggcgaagcgaaagaaaacattgcgggcgaagtgcgcaaaggctggggcatggcgggcggctttacccataacaaagcgtgcaaaagctttccgggcagcggctgggcgagcggc

SEQ ID NO: 396 - protein, 39 aa, Enterococcus faecium
TTKNYGNGVCNSVNWCQCGNVWASCNLATGCAAWLCKLA

SEQ ID NO: 397 - DNA, 117 bp, synthetic construct
  misc_feature 1..117: note = Polynucleotide encoding the polypeptide of SEQ ID NO: 396
  source 1..117: mol_type = other DNA
accaccaaaaactatggcaacggcgtgtgcaacagcgtgaactggtgccagtgcggcaacgtgtgggcgagctgcaacctggcgaccggctgcgcggcgtggctgtgcaaactggcg

SEQ ID NO: 398 - protein, 30 aa, Paenibacillus polymyxa
ASIIKTTIKVSKAVCKTLTCICTGSCSNCK

SEQ ID NO: 399 - DNA, 90 bp, synthetic construct
  misc_feature 1..90: note = Polynucleotide encoding the polypeptide of SEQ ID NO: 398
  source 1..90: mol_type = other DNA
gcgagcattattaaaaccaccattaaagtgagcaaagcggtgtgcaaaaccctgacctgcatttgcaccggcagctgcagcaactgcaaa

SEQ ID NO: 400 - protein, 31 aa, Staphylococcus epidermidis
SASIVKTTIKASKKLCRGFTLTCGCHFTGKK

SEQ ID NO: 401 - DNA, 93 bp, synthetic construct
  misc_feature 1..93: note = Polynucleotide encoding the polypeptide of SEQ ID NO: 400
  source 1..93: mol_type = other DNA
agcgcgagcattgtgaaaaccaccattaaagcgagcaaaaaactgtgccgcggctttaccctgacctgcggctgccattttaccggcaaaaaa

SEQ ID NO: 402 - protein, 43 aa, Enterococcus faecium
KYYGNGVSCNKKGCSVDWGKAIGIIGNNAAANLTTGGKAAWAC

SEQ ID NO: 403 - DNA, 129 bp, synthetic construct
  misc_feature 1..129: note = Polynucleotide encoding the polypeptide of SEQ ID NO: 402
  source 1..129: mol_type = other DNA
aaatattatggcaacggcgtgagctgcaacaaaaaaggctgcagcgtggattggggcaaagcgattggcattattggcaacaacgcggcggcgaacctgaccaccggcggcaaagcggcgtgggcgtgc

SEQ ID NO: 404 - protein, 39 aa, Paenibacillus polymyxa
ATYYGNGLYCNKQKHYTWVDWNKASREIGKITVNGWVQH

SEQ ID NO: 405 - DNA, 117 bp, synthetic construct
  misc_feature 1..117: note = Polynucleotide encoding the polypeptide of SEQ ID NO: 404
  source 1..117: mol_type = other DNA
gcgacctattatggcaacggcctgtattgcaacaaacagaaacattatacctgggtggattggaacaaagcgagccgcgaaattggcaaaattaccgtgaacggctgggtgcagcat

SEQ ID NO: 406 - protein, 35 aa, Bacillus circulans
VNYGNGVSCSKTKCSVNWGIITHQAFRVTSGVASG

SEQ ID NO: 407 - DNA, 105 bp, synthetic construct
  misc_feature 1..105: note = Polynucleotide encoding the polypeptide of SEQ ID NO: 406
  source 1..105: mol_type = other DNA
gtgaactatggcaacggcgtgagctgcagcaaaaccaaatgcagcgtgaactggggcattattacccatcaggcgtttcgcgtgaccagcggcgtggcgagcggc

SEQ ID NO: 408 - protein, 30 aa, Paenibacillus polymyxa
FVYGNGVTSILVQAQFLVNGQRRFFYTPDK

SEQ ID NO: 409 - DNA, 90 bp, synthetic construct
  misc_feature 1..90: note = Polynucleotide encoding the polypeptide of SEQ ID NO: 408
  source 1..90: mol_type = other DNA
tttgtgtatggcaacggcgtgaccagcattctggtgcaggcgcagtttctggtgaacggccagcgccgctttttttataccccggataaa

SEQ ID NO: 410 - protein, 13 aa, Lactobacillus rhamnosus
AVPAVRKTNETLD

SEQ ID NO: 411 - DNA, 39 bp, synthetic construct
  misc_feature 1..39: note = Polynucleotide encoding the polypeptide of SEQ ID NO: 410
  source 1..39: mol_type = other DNA
gcggtgccggcggtgcgcaaaaccaacgaaaccctggat

SEQ ID NO: 412 - protein, 69 aa, Bacillus licheniformis
MKNSAAREAFKGANHPAGMVSEEELKALVGGNDVNPETTPATTSSWTCITAGVTVSASLCPTTKCTSRC

SEQ ID NO: 413 - DNA, 207 bp, synthetic construct
  misc_feature 1..207: note = Polynucleotide encoding the polypeptide of SEQ ID NO: 412
  source 1..207: mol_type = other DNA
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgcccgaccaccaaatgcaccagccgctgc

SEQ ID NO: 414 - protein, 36 aa, Lactobacillus plantarum
KYYGNGLSCSKKGCTVNWGQAFSCGVNRVATAGHGK

SEQ ID NO: 415 - DNA, 108 bp, synthetic construct
  misc_feature 1..108: note = Polynucleotide encoding the polypeptide of SEQ ID NO: 414
  source 1..108: mol_type = other DNA
aaatattatggcaacggcctgagctgcagcaaaaaaggctgcaccgtgaactggggccaggcgtttagctgcggcgtgaaccgcgtggcgaccgcgggccatggcaaa

SEQ ID NO: 416 - protein, 24 aa, Lactobacillus acidophilus
GNPKVAHCASQIGRSTAWGAVSGA

SEQ ID NO: 417 - DNA, 72 bp, synthetic construct
  misc_feature 1..72: note = Polynucleotide encoding the polypeptide of SEQ ID NO: 416
  source 1..72: mol_type = other DNA
ggcaacccgaaagtggcgcattgcgcgagccagattggccgcagcaccgcgtggggcgcggtgagcggcgcg

SEQ ID NO: 418 - protein, 40 aa, Enterococcus faecalis; identical to SEQ ID NO: 1

SEQ ID NO: 419 - DNA, 120 bp, synthetic construct
  misc_feature 1..120: note = Polynucleotide encoding the polypeptide of SEQ ID NO: 418
  source 1..120: mol_type = other DNA
tggctgccgccggcgggcctgctgggccgctgcggccgctggtttcgcccgtggctgctgtggctgcagagcggcgcgcagtataaatggctgggcaacctgtttggcctgggcccgaaa

SEQ ID NO: 420 - protein, 40 aa, Anabaena variabilis
NLDQWLTEQVHEFQDMYLEPQAISNQDITFKLSDLDFIHN

SEQ ID NO: 421 - DNA, 124 bp, Anabaena variabilis
  source 1..124: mol_type = genomic DNA
taatttagatcagtggttaacagaacaagttcatgagtttcaagatatgtacttggaaccacaagcaatatccaatcaagacattaccttcaaactatctgacctagattttattcataattga

SEQ ID NO: 422 - protein, 40 aa, Nostoc sp; identical to SEQ ID NO: 420

SEQ ID NO: 423 - DNA, 123 bp, Nostoc sp
  source 1..123: mol_type = genomic DNA
aatttagatcaatggttaacagaacaagttcatgagtttcaagatatgtacttggaaccacaagcaatatccaatcaagacattaccttcaaactgtcagacctagattttattcataattga

SEQ ID NO: 424 - protein, 7 aa, Nostoc azollae
HREKKSA

SEQ ID NO: 425 - DNA, 24 bp, Nostoc azollae
  source 1..24: mol_type = genomic DNA
cacagagagaaaaaatcagcatag

SEQ ID NO: 426 - protein, 23 aa, Acaryochloris marina
TSNNWLAKNYLSMWNKKSSNPNL

SEQ ID NO: 427 - DNA, 72 bp, Acaryochloris marina
  source 1..72: mol_type = genomic DNA
acaagcaataactggctagccaaaaactatctttctatgtggaataaaaagagcagtaatccaaacctttag

SEQ ID NO: 428 - protein, 6 aa, Cyanothece sp.
FRYFWW

SEQ ID NO: 429 - DNA, 21 bp, Cyanothece sp.
  source 1..21: mol_type = genomic DNA
tttagatatttttggtggtaa

SEQ ID NO: 430 - protein, 6 aa, Cyanothece sp.
FRYFWW

SEQ ID NO: 431 - DNA, 21 bp, Cyanothece sp.
  source 1..21: mol_type = genomic DNA
tttagatatttttggtggtaa

SEQ ID NO: 432 - protein, 9 aa, Cyanothece sp.
CGEKWRIFS

SEQ ID NO: 433 - DNA, 27 bp, Cyanothece sp.
  source 1..27: mol_type = genomic DNA
tgtggagaaaaatggagaatttttagc

SEQ ID NO: 434 - protein, 8 aa, Cyanothece sp.
FRLQLWQF

SEQ ID NO: 435 - DNA, 24 bp, Cyanothece sp.
  source 1..24: mol_type = genomic DNA
tttcgcttacaactgtggcaattt

SEQ ID NO: 436 - protein, 16 aa, Cyanothece sp.
LGCNQSSIWSIFFWNH

SEQ ID NO: 437 - DNA, 51 bp, Cyanothece sp.
  source 1..51: mol_type = genomic DNA
ctaggatgtaaccagagcagtatctggtcaatttttttctggaatcattaa

SEQ ID NO: 438 - protein, 40 aa, Microcoleus chthonoplastes
YNLQGLPAIESEDCIPDSVAPSDDWFSGVSSLFNRLTGLG

SEQ ID NO: 439 - DNA, 123 bp, Microcoleus chthonoplastes
  source 1..123: mol_type = genomic DNA
tataacctacaggggttgccagcaattgagtcagaagactgtatcccagattctgtagcgccttcggatgattggttttcaggcgtatcgtctctgtttaaccgcttgactgggttgggttag

SEQ ID NO: 440 - protein, 37 aa, Nostoc sp
WMAIRRILRCHPFHPGGYDPVPELGEHCCHHDSGNKG

SEQ ID NO: 441 - DNA, 114 bp, Nostoc sp
  source 1..114: mol_type = genomic DNA
tggatggcgattcgccgcattttgcgttgtcatccattccacccagggggttatgatcctgtaccagagttgggtgagcattgttgtcatcatgatagcgggaataaggggtga

SEQ ID NO: 442 - protein, 35 aa, Anabaena variabilis
WMGIRRILRCHPFHPGGYDPVPEVGEHCCHHDSGK

SEQ ID NO: 443 - DNA, 108 bp, Anabaena variabilis
  source 1..108: mol_type = genomic DNA
tggatggggattcgccgcattttgcgttgtcatccattccacccaggcggttatgatcctgtaccagaggtgggtgagcattgttgtcatcatgatagcgggaagtag

SEQ ID NO: 444 - protein, 48 aa, Nodularia spumigena
WMATRRILRCHPFHPGGYDPVPEVKHNCCDQHLSDSGKQTTEDHHKGS

SEQ ID NO: 445 - DNA, 147 bp, Nodularia spumigena
  source 1..147: mol_type = genomic DNA
tggatggcgactcggcggattttgcgttgtcatcccttccatcctggtggatatgatccagttccagaggtaaaacacaattgctgcgatcagcatctgtccgattctgggaaacagaccacagaagaccatcacaaaggctcgtag

SEQ ID NO: 446 - protein, 34 aa, Nostoc azollae
WMATLRILRCHPFHPGGYDPVPGLAEKSCCDHHD

SEQ ID NO: 447 - DNA, 105 bp, Nostoc azollae
  source 1..105: mol_type = genomic DNA
tggatggcaactttgcggattttacgctgtcatcctttccatcctggtggttatgatcctgtaccaggactagcggaaaaatcctgttgtgaccatcatgattga

SEQ ID NO: 448 - protein, 28 aa, Synechococcus
WLTAKRFCRCHPLHPGGYDPVPEKKSVL

SEQ ID NO: 449 - DNA, 87 bp, Synechococcus
  source 1..87: mol_type = genomic DNA
tggctaacagccaagcgcttttgtcgctgtcatccgcttcatcctggcgggtatgatccggtaccggagaagaaatcggtactctaa

SEQ ID NO: 450 - protein, 23 aa, Prochlorococcus marinus
WLTLRRLSRCHPFTPCGCDPVPD

SEQ ID NO: 451 - DNA, 72 bp, Prochlorococcus marinus
  source 1..72: mol_type = genomic DNA
tggctcaccctgcggcgcctgtctcgttgccatccttttaccccctgtggttgcgacccggtgcctgattaa

SEQ ID NO: 452 - protein, 69 aa, Escherichia coli
MSYKKLYQLTAIFSLPLTILLVSLSSLRIVGEGNSYVDVFLSFIIFLGFIELIHGIRKILVWSGWKNGS

SEQ ID NO: 453 - DNA, 210 bp, Escherichia coli
  source 1..210: mol_type = genomic DNA
atgagttataaaaaactgtaccaattgacggctatatttagtttacctcttactatcttattggtttcactttcatcccttcggattgttggcgaagggaattcttatgttgacgtttttctaagctttataatatttcttggttttattgagctgattcatgggattcgaaagattttggtctggtcaggctggaaaaacggaagttaa

SEQ ID NO: 454 - protein, 85 aa, Escherichia coli
MGLKLDLTWFDKSTEDFKGEEYSKDFGDDGSVMESLGVPFKDNVNNGCFDVIAEWVPLLQPYFNHQIDISDNEYFVSFDYRDGDW

SEQ ID NO: 455 - DNA, 258 bp, Escherichia coli
  source 1..258: mol_type = genomic DNA
atgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtga

SEQ ID NO: 456 - protein, 113 aa, Escherichia coli
MSLRYYIKNILFGLYCTLIYIYLITKNSEGYYFLVSDKMLYAIVISTILCPYSKYAIEYIAFNFIKKDFFERRKNLNNAPVAKLNLFMLYNLLCLVLAIPFGLLGLFISIKNN

SEQ ID NO: 457 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagggtattatttccttgtgtcagataagatgctatatgcaatagtgataagcactattctatgtccatattcaaaatatgctattgaatacatagcttttaacttcataaagaaagattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 458 - protein, 85 aa, Escherichia coli
MGLKLHIHWFDKKTEEFKGGEYSKDFGDDGSVIESLGMPLKDNINNGWFDVEKPWVSILQPHFKNVIDISKFDYFVSFVYRDGNW

SEQ ID NO: 459 - DNA, 258 bp, Escherichia coli
  source 1..258: mol_type = genomic DNA
atggggcttaaattacatattcattggtttgataagaaaaccgaagagtttaaaggcggtgaatactcaaaagacttcggtgatgatggttctgtcattgaaagtctggggatgcctttaaaggataatattaataatggttggtttgatgttgaaaaaccatgggtttcgatattacagccacactttaaaaatgtaatcgatattagtaaatttgattactttgtatcctttgtttaccgggatggtaactggtaa

SEQ ID NO: 460 - protein, 86 aa, Escherichia coli
MELKHSISDYTEAEFLEFVKKICRAEGATEEDDNKLVREFERLTEHPDGSDLIYYPRDDREDSPEGIVKEIKEWRAANGKSGFKQG

SEQ ID NO: 461 - DNA, 261 bp, Escherichia coli
  source 1..261: mol_type = genomic DNA
atggaactgaaacatagtattagtgattataccgaggctgaatttctggagtttgtaaaaaaaatatgtagagctgaaggtgctactgaagaggatgacaataaattagtgagagagtttgagcgattaactgagcacccagatggttcagatctgatttattatcctcgcgatgacagggaagatagtcctgaagggattgtcaaggaaattaaagaatggcgagctgctaacggtaagtcaggatttaaacagggctga

SEQ ID NO: 462 - protein, 178 aa, Citrobacter freundii
MMNEHSIDTDNRKANNALYLFIIIGLIPLLCIFVVYYKTPDALLLRKIATSTENLPSITSSYNPLMTKVMDIYCKTAPFLALILYILTFKIRKLINNTDRNTVLRSCLLSPLVYAAIVYLFCFRNFELTTAGRPVRLMATNDATLLLFYIGLYSIIFFTTYITLFTPVTAFKLLKKRQ

SEQ ID NO: 463 - DNA, 537 bp, Citrobacter freundii
  source 1..537: mol_type = genomic DNA
atgatgaatgaacactcaatagatacggacaacagaaaggccaataacgcattgtatttatttataataatcggattaataccattattgtgcatttttgttgtttactacaaaacgccagacgctttacttttacgtaaaattgctacaagcactgagaatctcccgtcaataacatcctcctacaacccattaatgacaaaggttatggatatttattgtaaaacagcgcctttccttgccttaatactatacatcctaacctttaaaatcagaaaattaatcaacaacaccgacaggaacactgtacttagatcttgtttattaagtccattggtctatgcagcaattgtttatctattctgcttccgaaattttgagttaacaacagccggaaggcctgtcagattaatggccaccaatgacgcaacactattgttattttatattggtctgtactcaataattttctttacaacctatatcacgctattcacaccagtcactgcatttaaattattaaaaaaaaggcagtaa

SEQ ID NO: 464 - protein, 111 aa, Escherichia coli
MNRKYYFNNMWWGWVTGGYMLYMSWDYEFKYRLLFWCISLCGMVLYPVAKWYIEDTALKFTRPDFWNSGFFADTPGKMGLLAVYTGTVFILSLPLSMIYILSVIIKRLSVR

SEQ ID NO: 465 - DNA, 336 bp, Escherichia coli
  source 1..336: mol_type = genomic DNA
atgaacagaaaatattattttaataatatgtggtggggatgggtgacggggggatatatgctgtatatgtcatgggattatgagtttaaatacagattactgttctggtgtatttctctctgcggaatggttttgtatccggttgcaaaatggtatattgaagatacagctctaaaatttacccggcctgatttctggaacagcggtttttttgctgatacacctggaaaaatggggttgcttgcggtttatacgggtactgttttcatattatctcttccgttaagtatgatatatattctttctgttattataaaaaggctgtctgtaagatag

SEQ ID NO: 466 - protein, 115 aa, Escherichia coli
MKLDISVKYLLKSLIPILIILTVFYLGWKDNQENARMFYAFIGCIISAITFPFSMRIIQKMVIRFTGKEFWQKDFFTNPVGGSLTAIFELFCFVISVPVVAIYLIFILCKALSGK

SEQ ID NO: 467 - DNA, 348 bp, Escherichia coli
  source 1..348: mol_type = genomic DNA
atgaaactggatatatctgtaaagtatttactgaaaagcctgataccaatcctcattattcttacagttttttatctgggatggaaagataaccaggaaaatgcaagaatgttttatgcgttcatcggatgcattatcagtgccattacttttcctttttcaatgaggataatacagaaaatggtaataaggtttacagggaaagaattctggcaaaaagacttctttacaaatccagttggcggaagcttaactgcaatatttgaattattctgtttcgttatatcagttcctgtggttgccatttacttaatttttatactctgcaaagccctttcaggaaaatga

SEQ ID NO: 468 - protein, 131 aa, Escherichia coli
MHNTLLEKIIAYLSLPGFHSLNNPPLSEAFNLYVHTAPLAATSLFIFTHKELELKPKSSPLRALKILTPFTILYISMIYCFLLTDTELTLSSKTFVLIVKKRSVFVFFLYNTIYWDIYIHIFVLLVPYRNI

SEQ ID NO: 469 - DNA, 396 bp, Escherichia coli
  source 1..396: mol_type = genomic DNA
atgcacaatacactcctcgaaaaaatcatcgcatacctatccctaccaggatttcattcattaaacaacccgcccctaagcgaagcattcaatctctatgttcatacagcccctttagctgcaaccagcttattcatattcacacacaaagaattagagttaaaaccaaagtcgtcacctctgcgggcactaaagatattaactcctttcactattctttatatatccatgatatactgtttcttgctaactgacacagaactaaccttgtcatcaaaaacatttgtattaatagtcaaaaaacgatctgtttttgtcttttttctatataacactatatattgggatatatatattcacatatttgtacttttggttccttataggaacatataa

SEQ ID NO: 470 - protein, 85 aa, Escherichia coli
MELKNSISDYTETEFKKIIEDIINCEGDEKKQDDNLEHFISVTEHPSGSDLIYYPEGNNDGSPEAVIKEIKEWRAANGKSGFKQG

SEQ ID NO: 471 - DNA, 258 bp, Escherichia coli
  source 1..258: mol_type = genomic DNA
atggaactgaaaaacagcattagtgattacactgaaactgaattcaaaaaaattattgaagacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 472 - protein, 98 aa, Lactococcus lactis
  source 1..98: note = subsp. lactis
MKKKQIEFENELRSMLATALEKDISQEERNALNIAEKALDNSEYLPKIILNLRKALTPLAINRTLNHDLSELYKFITSSKASNKNLGGGLIMSWGRLF

SEQ ID NO: 473 - DNA, 297 bp, Lactococcus lactis
  source 1..297: mol_type = genomic DNA; note = subsp. lactis
atgaaaaaaaaacaaatagaatttgaaaacgagctaagaagtatgttggctaccgcccttgaaaaagacattagtcaagaggaaagaaatgctctgaatattgcagaaaaggcgcttgacaattctgaatatttaccaaaaattattttaaacctcagaaaagccctaactccattagctataaatcgaacacttaaccatgatttatctgaactgtataaattcattacaagttccaaagcatcaaacaaaaatttaggtggtggtttaattatgtcgtggggacgactattctaa

SEQ ID NO: 474 - protein, 98 aa, Lactococcus lactis; identical to SEQ ID NO: 472
  source 1..98: note = subsp. cremoris

SEQ ID NO: 475 - DNA, 297 bp, Lactococcus lactis; identical to SEQ ID NO: 473
  source 1..297: mol_type = genomic DNA; note = subsp. cremoris

SEQ ID NO: 476 - protein, 87 aa, Escherichia coli
MNKMAMIDLAKLFLASKITAIEFSERICVERRRLYGVKDLSPNILNCGEELFMAAERFEPDADRANYEIDDNGLKVEVRSILEKFKL

SEQ ID NO: 477 - DNA, 249 bp, Escherichia coli
  source 1..249: mol_type = genomic DNA
atgatcgatttggcgaaattatttttagcttcgaaaattacagtgattgagttttcagagcgaatttgtgttgaacggagaagattgtatggtgttaaggatttgtctccgaatatattaaattgtggggaagagttgtctatggctgctgagcgatttgagcctgatgcagatagggctaattatgaaattgatgataatggacttaaggtcgaggtccgatctatcttggaaaaacttaaatcataa

SEQ ID NO: 478 - protein, 83 aa, Escherichia coli
MKLSPKAAIEVCNEAAKKGLWILGIDGGHWLNPGFRIDSSASWTYDMPEEYKSKIPENNRLAIENIKDDIENGYTAFIITLKM

SEQ ID NO: 479 - DNA, 243 bp, Escherichia coli
  source 1..243: mol_type = genomic DNA
atgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggagaatacaaatcaaaaatccctgaaaataatagattggctattgaaaatattaaagatgatattgagaatggatacactgctttcattatcacgttaa

SEQ ID NO: 480 - protein, 85 aa, Escherichia coli
MGLKLHINWFDKRTEEFKGGEYSKDFGDDGSVIERLGMPFKDNINNGWFDVIAEWVPLLQPYFNHQIDISDNEYFVSFDYRDGDW

SEQ ID NO: 481 - DNA, 258 bp, Escherichia coli
  source 1..258: mol_type = genomic DNA
atggggcttaaattacatattaattggtttgataagacgaccgaggaatttaaaggtggtgagtattcaaaagattttggagatgatggctcggtcattgaacgtcttggaatgcctttaaaagataatatcaataatggttggtttgatgttatagctgaatgggtacctttgctacaaccatactttaatcatcaaattgatatttccgataatgagtattttgtttcgtttgattatcgtgatggtgattggtga

SEQ ID NO: 482 - protein, 85 aa, Escherichia coli
MELKKSIGDYTETEFKKIIENIINCEGDEKKQDDNLEHFISVTEHPSGSDLIYYPEGNNDGSPEAVIKEIKEWRAANGKSGFKQG

SEQ ID NO: 483 - DNA, 258 bp, Escherichia coli
  source 1..258: mol_type = genomic DNA
gtggagctaaagaaaagtattggtgattacactgaaaccgaattcaaaaaaattattgaaaacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 484 - protein, 86 aa, Escherichia coli [sequence omitted]

SEQ ID NO: 485 - DNA, 261 bp, Escherichia coli
  source 1..261: mol_type = genomic DNA
atggaactgaagcatagcattagtgattatacagaagctgaatttttacaacttgtaacaacaatttgtaatgcgaacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggctaa

SEQ ID NO: 486 - protein, 141 aa, Escherichia coli
MLTLYGYIRNVFLYRMNDRSCGDFMKVISMKFIFILTIIALAAVFFWSEDKGPACYQVSDEQARTFVKNDYLQRMKRWDNDVQLLGTEIPKITWEKIERSLTDVEDEKTLLVPFKAEGPDGKRMYYGMYHCEEGYVEYAND

SEQ ID NO: 487 - DNA, 354 bp, Escherichia coli
  source 1..354: mol_type = genomic DNA
atgaaagtaattagcatgaaatttatttttattttaacgattattgctcttgctgctgtttttttctggtctgaagataaaggtccggcatgctatcaggtcagcgatgaacaggccagaacgtttgtaaaaaatgattacctgcaaagaatgaaacgctgggacaacgatgtacaacttcttggtacagaaatcccgaaaattacatgggaaaagattgagagaagtttaacagatgttgaagatgaaaaaacacttcttgtcccatttaaagctgaaggcccggacggtaagagaatgtattatggcatgtaccattgtgaggagggatatgttgaatatgcgaatgactaa

SEQ ID NO: 488 - protein, 175 aa, Escherichia coli
MTSNKDKNKKANEILYAFSIIGIIPLMAILILRINDPYSQVLYYLYNKVAFLPSITSLHDPVMTTLMSNYNKTAPVMGILVFLCTYKTREIIKPVTRKLVVQSCFWGPVFYAILIYITLFYNLELTTAGGFFKLLSHNVITLFILYCSIYFTVLTMTYAILLMPLLVIKYFKGRQ

SEQ ID NO: 489 - DNA, 528 bp, Escherichia coli
  source 1..528: mol_type = genomic DNA
atgaccagcaataaagataagaacaagaaagcaaacgaaatattatatgcattttccataatcgggattattccattaatggctatattaatacttcgaataaatgatccatattctcaagtgctgtactacttatataataaggtggcatttctcccttctattacatcattgcatgatcccgtcatgacaacacttatgtcaaactacaacaagacagcgccagttatgggtattctcgtttttctttgcacatataagacaagagaaatcataaagccagtaacaagaaaacttgttgtgcaatcctgtttctgggggcccgttttttatgccattctgatttatatcacactgttctataatctggaactaacaacagcaggtggtttttttaaattattatctcataatgtcatcactctgtttattttatattgctccatttactttactgttttaaccatgacatatgcgattttactgatgccattacttgtcattaaatattttaaagggaggcagtaa

SEQ ID NO: 490 - protein, 78 aa, Escherichia coli
MDRKRTKLELLFAFIINATAIYIALAIYDCVFRGKDFLSMHTFCFSALMSAICYFVGDNYYSISDKIKRRSYENSDSK

SEQ ID NO: 491 - DNA, 237 bp, Escherichia coli
  source 1..237: mol_type = genomic DNA
atggatagaaaaagaacaaaattagagttgttatttgcatttataataaatgccaccgcaatatatattgcattagctatatatgattgtgtttttagaggaaaggactttttatccatgcatacattttgcttctctgcattaatgtctgcaatatgttactttgttggtgataattattattcaatatccgataagataaaaaggagatcatatgagaactctgactctaaatga

SEQ ID NO: 492 - protein, 113 aa, Shigella sonnei
MSLRYYIKNILFGLYCALIYIYLITKNNEGYYFLASDKMLYAIVISTILCPYSKYAIEHIFFKFIKKDFFRKRKNLNKCPRGKIKPYLCVYNLLCLVLAIPFGLLGLVYINKE

SEQ ID NO: 493 - DNA, 342 bp, Shigella sonnei
  source 1..342: mol_type = genomic DNA
atgagtttaagatactacataaaaaatattttgtttggcctatactgcgcacttatatatatataccttataacaaaaaacaacgaagggtattatttcctagcgtcagataagatgctatacgcaatagtgataagcactattctatgcccatattcaaaatatgctattgaacacatattttttaagttcataaagaaagattttttcagaaaaagaaaaaacctaaataaatgcccccgtggcaaaattaaaccgtatttatgcgtatacaatctactttgtttggtcctagcaatcccatttggattgctaggacttgtttatatcaataaagaataa

SEQ ID NO: 494 - protein, 113 aa, Escherichia coli
MSLRYYIKNILFGLYCTLIYIYLITKNSEEYYFLVTDKMLYAIVISTILCPYSKYAIEHIAFNFIKKHFFERRKNLNNAPVAKLNLFMLYNLLCLVLAIPFGLLGLFISIKNN

SEQ ID NO: 495 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagagtattatttccttgtgacagataagatgctatatgcaatagtgataagcactattctatgtccatattcaaaatatgctattgaacacatagcttttaacttcataaagaaacattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 496 - protein, 113 aa, Leuconostoc gelidum
MRKNNILLDDAKIYTNKLYLLLIDRKDDAGYGDICDVLFQVSKKLDSTKNVEALINRLVNYIRITASTNRIKFSKDEEAVIIELGVIGQKAGLNGQYMADFSDKSQFYSIFER

SEQ ID NO: 497 - DNA, 342 bp, Leuconostoc gelidum
  source 1..342: mol_type = genomic DNA
ttgagaaaaaataacattttattggacgatgctaaaatatacacgaacaaactctatttgctattaatcgatagaaaagatgacgctgggtatggagatatttgtgatgttttgtttcaggtatccaaaaaattagatagcacaaaaaatgtagaagcattgattaaccgattggtcaattatatacgaattaccgcttcaacaaacagaattaagttttcaaaagatgaagaggctgtaattatagaacttggtgtaattggtcagaaggctggattaaacggccaatacatggctgatttttctgacaaatctcagttttatagtatctttgaaagataa

SEQ ID NO: 498 - protein, 91 aa, Lactococcus lactis
  source 1..91: note = subsp. cremoris
MKKKVDTEKQITSWASDLASKNETKVQEKLILSSYIQDIENHVYFPKAMISLEKKLRDQNNICALSKEVNQFYFKVVEVNQRKSWMVGLIV

SEQ ID NO: 499 - DNA, 276 bp, Lactococcus lactis
  source 1..276: mol_type = genomic DNA; note = subsp. cremoris
atgaaaaaaaaagttgatacagaaaaacaaattacttcttgggcatctgacttagcttccaaaaatgaaacaaaggttcaagaaaaattaatactgtcttcttatattcaggacatcgaaaaccatgtttactttccaaaagcaatgatttctttagaaaaaaaattacgagaccaaaataatatttgcgctttatcaaaagaagtcaatcagttttattttaaagttgttgaagtaaatcaaagaaaatcctggatggtaggtttgatagtttaa

SEQ ID NO: 500 - protein, 112 aa, Pediococcus acidilactici
MNKTKSEHIKQQALDLFTRLQFLLQKHDTIEPYQYVLDILETGISKTKHNQQTPERQARVVYNKIASQALVDKLHFTAEENKVLAAINELAHSQKGWGEFNMLDTTNTWPSQ

SEQ ID NO: 501 - DNA, 339 bp, Pediococcus acidilactici
  source 1..339: mol_type = genomic DNA
atgaataagactaagtcggaacatattaaacaacaagctttggacttatttactaggctacagtttttactacagaagcacgatactatcgaaccttaccagtacgttttagatattctggagactggtatcagtaaaactaaacataaccagcaaacgcctgaacgacaagctcgtgtagtctacaacaagattgccagccaagcgttagtagataagttacattttactgccgaagaaaacaaagttctagcagccatcaatgaattggcgcattctcaaaaagggtggggcgagtttaacatgctagatactaccaatacgtggcctagccaatag

SEQ ID NO: 502 - protein, 88 aa, Carnobacterium maltaromaticum
MIKDEKINKIYALVKSALDNTDVKNDKKLSLLLMRIQETSINGELFYDYKKELQPAISMYSIQHNFRVPDDLVKLLALVQTPKAWSGF

SEQ ID NO: 503 - DNA, 267 bp, Carnobacterium maltaromaticum
  source 1..267: mol_type = genomic DNA
atgataaaagatgaaaaaataaataaaatctatgctttagttaagagcgcacttgataatacggatgttaagaatgataaaaaactttctttacttcttatgagaatacaagaaacatcaattaatggagaactattttacgattataaaaaagaattacagccagctattagtatgtactctattcaacataactttcgggttcctgacgatctagtaaaactgttagcattagttcaaacacctaaagcttggtcagggttttaa

SEQ ID NO: 504 - protein, 111 aa, Carnobacterium maltaromaticum
MDIKSQTLYLNLSEAYKDPEVKANEFLSKLVVQCAGKLTASNSENSYIEVISLLSRGISSYYLSHKRIIPSSMLTIYTQIQKDIKNGNIDTEKLRKYEIAKGLMSVPYIYF

SEQ ID NO: 505 - DNA, 336 bp, Carnobacterium maltaromaticum
  source 1..336: mol_type = genomic DNA
atggatataaagtctcaaacattatatttgaatctaagcgaggcatataaagaccctgaagtaaaagctaatgaattcttatcaaaattagttgtacaatgtgctgggaaattaacagcttcaaacagtgagaacagttatattgaagtaatatcattgctatctaggggtatttctagttattatttatcccataaacgtataattccttcaagtatgttaactatatatactcaaatacaaaaggatataaaaaacgggaatattgacaccgaaaaattaaggaaatatgagatagcaaaaggattaatgtccgttccttatatatatttctaa

SEQ ID NO: 506 - protein, 245 aa, Lactococcus lactis
  source 1..245: note = subsp. lactis
MRRYLILIVALIGITGLSGCYQTSHKKVRFDEGSYTNFIYDNKSYFVTDKEIPQENVNNSKVKFYKLLIVDMKSEKLLSSSNKNSVTLVLNNIYEASDKSLCMGINDRYYKILPESDKGAVKALRLQNFDVTSDISDDNFVIDKNDSRKIDYMGNIYSISDTTVSDEELGEYQDVLAEVRVFDSVSGKSIPRSEWGRIDKDGSNSKQSRTEWDYGEIHSIRGKSLTEAFAVEINDDFKLATKVGN

SEQ ID NO: 507 - DNA, 738 bp, Lactococcus lactis
  source 1..738: mol_type = genomic DNA; note = subsp. lactis
atgagaagatatttaatacttattgtggccttaatagggataacaggtttatcagggtgttatcaaacaagtcataaaaaggtgaggtttgacgaaggaagttatactaattttatttatgataataaatcgtatttcgtaactgataaggagattcctcaggagaacgttaacaattccaaagtaaaattttataagctgttgattgttgacatgaaaagtgagaaacttttatcaagtagcaacaaaaatagtgtgactttggtcttaaataatatttatgaggcttctgacaagtcgctatgtatgggtattaacgacagatactataagatacttccagaaagtgataagggggcggtcaaagctttgagattacaaaactttgatgtgacaagcgatatttctgatgataattttgttattgataaaaatgattcacgaaaaattgactatatgggaaatatttacagtatatcggacaccaccgtatctgatgaagaattgggagaatatcaggatgttttagctgaagtacgtgtgtttgattcagttagtggcaaaagtatcccgaggtctgaatgggggagaattgataaggatggttcaaattccaaacagagtaggacggaatgggattatggcgaaatccattctattagaggaaaatctcttactgaagcatttgccgttgagataaatgatgattttaagcttgcaacgaaggtaggaaactag

SEQ ID NO: 508 - protein, 261 aa, Rhizobium leguminosarum
  source 1..261: note = bv. trifolii
MNDEICLTGGGRTTVTRRGGVVYREGGPWSSTVISLLRHLEASGFAEAPSVVGTGFDERGRETLSFIEGEFVHPGPWSEEAFPQFGMMLRRLHDATASFKPPENSMWRDWFGRNLGEGQHVIGHCDTGPWNIVCRSGLPVGLIDWEVAGPVRADIELAQACWLNAQLYDDDIAERVGLGSVTMRAHQVRLLLDGYGLSRKQRGGFVDKLITFAVHDAAEQAKEAAVTPESNDAEPLWAIAWRTRSASWMLHHRQTLEAALA

SEQ ID NO: 509 - DNA, 786 bp, Rhizobium leguminosarum
  source 1..786: mol_type = genomic DNA; note = bv. trifolii
atgaatgatgagatttgcctgacaggtggcggacgaacgactgtcacgcggcgcggcggagtcgtgtatcgcgaaggcggcccgtggtcatcaaccgtcatttcgctcctgcggcatctggaagcctctggcttcgctgaagctccttccgttgtcggcaccggtttcgatgagcgcggccgggagacattatcgtttatcgagggtgagtttgttcacccaggcccttggtcggaggaggcttttccgcaatttggaatgatgttgcggcgactgcacgatgccaccgcctcgttcaaacctcccgaaaactcgatgtggcgcgattggttcgggcgtaacctcggtgagggtcaacacgtaataggacactgcgacacaggcccatggaacattgtttgccggtcaggattgcctgtcgggttgatagattgggaggtggctgggcctgtcagggcggatatcgaattggcccaggcttgttggctgaatgcccagctctacgatgacgacattgcggagagggtcggattaggctctgtgaccatgagagcgcatcaagttcgcctgctgcttgacggctatggtctgtctcggaagcaacgcggcggcttcgtcgacaagctaatcacgttcgcagttcacgatgcggccgagcaggcgaaagaggcggctgtcacgccagagtcgaacgatgcggaaccgctatgggcaattgcctggcgcactagaagtgcctcctggatgctccatcatcggcaaacactggaagcagcgctggcatag

SEQ ID NO: 510 - protein, 436 aa, Bacillus subtilis
MNNIIPIMSLLFKQLYSRQGKKDAIRIAAGLVILAVFEIGLIRQAGIDESVLRKTYIILALLLMNTYMVFLSVTSQWKESYMKLSCLLPISSRSFWLAQSVVLFVDTCLRRTLFFFILPLFLFGNGTLSGAQTLFWLGRFSFFTVYSIIFGVVLSNHFVKKKNLMFLLHAAIFACVCISAALMPAATIPLCAVHILWAVVIDFPVFLQAPPQQGKMHSFMRRSEFSFYKREWNRFISSKAMLLNYAVMAVFSGFFSFQMMNTGIFNQQVIYIVISALLLICSPIALLYSIEKNDRMLLITLPIKRKTMFWAKYRFYSGLLAGGFLLVVMIVGFISGRSISVLTFLQCIELLLAGAYIRLTADEKRPSFSWQTEQQLWSGFSKYRSYLFCLPLFLAILAGTAVSLAVIPIAGLVIVYYLQKQDGGFFDTSKRERLGS

SEQ ID NO: 511 - DNA, 1000 bp, Bacillus subtilis
  source 1..1000: mol_type = genomic DNA
atgaataacataatccctatcatgtctttgctgttcaaacagctttacagccggcaagggaaaaaggacgccatccgcattgccgcaggccttgtcattctggccgtgtttgaaatcgggctgatccgccaggccggcattgatgaatcggtgttgcgcaaaacgtatatcatactcgcgcttcttttgatgaacacatatatggtgtttctttccgtgacatcacaatggaaggaatcttatatgaagctgagctgcctgctgccgatttcttcacggagcttttggctcgcccagagtgtcgttttgtttgtcgatacctgtttgagaagaactttattcttttttattttaccgctgttcttatttggaaacggaacgctgtcaggggcgcaaacattgttttggctcggcaggttttcgttttttaccgtttactccattattttcggagttgtgctaagcaaccacttcgtcaaaaagaagaacttgatgtttctgctgcatgcggcgatattcgcctgtgtatgtatcagcgccgctttgatgccggccgccacgattccgctttgcgcggttcatatcctgtgggcggtggtcattgactttcctgtctttctgcaggcgcctccgcagcagggcaagatgcattcatttatgcggcgatctgaattttcgttttacaaaagagaatggaaccgatttatctcttctaaagcgatgctgttaaattacgcggtaatggcggtattcagcggcttcttttcgttccagatgatgaacaccggcatcttcaatcagcaagtgatttatatcgtgatttccgcgcttttgctcatctgctcgccgatcgcccttttgtattcgattgaaaaaaatgaccggatgctgctcatcacgcttccgatcaagcgaaaaacgatgttttgggcgaaatatcgcttttattcaggcctattggcaggcggatttctccttgtcgtgatgattgtgggtttca

SEQ ID NO: 512 - protein, 239 aa, Bacillus subtilis
MSILDIHDVSVWYERDNVILEQVDLHLEKGAVYGLLGVNGAGKTTLINTLTGVNRNFSGRFTLCGIEAEAGMPQKTSDQLKTHRYFAADYPLLFTEITAKDYVSFVHSLYQKDFSEQQFASLAEAFHFSKYINRRISELSLGNRQKVVLMTGLLLRAPLFILDEPLVGLDVESIEVFYQKMREYCEAGGTILFSSHLLDVVQRFCDYAAILHNKQIQKVIPIGEETDLRREFFEVIGHE

SEQ ID NO: 513 - DNA, 716 bp, Bacillus subtilis
  source 1..716: mol_type = genomic DNA
gcattttggatatacacgatgtatccgtttggtatgaacgggacaacgtcatcttagagcacgtggacttacacttagaaaaaggcgccgtttacggattgcttggggtaaacggtgccggcaaaacaacactgatcaatacgctgacaggagtgaaccgcaattacagcgggggctttacgctgtgcggcattgaagctgaggccggcatgccgcagaaaacatcagatcaactgaagattcaccgttacttcgccgctgattatccgctgctgtttacagaaattacggcgaaggactatgtgtctttcgtccattcgctttatcaaaaggatttttcagagcgacagtttgccagtttggctgaggcctttcatttttcaaaatacatcaacaggagaatctcggagctgtccttggggaacaggcaaaaggttgtgttgatgacaggattattgctgcgggctcccctgtttattttggatgagccgctcgtcggtttggatgtggaatcaatagaggtcttttatcagaaaatgcgggagtactgtgaggaaggcggaaccattttgttttcttcccatctgctcgatgtcgtgcagagattttgtgattttgcggccattctgcacaacaaacagatccaaaaggtcattccgattggggaggagaccgatctgcggcgggaattttttgaggttatcggccatgaataa

SEQ ID NO: 514 - protein, 53 aa, Bacillus subtilis
MSPAQRRILLYILSFIFVIGAVVYFVKSDYLFTLIFIAIAILFGMRARKADSR

SEQ ID NO: 515 - DNA, 162 bp, Bacillus subtilis
  source 1..162: mol_type = genomic DNA
ttgtcaccagcacaaagaagaattttactgtatatcctttcatttatctttgtcatcggcgcagtcgtctattttgtcaaaagcgattatctgtttacgctgattttcattgccattgccattctgttcgggatgcgcgcgcggaaggctgactcgcgatga

SEQ ID NO: 516 - protein, 87 aa, Escherichia coli
MELKNSISDYTEAEFVQLLKEIEKENVAATDDVLDVLLEHFVKITEHPDGTDLIYYPSDNRDDSPEGIVKEIKEWRAANGKPGFKQG

SEQ ID NO: 517 - DNA, 264 bp, Escherichia coli
  source 1..264: mol_type = genomic DNA
atggaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggctga

SEQ ID NO: 518 - protein, 87 aa, Pseudomonas aeruginosa
MKSKISEYTEKEFLEFVEDIYTNNKKKFPTEESHIQAVLEFKKLTEHPSGSDLLYYPNENREDSPAGVVKEVKEWRASKGLPGFKAG

SEQ ID NO: 519 - DNA, 264 bp, Pseudomonas aeruginosa
  source 1..264: mol_type = genomic DNA
atgaagtccaagatttccgaatatacggaaaaagagtttcttgagtttgttgaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 520 - protein, 87 aa, Pseudomonas aeruginosa
MKSKISEYTEKEFLEFVKDIYTNNKKKFPTEESHIQAVLEFKKLTEHPSGSDLLYYPNENREDSPAGVVKEVKEWRASKGLPGFKAG

SEQ ID NO: 521 - DNA, 264 bp, Pseudomonas aeruginosa
  source 1..264: mol_type = genomic DNA
atgaagtccaagatttccgaatatacggaaaaagagtttcttgagtttgttaaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 522 - protein, 95 aa, Enterococcus hirae
MDFTKEEKLLNAISKVYNEATIDDYPDLKEKLFLYSKEISEGKSVGEVSMKLSSFLGRYILKHKFGLPKSLIELQEIVSKESQVYRGWASIGIWS

SEQ ID NO: 523 - DNA, 288 bp, Enterococcus hirae
  source 1..288: mol_type = genomic DNA
atggattttactaaagaagaaaaacttttaaatgcaattagtaaagtatacaatgaagcaactatagatgactatcctgacttaaaagaaaagctctttctttattctaaagaaatcagtgagggaaaaagtgttggtgaagttagtatgaaattaagtagttttcttggaagatatattttaaaacataaatttggattacctaaatctttaatagaattacaagaaattgttagtaaggaatctcaagtatatagaggatgggcttctattggtatttggagttaa

SEQ ID NO: 524 - protein, 113 aa, Leuconostoc mesenteroides
MKKKYRYLEDSKNYTSTLYSLLVDNVDKPGYSDICDVLLQVSKKLDNTQSVEALINRLVNYIRITASTYKIIFSKKEEELIIKLGVIGQKAGLNGQYMADFSDKSQFYSVFDQ

SEQ ID NO: 525 - DNA, 342 bp, Leuconostoc mesenteroides
  source 1..342: mol_type = genomic DNA
ttgaaaaaaaagtatcggtatttagaagatagcaaaaattacactagtacactctattctctgttagttgataatgttgacaaacctggatactcagatatttgcgatgttttgcttcaagtttctaagaagttggataatactcaaagtgttgaagcgctaattaatcgattggttaattatattcgtattactgcttcaacatacaaaattattttttcaaaaaaagaagaggaattgattataaaacttggtgttattggacaaaaagctggacttaatggtcagtatatggctgatttttcagacaagtctcagttttacagcgttttcgatcagtaa

SEQ ID NO: 526 - protein, 93 aa, Escherichia coli
MSFLNFAFSPVFFSIMACYFIVWRNKRNEFVCNRLLSIIIISFLICFIYPWLNYKIEVKYYIFEQFYLFCFLSSLVAVVINLIVYFILYRRCI

SEQ ID NO: 527 - DNA, 282 bp, Escherichia coli
  source 1..282: mol_type = genomic DNA
atgagttttcttaattttgcattttctcctgtattcttctccattatggcgtgttatttcattgtatggagaaataaacgaaacgaatttgtctgcaatagattgctatcaattataataatatcttttttgatatgcttcatatatccatggctaaattacaaaatcgaagttaaatattatatatttgaacagttttatcttttttgttttttatcgtcactcgtggctgttgtaataaacctaattgtatactttatattatacaggagatgtatatga

SEQ ID NO: 528 - protein, 96 aa, Escherichia coli
MHLKYYLHNLPESLIPWILILIFNDNDNTPLLFIFISSIHVLLYPYSKLTISRYIKENTKLKKEPWYLCKLSALFYLLMAIPVGLPSFIYYTLKRN

SEQ ID NO: 529 - DNA, 291 bp, Escherichia coli
  source 1..291: mol_type = genomic DNA
atgcatttaaaatactacctacataatttacctgaatcacttataccatggattcttattttaatatttaacgacaatgataacactcctttgttatttatatttatatcatcaatacatgtattgctatatccatactctaaattaaccatatctagatatatcaaagaaaatacaaagttaaaaaaagaaccctggtacttatgcaagttatctgcattgttttatttattaatggcaatcccagtaggattgccaagtttcatatattacactctaaagagaaattaa

SEQ ID NO: 530 - protein, 344 aa, Escherichia coli
MMIQSHPLLAAPLAVGDTIGFFSSSAPATVTAKNRFFRGVEFLQRKGFKLVSGKLTGKTDFYRSGTIKERAQEFNELVYNPDITCIMSTIGGDNSNSLLPFLDYDAIIANPKIIIGYSDTTALLAGIYAKTGLITFYGPALIPSFGEHPPLVDITYESFIKILTRKQSGIYTYTLPEKWSDESINWNENKILRPKKLYKNNCAFYGSGKVEGRVIGGNLNTLTGIWGSEWMPEILNGDILFIEDSRKSIATIERLFSMLKLNRVFDKVSAIILGKHELFDCAGSKRRPYEVLTEVLDGKQIPVLDGFDCSHTHPMLTLPLGVKLAIDFDNKNISITEQYLSTEK

SEQ ID NO: 531 - DNA, 1000 bp, Escherichia coli
  source 1..1000: mol_type = genomic DNA
atgatgatacaatctcatccactactggccgctcccctggcagtaggagatacaattggtttcttttcatcatctgctccggcaacagttactgcaaaaaatcgtttttttcggggagttgagtttcttcagagaaagggatttaagctggtatcagggaagcttaccggtaaaacagatttttatcgttcaggtactattaaagaaagagctcaagaatttaatgagttagtctacaatcctgatattacctgtataatgtcaacgatcggtggagataacagtaattcactactaccgtttctggactatgatgctatcattgcaaaccccaaaattatcataggttactcagatacaactgctttattagcaggaatatatgcaaaaacagggttaataacattctatggaccagctcttattccttcgtttggtgaacatccacctcttgtggatataacatatgaatcatttattaaaatactaacaagaaaacaatcaggaatatatacctacacattacctgaaaagtggagtgatgagagcataaactggaatgaaaacaagatattaaggcctaagaagctatataaaaacaactgtgccttttatggttccggaaaagttgaggggcgtgtaattggaggaaatctaaatactttgacaggtatatgggggagtgaatggatgcctgaaattcttaatggagatatattgtttattgaggacagtcggaaaagcattgcaacaattgaacgattattctctatgctaaagcttaatcgcgtgtttgataaagttagtgcaataatactcgggaaacatgagctttttgattgtgcaggaagtaaacgcagaccatatgaagtattaacagaggtattagatgggaaacagattcctgtactggatggatttgattgttcacatacacatccaatgctaactcttccacttggtgtaaaattagctattgactttgacaacaaaaatatat

SEQ ID NO: 532 - protein, 90 aa, Lactobacillus sakei
MKADYKKINSILTYTSTALKNPKIIKDKDLVVLLTIIQEEAKQNRIFYDYKRKFRPAVTRFTIDNNFEIPDCLVKLLSAVETPKAWSGFS

SEQ ID NO: 533 - DNA, 268 bp, Lactobacillus sakei
  source 1..268: mol_type = genomic DNA
ggcagattataaaaaaataaattcaatactaacttacacatctactgctttaaaaaaccctaaaattataaaagataaagatttagtagtccttctaactattattcaagaagaagccaaacaaaatagaatcttttatgattataaaagaaaatttcgtccagcggttactcgctttacaattgataataattttgagattcctgattgtttggttaaactactgtcagctgttgaaacacctaaggcgtggtctggatttagttag

SEQ ID NO: 534 - protein, 83 aa, Escherichia coli
MKLSPKAAIEVCNEAAKKGLWILGIDGGHWLNPGFRIDSSASWTYDMPEEYKSKTPENNRLAIENIKDDIENGYTAFIITLKM

SEQ ID NO: 535 - DNA, 251 bp, Escherichia coli
  source 1..251: mol_type = genomic DNA
tgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggaggaatacaaatcaaaaacccctgaaaataatagattggctattgaaaatattaaagatgatattgagaatggatacactgctttcattatcacgttaaagatgtaa

SEQ ID NO: 536 - protein, 436 aa, Bacillus subtilis
MNNIFPIMSLLFKQLYSRQGKKDAIRIAAGLVILAVFEIGLIRQAGIDESVLGKTYIILALLLMNTYMVFLSVTSQWKESYMKLSCLLPISSRSFWLAQSVVLFVDTCLRRTLFFFILPLFLFGNGTLSGAQTLFWLGRFSFFTVYSILFGVMLSNHFVKKKNSMFLLHAAVFAFVCLSAAFMPAVTIPLCAVHMLWAVIIDFPVFLQAPPHQSKMHFFMRRSEFSFYKREWNRFISSKAMLLNYVVMAAFSGFFSFQMMNTGIFNQQVIYIVISALLLICSPIALLYSIEKNDRMLLITLPIKRRTMFWAKYRFYSGLLAGGFLLVAIIVGFISGRPISALTFVQCMELLLAGAFIRLTADEKRPSFGWQTEQQLWSGFSKYRSYLFCLPLFLATLAGTAVSLAVIPIAALIIVYYLQKQDGGFFDTSKRERIGS

SEQ ID NO: 537 - DNA, 1000 bp, Bacillus subtilis
  source 1..1000: mol_type = genomic DNA
ttggggaggagaccgatctgcggcgggaattttttgaggttatcggccatgaataacatattccccatcatgtcgttgctgttcaaacagctgtacagccggcaagggaaaaaggacgctatccgcattgctgcagggcttgtgattctcgccgtgtttgaaatcgggctgatccgacaagccggcattgacgaatcggtgttgggaaaaacgtatatcatattggcgcttctcttaatgaacacgtatatggtgtttctttccgtgacatcacaatggaaggaatcttatatgaagctgagctgtctgctgccgatttcatcacggagcttttggctcgcccagagtgtcgttctgtttgtcgatacctgtttgagaagaacgttattcttttttattttaccgctgttcttatttggaaacggaacgctgtcaggggcgcaaacattgttttggcttggcagattttcgttttttaccgtttactcgattctattcggagttatgctaagcaaccatttcgtcaaaaagaagaactcgatgtttctgctgcatgcggcggtattcgcctttgtatgcctcagtgccgcttttatgccggccgtcacgatcccgctatgcgcggttcacatgctatgggcggtgatcattgactttccggtctttctgcaggcgcctccgcatcagagcaagatgcatttttttatgcggcgatctgaattttcgttttacaaaagagaatggaaccgatttatttcttctaaagcgatgctgttaaattacgtggtgatggcggcgttcagcggattcttttcgttccagatgatgaacactggcatcttcaatcagcaagtgatttatattgtgatttccgctctattgctgatttgctcgccgatcgcccttttgtactctattgaaaaaaacgatcgcatgctgctcatcacgcttccaattaaaagaagaacgatgttttgggcgaaatatcgcttttattcag

SEQ ID NO: 538 - protein, 580 aa, Escherichia coli
MERKQKNSLFNYIYSLMDVRGKFLFFSMLFITSLSSIIISISPLILAKITDLLSGSLSNFSYEYLVLLACLYMFCVISNKASVFLFMILQSSLRINMQKKMSLKYLRELYNENITNLSKNNAGYTTQSLNQASNDIYILVRNVSQNILSPVIQLISTIVVVLSTKDWFSAGVFFLYILVFVIFNTRLTGSLASLRKHSMDITLNSYSLLSDTVDNMIAAKKNNALRLISERYEDALTQENNAQKKYWLLSSKVLLLNSLLAVILFGSVFIYNILGVLNGVVSIGHFIMITSYIILLSTPVENIGALLSEIRQSMSSLAGFIQRHAENKATSPSIPFLNMERKLNLSIRELSFSYSDDKKILNSVSLDLFTGKMYSLTGPSGSGKSTLVKIISGYYKNYFGDIYLNDISLRNISDEDLNDAIYYLTQDDYIFMDTLRFNLRLANYDASENEIFKVLKLANLSVVNNEPVSLDTHLINRGNNYSGGQKQRISLARLFLRKPAIIIIDEATSALDYINESEILSSIRTHFPDALIINISHRINLLECSDCVYVLNEGNIVASGHFRDLMVSNEYISGLASVTE

SEQ ID NO: 539 - DNA, 1000 bp, Escherichia coli
  source 1..1000: mol_type = genomic DNA
atggaaagaaaacagaaaaactcattatttaattatatttattcattaatggatgtaagaggtaaatttttattcttttccatgttattcattacatcattatcatcgataatcatatctatttcaccattgattcttgcaaagattacagatttactgtctggctcattgtcaaattttagttatgaatatctggttttacttgcctgtttatacatgttttgcgttatatctaataaagcaagtgtttttttatttatgatactgcaaagtagtctacgtattaacatgcagaaaaaaatgtcgctaaagtatttgagagaattgtataacgaaaatataactaacttgagtaaaaataatgctggatatacaacgcaaagtcttaaccaggcttcaaatgacatttatattcttgtgagaaatgtttcccagaatatcctgtcacctgttatacaacttatttccactattgttgttgttttatctacgaaggactggttttctgccggtgtgttttttctctatattctggtatttgtaatttttaataccagactgactggcagtttagcgtctctcagaaaacacagcatggatatcactcttaactcttatagtctgttatctgatactgttgataacatgatagcagctaaaaagaataatgcattaagacttatttctgaacgttatgaagatgctctcactcaggaaaacaatgctcagaaaaaatactggttactcagttctaaagttcttttattgaactctttacttgctgtaatattatttggttctgtattcatatataatattttaggtgtgctgaatggtgtagttagtatcggccacttcattatgattacatcatatatcattcttctttcaacgccagtggaaaatataggggcattgctaagtgagatcaggcagtcaatgtctagcctggcaggttttattcaacgtcatgccgagaataaagccacatctccttcaa

SEQ ID NO: 540 - protein, 95 aa, Klebsiella pneumoniae
MTLLSFGFSPVFFSVMAFCIISRSKFYPQRTRNKVIVLILLTFFICFLYPLTKVYLVGSYGIFDKFYLFCFISTLIAIAINVVILTINGAKNERN

SEQ ID NO: 541 - DNA, 288 bp, Klebsiella pneumoniae
  source 1..288: mol_type = genomic DNA
atgacattactttcatttggattttctcctgttttcttttcagtcatggcgttctgtatcatttcacgtagtaaattctatccgcagagaacgcgaaacaaagttattgttctgattttactaactttttttatttgttttttatatccattaacaaaagtgtatctggtgggaagttacggtatatttgacaaattctacctcttttgctttatttctacgttaattgcaatagcaattaacgtagtgatacttacaataaatggagctaagaatgagagaaattag

SEQ ID NO: 542 - RNA, 13 nt, synthetic construct
  misc_feature 1..13: note = Kozak sequence
  source 1..13: mol_type = other RNA
gccgccrccatgg

SEQ ID NO: 543 - skipped

SEQ ID NO: 544 - DNA, 30 bp, synthetic construct
  misc_feature 1..30: note = Lead promoter
  source 1..30: mol_type = other DNA
gaaaaccttgtcaatgaagagcgatctatg

SEQ ID NO: 545 - DNA, 30 bp, synthetic construct
  misc_feature 1..30: note = FecA promoter
  source 1..30: mol_type = other DNA
ttctcgttcgactcatagctgaacacaaca

SEQ ID NO: 546 - DNA, 16 bp, synthetic construct
  misc_feature 1..16: note = Cu-sensitive promoter
  source 1..16: mol_type = other DNA
atgacaaaattgtcat

SEQ ID NO: 547 - DNA, 30 bp, synthetic construct
  misc_feature 1..30: note = Fe promoter
  source 1..30: mol_type = other DNA
accaatgctgggaacggccagggcacctaa

SEQ ID NO: 548 - DNA, 30 bp, synthetic construct
  misc_feature 1..30: note = Fe and UV promoters
  source 1..30: mol_type = other DNA
ctgaaagcgcataccgctatggagggggtt

SEQ ID NO: 549 - DNA, 30 bp, synthetic construct
  misc_feature 1..30: note = PrFe (PI + PII rus operon)
  source 1..30: mol_type = other DNA
tagatatgcctgaaagcgcataccgctatg

SEQ ID NO: 550 - DNA, 30 bp, synthetic construct
  misc_feature 1..30: note = Lux cassette right promoter
  source 1..30: mol_type = other DNA
tgttatagtcgaatacctctggcggtgata

SEQ ID NO: 551 - DNA, 30 bp, synthetic construct
  misc_feature 1..30: note = P(Las) TetO
  source 1..30: mol_type = other DNA
ttttggtacactccctatcagtgatagaga

SEQ ID NO: 552 - DNA, 30 bp, synthetic construct
  misc_feature 1..30: note = P(Las) CIO
  source 1..30: mol_type = other DNA
ctttttggtacactacctctggcggtgata

SEQ ID NO: 553 - DNA, 30 bp, synthetic construct
  misc_feature 1..30: note = P(Rhl)
  source 1..30: mol_type = other DNA
tacgcaagaaaatggtttgttatagtcgaa

SEQ ID NO: 554 - DNA, 30 bp, synthetic construct
  misc_feature 1..30: note = Double Promoter (LuxR/HSL, positive / cI, negative)
  source 1..30: mol_type = other DNA
cgtgcgtgttgataacaccgtgcgtgttga

SEQ ID NO: 555 - DNA, 30 bp, synthetic construct
  misc_feature 1..30: note = P2 promoter in agr operon from S. aureus
  source 1..30: mol_type = other DNA
agattgtactaaatcgtataatgacagtga

SEQ ID NO: 556 - DNA, 30 bp, synthetic construct
  misc_feature 1..30: note = plux-cI hybrid promoter
  source 1..30: mol_type = other DNA
gtgttgatgcttttatcaccgccagtggta

SEQ ID NO: 557 - DNA, 30 bp, synthetic construct
  misc_feature 1..30: note = plux-lac hybrid promoter
  source 1..30: mol_type = other DNA
agtgtgtggaattgtgagcggataacaatt

SEQ ID NO: 558 - DNA, 30 bp, synthetic construct
  misc_feature 1..30: note = CinR, CinL and glucose controlled promotor
  source 1..30: mol_type = other DNA
acatcttaaaagttttagtatcatattcgt

SEQ ID NO: 559 - DNA, 30 bp, synthetic construct; identical to SEQ ID NO: 553
  misc_feature 1..30: note = RhIR promoter repressible by CI
  source 1..30: mol_type = other DNA

SEQ ID NO: 560 - DNA, 30 bp, synthetic construct
  misc_feature 1..30: note = Reverse Lux Promoter
  source 1..30: mol_type = other DNA
tcttgcgtaaacctgtacgatcctacaggt

SEQ ID NO: 561 - DNA, 30 bp, synthetic construct
  misc_feature 1..30: note = rhlI promoter
  source 1..30: mol_type = other DNA
atcctcctttagtcttccccctcatgtgtg

SEQ ID NO: 562 - DNA, 30 bp, synthetic construct
  misc_feature 1..30: note = lasI promoter
  source 1..30: mol_type = other DNA
taaaattatgaaatttgcataaattcttca

SEQ ID NO: 563 - DNA, 30 bp, synthetic construct
  misc_feature 1..30: note = LuxR+3OC6HSL independent R0065
  source 1..30: mol_type = other DNA
gtgttgactattttacctctggcggtgata

SEQ ID NO: 564 - DNA, 30 bp, synthetic construct
  misc_feature 1..30: note = LasR/LasI Inducible & RHLR/RHLI repressible Promoter
  source 1..30: mol_type = other DNA
gaaatctggcagtttttggtacacgaaagc

SEQ ID NO: 565 - DNA, 30 bp, synthetic construct
  misc_feature 1..30: note = pLux/cI Hybrid Promoter
  source 1..30: mol_type = other DNA
acaccgtgcgtgttgatatagtcgaataaa

SEQ ID NO: 566 - DNA, 30 bp, synthetic construct
  misc_feature 1..30: note = pLas promoter
  source 1..30: mol_type = other DNA
aaaattatgaaatttgtataaattcttcag

SEQ ID NO: 567 - DNA, 30 bp, synthetic construct
  misc_feature 1..30: note = pLas/cI Hybrid Promoter
  source 1..30: mol_type = other DNA
ggttctttttggtacctctggcggtgataa

SEQ ID NO: 568 - DNA, 30 bp, synthetic construct
  misc_feature 1..30: note = pLas/Lux Hybrid Promoter
  source 1..30: mol_type = other DNA
tgtaggatcgtacaggtataaattcttcag

SEQ ID NO: 569 - DNA, 30 bp, synthetic construct
  misc_feature 1..30: note = pLux
  source 1..30: mol_type = other DNA
caagaaaatggtttgttatagtcgaataaa

SEQ ID NO: 570 - DNA, 30 bp, synthetic construct
  misc_feature 1..30: note = pLux/Las Hybrid Promoter
  source 1..30: mol_type = other DNA
ctatctcatttgctagtatagtcgaataaa

SEQ ID NO: 571 - DNA, 30 bp, synthetic construct
  misc_feature 1..30: note = Hybrid promoter: HSL-LuxR activated, P22 C2 repressed
  source 1..30: mol_type = other DNA
tagtttataatttaagtgttctttaatttc

SEQ ID NO: 572 - DNA, 30 bp, synthetic construct
  misc_feature 1..30: note = PAI+LasR -> LuxI (AI)
  source 1..30: mol_type = other DNA
caccttcgggtgggcctttctgcgtttata

SEQ ID NO: 573 - DNA, 30 bp, synthetic construct
  misc_feature 1..30: note = PAI+LasR -> LasI & AI+LuxR --| LasI
  source 1..30: mol_type = other DNA
aataactctgatagtgctagtgtagatctc

SEQ ID NO: 574 - DNA, 30 bp, synthetic construct; identical to SEQ ID NO: 572
  misc_feature 1..30: note = PAI+LasR -> LasI+GFP & AI+LuxR --| LasI+GFP
  source 1..30: mol_type = other DNA

SEQ ID NO: 575 - DNA, 30 bp, synthetic construct; identical to SEQ ID NO: 572
  misc_feature 1..30: note = Complex QS -> LuxI & LasI circuit
  source 1..30: mol_type = other DNA

SEQ ID NO: 576 - DNA, 30 bp, synthetic construct; identical to SEQ ID NO: 569
  misc_feature 1..30: note = position 3 mutated promoter lux pR-3 (luxR & HSL regulated)
  source 1..30: mol_type = other DNA

SEQ ID NO: 577 - DNA, 30 bp, synthetic construct; identical to SEQ ID NO: 569
  misc_feature 1..30: note = position 5 mutated promoter lux pR-5 (luxR & HSL regulated)
  source 1..30: mol_type = other DNA

SEQ ID NO: 578 - DNA, 30 bp, synthetic construct; identical to SEQ ID NO: 569
  misc_feature 1..30: note = position 3&5 mutated promoter lux pR-3/5 (luxR & HSL regulated)
  source 1..30: mol_type = other DNA

SEQ ID NO: 579 - DNA, 30 bp, synthetic construct
  misc_feature 1..30: note = Promoter (HSL-mediated luxR repressor)
  source 1..30: mol_type = other DNA
ttgacacctgtaggatcgtacaggtataat

SEQ ID NO: 580 - DNA, 30 bp, synthetic construct; identical to SEQ ID NO: 569
  misc_feature 1..30: note = Promoter (luxR & HSL regulated -- lux pR)
  source 1..30: mol_type = other DNA

SEQ ID NO: 581 - DNA, 30 bp, synthetic construct
  misc_feature 1..30: note = Promoter (luxR & HSL regulated -- lux pL)
  source 1..30: mol_type = other DNA
cacgcaaaacttgcgacaaacaataggtaa

SEQ ID NO: 582 - DNA, 30 bp, synthetic construct
  misc_feature 1..30: note = Promoter (RhlR & C4-HSL regulated)
  source 1..30: mol_type = other DNA
gttagctttcgaattggctaaaaagtgttc

SEQ ID NO: 583 - DNA, 30 bp, synthetic construct
  misc_feature 1..30: note = Promoter (cinR and HSL regulated)
  source 1..30: mol_type = other DNA
ccattctgctttccacgaacttgaaaacgc

SEQ ID NO: 584 - DNA, 30 bp, synthetic construct
  misc_feature 1..30: note = Promoter (LasR & PAI regulated)
  source 1..30: mol_type = other DNA
ggccgcgggttctttttggtacacgaaagc

SEQ ID NO: 585 - DNA, 30 bp, synthetic construct
  misc_feature 1..30: note = Promoter, Standard (luxR and HSL regulated -- lux pR)
  source 1..30: mol_type = other DNA
aagaaaatggtttgttgatactcgaataaa

SEQ ID NO: 586 - DNA, 30 bp, synthetic construct
  misc_feature 1..30: note = P(Bla)
  source 1..30: mol_type = other DNA
gtttatacataggcgagtactctgttatgg

SEQ ID NO: 587 - DNA, 30 bp, synthetic construct
  misc_feature 1..30: note = P(Cat)
  source 1..30: mol_type = other DNA
agaggttccaactttcaccataatgaaaca

SEQ ID NO: 588 - DNA, 30 bp, synthetic construct
  misc_feature 1..30: note = P(Kat)
  source 1..30: mol_type = other DNA
taaacaactaacggacaattctacctaaca

SEQ ID NO: 589 - DNA, 30 bp, synthetic construct
  misc_feature 1..30: note = Template for Building Primer Family Member
  source 1..30: mol_type = other DNA
acatcaagccaaattaaacaggattaacac

SEQ ID NO: 590 - DNA, 30 bp, synthetic construct
  misc_feature 1..30: note = Reverse lambda cI-regulated promoter
  source 1..30: mol_type = other DNA
gaggtaaaatagtcaacacgcacggtgtta

SEQ ID NO: 591 - DNA, 30 bp, synthetic construct
  misc_feature 1..30: note = Key Promoter absorbs 3
  source 1..30: mol_type = other DNA
caggccggaataactccctataatgcgcca

SEQ ID NO: 592 - DNA, 30 bp, synthetic construct
  misc_feature 1..30: note = constitutive promoter family member
  source 1..30: mol_type = other DNA
ggctagctcagtcctaggtacagtgctagc

SEQ ID NO: 593 - DNA, 30 bp, synthetic construct
  misc_feature 1..30: note = constitutive promoter family member
  source 1..30: mol_type = other DNA
agctagctcagtcctaggtattatgctagc

SEQ ID NO: 594 - DNA, 30 bp, synthetic construct
  misc_feature 1..30: note = constitutive promoter family member
  source 1..30: mol_type = other DNA
agctagctcagtcctaggtactgtgctagc

SEQ ID NO: 595 - DNA, 30 bp, synthetic construct
  misc_feature 1..30: note = constitutive promoter family member
  source 1..30: mol_type = other DNA
agctagctcagtcctagggattatgctagc

SEQ ID NO: 596 - DNA, 30 bp, synthetic construct
  misc_feature 1..30: note = constitutive promoter family member
  source 1..30: mol_type = other DNA
agctagctcagtcctaggtattgtgctagc

SEQ ID NO: 597 - DNA, 30 bp, synthetic construct
  misc_feature 1..30: note = constitutive promoter family member
  source 1..30: mol_type = other DNA
ggctagctcagtcctaggtactatgctagc

SEQ ID NO: 598 - DNA, 30 bp, synthetic construct
  misc_feature 1..30: note = constitutive promoter family member
  source 1..30: mol_type = other DNA
ggctagctcagtcctaggtatagtgctagc

SEQ ID NO: 599 - DNA, 30 bp, synthetic construct
  misc_feature 1..30: note = constitutive promoter family member
  source 1..30: mol_type = other DNA
ggctagctcagccctaggtattatgctagc

SEQ ID NO: 600 - DNA, 30 bp, synthetic construct
  misc_feature 1..30: note = constitutive promoter family member
  source 1..30: mol_type = other DNA
agctagctcagtcctaggtataatgctagc

SEQ ID NO: 601 - DNA, 30 bp, synthetic construct
  misc_feature 1..30: note = constitutive promoter family member
  source 1..30: mol_type = other DNA
agctagctcagtcctagggactgtgctagc

SEQ ID NO: 602 - DNA, 30 bp, synthetic construct
  misc_feature 1..30: note = constitutive promoter family member
  source 1..30: mol_type = other DNA
ggctagctcagtcctaggtacaatgctagc

SEQ ID NO: 603 - DNA, 30 bp, synthetic construct; identical to SEQ ID NO: 598
  misc_feature 1..30: note = constitutive promoter family member
  source 1..30: mol_type = other DNA

SEQ ID NO: 604 - DNA, 30 bp, synthetic construct; identical to SEQ ID NO: 595
  misc_feature 1..30: note = constitutive promoter family member
  source 1..30: mol_type = other DNA

SEQ ID NO: 605 - DNA, 30 bp, synthetic construct
  misc_feature 1..30: note = constitutive promoter family member
  source 1..30: mol_type = other DNA
ggctagctcagtcctagggattatgctagc

SEQ ID NO: 606 - DNA, 30 bp, synthetic construct; identical to SEQ ID NO: 602
  misc_feature 1..30: note = constitutive promoter family member
  source 1..30: mol_type = other DNA

SEQ ID NO: 607 - DNA, 30 bp, synthetic construct
  misc_feature 1..30: note = constitutive promoter family member
  source 1..30: mol_type = other DNA
agctagctcagcccttggtacaatgctagc

SEQ ID NO: 608 - DNA, 30 bp, synthetic construct
  misc_feature 1..30: note = constitutive promoter family member
  source 1..30: mol_type = other DNA
agctagctcagtcctagggactatgctagc

SEQ ID NO: 609 - DNA, 30 bp, synthetic construct
  misc_feature 1..30: note = constitutive promoter family member
  source 1..30: mol_type = other DNA
agctagctcagtcctagggattgtgctagc

SEQ ID NO: 610 - DNA, 30 bp, synthetic construct
  misc_feature 1..30: note = constitutive promoter family member
  source 1..30: mol_type = other DNA
ggctagctcagtcctaggtattgtgctagc

SEQ ID NO: 611 - DNA, 30 bp, synthetic construct; identical to SEQ ID NO: 600
  misc_feature 1..30: note = constitutive promoter family member
  source 1..30: mol_type = other DNA

SEQ ID NO: 612 - DNA, 30 bp, synthetic construct
  misc_feature 1..30: note = 1bp mutant from J23107
  source 1..30: mol_type = other DNA
ggctagctcagtcctaggtattatgctagc

SEQ ID NO: 613 - DNA, 30 bp, synthetic construct; identical to SEQ ID NO: 602
  misc_feature 1..30: note = 1bp mutant from J23114
  source 1..30: mol_type = other DNA

SEQ ID NO: 614 - DNA, 30 bp, synthetic construct
  misc_feature 1..30: note = pBAD reverse
  source 1..30: mol_type = other DNA
aaagtgtgacgccgtgcaaataatcaatgt

SEQ ID NO: 615 - DNA, 30 bp, synthetic construct
  misc_feature 1..30: note = NikR promoter, a protein of the ribbon helix-helix family of trancription factors that repress expre
  source 1..30: mol_type = other DNA
gacgaatacttaaaatcgtcatacttattt

SEQ ID NO: 616 - DNA, 30 bp, synthetic construct
  misc_feature 1..30: note = lacq_Promoter
  source 1..30: mol_type = other DNA
aaacctttcgcggtatggcatgatagcgcc

SEQ ID NO: 617 - DNA, 30 bp, synthetic construct
  misc_feature 1..30: note = lacIQ - promoter sequence
  source 1..30: mol_type = other DNA
tgatagcgcccggaagagagtcaattcagg

SEQ ID NO: 618 - DNA, 30 bp, synthetic construct
  misc_feature 1..30: note = E. Coli CreABCD phosphate sensing operon promoter
  source 1..30: mol_type = other DNA
ttatttaccgtgacgaactaattgctcgtg

SEQ ID NO: 619 - DNA, 30 bp, synthetic construct
  misc_feature 1..30: note = GlnRS promoter
  source 1..30: mol_type = other DNA
catacgccgttatacgttgtttacgctttg

SEQ ID NO: 620 - DNA, 30 bp, synthetic construct
  misc_feature 1..30: note = Constitutive weak promoter of lacZ
  source 1..30: mol_type = other DNA
ttatgcttccggctcgtatgttgtgtggac

SEQ ID NO: 621 - DNA, 30 bp, synthetic construct
  misc_feature 1..30: note = Mutated LacZ promoter
  source 1..30: mol_type = other DNA
ttatgcttccggctcgtatggtgtgtggac

SEQ ID NO: 622 - DNA, 30 bp, synthetic construct
  misc_feature 1..30: note = constitutive promoter with (TA)10 between -10 and -35 elements
  source 1..30: mol_type = other DNA
atatatatatatatataatggaagcgtttt

SEQ ID NO: 623 - DNA, 30 bp, synthetic construct; identical to SEQ ID NO: 622
  misc_feature 1..30: note = constitutive promoter with (TA)9 between -10 and -35 elements
  source 1..30: mol_type = other DNA

SEQ ID NO: 624 - DNA, 30 bp, synthetic construct
  misc_feature 1..30: note = constitutive promoter with (C)10 between -10 and -35 elements
  source 1..30: mol_type = other DNA
ccccgaaagcttaagaatataattgtaagc

SEQ ID NO: 625 - DNA, 30 bp, synthetic construct; identical to SEQ ID NO: 624
  misc_feature 1..30: note = constitutive promoter with (C)12 between -10 and -35 elements
  source 1..30: mol_type = other DNA

SEQ ID NO: 626 - DNA, 30 bp, synthetic construct
  misc_feature 1..30: note = optimized (TA) repeat constitutive promoter with 13 bp between -10 and -35 elements
  source 1..30: mol_type = other DNA
tgacaatatatatatatatataatgctagc

SEQ ID NO: 627 - DNA, 30 bp, synthetic construct
  misc_feature 1..30: note = optimized (TA) repeat constitutive promoter with 15 bp between -10 and -35 elements
  source 1..30: mol_type = other DNA
acaatatatatatatatatataatgctagc

SEQ ID NO: 628 - DNA, 30 bp, synthetic construct
  misc_feature 1..30: note = optimized (TA) repeat constitutive promoter with 17 bp between -10 and -35 elements
  source 1..30: mol_type = other DNA
aatatatatatatatatatataatgctagc

SEQ ID NO: 629 - DNA, 30 bp, synthetic construct
  misc_feature 1..30: note = optimized (TA) repeat constitutive promoter with 19 bp between -10 and -35 elements
  source 1..30: mol_type = other DNA
tatatatatatatatatatataatgctagc

SEQ ID NO: 630 - DNA, 30 bp, synthetic construct; identical to SEQ ID NO: 629
  misc_feature 1..30: note = optimized (TA) repeat constitutive promoter with 21 bp between -10 and -35 elements
  source 1..30: mol_type = other DNA

SEQ ID NO: 631 - DNA, 30 bp, synthetic construct
  misc_feature 1..30: note = optimized (A) repeat constitutive promoter with 17 bp between -10 and -35 elements
  source 1..30: mol_type = other DNA
aaaaaaaaaaaaaaaaaatataatgctagc

SEQ ID NO: 632 - DNA, 30 bp, synthetic construct; identical to SEQ ID NO: 631
  misc_feature 1..30: note = optimized (A) repeat constitutive promoter with 18 bp between -10 and -35 elements
  source 1..30: mol_type = other DNA

SEQ ID NO: 633 - DNA, 30 bp, synthetic construct; identical to SEQ ID NO: 572
  misc_feature 1..30: note = J23101:GFP
  source 1..30: mol_type = other DNA

SEQ ID NO: 634 - DNA, 30 bp, synthetic construct; identical to SEQ ID NO: 572
  misc_feature 1..30: note = J23119:IFP
  source 1..30: mol_type = other DNA

SEQ ID NO: 635 - DNA, 30 bp, synthetic construct; identical to SEQ ID NO: 572
  misc_feature 1..30: note = J23119:HO1
  source 1..30: mol_type = other DNA

SEQ ID NO: 636 - DNA, 30 bp, synthetic construct; identical to SEQ ID NO: 572
  misc_feature 1..30: note = Infrared signal reporter (J23119:IFP:J23119:HO1)
  source 1..30: mol_type = other DNA

SEQ ID NO: 637 - DNA, 30 bp, synthetic construct; identical to SEQ ID NO: 592
  misc_feature 1..30: note = Double terminator + constitutive promoter
  source 1..30: mol_type = other DNA

SEQ ID NO: 638 - DNA, 30 bp, synthetic construct
  misc_feature 1..30: note = Double terminator + Constitutive promoter + Strong RBS
  source 1..30: mol_type = other DNA
tgctagctactagagattaaagaggagaaa

SEQ ID NO: 639 - DNA, 30 bp, synthetic construct
  misc_feature 1..30: note = IPTG inducible Lac promoter cassette
  source 1..30: mol_type = other DNA
ttgtgagcggataacaagatactgagcaca

SEQ ID NO: 640 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 641 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 642 - DNA, 30 bp, synthetic construct
  misc_feature 1..30: note = M13K07 gene I promoter
  source 1..30: mol_type = other DNA
cctgtttttatgttattctctctgtaaagg

SEQ ID NO: 643 - DNA, 30 bp, synthetic construct
  misc_feature 1..30: note = M13K07 gene II promoter
  source 1..30: mol_type = other DNA
aaatatttgcttatacaatcttcctgtttt

SEQ ID NO: 644 - DNA, 30 bp, synthetic construct
  misc_feature 1..30: note = M13K07 gene III promoter
  source 1..30: mol_type = other DNA
gctgataaaccgatacaattaaaggctcct

SEQ ID NO: 645 - DNA, 30 bp, synthetic construct
  misc_feature 1..30: note = M13K07 gene IV promoter
  source 1..30: mol_type = other DNA
ctcttctcagcgtcttaatctaagctatcg

SEQ ID NO: 646 - DNA, 30 bp, synthetic construct
  misc_feature 1..30: note = M13K07 gene V promoter
  source 1..30: mol_type = other DNA
atgagccagttcttaaaatcgcataaggta

SEQ ID NO: 647 - DNA, 30 bp, synthetic construct
  misc_feature 1..30: note = M13K07 gene VI promoter
  source 1..30: mol_type = other DNA
ctattgattgtgacaaaataaacttattcc

SEQ ID NO: 648 - DNA, 30 bp, synthetic construct
  misc_feature 1..30: note = M13K07 gene VIII promoter
  source 1..30: mol_type = other DNA
gtttcgcgcttggtataatcgctgggggtc

SEQ ID NO: 649 - DNA, 30 bp, synthetic construct
  misc_feature 1..30: note = M13110
  source 1..30: mol_type = other DNA
ctttgcttctgactataatagtcagggtaa

SEQ ID NO: 650 - DNA, 30 bp, synthetic construct
  misc_feature 1..30: note = Modified promoter sequence of g3.
  source 1..30: mol_type = other DNA
aaaccgatacaattaaaggctcctgctagc

SEQ ID NO: 651 - DNA, 30 bp, synthetic construct
  misc_feature 1..30: note = Constitutive Promoter I
  source 1..30: mol_type = other DNA
caccacactgatagtgctagtgtagatcac

SEQ ID NO: 652 - DNA, 30 bp, synthetic construct
  misc_feature 1..30: note = Constitutive Promoter II
  source 1..30: mol_type = other DNA
gccggaataactccctataatgcgccacca

SEQ ID NO: 653 - DNA, 30 bp, synthetic construct
  misc_feature 1..30: note = --Specify Parts List--
  source 1..30: mol_type = other DNA
ttgacaagcttttcctcagctccgtaaact

SEQ ID NO: 654 - DNA, 30 bp, synthetic construct
  misc_feature 1..30: note = Full-length stationary phase osmY promoter
  source 1..30: mol_type = other DNA
ggtttcaaaattgtgatctatatttaacaa

SEQ ID NO: 655 - DNA, 30 bp, synthetic construct; identical to SEQ ID NO: 654
  misc_feature 1..30: note = Minimal stationary phase osmY promoter
  source 1..30: mol_type = other DNA

SEQ ID NO: 656 - DNA, 30 bp, synthetic construct
  misc_feature 1..30: note = htpG Heat Shock Promoter
  source 1..30: mol_type = other DNA
tctattccaataaagaaatcttcctgcgtg

SEQ ID NO: 657 - DNA, 30 bp, synthetic construct
  misc_feature 1..30: note = Promoter veg a constitutive promoter for B. subtilis
  source 1..30: mol_type = other DNA
aaaaatgggctcgtgttgtacaataaatgt

SEQ ID NO: 658 - DNA, 30 bp, synthetic construct
  misc_feature 1..30: note = Promoter 43 a constitutive promoter for B. subtilis
  source 1..30: mol_type = other DNA
aaaaaaagcgcgcgattatgtaaaatataa

SEQ ID NO: 659 - DNA, 30 bp, synthetic construct
  misc_feature 1..30: note = Strong constitutive promoter for Bacillus subtilis
  source 1..30: mol_type = other DNA
aattgcagtaggcatgacaaaatggactca

SEQ ID NO: 660 - DNA, 30 bp, synthetic construct
  misc_feature 1..30: note = PliaG
  source 1..30: mol_type = other DNA
caagcttttcctttataatagaatgaatga

SEQ ID NO: 661 - DNA, 30 bp, synthetic construct
  misc_feature 1..30: note = PlepA
  source 1..30: mol_type = other DNA
tctaagctagtgtattttgcgtttaatagt

SEQ ID NO: 662 - DNA, 30 bp, synthetic construct
  misc_feature 1..30: note = Pveg
  source 1..30: mol_type = other DNA
aatgggctcgtgttgtacaataaatgtagt

SEQ ID NO: 663 - DNA, 30 bp, synthetic construct
  misc_feature 1..30: note = Promoter ctc for B. subtilis
  source 1..30: mol_type = other DNA
atccttatcgttatgggtattgtttgtaat

SEQ ID NO: 664 - DNA, 30 bp, synthetic construct
  misc_feature 1..30: note = Promoter gsiB for B. subtilis
  source 1..30: mol_type = other DNA
taaaagaattgtgagcgggaatacaacaac

SEQ ID NO: 665 - DNA, 30 bp, synthetic construct; identical to SEQ ID NO: 658
  misc_feature 1..30: note = Promoter 43 a constitutive promoter for B. subtilis
  source 1..30: mol_type = other DNA

SEQ ID NO: 666 - DNA, 30 bp, synthetic construct
  misc_feature 1..30: note = Pspv2 from Salmonella
  source 1..30: mol_type = other DNA
tacaaaataattcccctgcaaacattatca

SEQ ID NO: 667 - DNA, 30 bp, synthetic construct
  misc_feature 1..30: note = Pspv from Salmonella
  source 1..30: mol_type = other DNA
tacaaaataattcccctgcaaacattatcg

SEQ ID NO: 668 - DNA, 30 bp, synthetic construct
  misc_feature 1..30: note = T7 promoter (strong promoter from T7 bacteriophage)
  source 1..30: mol_type = other DNA
agggaatacaagctacttgttctttttgca

SEQ ID NO: 669 - DNA, 23 bp, synthetic construct
  misc_feature 1..23: note = T7 Promoter
  source 1..23: mol_type = other DNA
taatacgactcactatagggaga

SEQ ID NO: 670 - DNA, 28 bp, synthetic construct
  misc_feature 1..28: note = T7 Promoter
  source 1..28: mol_type = other DNA
gaatttaatacgactcactatagggaga

SEQ ID NO: 671 - DNA, 19 bp, synthetic construct
  misc_feature 1..19: note = T7 consensus -10 and rest
  source 1..19: mol_type = other DNA
taatacgactcactatagg

SEQ ID NO: 672 - DNA, 30 bp, synthetic construct
  misc_feature 1..30: note = overlapping T7 promoter
  source 1..30: mol_type = other DNA
gagtcgtattaatacgactcactatagggg

SEQ ID NO: 673 - DNA, 30 bp, synthetic construct
  misc_feature 1..30: note = more overlapping T7 promoter
  source 1..30: mol_type = other DNA
agtgagtcgtactacgactcactatagggg

SEQ ID NO: 674 - DNA, 30 bp, synthetic construct
  misc_feature 1..30: note = weaken overlapping T7 promoter
  source 1..30: mol_type = other DNA
gagtcgtattaatacgactctctatagggg

SEQ ID NO: 675 - DNA, 23 bp, synthetic construct
  misc_feature 1..23: note = T7 Consensus Promoter Sequence
  source 1..23: mol_type = other DNA
taatacgactcactatagggaga

SEQ ID NO: 676 - DNA, 23 bp, synthetic construct
  misc_feature 1..23: note = T7 RNAP promoter
  source 1..23: mol_type = other DNA
ttatacgactcactatagggaga

SEQ ID NO: 677 - DNA, 23 bp, synthetic construct
  misc_feature 1..23: note = T7 RNAP promoter
  source 1..23: mol_type = other DNA
gaatacgactcactatagggaga

SEQ ID NO: 678 - DNA, 23 bp, synthetic construct
  misc_feature 1..23: note = T7 RNAP promoter
  source 1..23: mol_type = other DNA
taatacgtctcactatagggaga

SEQ ID NO: 679 - DNA, 23 bp, synthetic construct
  misc_feature 1..23: note = T7 RNAP promoter
  source 1..23: mol_type = other DNA
tcatacgactcactatagggaga

SEQ ID NO: 680 - DNA, 30 bp, synthetic construct
  misc_feature 1..30: note = T7 strong promoter
  source 1..30: mol_type = other DNA
taatacgactcactatagggagaccacaac

SEQ ID NO: 681 - DNA, 30 bp, synthetic construct
  misc_feature 1..30: note = T7 weak binding and processivity
  source 1..30: mol_type = other DNA
taattgaactcactaaagggagaccacagc

SEQ ID NO: 682 - DNA, 30 bp, synthetic construct
  misc_feature 1..30: note = T7 weak binding promoter
  source 1..30: mol_type = other DNA
cgaagtaatacgactcactattagggaaga

SEQ ID NO: 683 - DNA, 30 bp, synthetic construct
  misc_feature 1..30: note = pCyc (Medium) Promoter
  source 1..30: mol_type = other DNA
acaaacacaaatacacacactaaattaata

SEQ ID NO: 684 - DNA, 30 bp, synthetic construct
  misc_feature 1..30: note = pAdh (Strong) Promoter
  source 1..30: mol_type = other DNA
ccaagcatacaatcaactatctcatataca

SEQ ID NO: 685 - DNA, 30 bp, synthetic construct
  misc_feature 1..30: note = pSte5 (Weak) Promoter
  source 1..30: mol_type = other DNA
gatacaggatacagcggaaacaacttttaa

SEQ ID NO: 686 - DNA, 30 bp, synthetic construct
  misc_feature 1..30: note = yeast ADH1 promoter
  source 1..30: mol_type = other DNA
tttcaagctataccaagcatacaatcaact

SEQ ID NO: 687 - DNA, 30 bp, synthetic construct
  misc_feature 1..30: note = cyc100 minimal promoter
  source 1..30: mol_type = other DNA
cctttgcagcataaattactatacttctat

SEQ ID NO: 688 - DNA, 30 bp, synthetic construct; identical to SEQ ID NO: 687
  misc_feature 1..30: note = cyc70 minimal promoter
  source 1..30: mol_type = other DNA

SEQ ID NO: 689 - DNA, 30 bp, synthetic construct; identical to SEQ ID NO: 687
  misc_feature 1..30: note = cyc43 minimal promoter
  source 1..30: mol_type = other DNA

SEQ ID NO: 690 - DNA, 30 bp, synthetic construct; identical to SEQ ID NO: 687
  misc_feature 1..30: note = cyc28 minimal promoter
  source 1..30: mol_type = other DNA

SEQ ID NO: 691 - DNA, 30 bp, synthetic construct; identical to SEQ ID NO: 687
  misc_feature 1..30: note = cyc16 minimal promoter
  source 1..30: mol_type = other DNA

SEQ ID NO: 692 - DNA, 30 bp, synthetic construct
  misc_feature 1..30: note = pPGK1
  source 1..30: mol_type = other DNA
ttatctactttttacaacaaatataaaaca

SEQ ID NO: 693 - DNA, 30 bp, synthetic construct; identical to SEQ ID NO: 683
  misc_feature 1..30: note = pCYC Yeast Promoter
  source 1..30: mol_type = other DNA

SEQ ID NO: 694 - DNA, 30 bp, synthetic construct
  misc_feature 1..30: note = Yeast GPD (TDH3) Promoter
  source 1..30: mol_type = other DNA
gtttcgaataaacacacataaacaaacaaa

SEQ ID NO: 695 - DNA, 30 bp, synthetic construct; identical to SEQ ID NO: 684
  misc_feature 1..30: note = yeast mid-length ADH1 promoter
  source 1..30: mol_type = other DNA

SEQ ID NO: 696 - DNA, 30 bp, synthetic construct
  misc_feature 1..30: note = Yeast CLB1 promoter region, G2/M cell cycle specific
  source 1..30: mol_type = other DNA
accatcaaaggaagctttaatcttctcata

SEQ ID NO: 697 - DNA, 30 bp, synthetic construct
  misc_feature 1..30: note = CMV promoter
  source 1..30: mol_type = other DNA
agaacccactgcttactggcttatcgaaat

SEQ ID NO: 698 - DNA, 30 bp, synthetic construct
  misc_feature 1..30: note = Ubc Promoter
  source 1..30: mol_type = other DNA
ggccgtttttggcttttttgttagacgaag

SEQ ID NO: 699 - protein, 9 aa, Lactobacillus curvatus
NIPQLTPTP

SEQ ID NO: 700 - DNA, 27 bp, Lactobacillus curvatus
  source 1..27: mol_type = genomic DNA
aacattccgcagctgaccccgaccccg

SEQ ID NO: 701 - protein, 18 aa, Bacillus thuringiensis
  VARIANT 4..10: note = Xaa = any amino acid
DWTXWSXLVXAACSVELL

SEQ ID NO: 702 - DNA, 54 bp, Bacillus thuringiensis; identical to SEQ ID NO: 389
  misc_feature 10..30: note = n = A,T,C or G
  source 1..54: mol_type = genomic DNA

SEQ ID NO: 703 - protein, 30 aa, Lactobacillus curvatus; identical to SEQ ID NO: 390
  source 1..30: note = L442

SEQ ID NO: 704 - DNA, 90 bp, Lactobacillus curvatus; identical to SEQ ID NO: 391
  source 1..90: mol_type = genomic DNA; note = L442

SEQ ID NO: 705 - protein, 43 aa, Carnobacterium divergens; identical to SEQ ID NO: 392

SEQ ID NO: 706 - DNA, 129 bp, Carnobacterium divergens; identical to SEQ ID NO: 393
  source 1..129: mol_type = genomic DNA

SEQ ID NO: 707 - protein, 24 aa, Microbispora sp.
  source 1..24: note = (strain 107891)
VTSWSLCTPGCTSPGGGSNCSFCC

SEQ ID NO: 708 - DNA, 72 bp, Microbispora sp.
  source 1..72: mol_type = genomic DNA; note = (strain 107891)
gtgaccagctggagcctgtgcaccccgggctgcaccagcccgggcggcggcagcaactgcagcttttgctgc

SEQ ID NO: 709 - protein, 62 aa, Enterococcus sp.; identical to SEQ ID NO: 394

SEQ ID NO: 710 - DNA, 186 bp, Enterococcus sp.; identical to SEQ ID NO: 395
  source 1..186: mol_type = genomic DNA

SEQ ID NO: 711 - protein, 39 aa, Enterococcus faecium; identical to SEQ ID NO: 396

SEQ ID NO: 712 - DNA, 117 bp, Enterococcus faecium; identical to SEQ ID NO: 397
  source 1..117: mol_type = genomic DNA

SEQ ID NO: 713 - protein, 30 aa, Paenibacillus polymyxa; identical to SEQ ID NO: 398

SEQ ID NO: 714 - DNA, 90 bp, Paenibacillus polymyxa; identical to SEQ ID NO: 399
  source 1..90: mol_type = genomic DNA

SEQ ID NO: 715 - protein, 31 aa, Staphylococcus epidermidis; identical to SEQ ID NO: 400

SEQ ID NO: 716 - DNA, 93 bp, Staphylococcus epidermidis; identical to SEQ ID NO: 401
  source 1..93: mol_type = genomic DNA

SEQ ID NO: 717 - protein, 58 aa, Enterococcus faecium
MEKLTVKEMSQVVGGKYYGNGVSCNKKGCSVDWGKAIGIIGNNAAANLTTGGKAGWKG

SEQ ID NO: 718 - DNA, 178 bp, Enterococcus faecium
  source 1..178: mol_type = genomic DNA
atggaaaaattaactgtgaaagaaatgtcgcaagtagttggcggaaagtactatggtaacggagtatcatgtaataaaaagggatgtagtgttgattggggaaaagctattggtattattggaaataatgctgctgctaatttaactactggcggaaaagcagggtggaaaggttaac

SEQ ID NO: 719 - protein, 39 aa, Paenibacillus polymyxa; identical to SEQ ID NO: 404

SEQ ID NO: 720 - DNA, 93 bp, Paenibacillus polymyxa; identical to SEQ ID NO: 401
  source 1..93: mol_type = genomic DNA

SEQ ID NO: 721 - protein, 35 aa, Bacillus circulans; identical to SEQ ID NO: 406

SEQ ID NO: 722 - DNA, 105 bp, Bacillus circulans; identical to SEQ ID NO: 407
  source 1..105: mol_type = genomic DNA

SEQ ID NO: 723 - protein, 30 aa, Paenibacillus polymyxa; identical to SEQ ID NO: 408

SEQ ID NO: 724 - DNA, 90 bp, Paenibacillus polymyxa; identical to SEQ ID NO: 409
  source 1..90: mol_type = genomic DNA

SEQ ID NO: 725 - protein, 13 aa, Lactobacillus rhamnosus
AVPAVRKTNETLD

SEQ ID NO: 726 - DNA, 39 bp, Lactobacillus rhamnosus; identical to SEQ ID NO: 411
  source 1..39: mol_type = genomic DNA

SEQ ID NO: 727 - protein, 60 aa, Bacillus licheniformis
MKNSAAREAFKGANHPAGMVSEEELKALVGGNDVNPETTPATTSSWTCITAGVTVSASLC

SEQ ID NO: 728 - DNA, 180 bp, Bacillus licheniformis
  source 1..180: mol_type = genomic DNA
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgc

SEQ ID NO: 729 - protein, 689 aa, Pseudomonas aeruginosa; identical to SEQ ID NO: 276

SEQ ID NO: 730 - DNA, 2070 bp, Pseudomonas aeruginosa; identical to SEQ ID NO: 277
  source 1..2070: mol_type = genomic DNA

SEQ ID NO: 731 - protein, 36 aa, Lactobacillus plantarum; identical to SEQ ID NO: 414

SEQ ID NO: 732 - DNA, 108 bp, Lactobacillus plantarum; identical to SEQ ID NO: 415
  source 1..108: mol_type = genomic DNA

SEQ ID NO: 733 - protein, 30 aa, Lactococcus lactis
MKTILRFVAGYDIASHKKKTGGYPWERGKA

SEQ ID NO: 734 - DNA, 93 bp, Lactococcus lactis
  source 1..93: mol_type = genomic DNA
atgaaaacaatcctacgttttgttgctggctacgatattgctagtcataaaaagaaaactggcggctatccatgggaacgtggaaaagcttaa

SEQ ID NO: 735 - protein, 24 aa, Lactobacillus acidophilus
GNPKVAHCASQIGRSTAWGAVSGA

SEQ ID NO: 736 - DNA, 72 bp, Lactobacillus acidophilus; identical to SEQ ID NO: 417
  source 1..72: mol_type = genomic DNA

SEQ ID NO: 737 - protein, 53 aa, Lactobacillus salivarius
  source 1..53: note = cp400
MFFNFMKKVDVKKNFGYKEVSRKDLAKVNGGKRKKHRCRVYNNGMPTGMYRWC

SEQ ID NO: 738 - DNA, 162 bp, Lactobacillus salivarius
  source 1..162: mol_type = genomic DNA; note = cp400
atgttttttaattttatgaaaaaagtagatgtgaagaagaattttggatataaagaagtttctagaaaagatctagctaaagtaaatggtggaaagagaaagaaacatcgttgcagagtttataataatggaatgcctacaggaatgtatcgttggtgctaa

SEQ ID NO: 739 - protein, 5 aa, synthetic construct
  REGION 1..5: note = Caspase 2 consensus sequence
DVADL

SEQ ID NO: 740 - protein, 5 aa, synthetic construct
  REGION 1..5: note = Caspase 2 consensus sequence
DVADI

SEQ ID NO: 741 - protein, 12 aa, synthetic construct
  REGION 1..12: note = Synthetic RAP-binding peptide, RBP
FHWWQTSPAHFS

SEQ ID NO: 742 - protein, 12 aa, synthetic construct
  REGION 1..12: note = Synthetic RAP-binding peptide, RBP
WPFAHWPWQYPR

SEQ ID NO: 743 - protein, 9 aa, synthetic construct
  REGION 1..9: note = Synthetic AgrC ligand
  VARIANT 5..9: note = Thiolacton linkage between C5 and F9
GDSVCASYF

SEQ ID NO: 744 - protein, 7 aa, synthetic construct
  REGION 1..7: note = Synthetic AgrC ligand
  VARIANT 3..7: note = Thiolacton linkage between C3 and F7
SVCASYF

SEQ ID NO: 745 - protein, 5 aa, synthetic construct
  REGION 1..5: note = Synthetic Cry1Aa ligand
SKADT

SEQ ID NO: 746 - protein, 5 aa, synthetic construct
  REGION 1..5: note = Synthetic Cry1Aa ligand
SKPAD

SEQ ID NO: 747 - protein, 11 aa, synthetic construct
  REGION 1..11: note = Synthetic Fsr ligand
  VARIANT 3..11: note = Lacton linkage between S3 and A11
QNSAAAFAAWA

SEQ ID NO: 748 - protein, 11 aa, synthetic construct
  REGION 1..11: note = Synthetic Fsr ligand
  VARIANT 3..11: note = Lacton linkage between S3 and A11
QNSAAAFGQWA

SEQ ID NO: 749 - protein, 7 aa, synthetic construct
  REGION 1..7: note = Synthetic AgrC1, AgrC2
  VARIANT 4..7: note = Thiolacton linkage between C4 and M7)
YSTCFIM

SEQ ID NO: 750 - protein, 7 aa, synthetic construct
  REGION 1..7: note = Synthetic AgrC1, AgrC2
  VARIANT 3..7: note = Thiolacton linkage between C3 and M7
STCAFIM

SEQ ID NO: 751 - protein, 64 aa, Bacillus amyloliquefaciens
  source 1..64: note = Amylocyclicin (Alc); note = Strain FZB42
LASTLGISTAAAKKAIDIIDAASTIASIISLIGIVTGAGAISYAIVATAKTMIKKYGKKYAAAW

SEQ ID NO: 752 - protein, 64 aa, Bacillus amyloliquefaciens
  source 1..64: note = Strain FZB42; note = Amylocyclicin (Alc)
STAAAKKAIDIIDAASTIASIISLIGIVTGAGAISYAIVATAKTMIKKYGKKYAAAWLASTLGI

SEQ ID NO: 753 - protein, 64 aa, Staphylococcus aureus
  source 1..64: note = Strain 3688STDY6124959; note = Bacteriocin
LTSTLGISSYAAKKAIDIIAAAGDVAAIVGLIGAVTGAGAIGAGILFTAKKLIKSYGKKYAAAW

SEQ ID NO: 754 - protein, 64 aa, Staphylococcus aureus
  source 1..64: note = Bacteriocin; note = Strain 3688STDY6124959
STLGISSYAAKKAIDIIAAAGDVAAIVGLIGAVTGAGAIGAGILFTAKKLIKSYGKKYAAAWLT

SEQ ID NO: 755 - protein, 70 aa, Enterococcus faecalis
  source 1..70: note = Strain S-48; note = Enterocin AS-48 (EntAS48)
MAKEFGIPAAVAGTVLNVVEAGGWVTTIVSILTAVGSGGLSLLAAAGRESIKAYLKKEIKKKGKRAVIAW

SEQ ID NO: 756 - protein, 70 aa, Enterococcus faecalis
  source 1..70: note = Enterocin AS-48 (EntAS48); note = Strain S-48
SIKAYLKKEIKKKGKRAVIAWMAKEFGIPAAVAGTVLNVVEAGGWVTTIVSILTAVGSGGLSLLAAAGRE

SEQ ID NO: 757 - protein, 70 aa, Listeria monocytogenes
  source 1..70: note = BCW 2997; note = Bacteriocin BCW 2997
LAKEFGIPGGVAATVLNIVEAGGWATTILSILTAVGSGGLSLIAAAGKESIKVYLKKQIKKKGRKAVIAW

SEQ ID NO: 758 - protein, 70 aa, Listeria monocytogenes
  source 1..70: note = Bacteriocin BCW 2997; note = Strain BCW 2997
SIKVYLKKQIKKKGRKAVIAWLAKEFGIPGGVAATVLNIVEAGGWATTILSILTAVGSGGLSLIAAAGKE

SEQ ID NO: 759 - protein, 70 aa, Clavibacter michiganensis
  source 1..70: note = Strain CF11; note = Bacteriocin CF11
LAQQFGIPSAVGGTILNLATAGGTAATIVGILVGLGSGGLGLLAAAGRETIKQFLLNEIKKKGRKAVIAW

SEQ ID NO: 760 - protein, 70 aa, Clavibacter michiganensis
  source 1..70: note = Bacteriocin CF11; note = Strain CF11
SAVGGTILNLATAGGTAATIVGILVGLGSGGLGLLAAAGRETIKQFLLNEIKKKGRKAVIAWLAQQFGIP

SEQ ID NO: 761 - protein, 70 aa, Paenibacillus chondroitinus
  source 1..70: note = Strain NBRC 15376; note = Bacteriocin NBRC 15376
LAKEFGVPAVVGNALVQALDWGLTAATIASIIGAFITGGLSIVAAAGTTALKTYLRNKLAEMGTKAFVAW

SEQ ID NO: 762 - protein, 70 aa, Paenibacillus chondroitinus
  source 1..70: note = Strain NBRC 15376; note = Bacteriocin NBRC 15376
SIIGAFITGGLSIVAAAGTTALKTYLRNKLAEMGTKAFVAWLAKEFGVPAVVGNALVQALDWGLTAATIA

SEQ ID NO: 763 - protein, 60 aa, Carnobacterium maltaromaticum
  source 1..60: note = Strain UAL307; note = Carnocyclin A
LVAYGIAQGTAEKVVSLINAGLTVGSIISILGGVTVGLSGVFTAVKAAIAKQGIKKAIQL

SEQ ID NO: 764 - protein, 60 aa, Carnobacterium maltaromaticum
  source 1..60: note = Strain UAL307; note = Carnocyclin A
SLINAGLTVGSIISILGGVTVGLSGVFTAVKAAIAKQGIKKAIQLLVAYGIAQGTAEKVV

SEQ ID NO: 765 - protein, 69 aa, Clostridium beijerinckii
  source 1..69: note = Circularin A (CirA); note = Strain ATCC 25752
VAGALGVQTAAATTIVNVILNAGTLVTVLGIIASIASGGAGTLMTIGWATFKATVQKLAKQSMARAIAY

SEQ ID NO: 766 - protein, 69 aa, Clostridium beijerinckii
  source 1..69: note = Strain ATCC 25752; note = Circularin A (CirA)
SIASGGAGTLMTIGWATFKATVQKLAKQSMARAIAYVAGALGVQTAAATTIVNVILNAGTLVTVLGIIA

SEQ ID NO: 767 - protein, 69 aa, Streptococcus suis
  source 1..69: note = Bacteriocin YS111; note = Strain YS111
VAAALGVPSAVASTVVNVILGAGTAVTILGIISSIASGGATTLLTVGWAGFKATVQRLAKQSMARAIAY

SEQ ID NO: 768 - protein, 69 aa, Streptococcus suis
  source 1..69: note = Strain YS111; note = Bacteriocin YS111
STVVNVILGAGTAVTILGIISSIASGGATTLLTVGWAGFKATVQRLAKQSMARAIAYVAAALGVPSAVA

SEQ ID NO: 769 - protein, 64 aa, Enterococcus faecium
  source 1..64: note = Strain NKR-5-3; note = Enterocin NKR-5-3B
LTANLGISSYAAKKVIDIINTGSAVATIIALVTAVVGGGLITAGIVATAKSLIKKYGAKYAAAW

SEQ ID NO: 770 - protein, 64 aa, Enterococcus faecium
  source 1..64: note = Enterocin NKR-5-3B; note = Strain NKR-5-3B
SSYAAKKVIDIINTGSAVATIIALVTAVVGGGLITAGIVATAKSLIKKYGAKYAAAWLTANLGI

SEQ ID NO: 771 - protein, 64 aa, Garciella nitratireducens
  source 1..64: note = Strain DSM 15102; note = Bacteriocin DSM 15102
LASMLGISTVAAKKVIDIIDTASTVATIISLIGVIVGVGGITAGLVTTAKAMIKKYGKKYATMW

SEQ ID NO: 772 - protein, 64 aa, Garciella nitratireducens
  source 1..64: note = Strain DSM 15102; note = Bacteriocin DSM 15102
SMLGISTVAAKKVIDIIDTASTVATIISLIGVIVGVGGITAGLVTTAKAMIKKYGKKYATMWLA

SEQ ID NO: 773 - protein, 60 aa, Lactococcus garvieae
  source 1..60: note = Garvicin ML (GarML); note = Strain DCC43
LVATGMAAGVAKTIVNAVSAGMDIATALSLFSGAFTAAGGIMALIKKYAQKKLWKQLIAA

SEQ ID NO: 774 - protein, 60 aa, Lactococcus garvieae
  source 1..60: note = Garvicin ML (GarML); note = Strain DCC43
SGAFTAAGGIMALIKKYAQKKLWKQLIAALVATGMAAGVAKTIVNAVSAGMDIATALSLF

SEQ ID NO: 775 - protein, 61 aa, Leuconostoc mesenteroides
  source 1..61: note = Leucocyclicin Q; note = Strain TK41401
LVNQLGISKSLANTILGAIAVGNLASWLLALVPGPGWATKAALATAETIVKHEGKAAAIAW

SEQ ID NO: 776 - protein, 61 aa, Leuconostoc mesenteroides
  source 1..61: note = Leucocyclicin Q; note = Strain TK41401
SKSLANTILGAIAVGNLASWLLALVPGPGWATKAALATAETIVKHEGKAAAIAWLVNQLGI

SEQ ID NO: 777 - protein, 61 aa, Bacillus toyonensis
  source 1..61: note = Strain AFS089278; note = Bacteriocin AFS089278
LINKLGISKSLAQDILTAIAVGNVASWLLVLIPGPGWATKAAISAAEIIVETSGEAAAVAY

SEQ ID NO: 778 - protein, 61 aa, Bacillus toyonensis
  source 1..61: note = Bacteriocin AFS089278; note = Strain AFS089278
SKSLAQDILTAIAVGNVASWLLVLIPGPGWATKAAISAAEIIVETSGEAAAVAYLINKLGI

SEQ ID NO: 779 - protein, 70 aa, Streptococcus uberis
  source 1..70: note = Strain 42; note = Uberolysin A (UberA)
LAGYTGIASGTAKKVVDAIDKGAAAFVIISIISTVISAGALGAVSASADFIILTVKNYISRNLKAQAVIW

SEQ ID NO: 780 - protein, 70 aa, Streptococcus uberis
  source 1..70: note = Strain 42; note = Uberolysin A (UberA)
SGTAKKVVDAIDKGAAAFVIISIISTVISAGALGAVSASADFIILTVKNYISRNLKAQAVIWLAGYTGIA

SEQ ID NO: 781 - protein, 70 aa, Bacillus vallismortis
  source 1..70: note = Bacteriocin TD3; note = Strain TD3
VAGVLSVDKGVATKVVNAVDAAGWAFVGGSTLLAIISGGTLSAASISIDALIYTIKNYLKRSLKAQAIAW

SEQ ID NO: 782 - protein, 70 aa, Bacillus vallismortis
  source 1..70: note = Strain TD3; note = Bacteriocin TD3
SVDKGVATKVVNAVDAAGWAFVGGSTLLAIISGGTLSAASISIDALIYTIKNYLKRSLKAQAIAWVAGVL

SEQ ID NO: 783 - protein, 66 aa, Streptomyces pathocidini
  source 1..66: note = Strain NRRL B-24287; note = Bacteriocin NRRL B-24287
ATTPNVLTGYFGLTGATANRVLNAIEAGTDIAAALSVLGGVSAAGGVAMWMLKQAIAKGGRKAVVA

SEQ ID NO: 784 - protein, 66 aa, Streptomyces pathocidini
  source 1..66: note = Bacteriocin NRRL B-24287; note = Strain NRRL B-24287
SVLGGVSAAGGVAMWMLKQAIAKGGRKAVVAATTPNVLTGYFGLTGATANRVLNAIEAGTDIAAAL

SEQ ID NO: 785 - protein, 58 aa, Alkalibacterium sp.
  source 1..58: note = Strain AK22; note = Bacteriocin AK22
ILFVADLFGIPFTTDIVDQVWGYIEGGAGTVTAISWVLGVTIPAWAVPIIGAVGVVSA

SEQ ID NO: 786 - protein, 58 aa, Alkalibacterium sp.
  source 1..58: note = Strain AK22; note = Bacteriocin AK22
SWVLGVTIPAWAVPIIGAVGVVSAILFVADLFGIPFTTDIVDQVWGYIEGGAGTVTAI

SEQ ID NO: 787 - protein, 58 aa, Gemella cuniculi
  source 1..58: note = Strain DSM 15828; note = Bacteriocin 15828
VYFIAGKLGITLAPGWYEKLVNYISAGGGAVDGVAMILGITLPGWAITVIGAFGLVSA

SEQ ID NO: 788 - protein, 58 aa, Gemella cuniculi
  source 1..58: note = Bacteriocin 15828; note = Strain DSM 15828
SAGGGAVDGVAMILGITLPGWAITVIGAFGLVSAVYFIAGKLGITLAPGWYEKLVNYI

SEQ ID NO: 789 - protein, 58 aa, Butyrivibrio fibrisolvens
  source 1..58: note = Butyrivibriocin AR10; note = Strain AR10
IYFIADKMGIQLAPAWYQDIVNWVSAGGTLTTGFAIIVGVTVPAWIAEAAAAFGIASA

SEQ ID NO: 790 - protein, 58 aa, Butyrivibrio fibrisolvens
  source 1..58: note = Strain AR10; note = Butyrivibriocin AR10
SAGGTLTTGFAIIVGVTVPAWIAEAAAAFGIASAIYFIADKMGIQLAPAWYQDIVNWV

SEQ ID NO: 791 - protein, 58 aa, Lactobacillus. paracesei
  source 1..58: note = Strain JCM 8130/ DSM 5622; note = Paracyclicin P (ParP)
IYFIANKLGIHLAPGWYQDMVNYVSAGGSLAGAFSVVAGVTLPAWIVPIATAFGAVSA

SEQ ID NO: 792 - protein, 58 aa, Lactobacillus. paracesei
  source 1..58: note = Paracyclicin P (ParP); note = Strain JCM 8130/ DSM 5622
SAGGSLAGAFSVVAGVTLPAWIVPIATAFGAVSAIYFIANKLGIHLAPGWYQDMVNYV

SEQ ID NO: 793 - protein, 58 aa, Streptococcus thermophilus
  source 1..58: note = Strain NCTC 12958; note = Bacteriocin NCTC 12958
IYWICGKFGIRLAPGWYQDIVDFVSAGGSIVDAFAIIAGITLPAWVAPVLAGFGVVSA

SEQ ID NO: 794 - protein, 58 aa, Streptococcus thermophilus
  source 1..58: note = Bacteriocin NCTC 12958; note = Strain NCTC 12958
SAGGSIVDAFAIIAGITLPAWVAPVLAGFGVVSAIYWICGKFGIRLAPGWYQDIVDFV

SEQ ID NO: 795 - protein, 58 aa, Streptococcus pneumoniae
  source 1..58: note = Strain UoS2029; note = Bacteriocin UoS2029
IYWVCGKFGIELAPGWYQDIVDFVSSGGTIVEAFAAVAGVTLPAWVGPVLAAFGLASA

SEQ ID NO: 796 - protein, 58 aa, Streptococcus pneumoniae
  source 1..58: note = Bacteriocin UoS2029; note = Strain UoS2029
SSGGTIVEAFAAVAGVTLPAWVGPVLAAFGLASAIYWVCGKFGIELAPGWYQDIVDFV

SEQ ID NO: 797 - protein, 58 aa, Staphylococcus felis
  source 1..58: note = Bacteriocin F9_V1*; note = Strain F9
VTWVLDKFGIKIGTPTINEIINYVANGGAIVTAFAVIAGITLPAWVGPAVAAFATTAA

SEQ ID NO: 798 - protein, 59 aa, Staphylococcus felis
  source 1..59: note = Bacteriocin F9_V1*; note = Strain F9
SVTWVLDKFGIKIGTPTINEIINYVANGGAIVTAFAVIAGITLPAWVGPAVAAFATTAA

SEQ ID NO: 799 - protein, 58 aa, Staphylococcus felis; identical to SEQ ID NO: 797
  source 1..58: note = Bacteriocin F9_V2*; note = Strain F9

SEQ ID NO: 800 - protein, 58 aa, Staphylococcus felis
  source 1..58: note = Bacteriocin F9_V2*; note = Strain F9
SAVTWVLDKFGIKIGTPTINEIINYVANGGAIVTAFAVIAGITLPAWVGPAVAAFATT

SEQ ID NO: 801 - protein, 58 aa, Lactobacillus gasseri
  source 1..58: note = Gassericin A (GasA); note = Strain LA39
IYWIADQFGIHLATGTARKLLDAMASGASLGTAFAAILGVTLPAWALAAAGALGATAA

SEQ ID NO: 802 - protein, 58 aa, Lactobacillus gasseri
  source 1..58: note = Strain LA39; note = Gassericin A (GasA)
SGASLGTAFAAILGVTLPAWALAAAGALGATAAIYWIADQFGIHLATGTARKLLDAMA

SEQ ID NO: 803 - protein, 58 aa, Lactobacillus plantarum
  source 1..58: note = Plantaricyclin A (PlcA); note = Strain NI326
IVWIAKQFGVHLTTSLTQKALDLLSAGSSLGTVAAAVLGVTLPAWAVAAAGALGGTAA

SEQ ID NO: 804 - protein, 58 aa, Lactobacillus plantarum
  source 1..58: note = Plantaricyclin A (PlcA); note = Strain NI326
SLTQKALDLLSAGSSLGTVAAAVLGVTLPAWAVAAAGALGGTAAIVWIAKQFGVHLTT

SEQ ID NO: 805 - protein, 70 aa, Bacillus sp
  source 1..70: note = Strain Xin1; note = Cerecyclin
VVSKLGWTGINIGTANALIGALMTGSDIWTAISVAGLAFGGGIGTAISTIGRKAIMEMVEKVGKKKAAQW

SEQ ID NO: 806 - protein, 70 aa, Bacillus sp
  source 1..70: note = Cerecyclin; note = Strain Xin1
SKLGWTGINIGTANALIGALMTGSDIWTAISVAGLAFGGGIGTAISTIGRKAIMEMVEKVGKKKAAQWVV

SEQ ID NO: 807 - protein, 58 aa, Paeniclostridium sordellii
  source 1..58: note = Strain R26833; note = Bacteriocin R26833
IYFIADKMGITLSTKMYRDIVDAVSAGGSIAGAFSLIVGVTLPGWLVVAAAGFGLTSA

SEQ ID NO: 808 - protein, 58 aa, Paeniclostridium sordellii
  source 1..58: note = Bacteriocin R26833; note = Strain R26833
STKMYRDIVDAVSAGGSIAGAFSLIVGVTLPGWLVVAAAGFGLTSAIYFIADKMGITL

SEQ ID NO: 809 - protein, 36 aa, Nostoc punctifrome
  source 1..36: note = C-terminal intein fragment from Nostoc punctifrome (Npu) DnaE split intein; note = Fragment Ic
MIKIATRKYLGKQNVYDIGVERYHNFALKNGFIASN

SEQ ID NO: 810 - protein, 104 aa, Nostoc punctifrome
  source 1..104: note = Fragment IN; note = N-terminal intein fragment from Nostoc punctifrome (Npu) DnaE split intein
CLSYDTEILTVEYGILPIGKIVEKRIECTVYSVDNNGNIYTQPVAQWHDRGEQEVFEYCLEDGCLIRATKDHKFMTVDGQMMPIDEIFERELDLMRVDNLPNGT

SEQ ID NO: 811 - protein, 64 aa, Bacillus amyloliquefaciens; identical to SEQ ID NO: 751
  source 1..64: note = Strain FZB42; note = Amylocyclicin (Alc)

SEQ ID NO: 812 - protein, 64 aa, Bacillus amyloliquefaciens; identical to SEQ ID NO: 752
  source 1..64: note = Strain FZB42; note = Amylocyclicin (Alc)

SEQ ID NO: 813 - protein, 64 aa, Staphylococcus aureus; identical to SEQ ID NO: 753
  source 1..64: note = Bacteriocin 3688STDY6124959; note = Strain 3688STDY6124959

SEQ ID NO: 814 - protein, 64 aa, Staphylococcus aureus; identical to SEQ ID NO: 754
  source 1..64: note = Bacteriocin 3688STDY6124959; note = Strain 3688STDY6124959

SEQ ID NO: 815 - protein, 70 aa, Enterococcus faecalis; identical to SEQ ID NO: 755
  source 1..70: note = Strain 48; note = Enterocin AS-48 (EntAS48)

SEQ ID NO: 816 - protein, 70 aa, Enterococcus faecalis; identical to SEQ ID NO: 756
  source 1..70: note = Enterocin AS-48 (EntAS48); note = Strain 48

SEQ ID NO: 817 - protein, 70 aa, Listeria monocytogenes; identical to SEQ ID NO: 757
  source 1..70: note = Strain BCW 2997; note = Bacteriocin BCW 2997

SEQ ID NO: 818 - protein, 70 aa, Listeria monocytogenes; identical to SEQ ID NO: 758
  source 1..70: note = Strain BCW 2997; note = Bacteriocin BCW 2997

SEQ ID NO: 819 - protein, 70 aa, Clavibacter michiganensis; identical to SEQ ID NO: 759
  source 1..70: note = Strain CF11; note = Bacteriocin CF11

SEQ ID NO: 820 - protein, 70 aa, Clavibacter michiganensis; identical to SEQ ID NO: 760
  source 1..70: note = Strain CF11; note = Bacteriocin CF11

SEQ ID NO: 821 - protein, 70 aa, Paenibacillus chondroitinus; identical to SEQ ID NO: 761
  source 1..70: note = Strain NBRC 15376; note = Bacteriocin NBRC 15376

SEQ ID NO: 822 - protein, 70 aa, Paenibacillus chondroitinus; identical to SEQ ID NO: 762
  source 1..70: note = Bacteriocin NBRC 15376; note = Strain NBRC 15376

SEQ ID NO: 823 - protein, 60 aa, Carnobacterium maltaromaticum; identical to SEQ ID NO: 763
  source 1..60: note = Carnocyclin A; note = Strain UAL307

SEQ ID NO: 824 - protein, 60 aa, Carnobacterium maltaromaticum; identical to SEQ ID NO: 764
  source 1..60: note = Carnocyclin A; note = Strain UAL307

SEQ ID NO: 825 - protein, 69 aa, Clostridium beijerinckii; identical to SEQ ID NO: 765
  source 1..69: note = Circularin A (CirA); note = Strain ATCC 25752

SEQ ID NO: 826 - protein, 69 aa, Clostridium beijerinckii; identical to SEQ ID NO: 766
  source 1..69: note = Circularin A (CirA); note = ATCC 25752

SEQ ID NO: 827 - protein, 69 aa, Streptococcus suis; identical to SEQ ID NO: 767
  source 1..69: note = Bacteriocin YS111; note = Strain YS111

SEQ ID NO: 828 - protein, 69 aa, Streptococcus suis; identical to SEQ ID NO: 768
  source 1..69: note = Strain YS111; note = Bacteriocin YS111

SEQ ID NO: 829 - protein, 64 aa, Enterococcus faecium; identical to SEQ ID NO: 769
  source 1..64: note = Enterocin NKR-5-3B; note = Strain NKR-5-3

SEQ ID NO: 830 - protein, 64 aa, Enterococcus faecium; identical to SEQ ID NO: 770
  source 1..64: note = Strain NKR-5-3; note = Enterocin NKR-5-3B

SEQ ID NO: 831 - protein, 64 aa, Garciella nitratireducens; identical to SEQ ID NO: 771
  source 1..64: note = Strain DSM 15102; note = Bacteriocin DSM 15102

SEQ ID NO: 832 - protein, 64 aa, Garciella nitratireducens; identical to SEQ ID NO: 772
  source 1..64: note = Bacteriocin DSM 15102; note = Strain DSM 15102

SEQ ID NO: 833 - protein, 60 aa, Lactococcus garvieae; identical to SEQ ID NO: 773
  source 1..60: note = Garvicin ML (GarML); note = Strain DCC43

SEQ ID NO: 834 - protein, 60 aa, Lactococcus garvieae; identical to SEQ ID NO: 774
  source 1..60: note = Garvicin ML (GarML); note = Strain DCC43

SEQ ID NO: 835 - protein, 61 aa, Leuconostoc mesenteroides; identical to SEQ ID NO: 775
  source 1..61: note = Leucocyclicin Q; note = Strain TK41401

SEQ ID NO: 836 - protein, 61 aa, Leuconostoc mesenteroides; identical to SEQ ID NO: 776
  source 1..61: note = Strain TK41401; note = Leucocyclicin Q

SEQ ID NO: 837 - protein, 61 aa, Bacillus toyonensis; identical to SEQ ID NO: 777
  source 1..61: note = Bacteriocin AFS089278; note = Strain AFS089278

SEQ ID NO: 838 - protein, 61 aa, Bacillus toyonensis; identical to SEQ ID NO: 778
  source 1..61: note = Strain AFS089278; note = Bacteriocin AFS089278

SEQ ID NO: 839 - protein, 70 aa, Streptococcus uberis; identical to SEQ ID NO: 779
  source 1..70: note = Uberolysin A (UberA); note = Strain 42

SEQ ID NO: 840 - protein, 70 aa, Streptococcus uberis; identical to SEQ ID NO: 780
  source 1..70: note = Uberolysin A (UberA); note = Strain 42

SEQ ID NO: 841 - protein, 70 aa, Bacillus vallismortis; identical to SEQ ID NO: 781
  source 1..70: note = Bacteriocin TD3; note = Strain TD3

SEQ ID NO: 842 - protein, 70 aa, Bacillus vallismortis; identical to SEQ ID NO: 782
  source 1..70: note = Strain TD3; note = Bacteriocin TD3

SEQ ID NO: 843 - protein, 66 aa, Streptomyces pathocidini; identical to SEQ ID NO: 783
  source 1..66: note = Strain NRRL B-24287; note = Bacteriocin NRRL B-24287

SEQ ID NO: 844 - protein, 66 aa, Streptomyces pathocidini; identical to SEQ ID NO: 784
  source 1..66: note = Strain NRRL B-24287; note = Bacteriocin NRRL B-24287

SEQ ID NO: 845 - protein, 58 aa, Alkalibacterium; identical to SEQ ID NO: 785
  source 1..58: note = Strain AK22; note = Bacteriocin AK22

SEQ ID NO: 846 - protein, 58 aa, Alkalibacterium; identical to SEQ ID NO: 786
  source 1..58: note = Bacteriocin AK22; note = Strain AK22

SEQ ID NO: 847 - protein, 58 aa, Gemella cuniculi; identical to SEQ ID NO: 787
  source 1..58: note = Strain 15828; note = Bacteriocin 15828

SEQ ID NO: 848 - protein, 58 aa, Gemella cuniculi; identical to SEQ ID NO: 788
  source 1..58: note = Bacteriocin 15828; note = Strain 15828

SEQ ID NO: 849 - protein, 58 aa, Butyrivibrio fibrisolvens; identical to SEQ ID NO: 789
  source 1..58: note = Butyrivibriocin AR10; note = Strain AR10

SEQ ID NO: 850 - protein, 58 aa, Butyrivibrio fibrisolvens; identical to SEQ ID NO: 790
  source 1..58: note = Strain AR10; note = Butyrivibriocin AR10

SEQ ID NO: 851 - protein, 58 aa, Lactobacillus paracesei; identical to SEQ ID NO: 791
  source 1..58: note = Paracyclicin P (ParP); note = subsp. Paracasei JCM 8130/ DSM 5622

SEQ ID NO: 852 - protein, 58 aa, Lactobacillus paracesei; identical to SEQ ID NO: 792
  source 1..58: note = Paracyclicin P (ParP); note = subsp. Paracasei JCM 8130/ DSM 5622

SEQ ID NO: 853 - protein, 58 aa, Streptococcus thermophilus; identical to SEQ ID NO: 793
  source 1..58: note = Bacteriocin NCTC 12958; note = Strain NCTC 12958

SEQ ID NO: 854 - protein, 58 aa, Streptococcus thermophilus; identical to SEQ ID NO: 794
  source 1..58: note = Bacteriocin NCTC 12958; note = Strain NCTC 12958

SEQ ID NO: 855 - protein, 58 aa, Streptococcus pneumoniae; identical to SEQ ID NO: 795
  source 1..58: note = Bacteriocin UoS2029; note = Strain UoS2029

SEQ ID NO: 856 - protein, 58 aa, Streptococcus pneumoniae; identical to SEQ ID NO: 796
  source 1..58: note = Bacteriocin UoS2029; note = Strain UoS2029

SEQ ID NO: 857 - protein, 58 aa, Staphylococcus felis; identical to SEQ ID NO: 797
  source 1..58: note = Bacteriocin F9_V1*; note = Strain F9

SEQ ID NO: 858 - protein, 59 aa, Staphylococcus felis; identical to SEQ ID NO: 798
  source 1..59: note = Bacteriocin F9_V1*; note = Strain F9

SEQ ID NO: 859 - protein, 58 aa, Staphylococcus felis; identical to SEQ ID NO: 797
  source 1..58: note = Bacteriocin F9_V2*; note = Strain F9

SEQ ID NO: 860 - protein, 58 aa, Staphylococcus felis; identical to SEQ ID NO: 800
  source 1..58: note = Strain F9; note = Bacteriocin F9_V2*

SEQ ID NO: 861 - protein, 58 aa, Lactobacillus gasseri; identical to SEQ ID NO: 801
  source 1..58: note = Strain LA39; note = Gassericin A (GasA)

SEQ ID NO: 862 - protein, 58 aa, Lactobacillus gasseri; identical to SEQ ID NO: 802
  source 1..58: note = Strain LA39; note = Gassericin A (GasA)

SEQ ID NO: 863 - protein, 58 aa, Lactobacillus plantarum; identical to SEQ ID NO: 803
  source 1..58: note = Plantaricyclin A (PlcA); note = Strain NI326

SEQ ID NO: 864 - protein, 58 aa, Lactobacillus plantarum; identical to SEQ ID NO: 804
  source 1..58: note = Plantaricyclin A (PlcA); note = Strain NI326

SEQ ID NO: 865 - protein, 70 aa, Bacillus sp; identical to SEQ ID NO: 805
  source 1..70: note = Strain Xin1; note = Cerecyclin

SEQ ID NO: 866 - protein, 70 aa, Bacillus sp; identical to SEQ ID NO: 806
  source 1..70: note = Strain Xin1; note = Cerecyclin

SEQ ID NO: 867 - protein, 58 aa, Paeniclostridium sordellii; identical to SEQ ID NO: 807
  source 1..58: note = Strain R26833; note = Bacteriocin R26833

SEQ ID NO: 868 - protein, 58 aa, Paeniclostridium sordellii; identical to SEQ ID NO: 808
  source 1..58: note = Bacteriocin R26833; note = Strain R26833

SEQ ID NO: 869 - protein, 36 aa, Nostoc punctifrome; identical to SEQ ID NO: 809
  source 1..36: note = C-terminal intein fragment from Nostoc punctifrome (Npu) DnaE split intein; note = Fragment IC

SEQ ID NO: 870 - protein, 104 aa, Nostoc punctifrome; identical to SEQ ID NO: 810
  source 1..104: note = IN; note = N-terminal intein fragment from Nostoc punctifrome (Npu) DnaE split intein

SEQ ID NO: 871 - protein, 36 aa, Nostoc punctifrome
  source 1..36: note = Fragment IC (N36D); note = C-terminal intein fragment from Nostoc punctifrome (Npu) DnaE split intein with a point substitution of essential residue D36 by an A
MIKIATRKYLGKQNVYDIGVERYHNFALKNGFIASD

SEQ ID NO: 872 - protein, 104 aa, Nostoc punctifrome
  source 1..104: note = N-terminal intein fragment from Nostoc punctifrome (Npu) DnaE split intein with a point substitution of essential residue C1 by an A; note = IN (C1A)
ALSYDTEILTVEYGILPIGKIVEKRIECTVYSVDNNGNIYTQPVAQWHDRGEQEVFEYCLEDGCLIRATKDHKFMTVDGQMMPIDEIFERELDLMRVDNLPNGT

SEQ ID NO: 873 - protein, 11 aa, synthetic construct
  source 1..11: note = Protein degradation tag; note = Fragment SsrA
AANDENYALAA

SEQ ID NO: 874 - protein, 211 aa, synthetic construct
MIKIATRKYLGKQNVYDIGVERYHNFALKNGFIASNSGAFTAAGGIMALIKKYAQKKLWKQLIAALVATGMAAGVAKTIVNAVSAGMDIATALSLFCLSYDTEILTVEYGILPIGKIVEKRIECTVYSVDNNGNIYTQPVAQWHDRGEQEVFEYCLEDGCLIRATKDHKFMTVDGQMMPIDEIFERELDLMRVDNLPNGTAANDENYALAA